SEQUENCE LISTING (40 sequences)

SEQ ID NO: 1 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-methyl nucleotide
  modified_base (4)..(4): 2′-O-methyl nucleotide
  modified_base (7)..(8): 2′-O-methyl nucleotide
  modified_base (14)..(14): 2′-O-methyl nucleotide
  modified_base (16)..(16): 2′-O-methyl nucleotide
  modified_base (20)..(21): Unlocked nucleobase analogue
agguauguugcccguuuguuu

SEQ ID NO: 2 - RNA, 21 nt, Artificial Sequence
  Description of Artificial Sequence Synthetic oligonucleotide
  modified_base (7)..(7): 2′-O-methyl nucleotide
  modified_base (15)..(15): 2′-O-methyl nucleotide
  modified_base (20)..(21): Unlocked nucleobase analogue
acaaacgggcaacauaccuuu

SEQ ID NO: 3 - RNA, 21 nt, Artificial Sequence
  Description of Artificial Sequence Synthetic oligonucleotide
  modified_base (1)..(1): Unlocked nucleobase analogue
  modified_base (3)..(4): 2′-O-methyl nucleotide
  modified_base (6)..(6): 2′-O-methyl nucleotide
  modified_base (18)..(18): 2′-O-methyl nucleotide
  modified_base (21)..(21): Unlocked nucleobase analogue
gcucaguuuacuagugccauu

SEQ ID NO: 4 - RNA, 21 nt, Artificial Sequence
  Description of Artificial Sequence Synthetic oligonucleotide
  modified_base (10)..(10): 2′-O-methyl nucleotide
  modified_base (18)..(18): 2′-O-methyl nucleotide
uggcacuaguaaacugagcuu

SEQ ID NO: 5 - RNA, 21 nt, Artificial Sequence
  Description of Artificial Sequence Synthetic oligonucleotide
  modified_base (1)..(1): Unlocked nucleobase analogue
  modified_base (5)..(6): 2′-O-methyl nucleotide
  modified_base (12)..(12): 2′-O-methyl nucleotide
  modified_base (16)..(17): 2′-O-methyl nucleotide
  modified_base (20)..(21): Unlocked nucleobase analogue
ccgugugcacuucgcuucauu

SEQ ID NO: 6 - RNA, 21 nt, Artificial Sequence
  Description of Artificial Sequence Synthetic oligonucleotide
  modified_base (12)..(12): 2′-O-methyl nucleotide
  modified_base (18)..(18): 2′-O-methyl nucleotide
  modified_base (20)..(21): Unlocked nucleobase analogue
ugaagcgaagugcacacgguu

SEQ ID NO: 7 - RNA, 21 nt, Artificial Sequence
  Description of Artificial Sequence Synthetic oligonucleotide
  modified_base (1)..(1): Unlocked nucleobase analogue
  modified_base (3)..(4): 2′-O-methyl nucleotide
  modified_base (6)..(6): 2′-O-methyl nucleotide
  modified_base (18)..(18): 2′-O-methyl nucleotide
  modified_base (20)..(21): Unlocked nucleobase analogue
gcucaguuuacuagugccauu

SEQ ID NO: 8 - RNA, 21 nt, Artificial Sequence
  Description of Artificial Sequence Synthetic oligonucleotide
  modified_base (10)..(10): 2′-O-methyl nucleotide
  modified_base (18)..(18): 2′-O-methyl nucleotide
  modified_base (20)..(21): Unlocked nucleobase analogue
uggcacuaguaaacugagcuu

SEQ ID NO: 9 - RNA, 21 nt, Artificial Sequence
  Description of Artificial Sequence Synthetic oligonucleotide
  modified_base (1)..(1): Unlocked nucleobase analogue
  modified_base (5)..(6): 2′-O-methyl nucleotide
  modified_base (12)..(12): 2′-O-methyl nucleotide
  modified_base (16)..(16): 2′-O-methyl nucleotide
  modified_base (21)..(21): Unlocked nucleobase analogue
ccgugugcacuucgcuucauu

SEQ ID NO: 10 - RNA, 21 nt, Artificial Sequence
  Description of Artificial Sequence Synthetic oligonucleotide
  modified_base (12)..(12): 2′-O-methyl nucleotide
  modified_base (18)..(18): 2′-O-methyl nucleotide
ugaagcgaagugcacacgguu

SEQ ID NO: 11 - RNA, 21 nt, Artificial Sequence
  Description of Artificial Sequence Synthetic oligonucleotide
  modified_base (1)..(1): Unlocked nucleobase analogue
  modified_base (2)..(4): 2′-O-methyl nucleotide
  modified_base (15)..(15): 2′-O-methyl nucleotide
  modified_base (17)..(17): 2′-O-methyl nucleotide
  modified_base (21)..(21): Unlocked nucleobase analogue
cuggcucaguuuacuaguguu

SEQ ID NO: 12 - RNA, 21 nt, Artificial Sequence
  Description of Artificial Sequence Synthetic oligonucleotide
  modified_base (6)..(6): 2′-O-methyl nucleotide
  modified_base (13)..(13): 2′-O-methyl nucleotide
  modified_base (15)..(15): 2′-O-methyl nucleotide
cacuaguaaacugagccaguu

SEQ ID NO: 13 - RNA, 21 nt, Artificial Sequence
  Description of Artificial Sequence Synthetic oligonucleotide
  modified_base (1)..(1): Unlocked nucleobase analogue
  modified_base (5)..(6): 2′-O-methyl nucleotide
  modified_base (12)..(12): 2′-O-methyl nucleotide
  modified_base (16)..(16): 2′-O-methyl nucleotide
  modified_base (20)..(21): Unlocked nucleobase analogue
ccgugugcacuucgcuucauu

SEQ ID NO: 14 - RNA, 21 nt, Artificial Sequence
  Description of Artificial Sequence Synthetic oligonucleotide
  modified_base (12)..(12): 2′-O-methyl nucleotide
  modified_base (18)..(18): 2′-O-methyl nucleotide
  modified_base (20)..(21): Unlocked nucleobase analogue
ugaagcgaagugcacacgguu

SEQ ID NO: 15 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-methyl nucleotide
  modified_base (6)..(6): 2′-O-methyl nucleotide
  modified_base (12)..(12): 2′-O-methyl nucleotide
  modified_base (14)..(14): 2′-O-methyl nucleotide
  modified_base (20)..(21): Unlocked nucleobase analogue
gcucaguuuacuagugccauu

SEQ ID NO: 16 - RNA, 21 nt, Artificial Sequence
  Description of Artificial Sequence Synthetic oligonucleotide
  modified_base (15)..(15): 2′-O-methyl nucleotide
  modified_base (20)..(21): Unlocked nucleobase analogue
uggcacuaguaaacugagcuu

SEQ ID NO: 17 - RNA, 21 nt, Artificial Sequence
  Description of Artificial Sequence Synthetic oligonucleotide
  modified_base (1)..(1): Unlocked nucleobase analogue
  modified_base (2)..(2): 2′-O-methyl nucleotide
  modified_base (4)..(4): 2′-O-methyl nucleotide
  modified_base (8)..(8): 2′-O-methyl nucleotide
  modified_base (14)..(14): 2′-O-methyl nucleotide
  modified_base (16)..(16): 2′-O-methyl nucleotide
  modified_base (20)..(21): Unlocked nucleobase analogue
agguauguugcccguuuguuu

SEQ ID NO: 18 - RNA, 21 nt, Artificial Sequence
  Description of Artificial Sequence Synthetic oligonucleotide
  modified_base (7)..(7): 2′-O-methyl nucleotide
  modified_base (15)..(15): 2′-O-methyl nucleotide
  modified_base (19)..(19): 2′-O-methyl nucleotide
  modified_base (20)..(21): Unlocked nucleobase analogue
acaaacgggcaacauaccuuu

SEQ ID NO: 19 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-methyl nucleotide
  modified_base (6)..(6): 2′-O-methyl nucleotide
  modified_base (13)..(14): 2′-O-methyl nucleotide
  modified_base (16)..(17): 2′-O-methyl nucleotide
  modified_base (20)..(21): Unlocked nucleobase analogue
gccgauccauacugcggaauu

SEQ ID NO: 20 - RNA, 21 nt, Artificial Sequence
  Description of Artificial Sequence Synthetic oligonucleotide
  modified_base (8)..(8): 2′-O-methyl nucleotide
  modified_base (13)..(13): 2′-O-methyl nucleotide
  modified_base (18)..(18): 2′-O-methyl nucleotide
  modified_base (20)..(21): Unlocked nucleobase analogue
uuccgcaguauggaucggcuu

SEQ ID NO: 21 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-methyl nucleotide
  modified_base (6)..(6): 2′-O-methyl nucleotide
  modified_base (13)..(14): 2′-O-methyl nucleotide
  modified_base (16)..(17): 2′-O-methyl nucleotide
  modified_base (21)..(21): Unlocked nucleobase analogue
gccgauccauacugcggaauu

SEQ ID NO: 22 - RNA, 21 nt, Artificial Sequence
  Description of Artificial Sequence Synthetic oligonucleotide
  modified_base (8)..(8): 2′-O-methyl nucleotide
  modified_base (13)..(13): 2′-O-methyl nucleotide
  modified_base (18)..(18): 2′-O-methyl nucleotide
uuccgcaguauggaucggcuu

SEQ ID NO: 23 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-methyl nucleotide
  modified_base (6)..(6): 2′-O-methyl nucleotide
  modified_base (13)..(14): 2′-O-methyl nucleotide
  modified_base (17)..(17): 2′-O-methyl nucleotide
  modified_base (21)..(21): Unlocked nucleobase analogue
gccgauccauacugcggaauu

SEQ ID NO: 24 - RNA, 21 nt, Artificial Sequence
  Description of Artificial Sequence Synthetic oligonucleotide
  modified_base (8)..(8): 2′-O-methyl nucleotide
  modified_base (13)..(13): 2′-O-methyl nucleotide
  modified_base (18)..(18): 2′-O-methyl nucleotide
uuccgcaguauggaucggcuu

SEQ ID NO: 25 - RNA, 21 nt, Artificial Sequence
  Description of Artificial Sequence Synthetic oligonucleotide
  modified_base (1)..(1): Unlocked nucleobase analogue
  modified_base (4)..(4): 2′-O-methyl nucleotide
  modified_base (6)..(6): 2′-O-methyl nucleotide
  modified_base (13)..(14): 2′-O-methyl nucleotide
  modified_base (17)..(17): 2′-O-methyl nucleotide
  modified_base (20)..(21): Unlocked nucleobase analogue
gccgauccauacugcggaauu

SEQ ID NO: 26 - RNA, 21 nt, Artificial Sequence
  Description of Artificial Sequence Synthetic oligonucleotide
  modified_base (8)..(8): 2′-O-methyl nucleotide
  modified_base (13)..(13): 2′-O-methyl nucleotide
  modified_base (18)..(18): 2′-O-methyl nucleotide
  modified_base (20)..(21): Unlocked nucleobase analogue
uuccgcaguauggaucggcuu

SEQ ID NO: 27 - RNA, 21 nt, Artificial Sequence
  Description of Artificial Sequence Synthetic oligonucleotide
  modified_base (1)..(1): Unlocked nucleobase analogue
  modified_base (3)..(3): 2′-O-methyl nucleotide
  modified_base (6)..(6): 2′-O-methyl nucleotide
  modified_base (12)..(12): 2′-O-methyl nucleotide
  modified_base (14)..(14): 2′-O-methyl nucleotide
  modified_base (21)..(21): Unlocked nucleobase analogue
gcucaguuuacuagugccauu

SEQ ID NO: 28 - RNA, 21 nt, Artificial Sequence
  Description of Artificial Sequence Synthetic oligonucleotide
  modified_base (15)..(15): 2′-O-methyl nucleotide
uggcacuaguaaacugagcuu

SEQ ID NO: 29 - RNA, 21 nt, Artificial Sequence
  Description of Artificial Sequence Synthetic oligonucleotide
  modified_base (1)..(1): Unlocked nucleobase analogue
  modified_base (2)..(3): 2′-O-methyl nucleotide
  modified_base (6)..(6): 2′-O-methyl nucleotide
  modified_base (12)..(12): 2′-O-methyl nucleotide
  modified_base (20)..(21): Unlocked nucleobase analogue
cuggcucaguuuacuaguguu

SEQ ID NO: 30 - RNA, 21 nt, Artificial Sequence
  Description of Artificial Sequence Synthetic oligonucleotide
  modified_base (15)..(15): 2′-O-methyl nucleotide
  modified_base (20)..(21): Unlocked nucleobase analogue
cacuaguaaacugagccaguu

SEQ ID NO: 31 - RNA, 21 nt, Artificial Sequence
  Description of Artificial Sequence Synthetic oligonucleotide
  modified_base (3)..(3): 2′-O-methyl nucleotide
  modified_base (5)..(6): 2′-O-methyl nucleotide
  modified_base (12)..(12): 2′-O-methyl nucleotide
  modified_base (14)..(14): 2′-O-methyl nucleotide
  modified_base (16)..(17): 2′-O-methyl nucleotide
ccgugugcacuucgcuucauu

SEQ ID NO: 32 - RNA, 21 nt, Artificial Sequence
  Description of Artificial Sequence Synthetic oligonucleotide
  modified_base (5)..(5): 2′-O-methyl nucleotide
  modified_base (7)..(7): 2′-O-methyl nucleotide
  modified_base (11)..(12): 2′-O-methyl nucleotide
  modified_base (15)..(15): 2′-O-methyl nucleotide
  modified_base (17)..(18): 2′-O-methyl nucleotide
ugaagcgaagugcacacgguu

SEQ ID NO: 33 - RNA, 21 nt, Artificial Sequence
  Description of Artificial Sequence Synthetic oligonucleotide
  modified_base (2)..(4): 2′-O-methyl nucleotide
  modified_base (6)..(6): 2′-O-methyl nucleotide
  modified_base (10)..(10): 2′-O-methyl nucleotide
  modified_base (12)..(12): 2′-O-methyl nucleotide
  modified_base (14)..(15): 2′-O-methyl nucleotide
  modified_base (17)..(19): 2′-O-methyl nucleotide
cuggcucaguuuacuaguguu

SEQ ID NO: 34 - RNA, 21 nt, Artificial Sequence
  Description of Artificial Sequence Synthetic oligonucleotide
  modified_base (6)..(7): 2′-O-methyl nucleotide
  modified_base (11)..(11): 2′-O-methyl nucleotide
  modified_base (13)..(13): 2′-O-methyl nucleotide
  modified_base (15)..(15): 2′-O-methyl nucleotide
  modified_base (17)..(17): 2′-O-methyl nucleotide
cacuaguaaacugagccaguu

SEQ ID NO: 35 - RNA, 21 nt, Artificial Sequence
  Description of Artificial Sequence Synthetic oligonucleotide
  modified_base (2)..(2): 2′-O-methyl nucleotide
  modified_base (4)..(4): 2′-O-methyl nucleotide
  modified_base (6)..(6): 2′-O-methyl nucleotide
  modified_base (8)..(8): 2′-O-methyl nucleotide
  modified_base (10)..(10): 2′-O-methyl nucleotide
  modified_base (12)..(12): 2′-O-methyl nucleotide
  modified_base (14)..(14): 2′-O-methyl nucleotide
accucugccuaaucaucucuu

SEQ ID NO: 36 - RNA, 21 nt, Artificial Sequence
  Description of Artificial Sequence Synthetic oligonucleotide
  modified_base (7)..(7): 2′-O-methyl nucleotide
  modified_base (9)..(9): 2′-O-methyl nucleotide
  modified_base (13)..(13): 2′-O-methyl nucleotide
  modified_base (15)..(15): 2′-O-methyl nucleotide
  modified_base (17)..(17): 2′-O-methyl nucleotide
gagaugauuaggcagagguuu

SEQ ID NO: 37 - DNA, 21 bp, Artificial Sequence
  Description of Artificial Sequence Synthetic primer
gacaaacgggcaacatacctt

SEQ ID NO: 38 - DNA, 20 bp, Artificial Sequence
  Description of Artificial Sequence Synthetic primer
gtgtctgcggcgttttatca

SEQ ID NO: 39 - DNA, 28 bp, Artificial Sequence
  Description of Artificial Sequence Synthetic probe
  5′ FAM
  3′ TAMRA
cctctkcatcctgctgctatgcctcatc

SEQ ID NO: 40 - DNA, 20 bp, Artificial Sequence
  Description of Artificial Sequence Synthetic oligonucleotide
tccatgacgttcctgacgtt

(19) United States
(12) Patent Application Publication
(10) Pub. No.: US 2022/0265817 A1
(43) Pub. Date: Aug. 25, 2022

(54) THERAPEUTIC COMPOSITIONS AND METHODS FOR TREATING HEPATITIS B

(71) Applicant: ARBUTUS BIOPHARMA CORPORATION, Burnaby (CA)

(72) Inventors: Amy C. H. LEE, Burnaby (CA); Emily P. THI, Coquitlam (CA)

(73) Assignee: ARBUTUS BIOPHARMA CORPORATION, Burnaby, BC (CA)

(21) Appl. No.: 16/617,384

(22) PCT Filed: May 31, 2018
(86) PCT No.: PCT/US2018/035452

§ 371 Date: Nov. 26, 2019

Related U.S. Application Data
(60) Provisional application No. 62/513,261, filed on May 31, 2017.

(51) Int. Cl. A61K 39/29; A61K 39/395; A61P 31/20

(52) U.S. Cl. CPC A61K 39/29; A61K 39/39541; A61K 2039/505; A61P 31/20; A61K 39/3955

(57) ABSTRACT

The invention provides therapeutic combinations and therapeutic methods that are useful for treating hepatitis B.

### CROSS-REFERENCE TO RELATED APPLICATION(S)

This patent application claims the benefit of priority of U.S. application Ser. No. 62/513,261 filed May 31, 2017, which application is herein incorporated by reference.

### SEQUENCE LISTING

The instant application contains a Sequence Listing which has been submitted in ASCII format via EFS-Web and is hereby incorporated by reference in its entirety. Said ASCII copy, created on Jun. 19, 2018, is named 08155_069WO1_SL.txt and is 22,382 bytes in size.

### BACKGROUND

Hepatitis B virus (abbreviated as “HBV”) is a member of the Hepadnavirus family. The virus particle (sometimes referred to as a virion) includes an outer lipid envelope and an icosahedral nucleocapsid core composed of protein. The nucleocapsid encloses the viral DNA and a DNA polymerase that has reverse transcriptase activity. The outer envelope contains embedded proteins that are involved in viral binding of, and entry into, susceptible cells, typically liver hepatocytes. In addition to the infectious viral particles, filamentous and spherical bodies lacking a core can be found in the serum of infected individuals. These particles are not infectious and are composed of the lipid and protein that forms part of the surface of the virion, which is called the surface antigen (HBsAg), and is produced in excess during the life cycle of the virus.

The genome of HBV is made of circular DNA, but it is unusual because the DNA is not fully double-stranded. One end of the full-length strand is linked to the viral DNA polymerase. The genome is 3020-3320 nucleotides long (for the full-length strand) and 1700-2800 nucleotides long (for the shorter strand). The negative-sense (non-coding) is complementary to the viral mRNA. The viral DNA is found in the nucleus soon after infection of the cell. There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small are produced. The function of the protein coded for by gene X is not fully understood but it is associated with the development of liver cancer. Replication of HBV is a complex process. Although replication takes place in the liver, the virus spreads to the blood where viral proteins and antibodies against them are found in infected people. The structure, replication and biology of HBV is reviewed in D. Glebe and C. M. Bremer, Seminars in Liver Disease, Vol. 33, No. 2, pages 103-112 (2013).

Infection of humans with HBV can cause an infectious inflammatory illness of the liver. Infected individuals may not exhibit symptoms for many years. It is estimated that about a third of the world population has been infected at one point in their lives, including 350 million who are chronic carriers.

The virus is transmitted by exposure to infectious blood or body fluids. Perinatal infection can also be a major route of infection. The acute illness causes liver inflammation, vomiting, jaundice, and possibly death. Chronic hepatitis B may eventually cause cirrhosis and liver cancer.

Although most people who are infected with HBV clear the infection through the action of their immune system, some infected people suffer an aggressive course of infection (fulminant hepatitis); while others are chronically infected thereby increasing their chance of liver disease. Several medications are currently approved for treatment of HBV infection, but infected individuals respond with various degrees of success to these medications, and none of these medications clear the virus from the infected person.

Hepatitis D virus (HDV) is a small circular enveloped RNA virus that can propagate only in the presence of the hepatitis B virus (HBV). In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Thus, there is a continuing need for compositions and methods for the treatment of HBV infection humans, as well as for the treatment of HBV/HDV infection in humans.

### Summary

The present invention provides therapeutic combinations and therapeutic methods that are useful for treating viral infections such as HBV and/or HDV. As such, in certain embodiments, the following are provided.

Certain embodiments provide a method for treating hepatitis B in a human, comprising administering to the human:

an siRNA that targets a portion of the HBV genome;

an inhibitor of PD-L1; and

an anti-HBV vaccine.

Certain embodiments provide a method for treating hepatitis B in a human, comprising administering to the human at least one agent from at least three of the following categories of agents:

(A) an agent that controls viral replication;

(B) an agent that reduces viral Ags;

(C) an immune enhancer; and

(D) an immune stimulant.

Certain embodiments provide a method for treating hepatitis D in a human, comprising administering to the human:

an siRNA that targets a portion of the HBV genome;

an inhibitor of PD-L1; and

an anti-HBV vaccine.

Certain embodiments provide a method for treating hepatitis D in a human, comprising administering to the human at least one agent from at least three of the following categories of agents:

(A) an agent that controls viral replication;

(B) an agent that reduces viral Ags;

(C) an immune enhancer; and

(D) an immune stimulant.

Certain embodiments provide a method for treating hepatitis B and/or hepatitis D in a human, comprising first administering to the human an agent that reduces viral Ags and then administering an agent that improves the immune response to the hepatitis B virus.

In certain embodiments, the method(s) may be used to treat both HBV and HDV.

In certain embodiments, the method further comprises administering an agent that controls viral replication.

In certain embodiments, the inhibitor of PD-L1 is an anti-PD-L1 mAb.

In certain embodiments, the anti-HBV vaccine is a vaccine that targets the HBV surface antigen.

In certain embodiments, the siRNA that targets a portion of the HBV genome and the inhibitor of PD-L1 are administered concurrently.

In certain embodiments, the anti-HBV vaccine is administered subsequent to the administration of the siRNA and the inhibitor of PD-L1.

In certain embodiments, the agent that controls viral replication is administered concurrently with or prior to the administration of the siRNA and the inhibitor of PD-L1.

In certain embodiments, the agent that controls viral replication is administered concurrently with the administration of the siRNA and the inhibitor of PD-L1.

In certain embodiments, the agent that controls viral replication is administered prior to the administration of the siRNA and the inhibitor of PD-L1.

In certain embodiments, the siRNA, the inhibitor of PD-L1 and the anti-HBV vaccine are administered concurrently.

In certain embodiments, an agent that controls viral replication is administered concurrently with or prior to the administration of the siRNA, the inhibitor of PD-L1, and the anti-HBV vaccine.

In certain embodiments, an agent that controls viral replication is administered concurrently with the administration of the siRNA, the inhibitor of PD-L1, and the anti-HBV vaccine.

In certain embodiments, an agent that controls viral replication is administered prior to the administration of the siRNA, the inhibitor of PD-L1, and the anti-HBV vaccine.

In certain embodiments, the siRNA is administered, administration of the inhibitor of PD-L1 is started subsequent to the start of siRNA administration, and administration of the anti-HBV vaccine is started subsequent to the start of to the inhibitor of PD-L1. These administrations may overlap in certain embodiments.

In certain embodiments, an agent that controls viral replication is also administered.

In certain embodiments, the agent that controls viral replication is a reverse transcriptase inhibitor, a capsid inhibitor, a cccDNA inhibitor or an entry inhibitor.

In certain embodiments, the agent that controls viral replication is a reverse transcriptase inhibitor.

In certain embodiments, the agent that controls viral replication is a capsid inhibitor.

In certain embodiments, the agent that controls viral replication is a cccDNA inhibitor.

In certain embodiments, the agent that controls viral replication is an entry inhibitor.

In certain embodiments, the agent that controls viral replication is entecavir, clevudine, telbivudine, lamivudine, adefovir, and tenofovir, tenofovir disoproxil, tenofovir alafenamide, tenofovir disoproxil fumarate, adefovir dipivoxil, (1R,2R,3R,5R)-3-(6-amino-9H-9-purinyl)-2-fluoro-5-(hydroxymethyl)-4-methylenecyclopentan-1-ol, emtricitabine, abacavir, elvucitabine, ganciclovir, lobucavir, famciclovir, penciclovir, amdoxovir or CMX157 (tenofovir exalidex).

In certain embodiments, the agent that reduces viral Ags is an siRNA that targets a portion of the HBV genome (e.g., an siRNA, or a two-way or three-way combination of siRNA molecules described herein).

In certain embodiments, the agent that reduces viral Ags is an sAg secretion inhibitor.

In certain embodiments, the agent that reduces viral Ags is an anti-HBsAg agent.

In certain embodiments, the immune enhancer is a checkpoint inhibitor.

In certain embodiments, the immune enhancer is an inhibitor of PD-L1.

In certain embodiments, the immune enhancer is an anti-PD-1 mAb, an anti-PD-L1 mAb, an anti-PD-L2 mAb, an anti-CTLA4 mAb, an anti-VISTA mAb, an anti-LAG3 mAb, an anti-TIM3 mAb, or a peptidomimetic.

In certain embodiments, the immune stimulant is an anti-HBV vaccine, an interferon, a RIG-I agonist, a STING agonist, a TLR9 agonist, a TLR7 agonist, a TLR8 agonist, a TLR3 agonist, IL-7, IL-2, an OX-40 agonist, or an anti-GITR agonist.

In certain embodiments, the agent that reduces viral Ags and the immune enhancer are administered, concurrently.

In certain embodiments, the immune stimulant is administered, subsequent to the administration of the agent that reduces viral Ags and the immune enhancer.

In certain embodiments, the agent that controls viral replication is administered, concurrently with or prior to the administration of the agent that reduces viral Ags and the immune enhancer.

In certain embodiments, the agent that controls viral replication is administered concurrently with the administration of the agent that reduces viral Ags and the immune enhancer.

In certain embodiments, the agent that controls viral replication is administered prior to the administration of the agent that reduces viral Ags and the immune enhancer.

In certain embodiments, the agent that reduces viral Ags, the immune enhancer and the immune stimulant are administered, concurrently.

In certain embodiments, an agent that controls viral replication is administered concurrently with or prior to the administration of the agent that reduces viral Ags, the immune enhancer, and the immune stimulant.

In certain embodiments, an agent that controls viral replication is administered concurrently with the administration of the agent that reduces viral Ags, the immune enhancer, and the immune stimulant.

In certain embodiments, an agent that controls viral replication is administered prior to the administration of the agent that reduces viral Ags, the immune enhancer, and the immune stimulant.

In certain embodiments, the agent that reduces viral Ags is administered, administration of the immune enhancer is started subsequent to the start of agent that reduces viral Ags administration, and administration of the immune stimulant is started subsequent to the start of to the immune enhancer.

In certain embodiments, at least one agent from each of the four categories of agents is administered.

In certain embodiments, the agent that improves the immune response is an immune enhancer.

In certain embodiments, the agent that improves the immune response is an immune stimulant.

In certain embodiments, the method further comprises administering an agent that controls viral replication.

The Examples presented herein disclose the results of numerous combination (e.g., three-way combinations) studies using agents having differing mechanisms of action against HBV. As described herein, several combinations of agents showed unexpected, synergistic interactions, and combinations generally lacked antagonism.

### DETAILED DESCRIPTION

The present invention provides therapeutic combinations and therapeutic methods that are useful for treating viral infections such as HBV and/or HDV. The following categories of therapeutics treatments can be administered, in certain embodiments in specific orders, to optimize the treatment of HBV, as described herein.

I. Agents that Control Viral Replication

Category I treatments are directed to the use of agents that control, e.g., inhibit, viral replication.

A. Reverse Transcriptase Inhibitors

In certain embodiments, the reverse transcriptase inhibitor is a nucleoside analog.

In certain embodiments, the reverse transcriptase inhibitor is a nucleoside analog reverse-transcriptase inhibitor (NARTI or NRTI).

In certain embodiments, the reverse transcriptase inhibitor is a nucleotide analog reverse-transcriptase inhibitor (NtARTI or NtRTI).

The term reverse transcriptase inhibitor includes, but is not limited to: entecavir, clevudine, telbivudine, lamivudine, adefovir, and tenofovir, tenofovir disoproxil, tenofovir alafenamide, tenofovir disoproxil fumarate, adefovir dipivoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074), emtricitabine, abacavir, elvucitabine, ganciclovir, lobucavir, famciclovir, penciclovir, amdoxovir and CMX157 (tenofovir exalidex).

The term reverse transcriptase inhibitor includes, but is not limited to, entecavir, lamivudine, and (1R,2R,3R,5R)-3-(6-amino-9H-9-purinyl)-2-fluoro-5-(hydroxymethyl)-4-methylenecyclopentan-1-ol.

The term reverse transcriptase inhibitor includes, but is not limited to a covalently bound phosphoramidate or phosphonamidate moiety of the above-mentioned reverse transcriptase inhibitors, or as described in, for example, U.S. Pat. No. 8,816,074, US 2011/0245484 A1, and US 2008/0286230A1.

The term reverse transcriptase inhibitor includes, but is not limited to, nucleotide analogs that comprise a phosphoramidate moiety, such as, methyl ((((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy)phosphoryl)-(D or L)-alaninate. Also included are the individual diastereomers thereof, which includes, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate.

The term reverse transcriptase inhibitor includes, but is not limited to a phosphonamidate moiety, such as, tenofovir alafenamide, as well as those described in US 2008/0286230 A1. Methods for preparing stereoselective phosphoramidate or phosphonamidate containing actives are described in, for example, U.S. Pat. No. 8,816,074, as well as US 2011/0245484 A1 and US 2008/0286230 A1.

B. Capsid Inhibitors

As described herein the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA. Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

The term capsid inhibitor includes compounds described in International Patent Applications Publication Numbers WO2013006394, WO2014106019, and WO2014089296, including the following compounds:

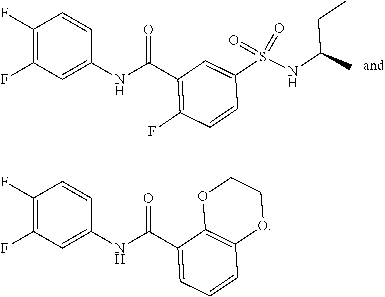 [SMILES: CC[C@@H](C)NS(=O)(=O)c1ccc(F)c(C(=O)Nc2ccc(F)c(F)c2)c1 ; O=C(Nc1ccc(F)c(F)c1)c1cccc2c1OCCO2]

The term capsid inhibitor also includes the compounds Bay-41-4109 (see International Patent Application Publication Number WO/2013/144129), AT-61 (see International Patent Application Publication Number WO/1998/33501; and King, R W, et al., Antimicrob Agents Chemother., 1998, 42, 12, 3179-3186), DVR-01 and DVR-23 (see International Patent Application Publication Number WO 2013/006394; and Campagna, M R, et al., J. of Virology, 2013, 87, 12, 6931, and pharmaceutically acceptable salts thereof:

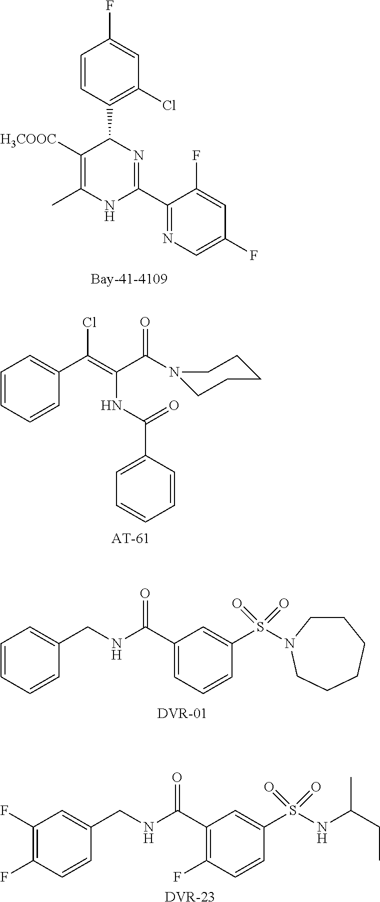

The term capsid inhibitor also includes the compounds Compound 3, GLS-4, and NVR 3-778.

C. cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

The term cccDNA formation inhibitor includes compounds described in International Patent Application Publication Number WO2013130703, including the following compound:

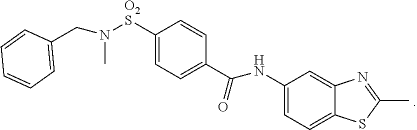 [SMILES: Cc1nc2cc(NC(=O)c3ccc(S(=O)(=O)N(C)Cc4ccccc4)cc3)ccc2s1]

The term cccDNA formation inhibitor includes, but is not limited to those generally and specifically described in United States Patent Application Publication Number US 2015/0038515 A1. The term cccDNA formation inhibitor includes, but is not limited to, 1-(phenylsulfonyl)-N-(pyridin-4-ylmethyl)-1H-indole-2-carboxamide; 1-Benzenesulfonyl-pyrrolidine-2-carboxylic acid (pyridin-4-ylmethyl)-amide; 2-(2-chloro-N-(2-chloro-5-(trifluoromethyl)phenyl)-4-(trifluoromethyl)phenylsulfonamido)-N-(pyridin-4-ylmethyl)acetamide; 2-(4-chloro-N-(2-chloro-5-(trifluoromethyl)phenyl)phenylsulfonamido)-N-(pyridin-4-ylmethyl)acetamide; 2-(N-(2-chloro-5-(trifluoromethyl)phenyl)-4-(trifluoromethyl)phenylsulfonamido)-N-(pyridin-4-ylmethyl)acetamide; 2-(N-(2-chloro-5-(trifluoromethyl)phenyl)-4-methoxyphenylsulfonamido)-N-(pyridin-4-ylmethyl)acetamide; 2-(N-(2-chloro-5-(trifluoromethyl)phenyl)phenylsulfonamido)-N-((1-methylpiperidin-4-yl)methyl)acetamide; 2-(N-(2-chloro-5-(trifluoromethyl)phenyl)phenylsulfonamido)-N-(piperidin-4-ylmethyl)acetamide; 2-(N-(2-chloro-5-(trifluoromethyl)phenyl)phenylsulfonamido)-N-(pyridin-4-ylmethyl)propanamide; 2-(N-(2-chloro-5-(trifluoromethyl)phenyl)phenylsulfonamido)-N-(pyridin-3-ylmethyl)acetamide; 2-(N-(2-chloro-5-(trifluoromethyl)phenyl)phenylsulfonamido)-N-(pyrimidin-5-ylmethyl)acetamide; 2-(N-(2-chloro-5-(trifluoromethyl)phenyl)phenylsulfonamido)-N-(pyrimidin-4-ylmethyl)acetamide; 2-(N-(5-chloro-2-fluorophenyl)phenylsulfonamido)-N-(pyridin-4-ylmethyl)acetamide; 2-[(2-chloro-5-trifluoromethyl-phenyl)-(4-fluoro-benzenesulfonyl)-amino]-N-pyridin-4-ylmethyl-acetamide; 2-[(2-chloro-5-trifluoromethyl-phenyl)-(toluene-4-sulfonyl)-amino]-N-pyridin-4-ylmethyl-acetamide; 2-[benzenesulfonyl-(2-bromo-5-trifluoromethyl-phenyl)-amino]-N-pyridin-4-ylmethyl-acetamide; 2-[benzenesulfonyl-(2-chloro-5-trifluoromethyl-phenyl)-amino]-N-(2-methyl-benzothiazol-5-yl)-acetamide; 2-[benzenesulfonyl-(2-chloro-5-trifluoromethyl-phenyl)-amino]-N-[4-(4-methyl-piperazin-1-yl)-benzyl]-acetamide; 2-[benzenesulfonyl-(2-chloro-5-trifluoromethyl-phenyl)-amino]-N-[3-(4-methyl-piperazin-1-yl)-benzyl]-acetamide; 2-[benzenesulfonyl-(2-chloro-5-trifluoromethyl-phenyl)-amino]-N-benzyl-acetamide; 2-[benzenesulfonyl-(2-chloro-5-trifluoromethyl-phenyl)-amino]-N-pyridin-4-ylmethyl-acetamide; 2-[benzenesulfonyl-(2-chloro-5-trifluoromethyl-phenyl)-amino]-N-pyridin-4-ylmethyl-propionamide; 2-[benzenesulfonyl-(2-fluoro-5-trifluoromethyl-phenyl)-amino]-N-pyridin-4-ylmethyl-acetamide; 4 (N-(2-chloro-5-(trifluoromethyl)phenyl)phenylsulfonamido)-N-(pyridin-4-yl-methyl)butanamide; 4-((2-(N-(2-chloro-5-(trifluoromethyl)phenyl)phenylsulfonamido)-acetamido)-methyl)-1,1-dimethylpiperidin-1-ium chloride; 4-(benzyl-methyl-sulfamoyl)-N-(2-chloro-5-trifluoromethyl-phenyl)-benzamide; 4-(benzyl-methyl-sulfamoyl)-N-(2-methyl-1H-indol-5-yl)-benzamide; 4-(benzyl-methyl-sulfamoyl)-N-(2-methyl-1H-indol-5-yl)-benzamide; 4-(benzyl-methyl-sulfamoyl)-N-(2-methyl-benzothiazol-5-yl)-benzamide; 4-(benzyl-methyl-sulfamoyl)-N-(2-methyl-benzothiazol-6-yl)-benzamide; 4-(benzyl-methyl-sulfamoyl)-N-(2-methyl-benzothiazol-6-yl)-benzamide; 4-(benzyl-methyl-sulfamoyl)-N-pyridin-4-ylmethyl-benzamide; N-(2-aminoethyl)-2-(N-(2-chloro-5-(trifluoromethyl)phenyl)phenylsulfonamido)-acetamide; N-(2-chloro-5-(trifluoromethyl)phenyl)-N-(2-(3,4-dihydro-2,6-naphthyridin-2(1H)-yl)-2-oxoethyl)benzenesulfonamide; N-benzothiazol-6-yl-4-(benzyl-methyl-sulfamoyl)-benzamide; N-benzothiazol-6-yl-4-(benzyl-methyl-sulfamoyl)-benzamide; tert-butyl (2-(2-(N-(2-chloro-5-(trifluoromethyl)phenyl)phenylsulfonamido)acetamido)-ethyl)carbamate; and tert-butyl 4-((2-(N-(2-chloro-5-(trifluoromethyl)phenyl)phenylsulfonamido)-acetamido)-methyl)piperidine-1-carboxylate, and optionally, combinations thereof.

D. Entry Inhibitors

Certain embodiments of the invention are directed to the use of agents that are HBV entry inhibitors. Entry inhibitors include Myrcludex-B, NTCP inhibitor small molecules, and FXR agonist EYP001 (see, e.g., Gripon, P., Cannie, I. and Urban, S. Efficient Inhibition of Hepatitis B Virus Infection by Acylated Peptides Derived from the Large Viral Surface Protein. Journal of Virology, 79(3): 1613-1622; Volz, T., Allweiss, L., MBarek, M., Warlich, M., Lohse, A., Pollok, J., Alexandrov, A., Urban, S., Petersen, J., Lutgehetmann, M., Dandri, M. The entry inhibitor Myrcludex-B efficiently blocks intrahepatic virus spreading in humanized mice previously infected with hepatitis B virus. Journal of Hepatology, 58(5): 861-867; Radreau, P., Procherot, M., Vonderscher, J., Lotteau, V., Andre, P. Effect of a novel synthetic FXR agonist EYP001 on hepatitis B virus replication in HepaRG cell line and primary human hepatocytes. AASLD LiverLearning, Abstract 1652, Nov. 16, 2015; WO 2015/036442; WO 00/37077; US2007/0015796) For example, the hepatitis B virus uses its surface lipopeptide pre-S1 for docking to mature liver cells via their sodium/bile acid cotransporter (NTCP) and subsequently entering the cells. Myrcludex B is a synthetic N-acylated pre-S1 that can also dock to NTCP, blocking the virus's entry mechanism.

II. Agents that Reduce Viral Ags

Category II treatments are directed to the use of agents that reduce viral antigens.

A. Oligomeric Nucleotides

The oligomeric nucleotides can be designed to target one or more genes and/or transcripts of the HBV genome. Examples of such siRNA molecules are the siRNA molecules set forth in Table A and the Examples herein, including the specific combinations of siRNA molecules described herein, e.g., the two-way and three-way combinations of siRNA molecules.

The term oligomeric nucleotide targeted to the Hepatitis B genome includes Arrowhead-ARC-520 (see U.S. Pat. No. 8,809,293; and Wooddell C I, et al., Molecular Therapy, 2013, 21, 5, 973-985).

The term oligomeric nucleotide targeted to the Hepatitis B genome also includes isolated, double stranded, siRNA molecules, that each include a sense strand and an antisense strand that is hybridized to the sense strand. The siRNA target one or more genes and/or transcripts of the HBV genome. Examples of siRNA molecules are the siRNA molecules set forth in Table A and the Examples herein, including the specific combinations of siRNA molecules described herein, e.g., the two-way and three-way combinations of siRNA molecules herein.

In another aspect, this term includes the isolated sense and antisense strands are set forth in Table B herein.

In another aspect, this term includes siRNA molecules that target GalNAc and REP 2139, REP-2165 (see, e.g., WO 2016/077321, Al-Mathtab et al., PLoS ONE 11(6):e0156667. doi:10.1371/journal.pone.0156667 and Guillot et al., Poster P0556, EASL, 2015).

B. sAg Secretion Inhibitors

As described herein the term “sAg secretion inhibitor” includes compounds that are capable of inhibiting, either directly or indirectly, the secretion of sAg (S, M and/or L surface antigens) bearing subviral particles and/or DNA containing viral particles from HBV-infected cells. For example, in certain embodiments, the inhibitor detectably inhibits the secretion of sAg as measured, e.g., using assays known in the art or described herein, e.g., ELISA assay or by Western Blot. In certain embodiments, the inhibitor inhibits the secretion of sAg by at least 5%, at least 10%, at least 20%, at least 50%, at least 75%, or at least 90%. In certain embodiments, the inhibitor reduces serum levels of sAg in a patient by at least 5%, at least 10%, at least 20%, at least 50%, at least 75%, or at least 90%.

The term sAg secretion inhibitor includes compounds described in U.S. Pat. No. 8,921,381, as well as compounds described in United States Patent Application Publication Numbers 2015/0087659 and 2013/0303552. For example, the term includes the compounds PBHBV-001 and PBHBV-2-15, and pharmaceutically acceptable salts thereof:

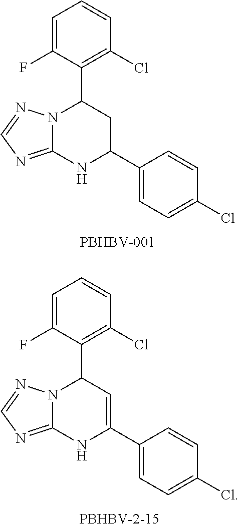 [SMILES: Fc1cccc(Cl)c1C1CC(c2ccc(Cl)cc2)Nc2ncnn21 ; Fc1cccc(Cl)c1C1C=C(c2ccc(Cl)cc2)Nc2ncnn21]

C. Anti-HBsAg Agents

Certain aspects of the invention are directed to the use of anti-HBsAg antibodies, e.g., mAbs. Certain aspects of the invention are directed to the use of hepatitis B immune globulin (HBIG).

III. Agents that Improve Immune Response

Category III treatments are directed to the use of agents that improve the immune response against viral infection. In certain embodiments, at least one ‘immune enhancer’ agent is used in combination with at least one ‘immune stimulant agent’. Such a combination can be used in further combination with at least one agent that controls viral replication and/or at least one agent that reduces the viral antigens.

A. Immune Enhancers

Certain aspects of the invention are directed to the use of agents that act to improve an immune response by reducing or eliminating immune exhaustion, e.g., by using checkpoint inhibitors, thereby enhancing the immune response.

In certain embodiments, an immune enhancer is a PD-L1 inhibitor. PD-L1 inhibitors are a group of agents that act to inhibit the association of the programmed death-ligand 1 (PD-L1) with its receptor, programmed cell death protein 1 (PD-1).

Immune enhancers include the following:

anti-PD-1 mAbs (e.g., Nivolumab, Pembrolizumab;

anti-PD-L1 mAbs (e.g., Atezolizumab, Avelumab);

anti-PD-L2 mAbs;

anti-CTLA4 mAbs (e.g., Ipilimumab);

anti-VISTA mAbs (e.g., JNJ-61610588);

anti-LAG3 mAbs (e.g., BMS-986016);

anti-TIM3 mAbs (e.g., TSR-022);

peptidomimetics (e.g., AUNP-12); and

small molecule compounds (see, e.g., Zak et al., Oncotarget, 2016, 7(21):30323-35)

B. Immune Stimulants

The term “immune stimulant” includes compounds that are capable of modulating an immune response (e.g., stimulating an innate and/or adaptive immune response (e.g., an adjuvant)). The term immune stimulant includes polyinosinic:polycytidylic acid (poly I:C) and interferons.

The term immune stimulant includes agonists of stimulator of IFN genes (STING) and interleukins. The term also includes HBsAg release inhibitors, TLR-7 agonists (GS-9620, RG-7795), T-cell and/or B-cell stimulators (GS-4774, OX-40 agonists (BMS 986178), anti-GITR agonists (BMS-986156)), RIG-1 inhibitors (SB-9200), and SMAC-mimetics (Birinapant).

The term also includes the following:

anti-HBV vaccines (Engerix-B, RECOMBIVAX HB, GS-4744, Heplisav-B);

interferons (Pegylated IFN-α2a, Peglyated IFN-α2b, IFN-α, IFN-λ);

RIG-I agonists (SB-9200);

STING agonists (cGAMP, cGAMP bisphosphorothioate, ADU S100, and other small molecule compounds);

TLR9 agonists (CYT-009, CpG dinucleotides);

TLR7 agonists (GS-9620);

TLR8 agonists (GS-9688);

TLR3 agonists (Ampligen/poly I:C12U);

IL-7 (CYT107); and

IL-2 (aldesleukin).

The term “Hepatitis B virus” (abbreviated as HBV) refers to a virus species of the genus Orthohepadnavirus, which is a part of the Hepadnaviridae family of viruses, and that is capable of causing liver inflammation in humans.

The term “Hepatitis D virus” (abbreviated as HDV) refers to a virus species of the genus Deltaviridae, which is capable of causing liver inflammation in humans.

As used herein, “treatment” (and grammatical variations thereof such as “treat” or “treating”) refers to clinical intervention in an attempt to alter the typical disease course of the individual being treated. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and is preferably about 20-24, 21-22, or 21-23 (duplex) nucleotides in length. siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA to silence, reduce, or inhibit expression of a target gene (e.g., a gene within the HBV genome). To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art. An “effective amount” or “therapeutically effective amount” of a therapeutic nucleic acid such as a siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Accordingly, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

An “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “unlocked nucleobase analogue” (abbreviated as “UNA”) refers to an acyclic nucleobase in which the C2′ and C3′ atoms of the ribose ring are not covalently linked. The term “unlocked nucleobase analogue” includes nucleobase analogues having the following structure identified as Structure A:

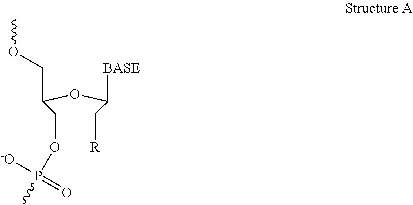

wherein R is hydroxyl, and Base is any natural or unnatural base such as, for example, adenine (A), cytosine (C), guanine (G) and thymine (T). UNA include the molecules identified as acyclic 2′-3′-seco-nucleotide monomers in U.S. patent serial number 8,314,227.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid particle” includes a lipid formulation that can be used to deliver a therapeutic nucleic acid (e.g., siRNA) to a target site of interest (e.g., cell, tissue, organ, and the like). In preferred embodiments, the lipid particle is typically formed from a cationic lipid, a non-cationic lipid, and optionally a conjugated lipid that prevents aggregation of the particle. A lipid particle that includes a nucleic acid molecule (e.g., siRNA molecule) is referred to as a nucleic acid-lipid particle. Typically, the nucleic acid is fully encapsulated within the lipid particle, thereby protecting the nucleic acid from enzymatic degradation.

In certain instances, nucleic acid-lipid particles are extremely useful for systemic applications, as they can exhibit extended circulation lifetimes following intravenous (i.v.) injection, they can accumulate at distal sites (e.g., sites physically separated from the administration site), and they can mediate silencing of target gene expression at these distal sites. The nucleic acid may be complexed with a condensing agent and encapsulated within a lipid particle as set forth in PCT Publication No. WO 00/03683, the disclosure of which is herein incorporated by reference in its entirety for all purposes.

The lipid particles typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In addition, nucleic acids, when present in the lipid particles, are resistant in aqueous solution to degradation with a nuclease. Nucleic acid-lipid particles and their method of preparation are disclosed in, e.g., U.S. Patent Publication Nos. 20040142025 and 20070042031, the disclosures of which are herein incorporated by reference in their entirety for all purposes.

As used herein, “lipid encapsulated” can refer to a lipid particle that provides a therapeutic nucleic acid such as a siRNA, with full encapsulation, partial encapsulation, or both. In a preferred embodiment, the nucleic acid (e.g., siRNA) is fully encapsulated in the lipid particle (e.g., to form a nucleic acid-lipid particle).

The term “lipid conjugate” refers to a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used.

The term “amphipathic lipid” refers, in part, to any suitable material wherein the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

The terms “cationic lipid” and “amino lipid” are used interchangeably herein to include those lipids and salts thereof having one, two, three, or more fatty acid or fatty alkyl chains and a pH-titratable amino head group (e.g., an alkylamino or dialkylamino head group). The cationic lipid is typically protonated (i.e., positively charged) at a pH below the pKₐ of the cationic lipid and is substantially neutral at a pH above the pKₐ. The cationic lipids may also be termed titratable cationic lipids. In some embodiments, the cationic lipids comprise: a protonatable tertiary amine (e.g., pH-titratable) head group; C₁₈ alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, γ-DLenDMA, DLin-K-DMA, DLin-K-C2-DMA (also known as DLin-C2K-DMA, XTC2, and C2K), DLin-K-C3-DMA, DLin-K-C4-DMA, DLen-C2K-DMA, γ-DLen-C2K-DMA, DLin-M-C2-DMA (also known as MC2), and DLin-M-C3-DMA (also known as MC3).

Administration of a compound as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The term “salts” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfate, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

The term “alkenyl” includes an alkyl, as defined above, containing at least one double bond between adjacent carbon atoms. Alkenyls include both cis and trans isomers. Representative straight chain and branched alkenyls include, but are not limited to, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

The term “alkynyl” includes any alkyl or alkenyl, as defined above, which additionally contains at least one triple bond between adjacent carbons. Representative straight chain and branched alkynyls include, without limitation, acetylenyl, propynyl, 1-butynyl, 2-butynyl, 1-pentynyl, 2-pentynyl, 3-methyl-1 butynyl, and the like.

The term “acyl” includes any alkyl, alkenyl, or alkynyl wherein the carbon at the point of attachment is substituted with an oxo group, as defined below. The following are non-limiting examples of acyl groups: —C(═O)alkyl, —C(═O)alkenyl, and —C(═O)alkynyl.

The term “heterocycle” includes a 5- to 7-membered monocyclic, or 7- to 10-membered bicyclic, heterocyclic ring which is either saturated, unsaturated, or aromatic, and which contains from 1 or 2 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include, but are not limited to, heteroaryls as defined below, as well as morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The terms “optionally substituted alkyl”, “optionally substituted alkenyl”, “optionally substituted alkynyl”, “optionally substituted acyl”, and “optionally substituted heterocycle” mean that, when substituted, at least one hydrogen atom is replaced with a substituent. In the case of an oxo substituent (═O), two hydrogen atoms are replaced. In this regard, substituents include, but are not limited to, oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}, wherein n is 0, 1, or 2, R^{x} and R^{y} are the same or different and are independently hydrogen, alkyl, or heterocycle, and each of the alkyl and heterocycle substituents may be further substituted with one or more of oxo, halogen, —OH, —CN, alkyl, —OR^{x}, heterocycle, —NR″R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x}, and —SOₙNR^{x}R^{y}. The term “optionally substituted,” when used before a list of substituents, means that each of the substituents in the list may be optionally substituted as described herein.

The term “halogen” includes fluoro, chloro, bromo, and iodo.

The term “fusogenic” refers to the ability of a lipid particle to fuse with the membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

The term “electron dense core”, when used to describe a lipid particle, refers to the dark appearance of the interior portion of a lipid particle when visualized using cryo transmission electron microscopy (“cyroTEM”). Some lipid particles have an electron dense core and lack a lipid bilayer structure. Some lipid particles have an elctron dense core, lack a lipid bilayer structure, and have an inverse Hexagonal or Cubic phase structure. While not wishing to be bound by theory, it is thought that the non-bilayer lipid packing provides a 3-dimensional network of lipid cylinders with water and nucleic acid on the inside, i.e., essentially a lipid droplet interpenetrated with aqueous channels containing the nucleic acid.

“Distal site,” as used herein, refers to a physically separated site, which is not limited to an adjacent capillary bed, but includes sites broadly distributed throughout an organism.

“Serum-stable” in relation to nucleic acid-lipid particles means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of lipid particles that leads to a broad biodistribution of an active agent such as a siRNA within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

“Local delivery,” as used herein, refers to delivery of an active agent such as a siRNA directly to a target site within an organism. For example, an agent can be locally delivered by direct injection into a disease site, other target site, or a target organ such as the liver, heart, pancreas, kidney, and the like.

The term “virus particle load”, as used herein, refers to a measure of the number of virus particles (e.g., HBV and/or HDV) present in a bodily fluid, such as blood. For example, particle load may be expressed as the number of virus particles per milliliter of, e.g., blood. Particle load testing may be performed using nucleic acid amplification based tests, as well as non-nucleic acid-based tests (see, e.g., Puren et al., The Journal of Infectious Diseases, 201:S27-36 (2010)).

TABLE A

| | | IC 50 |
|---|---|---|
| Name | Duplex Sequences | (nM) |
| 1m | 5′ A g G u A U g u U G C C C g U u U G U U U3′ (SEQ ID NO: 1) | 1.43 |
| | 3′ U U U C C A u A C A A C G G g C A A A C A 5′ (SEQ ID NO: 2) | |
| 2m | 5′ G C u c A g U U U A C U A G U G C c A U U3′ (SEQ ID NO: 3) | 0.37 |
| | 3′ U U C g A G U C A A A u G A U C A C G G U 5′ (SEQ ID NO: 4) | |
| 3m | 5′ C C G U g u G C A C U u C G C u u C A U U3′ (SEQ ID NO: 5) | 0.06 |
| | 3′ U U G g C A C A C g U G A A G C G A A G U 5′ (SEQ ID NO: 6) | |
| 4m | 5′ G C u c A g U U U A C U A G U G C c A U U3′ (SEQ ID NO: 7) | 0.31 |
| | 3′ U U C g A G U C A A A u G A U C A C G G U 5′ (SEQ ID NO: 8) | |
| 5m | 5′ C C G U g u G C A C U u C G C u U C A U U3′ (SEQ ID NO: 9) | 0.06 |
| | 3′ U U G g C A C A C g U G A A G C G A A G U 5′ (SEQ ID NO: 10) | |
| 6m | 5′ C u g g C U C A G U U U A C u A g U G U U3′ (SEQ ID NO: 11) | 0.05 |
| | 3′ U U G A C C g A g U C A A A U g A U C A C 5′ (SEQ ID NO: 12) | |
| 7m | 5′ C C G U g u G C A C U u C G C u U C A U U3′ (SEQ ID NO: 13) | 0.06 |
| | 3′ U U G g C A C A C g U G A A G C G A A G U 5′ (SEQ ID NO: 14) | |
| 8m | 5′ G C u C A g U U U A C u A g U G C C A U U3′ (SEQ ID NO: 15) | 0.24 |
| | 3′ U U C G A G u C A A A U G A U C A C G G U 5′ (SEQ ID NO: 16) | |
| 9m | 5′ A g G u A U G u U G C C C g U u U G U U U3′ (SEQ ID NO: 17) | 0.13 |
| | 3′ U U u C C A u A C A A C G G g C A A A C A 5′ (SEQ ID NO: 18) | |
| 10m | 5′ G C C g A u C C A U A C u g C g g A A U U3′ (SEQ ID NO: 19) | 0.34 |
| | 3′ U U C g G C U A g G U A U g A C G C C U U 5′ (SEQ ID NO: 20) | |
| 11m | 5′ G C C g A u C C A U A C u g C g g A A U U3′ (SEQ ID NO: 21) | 0.31 |
| | 3′ U U C g G C U A g G U A U g A C G C C U U 5′ (SEQ ID NO: 22) | |
| 12m | 5′ G C C g A u C C A U A C u g C G g A A U U3′ (SEQ ID NO: 23) | 0.16 |
| | 3′ U U C g G C U A g G U A U g A C G C C U U 5′ (SEQ ID NO: 24) | |
| 13m | 5′ G C C g A u C C A U A C u g C G g A A U U3′ (SEQ ID NO: 25) | 0.2 |
| | 3′ U U C g G C U A g G U A U g A C G C C U U 5′ (SEQ ID NO: 26) | |
| 14m | 5′ G C u C A g U U U A C u A g U G C C A U U3′ (SEQ ID NO: 27) | 0.16 |
| | 3′ U U C G A G u C A A A U G A U C A C G G U 5′ (SEQ ID NO: 28) | |
| 15m | 5′ C u g G C u C A G U U u A C U A G U G U U3′ (SEQ ID NO: 29) | 0.17 |
| | 3′ U U G A C C g A G U C A A A U G A U C A C 5′ (SEQ ID NO: 30) | |

lower case = 2′O-methyl modification
Underline = UNA moiety

The oligonucleotides (such as the sense and antisense RNA strands set forth in Table B) specifically hybridize to or is complementary to a target polynucleotide sequence. The terms “specifically hybridizable” and “complementary” as used herein indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. In preferred embodiments, an oligonucleotide is specifically hybridizable when binding of the oligonucleotide to the target sequence interferes with the normal function of the target sequence to cause a loss of utility or expression therefrom, and there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under conditions in which the assays are conducted. Thus, the oligonucleotide may include 1, 2, 3, or more base substitutions as compared to the region of a gene or mRNA sequence that it is targeting or to which it specifically hybridizes.

TABLE B

| Name | Sense Sequence (5′-3′) | Antisense Sequence (5′-3′) |
|---|---|---|
| 1m | AgGuAUguUGCCCgUuUGUUU (SEQ ID NO: 1) | ACAAACgGGCAACAuACCUUU (SEQ ID NO: 2) |
| 2m | GCucAgUUUACUAGUGCcAUU (SEQ ID NO: 3) | UGGCACUAGuAAACUGAgCUU (SEQ ID NO: 4) |
| 3m | CCGUguGCACUuCGCuuCAUU (SEQ ID NO: 5) | UGAAGCGAAGUgCACACgGUU (SEQ ID NO: 6) |
| 4m | GCucAgUUUACUAGUGCcAUU (SEQ ID NO: 7) | UGGCACUAGuAAACUGAgCUU (SEQ ID NO: 8) |
| 5m | CCGUguGCACUuCGCuUCAUU (SEQ ID NO: 9) | UGAAGCGAAGUgCACACgGUU (SEQ ID NO: 10) |
| 6m | CuggCUCAGUUUACuAgUGUU (SEQ ID NO: 11) | CACUAgUAAACUgAgCCAGUU (SEQ ID NO: 12) |
| 7m | CCGUguGCACUuCGCuUCAUU (SEQ ID NO: 13) | UGAAGCGAAGUgCACACgGUU (SEQ ID NO: 14) |
| 8m | GCuCAgUUUACuAgUGCCAUU (SEQ ID NO: 15) | UGGCACUAGUAAACuGAGCUU (SEQ ID NO: 16) |
| 9m | AgGuAUGuUGCCCgUuUGUUU (SEQ ID NO: 17) | ACAAACgGGCAACAuACCuUU (SEQ ID NO: 18) |
| 10m | GCCgAuCCAUACugCggAAUU (SEQ ID NO: 19) | UUCCGCAgUAUGgAUCGgCUU (SEQ ID NO: 20) |
| 11m | GCCgAuCCAUACugCggAAUU (SEQ ID NO: 21) | UUCCGCAgUAUGgAUCGgCUU (SEQ ID NO: 22) |
| 12m | GCCgAuCCAUACugCGgAAUU (SEQ ID NO: 23) | UUCCGCAgUAUGgAUCGgCUU (SEQ ID NO: 24) |
| 13m | GCCgAuCCAUACugCGgAAUU (SEQ ID NO: 25) | UUCCGCAgUAUGgAUCGgCUU (SEQ ID NO: 26) |
| 14m | GCuCAgUUUACuAgUGCCAUU (SEQ ID NO: 27) | UGGCACUAGUAAACuGAGCUU (SEQ ID NO: 28) |
| 15m | CugGCuCAGUUuACUAGUGUU (SEQ ID NO: 29) | CACUAGUAAACUGAgCCAGUU (SEQ ID NO: 30) |

lower case = 2′O-methyl modification
Underline = UNA moiety

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Carrier Systems Containing Therapeutic Nucleic Acids

Lipid Particles

The lipid particles can comprise one or more siRNA (e.g., an siRNA molecules described in Table A or the Examples herein, including the specific combinations of siRNA molecules described herein, e.g., the two-way and three-way combinations of siRNA molecules), a cationic lipid, a non-cationic lipid, and a conjugated lipid that inhibits aggregation of particles. In some embodiments, the siRNA molecule is fully encapsulated within the lipid portion of the lipid particle such that the siRNA molecule in the lipid particle is resistant in aqueous solution to nuclease degradation. In other embodiments, the lipid particles described herein are substantially non-toxic to humans. The lipid particles typically have a mean diameter of from about 30 nm to about 150 nm, from about 40 nm to about 150 nm, from about 50 nm to about 150 nm, from about 60 nm to about 130 nm, from about 70 nm to about 110 nm, or from about 70 to about 90 nm. In certain embodiments, the lipid particles have a median diameter of from about 30 nm to about 150 nm. The lipid particles also typically have a lipid:nucleic acid ratio (e.g., a lipid:siRNA ratio) (mass/mass ratio) of from about 1:1 to about 100:1, from about 1:1 to about 50:1, from about 2:1 to about 25:1, from about 3:1 to about 20:1, from about 5:1 to about 15:1, or from about 5:1 to about 10:1. In certain embodiments, the nucleic acid-lipid particle has a lipid:siRNA mass ratio of from about 5:1 to about 15:1.

The lipid particles include serum-stable nucleic acid-lipid particles which comprise one or more siRNA molecules (e.g., a siRNA molecule as described in Table A or the Examples herein, including the specific combinations of siRNA molecules described herein, e.g., the two-way and three-way combinations of siRNA molecules), a cationic lipid (e.g., one or more cationic lipids of Formula I-III or salts thereof as set forth herein), a non-cationic lipid (e.g., mixtures of one or more phospholipids and cholesterol), and a conjugated lipid that inhibits aggregation of the particles (e.g., one or more PEG-lipid conjugates). The lipid particle may comprise at least 1, 2, 3, 4, 5, 6, 7, 8, 9, 10, or more siRNA molecules (e.g., siRNA molecules described in Table A or the Examples herein, including the specific combinations of siRNA molecules described herein, e.g., the two-way and three-way combinations of siRNA molecules) that target one or more of the genes described herein. Nucleic acid-lipid particles and their method of preparation are described in, e.g., U.S. Pat. Nos. 5,753,613; 5,785,992; 5,705,385; 5,976,567; 5,981,501; 6,110,745; and 6,320,017; and PCT Publication No. WO 96/40964, the disclosures of which are each herein incorporated by reference in their entirety for all purposes.

In the nucleic acid-lipid particles, the one or more siRNA molecules (e.g., an siRNA molecule as described in Table A or the Examples herein, including the specific combinations of siRNA molecules described herein, e.g., the two-way and three-way combinations of siRNA molecules) may be fully encapsulated within the lipid portion of the particle, thereby protecting the siRNA from nuclease degradation. In certain instances, the siRNA in the nucleic acid-lipid particle is not substantially degraded after exposure of the particle to a nuclease at 37° C. for at least about 20, 30, 45, or 60 minutes. In certain other instances, the siRNA in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the siRNA is complexed with the lipid portion of the particle. One of the benefits of the formulations is that the nucleic acid-lipid particle compositions are substantially non-toxic to humans.

The term “fully encapsulated” indicates that the siRNA (e.g., a siRNA molecule as described in Table A or the Examples herein, including the specific combinations of siRNA molecules described herein, e.g., the two-way and three-way combinations of siRNA molecules) in the nucleic acid-lipid particle is not significantly degraded after exposure to serum or a nuclease assay that would significantly degrade free DNA or RNA. In a fully encapsulated system, preferably less than about 25% of the siRNA in the particle is degraded in a treatment that would normally degrade 100% of free siRNA, more preferably less than about 10%, and most preferably less than about 5% of the siRNA in the particle is degraded. “Fully encapsulated” also indicates that the nucleic acid-lipid particles are serum-stable, that is, that they do not rapidly decompose into their component parts upon in vivo administration.

In the context of nucleic acids, full encapsulation may be determined by performing a membrane-impermeable fluorescent dye exclusion assay, which uses a dye that has enhanced fluorescence when associated with nucleic acid. Specific dyes such as OliGreen® and RiboGreen® (Invitrogen Corp.; Carlsbad, Calif.) are available for the quantitative determination of plasmid DNA, single-stranded deoxyribonucleotides, and/or single- or double-stranded ribonucleotides. Encapsulation is determined by adding the dye to a liposomal formulation, measuring the resulting fluorescence, and comparing it to the fluorescence observed upon addition of a small amount of nonionic detergent. Detergent-mediated disruption of the liposomal bilayer releases the encapsulated nucleic acid, allowing it to interact with the membrane-impermeable dye. Nucleic acid encapsulation may be calculated as E=(Iₒ−I)/Iₒ, where I and Iₒ refer to the fluorescence intensities before and after the addition of detergent (see, Wheeler et al., Gene Ther., 6:271-281 (1999)).

In some instances, the nucleic acid-lipid particle composition comprises a siRNA molecule that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have the siRNA encapsulated therein.

In other instances, the nucleic acid-lipid particle composition comprises siRNA that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the input siRNA is encapsulated in the particles.

Depending on the intended use of the lipid particles, the proportions of the components can be varied and the delivery efficiency of a particular formulation can be measured using, e.g., an endosomal release parameter (ERP) assay.

Cationic Lipids

Any of a variety of cationic lipids or salts thereof may be used in the lipid particles either alone or in combination with one or more other cationic lipid species or non-cationic lipid species. The cationic lipids include the (R) and/or (S) enantiomers thereof.

In one aspect, the cationic lipid is a dialkyl lipid. For example, dialkyl lipids may include lipids that comprise two saturated or unsaturated alkyl chains, wherein each of the alkyl chains may be substituted or unsubstituted. In certain embodiments, each of the two alkyl chains comprise at least, e.g., 8 carbon atoms, 10 carbon atoms, 12 carbon atoms, 14 carbon atoms, 16 carbon atoms, 18 carbon atoms, 20 carbon atoms, 22 carbon atoms or 24 carbon atoms.

In one aspect, the cationic lipid is a trialkyl lipid. For example, trialkyl lipids may include lipids that comprise three saturated or unsaturated alkyl chains, wherein each of the alkyl chains may be substituted or unsubstituted. In certain embodiments, each of the three alkyl chains comprise at least, e.g., 8 carbon atoms, 10 carbon atoms, 12 carbon atoms, 14 carbon atoms, 16 carbon atoms, 18 carbon atoms, 20 carbon atoms, 22 carbon atoms or 24 carbon atoms.

In one aspect, cationic lipids of Formula I having the following structure are useful:

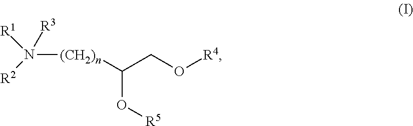

or salts thereof, wherein:

R¹ and R² are either the same or different and are independently hydrogen (H) or an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof;

R³ is either absent or is hydrogen (H) or a C₁-C₆ alkyl to provide a quaternary amine;

R⁴ and R⁵ are either the same or different and are independently an optionally substituted C₁₀-C₂₄ alkyl, C₁₀-C₂₄ alkenyl, C₁₀-C₂₄ alkynyl, or C₁₀-C₂₄ acyl, wherein at least one of R⁴ and R⁵ comprises at least two sites of unsaturation; and

n is 0, 1, 2, 3, or 4.

In some embodiments, R¹ and R² are independently an optionally substituted C₁-C₄ alkyl, C₂-C₄ alkenyl, or C₂-C₄ alkynyl. In one preferred embodiment, R¹ and R² are both methyl groups. In other preferred embodiments, n is 1 or 2. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₁₂-C₂₀ or C₁₄-C₂₂ alkyl, C₁₂-C₂₀ or C₁₄-C₂₂ alkenyl, C₁₂-C₂₀ or C₁₄-C₂₂ alkynyl, or C₁₂-C₂₀ or C₁₄-C₂₂ acyl, wherein at least one of R⁴ and R⁵ comprises at least two sites of unsaturation.

In certain embodiments, R⁴ and R⁵ are independently selected from the group consisting of a dodecadienyl moiety, a tetradecadienyl moiety, a hexadecadienyl moiety, an octadecadienyl moiety, an icosadienyl moiety, a dodecatrienyl moiety, a tetradectrienyl moiety, a hexadecatrienyl moiety, an octadecatrienyl moiety, an icosatrienyl moiety, an arachidonyl moiety, and a docosahexaenoyl moiety, as well as acyl derivatives thereof (e.g., linoleoyl, linolenoyl, γ-linolenoyl, etc.). In some instances, one of R⁴ and R⁵ comprises a branched alkyl group (e.g., a phytanyl moiety) or an acyl derivative thereof (e.g., a phytanoyl moiety). In certain instances, the octadecadienyl moiety is a linoleyl moiety. In certain other instances, the octadecatrienyl moiety is a linolenyl moiety or a γ-linolenyl moiety. In certain embodiments, R⁴ and R⁵ are both linoleyl moieties, linolenyl moieties, or γ-linolenyl moieties. In particular embodiments, the cationic lipid of Formula I is 1,2-dilinoleyloxy-N,N-dimethylaminopropane (DLinDMA), 1,2-dilinolenyloxy-N,N-dimethylaminopropane (DLenDMA), 1,2-dilinoleyloxy-(N,N-dimethyl)-butyl-4-amine (C2-DLinDMA), 1,2-dilinoleoyloxy-(N,N-dimethyl)-butyl-4-amine (C2-DLinDAP), or mixtures thereof.

In some embodiments, the cationic lipid of Formula I forms a salt (preferably a crystalline salt) with one or more anions. In one particular embodiment, the cationic lipid of Formula I is the oxalate (e.g., hemioxalate) salt thereof, which is preferably a crystalline salt.

The synthesis of cationic lipids such as DLinDMA and DLenDMA, as well as additional cationic lipids, is described in U.S. Patent Publication No. 20060083780, the disclosure of which is herein incorporated by reference in its entirety for all purposes. The synthesis of cationic lipids such as C₂-DLinDMA and C₂-DLinDAP, as well as additional cationic lipids, is described in international patent application number WO2011/000106 the disclosure of which is herein incorporated by reference in its entirety for all purposes.

In another aspect, cationic lipids of Formula II having the following structure (or salts thereof) are useful:

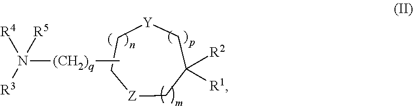

wherein R¹ and R² are either the same or different and are independently an optionally substituted C₁₂-C₂₄ alkyl, C₁₂-C₂₄ alkenyl, C₁₂-C₂₄ alkynyl, or C₁₂-C₂₄ acyl; R³ and R⁴ are either the same or different and are independently an optionally substituted C₁-C₆ alkyl, C₂-C₆ alkenyl, or C₂-C₆ alkynyl, or R³ and R⁴ may join to form an optionally substituted heterocyclic ring of 4 to 6 carbon atoms and 1 or 2 heteroatoms chosen from nitrogen and oxygen; R⁵ is either absent or is hydrogen (H) or a C₁-C₆ alkyl to provide a quaternary amine; m, n, and p are either the same or different and are independently either 0, 1, or 2, with the proviso that m, n, and p are not simultaneously 0; q is 0, 1, 2, 3, or 4; and Y and Z are either the same or different and are independently 0, S, or NH. In a preferred embodiment, q is 2.

In some embodiments, the cationic lipid of Formula II is 2,2-dilinoleyl-4-(2-dimethylaminoethyl)-[1,3]-dioxolane (DLin-K-C2-DMA; “XTC2” or “C2K”), 2,2-dilinoleyl-4-(3-dimethylaminopropyl)-[1,3]-dioxolane (DLin-K-C3-DMA; “C3K”), 2,2-dilinoleyl-4-(4-dimethylaminobutyl)-[1,3]-dioxolane (DLin-K-C4-DMA; “C4K”), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 2,2-dioleoyl-4-dimethylaminomethyl-[1,3]-dioxolane (DO-K-DMA), 2,2-distearoyl-4-dimethylaminomethyl-[1,3]-dioxolane (DS-K-DMA), 2,2-dilinoleyl-4-N-morpholino-[1,3]-dioxolane (DLin-K-MA), 2,2-Dilinoleyl-4-trimethylamino-[1,3]-dioxolane chloride (DLin-K-TMA.C1), 2,2-dilinoleyl-4,5-bis(dimethylaminomethyl)-[1,3]-dioxolane (DLin-K²-DMA), 2,2-dilinoleyl-4-methylpiperzine-[1,3]-dioxolane (D-Lin-K-N-methylpiperzine), or mixtures thereof. In one embodiment the cationic lipid of Formula II is DLin-K-C2-DMA.

In some embodiments, the cationic lipid of Formula II forms a salt (preferably a crystalline salt) with one or more anions. In one particular embodiment, the cationic lipid of Formula II is the oxalate (e.g., hemioxalate) salt thereof, which is preferably a crystalline salt.

The synthesis of cationic lipids such as DLin-K-DMA, as well as additional cationic lipids, is described in PCT Publication No. WO 09/086558, the disclosure of which is herein incorporated by reference in its entirety for all purposes. The synthesis of cationic lipids such as DLin-K-C2-DMA, DLin-K-C3-DMA, DLin-K-C4-DMA, DLin-K6-DMA, DLin-K-MPZ, DO-K-DMA, DS-K-DMA, DLin-K-MA, DLin-K-TMA.C1, DLin-K²-DMA, and D-Lin-K-N-methylpiperzine, as well as additional cationic lipids, is described in PCT Application No. PCT/US2009/060251, entitled “Improved Amino Lipids and Methods for the Delivery of Nucleic Acids,” filed Oct. 9, 2009, the disclosure of which is incorporated herein by reference in its entirety for all purposes.

In a further aspect, cationic lipids of Formula III having the following structure are useful:

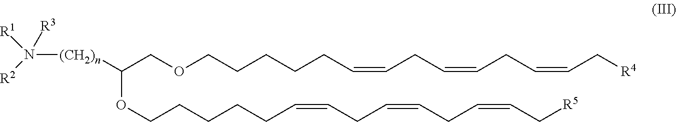

or salts thereof, wherein: R¹ and R² are either the same or different and are independently an optionally substituted C₁-C₆ alkyl, C₂-C₆ alkenyl, or C₂-C₆ alkynyl, or R¹ and R² may join to form an optionally substituted heterocyclic ring of 4 to 6 carbon atoms and 1 or 2 heteroatoms selected from the group consisting of nitrogen (N), oxygen (O), and mixtures thereof; R³ is either absent or is hydrogen (H) or a C₁-C₆ alkyl to provide a quaternary amine; R⁴ and R⁵ are either absent or present and when present are either the same or different and are independently an optionally substituted C₁-C₁₀ alkyl or C₂-C₁₀ alkenyl; and n is 0, 1, 2, 3, or 4.

In some embodiments, R¹ and R² are independently an optionally substituted C₁-C₄ alkyl, C₂-C₄ alkenyl, or C₂-C₄ alkynyl. In a preferred embodiment, R¹ and R² are both methyl groups. In another preferred embodiment, R⁴ and R⁵ are both butyl groups. In yet another preferred embodiment, n is 1. In other embodiments, R³ is absent when the pH is above the pKₐ of the cationic lipid and R³ is hydrogen when the pH is below the pKₐ of the cationic lipid such that the amino head group is protonated. In an alternative embodiment, R³ is an optionally substituted C₁-C₄ alkyl to provide a quaternary amine. In further embodiments, R⁴ and R⁵ are independently an optionally substituted C₂-C₆ or C₂-C₄ alkyl or C₂-C₆ or C₂-C₄ alkenyl.

In an alternative embodiment, the cationic lipid of Formula III comprises ester linkages between the amino head group and one or both of the alkyl chains. In some embodiments, the cationic lipid of Formula III forms a salt (preferably a crystalline salt) with one or more anions. In one particular embodiment, the cationic lipid of Formula III is the oxalate (e.g., hemioxalate) salt thereof, which is preferably a crystalline salt.

Although each of the alkyl chains in Formula III contains cis double bonds at positions 6, 9, and 12 (i.e., cis,cis,cis-Δ⁶, Δ⁹, Δ¹²) in an alternative embodiment, one, two, or three of these double bonds in one or both alkyl chains may be in the trans configuration.

In a particular embodiment, the cationic lipid of Formula III has the structure:

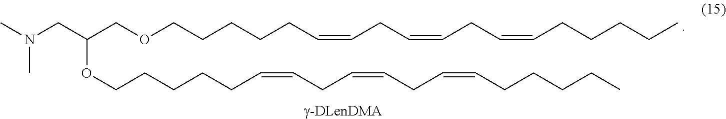 [SMILES: CCCCC/C=C\C/C=C\C/C=C\CCCCCOCC(CN(C)C)OCCCCC/C=C\C/C=C\C/C=C\CCCCC]

The synthesis of cationic lipids such as γ-DLenDMA (15), as well as additional cationic lipids, is described in U.S. Provisional Application No. 61/222,462, entitled “Improved Cationic Lipids and Methods for the Delivery of Nucleic Acids,” filed Jul. 1, 2009, the disclosure of which is herein incorporated by reference in its entirety for all purposes.

The synthesis of cationic lipids such as DLin-M-C3-DMA (“MC3”), as well as additional cationic lipids (e.g., certain analogs of MC3), is described in U.S. Provisional Application No. 61/185,800, entitled “Novel Lipids and Compositions for the Delivery of Therapeutics,” filed Jun. 10, 2009, and U.S. Provisional Application No. 61/287,995, entitled “Methods and Compositions for Delivery of Nucleic Acids,” filed Dec. 18, 2009, the disclosures of which are herein incorporated by reference in their entirety for all purposes.

Examples of other cationic lipids or salts thereof which may be included in the lipid particles include, but are not limited to, cationic lipids such as those described in WO2011/000106, the disclosure of which is herein incorporated by reference in its entirety for all purposes, as well as cationic lipids such as N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), 1,2-dioeylcarbamoyloxy-3-dimethylaminopropane (DO-C-DAP), 1,2-dimyristoleoyl-3-dimethylaminopropane (DMDAP), 1,2-dioleoyl-3-trimethylaminopropane chloride (DOTAP.C1), dilinoleylmethyl-3-dimethylaminopropionate (DLin-M-C2-DMA; also known as DLin-M-K-DMA or DLin-M-DMA), and mixtures thereof. Additional cationic lipids or salts thereof which may be included in the lipid particles are described in U.S. Patent Publication No. 20090023673, the disclosure of which is herein incorporated by reference in its entirety for all purposes.

The synthesis of cationic lipids such as CLinDMA, as well as additional cationic lipids, is described in U.S. Patent Publication No. 20060240554, the disclosure of which is herein incorporated by reference in its entirety for all purposes. The synthesis of cationic lipids such as DLin-C-DAP, DLinDAC, DLinMA, DLinDAP, DLin-S-DMA, DLin-2-DMAP, DLinTMA.C1, DLinTAP.C1, DLinMPZ, DLinAP, DOAP, and DLin-EG-DMA, as well as additional cationic lipids, is described in PCT Publication No. WO 09/086558, the disclosure of which is herein incorporated by reference in its entirety for all purposes. The synthesis of cationic lipids such as DO-C-DAP, DMDAP, DOTAP.C1, DLin-M-C2-DMA, as well as additional cationic lipids, is described in PCT Application No. PCT/US2009/060251, entitled “Improved Amino Lipids and Methods for the Delivery of Nucleic Acids,” filed Oct. 9, 2009, the disclosure of which is incorporated herein by reference in its entirety for all purposes. The synthesis of a number of other cationic lipids and related analogs has been described in U.S. Pat. Nos. 5,208,036; 5,264,618; 5,279,833; 5,283,185; 5,753,613; and 5,785,992; and PCT Publication No. WO 96/10390, the disclosures of which are each herein incorporated by reference in their entirety for all purposes. Additionally, a number of commercial preparations of cationic lipids can be used, such as, e.g., LIPOFECTIN® (including DOTMA and DOPE, available from Invitrogen); LIPOFECTAMINE® (including DOSPA and DOPE, available from Invitrogen); and TRANSFECTAM® (including DOGS, available from Promega Corp.).

In some embodiments, the cationic lipid comprises from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, from about 50 mol % to about 60 mol %, from about 55 mol % to about 65 mol %, or from about 55 mol % to about 70 mol % (or any fraction thereof or range therein) of the total lipid present in the particle. In particular embodiments, the cationic lipid comprises about 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, or 65 mol % (or any fraction thereof) of the total lipid present in the particle.

In other embodiments, the cationic lipid comprises from about 2 mol % to about 60 mol %, from about 5 mol % to about 50 mol %, from about 10 mol % to about 50 mol %, from about 20 mol % to about 50 mol %, from about 20 mol % to about 40 mol %, from about 30 mol % to about 40 mol %, or about 40 mol % (or any fraction thereof or range therein) of the total lipid present in the particle.

Additional percentages and ranges of cationic lipids suitable for use in the lipid particles are described in PCT Publication No. WO 09/127060, U.S. Published Application No. US 2011/0071208, PCT Publication No. WO2011/000106, and U.S. Published Application No. US 2011/0076335, the disclosures of which are herein incorporated by reference in their entirety for all purposes.

It should be understood that the percentage of cationic lipid present in the lipid particles is a target amount, and that the actual amount of cationic lipid present in the formulation may vary, for example, by ±5 mol %. For example, in one exemplary lipid particle formulation, the target amount of cationic lipid is 57.1 mol %, but the actual amount of cationic lipid may be ±5 mol %, ±4 mol %, ±3 mol %, ±2 mol %, 1 mol %, 0.75 mol %, 0.5 mol %, ±0.25 mol %, or ±0.1 mol % of that target amount, with the balance of the formulation being made up of other lipid components (adding up to 100 mol % of total lipids present in the particle; however, one skilled in the art will understand that the total mol % may deviate slightly from 100% due to rounding, for example, 99.9 mol % or 100.1 mol %).

Further examples of cationic lipids useful for inclusion in lipid particles are shown below:

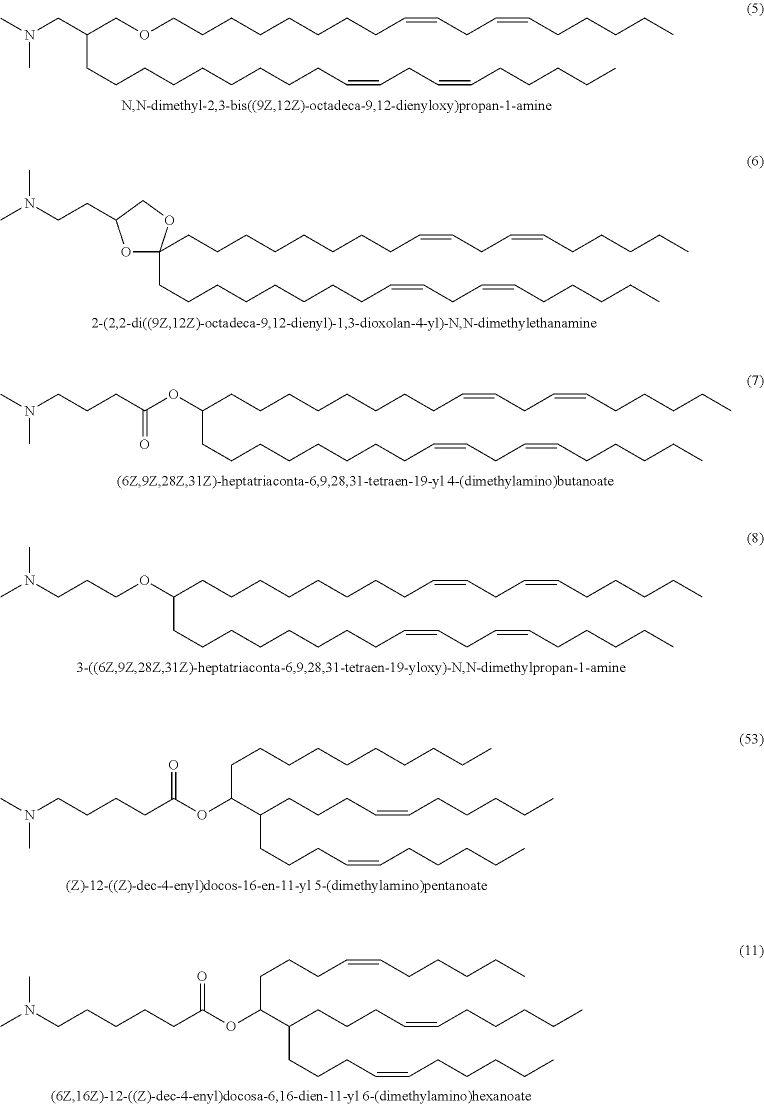

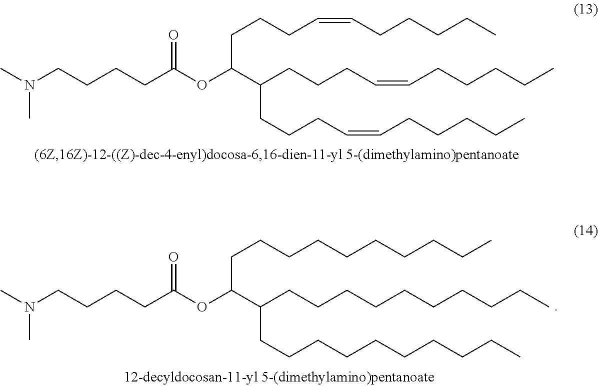

Non-Cationic Lipids

The non-cationic lipids used in the lipid particles can be any of a variety of neutral uncharged, zwitterionic, or anionic lipids capable of producing a stable complex.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In preferred embodiments, the cholesterol derivative is a polar analogue such as cholesteryl-(4′-hydroxy)-butyl ether. The synthesis of cholesteryl-(2′-hydroxy)-ethyl ether is described in PCT Publication No. WO 09/127060, the disclosure of which is herein incorporated by reference in its entirety for all purposes.

In some embodiments, the non-cationic lipid present in the lipid particles comprises or consists of a mixture of one or more phospholipids and cholesterol or a derivative thereof. In other embodiments, the non-cationic lipid present in the lipid particles comprises or consists of one or more phospholipids, e.g., a cholesterol-free lipid particle formulation. In yet other embodiments, the non-cationic lipid present in the lipid particles comprises or consists of cholesterol or a derivative thereof, e.g., a phospholipid-free lipid particle formulation.

Other examples of non-cationic lipids suitable for use include nonphosphorous containing lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid comprises from about 10 mol % to about 60 mol %, from about 20 mol % to about 55 mol %, from about 20 mol % to about 45 mol %, from about 20 mol % to about 40 mol %, from about 25 mol % to about 50 mol %, from about 25 mol % to about 45 mol %, from about 30 mol % to about 50 mol %, from about 30 mol % to about 45 mol %, from about 30 mol % to about 40 mol %, from about 35 mol % to about 45 mol %, from about 37 mol % to about 45 mol %, or about 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipid present in the particle.

In embodiments where the lipid particles contain a mixture of phospholipid and cholesterol or a cholesterol derivative, the mixture may comprise up to about 40 mol %, 45 mol %, 50 mol %, 55 mol %, or 60 mol % of the total lipid present in the particle.

In some embodiments, the phospholipid component in the mixture may comprise from about 2 mol % to about 20 mol %, from about 2 mol % to about 15 mol %, from about 2 mol % to about 12 mol %, from about 4 mol % to about 15 mol %, or from about 4 mol % to about 10 mol % (or any fraction thereof or range therein) of the total lipid present in the particle. In an certain embodiments, the phospholipid component in the mixture comprises from about 5 mol % to about 17 mol %, from about 7 mol % to about 17 mol %, from about 7 mol % to about 15 mol %, from about 8 mol % to about 15 mol %, or about 8 mol %, 9 mol %, 10 mol %, 11 mol %, 12 mol %, 13 mol %, 14 mol %, or 15 mol % (or any fraction thereof or range therein) of the total lipid present in the particle. As a non-limiting example, a lipid particle formulation comprising a mixture of phospholipid and cholesterol may comprise a phospholipid such as DPPC or DSPC at about 7 mol % (or any fraction thereof), e.g., in a mixture with cholesterol or a cholesterol derivative at about 34 mol % (or any fraction thereof) of the total lipid present in the particle. As another non-limiting example, a lipid particle formulation comprising a mixture of phospholipid and cholesterol may comprise a phospholipid such as DPPC or DSPC at about 7 mol % (or any fraction thereof), e.g., in a mixture with cholesterol or a cholesterol derivative at about 32 mol % (or any fraction thereof) of the total lipid present in the particle.

By way of further example, a lipid formulation useful has a lipid to drug (e.g., siRNA) ratio of about 10:1 (e.g., a lipid:drug ratio of from 9.5:1 to 11:1, or from 9.9:1 to 11:1, or from 10:1 to 10.9:1). In certain other embodiments, a lipid formulation useful has a lipid to drug (e.g., siRNA) ratio of about 9:1 (e.g., a lipid:drug ratio of from 8.5:1 to 10:1, or from 8.9:1 to 10:1, or from 9:1 to 9.9:1, including 9.1:1, 9.2:1, 9.3:1, 9.4:1, 9.5:1, 9.6:1, 9.7:1, and 9.8:1).

In other embodiments, the cholesterol component in the mixture may comprise from about 25 mol % to about 45 mol %, from about 25 mol % to about 40 mol %, from about 30 mol % to about 45 mol %, from about 30 mol % to about 40 mol %, from about 27 mol % to about 37 mol %, from about 25 mol % to about 30 mol %, or from about 35 mol % to about 40 mol % (or any fraction thereof or range therein) of the total lipid present in the particle. In certain preferred embodiments, the cholesterol component in the mixture comprises from about 25 mol % to about 35 mol %, from about 27 mol % to about 35 mol %, from about 29 mol % to about 35 mol %, from about 30 mol % to about 35 mol %, from about 30 mol % to about 34 mol %, from about 31 mol % to about 33 mol %, or about 30 mol %, 31 mol %, 32 mol %, 33 mol %, 34 mol %, or 35 mol % (or any fraction thereof or range therein) of the total lipid present in the particle.

In embodiments where the lipid particles are phospholipid-free, the cholesterol or derivative thereof may comprise up to about 25 mol %, 30 mol %, 35 mol %, 40 mol %, 45 mol %, 50 mol %, 55 mol %, or 60 mol % of the total lipid present in the particle.

In some embodiments, the cholesterol or derivative thereof in the phospholipid-free lipid particle formulation may comprise from about 25 mol % to about 45 mol %, from about 25 mol % to about 40 mol %, from about 30 mol % to about 45 mol %, from about 30 mol % to about 40 mol %, from about 31 mol % to about 39 mol %, from about 32 mol % to about 38 mol %, from about 33 mol % to about 37 mol %, from about 35 mol % to about 45 mol %, from about 30 mol % to about 35 mol %, from about 35 mol % to about 40 mol %, or about 30 mol %, 31 mol %, 32 mol %, 33 mol %, 34 mol %, 35 mol %, 36 mol %, 37 mol %, 38 mol %, 39 mol %, or 40 mol % (or any fraction thereof or range therein) of the total lipid present in the particle. As a non-limiting example, a lipid particle formulation may comprise cholesterol at about 37 mol % (or any fraction thereof) of the total lipid present in the particle. As another non-limiting example, a lipid particle formulation may comprise cholesterol at about 35 mol % (or any fraction thereof) of the total lipid present in the particle.

In other embodiments, the non-cationic lipid comprises from about 5 mol % to about 90 mol %, from about 10 mol % to about 85 mol %, from about 20 mol % to about 80 mol %, about 10 mol % (e.g., phospholipid only), or about 60 mol % (e.g., phospholipid and cholesterol or derivative thereof) (or any fraction thereof or range therein) of the total lipid present in the particle.

Additional percentages and ranges of non-cationic lipids suitable for use in the lipid particles are described in PCT Publication No. WO 09/127060, U.S. Published Application No. US 2011/0071208, PCT Publication No. WO2011/000106, and U.S. Published Application No. US 2011/0076335, the disclosures of which are herein incorporated by reference in their entirety for all purposes.

It should be understood that the percentage of non-cationic lipid present in the lipid particles is a target amount, and that the actual amount of non-cationic lipid present in the formulation may vary, for example, by ±5 mol %, ±4 mol %, ±3 mol %, ±2 mol %, ±1 mol %, ±0.75 mol %, 0.5 mol %, 0.25 mol %, or ±0.1 mol %.

Lipid Conjugates

In addition to cationic and non-cationic lipids, the lipid particles may further comprise a lipid conjugate. The conjugated lipid is useful in that it prevents the aggregation of particles. Suitable conjugated lipids include, but are not limited to, PEG-lipid conjugates, POZ-lipid conjugates, ATTA-lipid conjugates, cationic-polymer-lipid conjugates (CPLs), and mixtures thereof. In certain embodiments, the particles comprise either a PEG-lipid conjugate or an ATTA-lipid conjugate together with a CPL.

In a preferred embodiment, the lipid conjugate is a PEG-lipid. Examples of PEG-lipids include, but are not limited to, PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides as described in, e.g., U.S. Pat. No. 5,885,613, PEG conjugated to cholesterol or a derivative thereof, and mixtures thereof. The disclosures of these patent documents are herein incorporated by reference in their entirety for all purposes.

Additional PEG-lipids suitable for use include, without limitation, mPEG2000-1,2-di-O-alkyl-sn3-carbomoylglyceride (PEG-C-DOMG). The synthesis of PEG-C-DOMG is described in PCT Publication No. WO 09/086558, the disclosure of which is herein incorporated by reference in its entirety for all purposes. Yet additional suitable PEG-lipid conjugates include, without limitation, 1-[8′-(1,2-dimyristoyl-3-propanoxy)-carboxamido-3′,6′-dioxaoctanyl]carbamoyl-w-methyl-poly(ethylene glycol) (2KPEG-DMG). The synthesis of 2KPEG-DMG is described in U.S. Pat. No. 7,404,969, the disclosure of which is herein incorporated by reference in its entirety for all purposes.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, but are not limited to, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH2, etc.). Other PEGs such as those described in U.S. Pat. Nos. 6,774,180 and 7,053,150 (e.g., mPEG (20 KDa) amine) are also useful for preparing the PEG-lipid conjugates. The disclosures of these patents are herein incorporated by reference in their entirety for all purposes. In addition, monomethoxypolyethyleneglycol-acetic acid (MePEG-CH₂COOH) is particularly useful for preparing PEG-lipid conjugates including, e.g., PEG-DAA conjugates.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In preferred embodiments, the PEG moiety has an average molecular weight of about 2,000 daltons or about 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to the lipid. Suitable ester containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available, or can be isolated or synthesized using conventional techniques known to those of skill in the art. Phosphatidyl-ethanolamines containing saturated or unsaturated fatty acids with carbon chain lengths in the range of C₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

The term “ATTA” or “polyamide” includes, without limitation, compounds described in U.S. Pat. Nos. 6,320,017 and 6,586,559, the disclosures of which are herein incorporated by reference in their entirety for all purposes. These compounds include a compound having the formula:

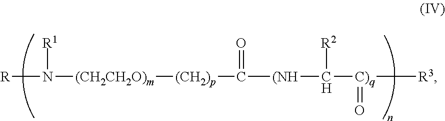

wherein R is a member selected from the group consisting of hydrogen, alkyl and acyl; R¹ is a member selected from the group consisting of hydrogen and alkyl; or optionally, R and Wand the nitrogen to which they are bound form an azido moiety; R² is a member of the group selected from hydrogen, optionally substituted alkyl, optionally substituted aryl and a side chain of an amino acid; R³ is a member selected from the group consisting of hydrogen, halogen, hydroxy, alkoxy, mercapto, hydrazino, amino and NR⁴R⁵, wherein R⁴ and R⁵ are independently hydrogen or alkyl; n is 4 to 80; m is 2 to 6; p is 1 to 4; and q is 0 or 1. It will be apparent to those of skill in the art that other polyamides can be.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (C₁₈), and icosoyl (C₂₀). In preferred embodiments, R¹ and R² are the same, i.e., R¹ and R² are both myristoyl (i.e., dimyristoyl), R¹ and R² are both stearoyl (i.e., distearoyl), etc. Diacylglycerols have the following general formula:

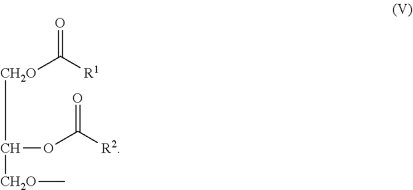

The term “dialkyloxypropyl” or “DAA” includes a compound having 2 alkyl chains, R¹ and R², both of which have independently between 2 and 30 carbons. The alkyl groups can be saturated or have varying degrees of unsaturation. Dialkyloxypropyls have the following general formula:

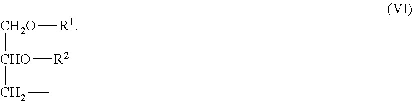

In a preferred embodiment, the PEG-lipid is a PEG-DAA conjugate having the following formula:

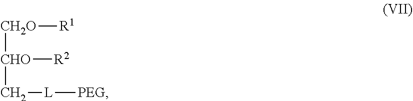

wherein R¹ and R² are independently selected and are long-chain alkyl groups having from about 10 to about 22 carbon atoms; PEG is a polyethyleneglycol; and L is a non-ester containing linker moiety or an ester containing linker moiety as described above. The long-chain alkyl groups can be saturated or unsaturated. Suitable alkyl groups include, but are not limited to, decyl (C₁₀), lauryl (C₁₂), myristyl (C₁₄), palmityl (C₁₆), stearyl (C₁₈), and icosyl (C₂₀). In preferred embodiments, R¹ and R² are the same, i.e., R¹ and R² are both myristyl (i.e., dimyristyl), R¹ and R² are both stearyl (i.e., distearyl), etc.

In Formula VII above, the PEG has an average molecular weight ranging from about 550 daltons to about 10,000 daltons. In certain instances, the PEG has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In preferred embodiments, the PEG has an average molecular weight of about 2,000 daltons or about 750 daltons. The PEG can be optionally substituted with alkyl, alkoxy, acyl, or aryl groups. In certain embodiments, the terminal hydroxyl group is substituted with a methoxy or methyl group.

In a preferred embodiment, “L” is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a disulphide linker moiety, a succinamidyl linker moiety, and combinations thereof. In a preferred embodiment, the non-ester containing linker moiety is a carbamate linker moiety (i.e., a PEG-C-DAA conjugate). In another preferred embodiment, the non-ester containing linker moiety is an amido linker moiety (i.e., a PEG-A-DAA conjugate). In yet another preferred embodiment, the non-ester containing linker moiety is a succinamidyl linker moiety (i.e., a PEG-S-DAA conjugate).

In particular embodiments, the PEG-lipid conjugate is selected from:

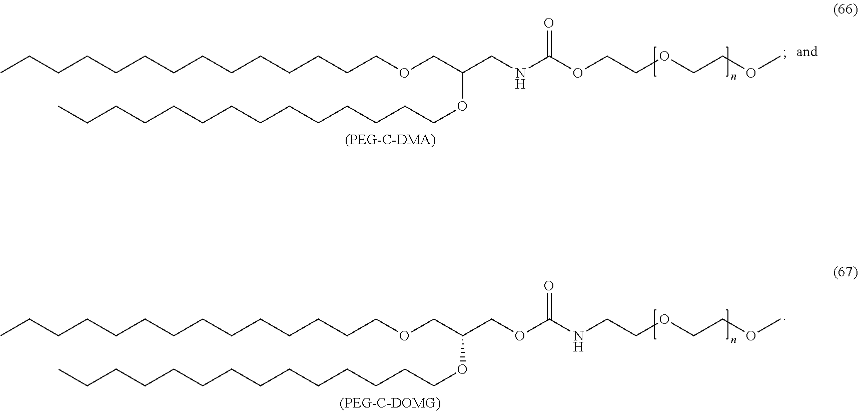

The PEG-DAA conjugates are synthesized using standard techniques and reagents known to those of skill in the art. It will be recognized that the PEG-DAA conjugates will contain various amide, amine, ether, thio, carbamate, and urea linkages. Those of skill in the art will recognize that methods and reagents for forming these bonds are well known and readily available. See, e.g., March, ADVANCED ORGANIC CHEMISTRY (Wiley 1992); Larock, COMPREHENSIVE ORGANIC TRANSFORMATIONS (VCH 1989); and Furniss, VOGEL'S TEXTBOOK OF PRACTICAL ORGANIC CHEMISTRY, 5th ed. (Longman 1989). It will also be appreciated that any functional groups present may require protection and deprotection at different points in the synthesis of the PEG-DAA conjugates. Those of skill in the art will recognize that such techniques are well known. See, e.g., Green and Wuts, PROTECTIVE GROUPS IN ORGANIC SYNTHESIS (Wiley 1991).

Preferably, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 or about 2,000 daltons. In one particularly preferred embodiment, the PEG-lipid conjugate comprises PEG2000-C-DMA, wherein the “2000” denotes the average molecular weight of the PEG, the “C” denotes a carbamate linker moiety, and the “DMA” denotes dimyristyloxypropyl. In another particularly preferred embodiment, the PEG-lipid conjugate comprises PEG750-C-DMA, wherein the “750” denotes the average molecular weight of the PEG, the “C” denotes a carbamate linker moiety, and the “DMA” denotes dimyristyloxypropyl. In particular embodiments, the terminal hydroxyl group of the PEG is substituted with a methyl group. Those of skill in the art will readily appreciate that other dialkyloxypropyls can be used in the PEG-DAA conjugates.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the lipid particles can further comprise cationic poly(ethylene glycol) (PEG) lipids or CPLs (see, e.g., Chen et al., Bioconj. Chem., 11:433-437 (2000); U.S. Pat. No. 6,852,334; PCT Publication No. WO 00/62813, the disclosures of which are herein incorporated by reference in their entirety for all purposes).

Suitable CPLs include compounds of Formula VIII:

A-W-Y (VIII),

wherein A, W, and Y are as described below.

With reference to Formula VIII, “A” is a lipid moiety such as an amphipathic lipid, a neutral lipid, or a hydrophobic lipid that acts as a lipid anchor. Suitable lipid examples include, but are not limited to, diacylglycerolyls, dialkylglycerolyls, N-N-dialkylaminos, 1,2-diacyloxy-3-aminopropanes, and 1,2-dialkyl-3-aminopropanes.

“W” is a polymer or an oligomer such as a hydrophilic polymer or oligomer. Preferably, the hydrophilic polymer is a biocompatable polymer that is nonimmunogenic or possesses low inherent immunogenicity. Alternatively, the hydrophilic polymer can be weakly antigenic if used with appropriate adjuvants. Suitable nonimmunogenic polymers include, but are not limited to, PEG, polyamides, polylactic acid, polyglycolic acid, polylactic acid/polyglycolic acid copolymers, and combinations thereof. In a preferred embodiment, the polymer has a molecular weight of from about 250 to about 7,000 daltons.

“Y” is a polycationic moiety. The term polycationic moiety refers to a compound, derivative, or functional group having a positive charge, preferably at least 2 positive charges at a selected pH, preferably physiological pH. Suitable polycationic moieties include basic amino acids and their derivatives such as arginine, asparagine, glutamine, lysine, and histidine; spermine; spermidine; cationic dendrimers; polyamines; polyamine sugars; and amino polysaccharides. The polycationic moieties can be linear, such as linear tetralysine, branched or dendrimeric in structure. Polycationic moieties have between about 2 to about 15 positive charges, preferably between about 2 to about 12 positive charges, and more preferably between about 2 to about 8 positive charges at selected pH values. The selection of which polycationic moiety to employ may be determined by the type of particle application which is desired.

The charges on the polycationic moieties can be either distributed around the entire particle moiety, or alternatively, they can be a discrete concentration of charge density in one particular area of the particle moiety e.g., a charge spike. If the charge density is distributed on the particle, the charge density can be equally distributed or unequally distributed. All variations of charge distribution of the polycationic moiety are encompassed.

The lipid “A” and the nonimmunogenic polymer “W” can be attached by various methods and preferably by covalent attachment. Methods known to those of skill in the art can be used for the covalent attachment of “A” and “W.” Suitable linkages include, but are not limited to, amide, amine, carboxyl, carbonate, carbamate, ester, and hydrazone linkages. It will be apparent to those skilled in the art that “A” and “W” must have complementary functional groups to effectuate the linkage. The reaction of these two groups, one on the lipid and the other on the polymer, will provide the desired linkage. For example, when the lipid is a diacylglycerol and the terminal hydroxyl is activated, for instance with NHS and DCC, to form an active ester, and is then reacted with a polymer which contains an amino group, such as with a polyamide (see, e.g., U.S. Pat. Nos. 6,320,017 and 6,586,559, the disclosures of which are herein incorporated by reference in their entirety for all purposes), an amide bond will form between the two groups.

In certain instances, the polycationic moiety can have a ligand attached, such as a targeting ligand or a chelating moiety for complexing calcium. Preferably, after the ligand is attached, the cationic moiety maintains a positive charge. In certain instances, the ligand that is attached has a positive charge. Suitable ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

In some embodiments, the lipid conjugate (e.g., PEG-lipid) comprises from about 0.1 mol % to about 3 mol %, from about 0.5 mol % to about 3 mol %, or about 0.6 mol %, 0.7 mol %, 0.8 mol %, 0.9 mol %, 1.0 mol %, 1.1 mol %, 1.2 mol %, 1.3 mol %, 1.4 mol %, 1.5 mol %, 1.6 mol %, 1.7 mol %, 1.8 mol %, 1.9 mol %, 2.0 mol %, 2.1 mol %, 2.2 mol %, 2.3 mol %, 2.4 mol %, 2.5 mol %, 2.6 mol %, 2.7 mol %, 2.8 mol %, 2.9 mol % or 3 mol % (or any fraction thereof or range therein) of the total lipid present in the particle.

In other embodiments, the lipid conjugate (e.g., PEG-lipid) comprises from about 0 mol % to about 20 mol %, from about 0.5 mol % to about 20 mol %, from about 2 mol % to about 20 mol %, from about 1.5 mol % to about 18 mol %, from about 2 mol % to about 15 mol %, from about 4 mol % to about 15 mol %, from about 2 mol % to about 12 mol %, from about 5 mol % to about 12 mol %, or about 2 mol % (or any fraction thereof or range therein) of the total lipid present in the particle.

In further embodiments, the lipid conjugate (e.g., PEG-lipid) comprises from about 4 mol % to about 10 mol %, from about 5 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle.

It should be understood that the percentage of lipid conjugate present in the lipid particles is a target amount, and that the actual amount of lipid conjugate present in the formulation may vary, for example, by ±5 mol %, ±4 mol %, ±3 mol %, ±2 mol %, ±1 mol %, ±0.75 mol %, 0.5 mol %, 0.25 mol %, or ±0.1 mol %.

Additional percentages and ranges of lipid conjugates suitable for use in the lipid particles are described in PCT Publication No. WO 09/127060, U.S. Published Application No. US 2011/0071208, PCT Publication No. WO2011/000106, and U.S. Published Application No. US 2011/0076335, the disclosures of which are herein incorporated by reference in their entirety for all purposes.

One of ordinary skill in the art will appreciate that the concentration of the lipid conjugate can be varied depending on the lipid conjugate employed and the rate at which the lipid particle is to become fusogenic.

By controlling the composition and concentration of the lipid conjugate, one can control the rate at which the lipid conjugate exchanges out of the lipid particle and, in turn, the rate at which the lipid particle becomes fusogenic. For instance, when a PEG-DAA conjugate is used as the lipid conjugate, the rate at which the lipid particle becomes fusogenic can be varied, for example, by varying the concentration of the lipid conjugate, by varying the molecular weight of the PEG, or by varying the chain length and degree of saturation of the alkyl groups on the PEG-DAA conjugate. In addition, other variables including, for example, pH, temperature, ionic strength, etc. can be used to vary and/or control the rate at which the lipid particle becomes fusogenic. Other methods which can be used to control the rate at which the lipid particle becomes fusogenic will become apparent to those of skill in the art upon reading this disclosure. Also, by controlling the composition and concentration of the lipid conjugate, one can control the lipid particle size.

Additional Carrier Systems

Non-limiting examples of additional lipid-based carrier systems suitable for use include lipoplexes (see, e.g., U.S. Patent Publication No. 20030203865; and Zhang et al., J. Control Release, 100:165-180 (2004)), pH-sensitive lipoplexes (see, e.g., U.S. Patent Publication No. 20020192275), reversibly masked lipoplexes (see, e.g., U.S. Patent Publication Nos. 20030180950), cationic lipid-based compositions (see, e.g., U.S. Pat. No. 6,756,054; and U.S. Patent Publication No. 20050234232), cationic liposomes (see, e.g., U.S. Patent Publication Nos. 20030229040, 20020160038, and 20020012998; U.S. Pat. No. 5,908,635; and PCT Publication No. WO 01/72283), anionic liposomes (see, e.g., U.S. Patent Publication No. 20030026831), pH-sensitive liposomes (see, e.g., U.S. Patent Publication No. 20020192274; and AU 2003210303), antibody-coated liposomes (see, e.g., U.S. Patent Publication No. 20030108597; and PCT Publication No. WO 00/50008), cell-type specific liposomes (see, e.g., U.S. Patent Publication No. 20030198664), liposomes containing nucleic acid and peptides (see, e.g., U.S. Pat. No. 6,207,456), liposomes containing lipids derivatized with releasable hydrophilic polymers (see, e.g., U.S. Patent Publication No. 20030031704), lipid-entrapped nucleic acid (see, e.g., PCT Publication Nos. WO 03/057190 and WO 03/059322), lipid-encapsulated nucleic acid (see, e.g., U.S. Patent Publication No. 20030129221; and U.S. Pat. No. 5,756,122), other liposomal compositions (see, e.g., U.S. Patent Publication Nos. 20030035829 and 20030072794; and U.S. Pat. No. 6,200,599), stabilized mixtures of liposomes and emulsions (see, e.g., EP1304160), emulsion compositions (see, e.g., U.S. Pat. No. 6,747,014), and nucleic acid micro-emulsions (see, e.g., U.S. Patent Publication No. 20050037086).

Examples of polymer-based carrier systems suitable for use include, but are not limited to, cationic polymer-nucleic acid complexes (i.e., polyplexes). To form a polyplex, a nucleic acid (e.g., a siRNA molecule, such as an siRNA molecule described in Table A or the Examples herein, including the specific combinations of siRNA molecules described herein, e.g., the two-way and three-way combinations of siRNA molecules) is typically complexed with a cationic polymer having a linear, branched, star, or dendritic polymeric structure that condenses the nucleic acid into positively charged particles capable of interacting with anionic proteoglycans at the cell surface and entering cells by endocytosis. In some embodiments, the polyplex comprises nucleic acid (e.g., a siRNA molecule, such as an siRNA molecule described in Table A or the Examples herein, including the specific combinations of siRNA molecules described herein, e.g., the two-way and three-way combinations of siRNA molecules) complexed with a cationic polymer such as polyethylenimine (PEI) (see, e.g., U.S. Pat. No. 6,013,240; commercially available from Qbiogene, Inc. (Carlsbad, Calif.) as In vivo jetPEI™, a linear form of PEI), polypropylenimine (PPI), polyvinylpyrrolidone (PVP), poly-L-lysine (PLL), diethylaminoethyl (DEAE)-dextran, poly(β-amino ester) (PAE) polymers (see, e.g., Lynn et al., J. Am. Chem. Soc., 123:8155-8156 (2001)), chitosan, polyamidoamine (PAMAM) dendrimers (see, e.g., Kukowska-Latallo et al., Proc. Natl. Acad. Sci. USA, 93:4897-4902 (1996)), porphyrin (see, e.g., U.S. Pat. No. 6,620,805), polyvinylether (see, e.g., U.S. Patent Publication No. 20040156909), polycyclic amidinium (see, e.g., U.S. Patent Publication No. 20030220289), other polymers comprising primary amine, imine, guanidine, and/or imidazole groups (see, e.g., U.S. Pat. No. 6,013,240; PCT Publication No. WO/9602655; PCT Publication No. WO95/21931; Zhang et al., J. Control Release, 100:165-180 (2004); and Tiera et al., Curr. Gene Ther., 6:59-71 (2006)), and a mixture thereof. In other embodiments, the polyplex comprises cationic polymer-nucleic acid complexes as described in U.S. Patent Publication Nos. 20060211643, 20050222064, 20030125281, and 20030185890, and PCT Publication No. WO 03/066069; biodegradable poly(β-amino ester) polymer-nucleic acid complexes as described in U.S. Patent Publication No. 20040071654; microparticles containing polymeric matrices as described in U.S. Patent Publication No. 20040142475; other microparticle compositions as described in U.S. Patent Publication No. 20030157030; condensed nucleic acid complexes as described in U.S. Patent Publication No. 20050123600; and nanocapsule and microcapsule compositions as described in AU 2002358514 and PCT Publication No. WO 02/096551.

In certain instances, the siRNA may be complexed with cyclodextrin or a polymer thereof. Non-limiting examples of cyclodextrin-based carrier systems include the cyclodextrin-modified polymer-nucleic acid complexes described in U.S. Patent Publication No. 20040087024; the linear cyclodextrin copolymer-nucleic acid complexes described in U.S. Pat. Nos. 6,509,323, 6,884,789, and 7,091,192; and the cyclodextrin polymer-complexing agent-nucleic acid complexes described in U.S. Pat. No. 7,018,609. In certain other instances, the siRNA may be complexed with a peptide or polypeptide. An example of a protein-based carrier system includes, but is not limited to, the cationic oligopeptide-nucleic acid complex described in PCT Publication No. WO95/21931.

Preparation of Lipid Particles

The nucleic acid-lipid particles, in which a nucleic acid (e.g., a siRNA as described in Table A or the Examples herein, including the specific combinations of siRNA molecules described herein, e.g., the two-way and three-way combinations of siRNA molecules) is entrapped within the lipid portion of the particle and is protected from degradation, can be formed by any method known in the art including, but not limited to, a continuous mixing method, a direct dilution process, and an in-line dilution process.

In particular embodiments, the cationic lipids may comprise lipids of Formula I-III or salts thereof, alone or in combination with other cationic lipids. In other embodiments, the non-cationic lipids are egg sphingomyelin (ESM), distearoylphosphatidylcholine (DSPC), dioleoylphosphatidylcholine (DOPC), 1-palmitoyl-2-oleoyl-phosphatidylcholine (POPC), dipalmitoyl-phosphatidylcholine (DPPC), monomethyl-phosphatidylethanolamine, dimethyl-phosphatidylethanolamine, 14:0 PE (1,2-dimyristoyl-phosphatidylethanolamine (DMPE)), 16:0 PE (1,2-dipalmitoyl-phosphatidylethanolamine (DPPE)), 18:0 PE (1,2-distearoyl-phosphatidylethanolamine (DSPE)), 18:1 PE (1,2-dioleoyl-phosphatidylethanolamine (DOPE)), 18:1 trans PE (1,2-dielaidoyl-phosphatidylethanolamine (DEPE)), 18:0-18:1 PE (1-stearoyl-2-oleoyl-phosphatidylethanolamine (SOPE)), 16:0-18:1 PE (1-palmitoyl-2-oleoyl-phosphatidylethanolamine (POPE)), polyethylene glycol-based polymers (e.g., PEG 2000, PEG 5000, PEG-modified diacylglycerols, or PEG-modified dialkyloxypropyls), cholesterol, derivatives thereof, or combinations thereof.

In certain embodiments, the nucleic acid-lipid particles produced via a continuous mixing method, e.g., a process that includes providing an aqueous solution comprising a siRNA in a first reservoir, providing an organic lipid solution in a second reservoir (wherein the lipids present in the organic lipid solution are solubilized in an organic solvent, e.g., a lower alkanol such as ethanol), and mixing the aqueous solution with the organic lipid solution such that the organic lipid solution mixes with the aqueous solution so as to substantially instantaneously produce a lipid vesicle (e.g., liposome) encapsulating the siRNA within the lipid vesicle. This process and the apparatus for carrying out this process are described in detail in U.S. Patent Publication No. 20040142025, the disclosure of which is herein incorporated by reference in its entirety for all purposes.

The action of continuously introducing lipid and buffer solutions into a mixing environment, such as in a mixing chamber, causes a continuous dilution of the lipid solution with the buffer solution, thereby producing a lipid vesicle substantially instantaneously upon mixing. As used herein, the phrase “continuously diluting a lipid solution with a buffer solution” (and variations) generally means that the lipid solution is diluted sufficiently rapidly in a hydration process with sufficient force to effectuate vesicle generation. By mixing the aqueous solution comprising a nucleic acid with the organic lipid solution, the organic lipid solution undergoes a continuous stepwise dilution in the presence of the buffer solution (i.e., aqueous solution) to produce a nucleic acid-lipid particle.

The nucleic acid-lipid particles formed using the continuous mixing method typically have a size of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm (or any fraction thereof or range therein). The particles thus formed do not aggregate and are optionally sized to achieve a uniform particle size.

In another embodiment, the nucleic acid-lipid particles produced via a direct dilution process that includes forming a lipid vesicle (e.g., liposome) solution and immediately and directly introducing the lipid vesicle solution into a collection vessel containing a controlled amount of dilution buffer. In preferred aspects, the collection vessel includes one or more elements configured to stir the contents of the collection vessel to facilitate dilution. In one aspect, the amount of dilution buffer present in the collection vessel is substantially equal to the volume of lipid vesicle solution introduced thereto. As a non-limiting example, a lipid vesicle solution in 45% ethanol when introduced into the collection vessel containing an equal volume of dilution buffer will advantageously yield smaller particles.

In yet another embodiment, the nucleic acid-lipid particles produced via an in-line dilution process in which a third reservoir containing dilution buffer is fluidly coupled to a second mixing region. In this embodiment, the lipid vesicle (e.g., liposome) solution formed in a first mixing region is immediately and directly mixed with dilution buffer in the second mixing region. In preferred aspects, the second mixing region includes a T-connector arranged so that the lipid vesicle solution and the dilution buffer flows meet as opposing 180° flows; however, connectors providing shallower angles can be used, e.g., from about 27° to about 180° (e.g., about 90°). A pump mechanism delivers a controllable flow of buffer to the second mixing region. In one aspect, the flow rate of dilution buffer provided to the second mixing region is controlled to be substantially equal to the flow rate of lipid vesicle solution introduced thereto from the first mixing region. This embodiment advantageously allows for more control of the flow of dilution buffer mixing with the lipid vesicle solution in the second mixing region, and therefore also the concentration of lipid vesicle solution in buffer throughout the second mixing process. Such control of the dilution buffer flow rate advantageously allows for small particle size formation at reduced concentrations.

These processes and the apparatuses for carrying out these direct dilution and in-line dilution processes are described in detail in U.S. Patent Publication No. 20070042031, the disclosure of which is herein incorporated by reference in its entirety for all purposes.

The nucleic acid-lipid particles formed using the direct dilution and in-line dilution processes typically have a size of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, less than about 120 nm, 110 nm, 100 nm, 90 nm, or 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm (or any fraction thereof or range therein). The particles thus formed do not aggregate and are optionally sized to achieve a uniform particle size.

The lipid particles can be sized by any of the methods available for sizing liposomes. The sizing may be conducted in order to achieve a desired size range and relatively narrow distribution of particle sizes.

Several techniques are available for sizing the particles to a desired size. One sizing method, used for liposomes and equally applicable to the present particles, is described in U.S. Pat. No. 4,737,323, the disclosure of which is herein incorporated by reference in its entirety for all purposes. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and about 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In some embodiments, the nucleic acids present in the particles (e.g., the siRNA molecules) are precondensed as described in, e.g., U.S. patent application Ser. No. 09/744,103, the disclosure of which is herein incorporated by reference in its entirety for all purposes.

In other embodiments, the methods may further comprise adding non-lipid polycations which are useful to effect the lipofection of cells using the present compositions. Examples of suitable non-lipid polycations include, hexadimethrine bromide (sold under the brand name POLYBRENE®, from Aldrich Chemical Co., Milwaukee, Wis., USA) or other salts of hexadimethrine. Other suitable polycations include, for example, salts of poly-L-ornithine, poly-L-arginine, poly-L-lysine, poly-D-lysine, polyallylamine, and polyethyleneimine. Addition of these salts is preferably after the particles have been formed.

In some embodiments, the nucleic acid (e.g., siRNA) to lipid ratios (mass/mass ratios) in a formed nucleic acid-lipid particle will range from about 0.01 to about 0.2, from about 0.05 to about 0.2, from about 0.02 to about 0.1, from about 0.03 to about 0.1, or from about 0.01 to about 0.08. The ratio of the starting materials (input) also falls within this range. In other embodiments, the particle preparation uses about 400 μg nucleic acid per 10 mg total lipid or a nucleic acid to lipid mass ratio of about 0.01 to about 0.08 and, more preferably, about 0.04, which corresponds to 1.25 mg of total lipid per 50 μg of nucleic acid. In other preferred embodiments, the particle has a nucleic acid:lipid mass ratio of about 0.08.

In other embodiments, the lipid to nucleic acid (e.g., siRNA) ratios (mass/mass ratios) in a formed nucleic acid-lipid particle will range from about 1 (1:1) to about 100 (100:1), from about 5 (5:1) to about 100 (100:1), from about 1 (1:1) to about 50 (50:1), from about 2 (2:1) to about 50 (50:1), from about 3 (3:1) to about 50 (50:1), from about 4 (4:1) to about 50 (50:1), from about 5 (5:1) to about 50 (50:1), from about 1 (1:1) to about 25 (25:1), from about 2 (2:1) to about 25 (25:1), from about 3 (3:1) to about 25 (25:1), from about 4 (4:1) to about 25 (25:1), from about 5 (5:1) to about 25 (25:1), from about 5 (5:1) to about 20 (20:1), from about 5 (5:1) to about 15 (15:1), from about 5 (5:1) to about 10 (10:1), or about 5 (5:1), 6 (6:1), 7 (7:1), 8 (8:1), 9 (9:1), 10 (10:1), 11 (11:1), 12 (12:1), 13 (13:1), 14 (14:1), 15 (15:1), 16 (16:1), 17 (17:1), 18 (18:1), 19 (19:1), 20 (20:1), 21 (21:1), 22 (22:1), 23 (23:1), 24 (24:1), or 25 (25:1), or any fraction thereof or range therein. The ratio of the starting materials (input) also falls within this range.

As previously discussed, the conjugated lipid may further include a CPL. A variety of general methods for making lipid particle-CPLs (CPL-containing lipid particles) are discussed herein. Two general techniques include the “post-insertion” technique, that is, insertion of a CPL into, for example, a pre-formed lipid particle, and the “standard” technique, wherein the CPL is included in the lipid mixture during, for example, the lipid particle formation steps. The post-insertion technique results in lipid particles having CPLs mainly in the external face of the lipid particle bilayer membrane, whereas standard techniques provide lipid particles having CPLs on both internal and external faces. The method is especially useful for vesicles made from phospholipids (which can contain cholesterol) and also for vesicles containing PEG-lipids (such as PEG-DAAs and PEG-DAGs). Methods of making lipid particle-CPLs are taught, for example, in U.S. Pat. Nos. 5,705,385; 6,586,410; 5,981,501; 6,534,484; and 6,852,334; U.S. Patent Publication No. 20020072121; and PCT Publication No. WO 00/62813, the disclosures of which are herein incorporated by reference in their entirety for all purposes.

Administration of Lipid Particles

The lipid particles (e.g., a nucleic-acid lipid particle) can be adsorbed to almost any cell type with which they are mixed or contacted. Once adsorbed, the particles can either be endocytosed by a portion of the cells, exchange lipids with cell membranes, or fuse with the cells. Transfer or incorporation of the siRNA portion of the particle can take place via any one of these pathways. In particular, when fusion takes place, the particle membrane is integrated into the cell membrane and the contents of the particle combine with the intracellular fluid.

The lipid particles (e.g., nucleic acid-lipid particles) can be administered either alone or in a mixture with a pharmaceutically acceptable carrier (e.g., physiological saline or phosphate buffer) selected in accordance with the route of administration and standard pharmaceutical practice. Generally, normal buffered saline (e.g., 135-150 mM NaCl) will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. Additional suitable carriers are described in, e.g., REMINGTON'S PHARMACEUTICAL SCIENCES, Mack Publishing Company, Philadelphia, Pa., 17th ed. (1985). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The phrase “pharmaceutically acceptable” refers to molecular entities and compositions that do not produce an allergic or similar untoward reaction when administered to a human.

The pharmaceutically acceptable carrier is generally added following lipid particle formation. Thus, after the lipid particle is formed, the particle can be diluted into pharmaceutically acceptable carriers such as normal buffered saline.

The concentration of particles in the pharmaceutical formulations can vary widely, i.e., from less than about 0.05%, usually at or at least about 2 to 5%, to as much as about 10 to 90% by weight, and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

The pharmaceutical compositions may be sterilized by conventional, well-known sterilization techniques. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol, and water-soluble iron-specific chelators, such as ferrioxamine, are suitable.

In Vivo Administration

Systemic delivery for in vivo therapy, e.g., delivery of a siRNA molecule described herein, such as an siRNA described in Table A or the Examples herein, including the specific combinations of siRNA molecules described herein, e.g., the two-way and three-way combinations of siRNA molecules, to a distal target cell via body systems such as the circulation, has been achieved using nucleic acid-lipid particles such as those described in PCT Publication Nos. WO 05/007196, WO 05/121348, WO 05/120152, and WO 04/002453, the disclosures of which are herein incorporated by reference in their entirety for all purposes.

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol., 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993). Still other methods of administering lipid-based therapeutics are described in, for example, U.S. Pat. Nos. 3,993,754; 4,145,410; 4,235,871; 4,224,179; 4,522,803; and 4,588,578. The lipid particles can be administered by direct injection at the site of disease or by injection at a site distal from the site of disease (see, e.g., Culver, HUMAN GENE THERAPY, MaryAnn Liebert, Inc., Publishers, New York. pp. 70-71(1994)). The disclosures of the above-described references are herein incorporated by reference in their entirety for all purposes.

In embodiments where the lipid particles are administered intravenously, at least about 5%, 10%, 15%, 20%, or 25% of the total injected dose of the particles is present in plasma about 8, 12, 24, 36, or 48 hours after injection. In other embodiments, more than about 20%, 30%, 40% and as much as about 60%, 70% or 80% of the total injected dose of the lipid particles is present in plasma about 8, 12, 24, 36, or 48 hours after injection. In certain instances, more than about 10% of a plurality of the particles is present in the plasma of a human about 1 hour after administration. In certain other instances, the presence of the lipid particles is detectable at least about 1 hour after administration of the particle. In some embodiments, the presence of a siRNA molecule is detectable in cells at about 8, 12, 24, 36, 48, 60, 72 or 96 hours after administration. In other embodiments, downregulation of expression of a target sequence, such as a viral or host sequence, by a siRNA molecule is detectable at about 8, 12, 24, 36, 48, 60, 72 or 96 hours after administration. In yet other embodiments, downregulation of expression of a target sequence, such as a viral or host sequence, by a siRNA molecule occurs preferentially in infected cells and/or cells capable of being infected. In further embodiments, the presence or effect of a siRNA molecule in cells at a site proximal or distal to the site of administration is detectable at about 12, 24, 48, 72, or 96 hours, or at about 6, 8, 10, 12, 14, 16, 18, 19, 20, 22, 24, 26, or 28 days after administration. In additional embodiments, the lipid particles are administered parenterally or intraperitoneally.

The compositions, either alone or in combination with other suitable components, can be made into aerosol formulations (i.e., they can be “nebulized”) to be administered via inhalation (e.g., intranasally or intratracheally) (see, Brigham et al., Am. J. Sci., 298:278 (1989)). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045. The disclosures of the above-described patents are herein incorporated by reference in their entirety for all purposes.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Generally, when administered intravenously, the lipid particle formulations are formulated with a suitable pharmaceutical carrier. Suitable formulations are found, for example, in REMINGTON'S PHARMACEUTICAL SCIENCES, Mack Publishing Company, Philadelphia, Pa., 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

In certain applications, the lipid particles disclosed herein may be delivered via oral administration to the individual. The particles may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451, the disclosures of which are herein incorporated by reference in their entirety for all purposes). These oral dosage forms may also contain the following: binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

Typically, these oral formulations may contain at least about 0.1% of the lipid particles or more, although the percentage of the particles may, of course, be varied and may conveniently be between about 1% or 2% and about 60% or 70% or more of the weight or volume of the total formulation. Naturally, the amount of particles in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Formulations suitable for oral administration can consist of: (a) liquid solutions, such as an effective amount of a packaged siRNA molecule (e.g., a siRNA molecule described in Table A or the Examples herein, including the specific combinations of siRNA molecules described herein, e.g., the two-way and three-way combinations of siRNA molecules) suspended in diluents such as water, saline, or PEG 400; (b) capsules, sachets, or tablets, each containing a predetermined amount of a siRNA molecule, as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a siRNA molecule in a flavor, e.g., sucrose, as well as pastilles comprising the therapeutic nucleic acid in an inert base, such as gelatin and glycerin or sucrose and acacia emulsions, gels, and the like containing, in addition to the siRNA molecule, carriers known in the art.

In another example of their use, lipid particles can be incorporated into a broad range of topical dosage forms. For instance, a suspension containing nucleic acid-lipid particles can be formulated and administered as gels, oils, emulsions, topical creams, pastes, ointments, lotions, foams, mousses, and the like.

The amount of particles administered will depend upon the ratio of siRNA molecules to lipid, the particular siRNA used, the strain of HBV being treated, the age, weight, and condition of the patient, and the judgment of the clinician, but will generally be between about 0.01 and about 50 mg per kilogram of body weight, preferably between about 0.1 and about 5 mg/kg of body weight, or about 10⁸-10¹⁰ particles per administration (e.g., injection).

The following describes possible “two way” and “three-way” combinations of different siRNAs selected from the group of siRNAs named 1m thru 15m (see, Table A). Other combinations, e.g., three-way combinations, are described in the Examples. The term “combination”, means that the combined siRNA molecules are present together in the same composition of matter (e.g., dissolved together within the same solution; or present together within the same lipid particle; or present together in the same pharmaceutical formulation of lipid particles, although each lipid particle within the pharmaceutical formulation may or may not include each different siRNA of the siRNA combination). The combined siRNA molecules usually are not covalently linked together.

Certain individual siRNAs are each identified with a name, 1m thru 15m, as shown in Table A. Each siRNA number within a combination is separated with a dash (-); for example, the notation “1m-2m” represents the combination of siRNA number 1m and siRNA number 2m. The dash does not mean that the different siRNA molecules within the combination are covalently linked to each other. Different siRNA combinations are separated by a semicolon. The order of the siRNA numbers in a combination is not significant. For example, the combination 1m-2m is equivalent to the combination 2m-1m because both of these notations describe the same combination of siRNA number 1m with siRNA number 2m.

The two way siRNA combinations of siRNAs 1m thru 15m are: 1m-2m; 1m-3m; 1m-4m; 1m-5m; 1m-6m; 1m-7m; 1m-8m; 1m-9m; 1m-10m; 1m-11m; 1m-12m; 1m-13m; 1m-14m; 1m-15m; 2m-3m; 2m-4m; 2m-5m; 2m-6m; 2m-7m; 2m-8m; 2m-9m; 2m-10m; 2m-11m; 2m-12m; 2m-13m; 2m-14m; 2m-15m; 3m-4m; 3m-5m; 3m-6m; 3m-7m; 3m-8m; 3m-9m; 3m-10m; 3m-11m; 3m-12m; 3m-13m; 3m-14m; 3m-15m; 4m-5m; 4m-6m; 4m-7m; 4m-8m; 4m-9m; 4m-10m; 4m-11m; 4m-12m; 4m-13m; 4m-14m; 4m-15m; 5m-6m; 5m-7m; 5m-8m; 5m-9m; 5m-10m; 5m-11m; 5m-12m; 5m-13m; 5m-14m; 5m-15m; 6m-7m; 6m-8m; 6m-9m; 6m-10m; 6m-11m; 6m-12m; 6m-13m; 6m-14m; 6m-15m; 7m-8m; 7m-9m; 7m-10m; 7m-11m; 7m-12m; 7m-13m; 7m-14m; 7m-15m; 8m-9m; 8m-10m; 8m-11m; 8m-12m; 8m-13m; 8m-14m; 8m-15m; 9m-10m; 9m-11m; 9m-12m; 9m-13m; 9m-14m; 9m-15m; 10m-11m; 10m-12m; 10m-13m; 10m-14m; 10m-15m; 11m-12m; 11m-13m; 11m-14m; 11m-15m; 12m-13m; 12m-14m; 12m-15m; 13m-14m; 13m-15m; and 14m-15m.

The three way siRNA combinations of siRNAs 1m thru 15m are: 1m-2m-3m; 1m-2m-4m; 1m-2m-5m; 1m-2m-6m; 1m-2m-7m; 1m-2m-8m; 1m-2m-9m; 1m-2m-10m; 1m-2m-11m; 1m-2m-12m; 1m-2m-13m; 1m-2m-14m; 1m-2m-15m; 1m-3m-4m; 1m-3m-5m; 1m-3m-6m; 1m-3m-7m; 1m-3m-8m; 1m-3m-9m; 1m-3m-10m; 1m-3m-11m; 1m-3m-12m; 1m-3m-13m; 1m-3m-14m; 1m-3m-15m; 1m-4m-5m; 1m-4m-6m; 1m-4m-7m; 1m-4m-8m; 1m-4m-9m; 1m-4m-10m; 1m-4m-11m; 1m-4m-12m; 1m-4m-13m; 1m-4m-14m; 1m-4m-15m; 1m-5m-6m; 1m-5m-7m; 1m-5m-8m; 1m-5m-9m; 1m-5m-10m; 1m-5m-11m; 1m-5m-12m; 1m-5m-13m; 1m-5m-14m; 1m-5m-15m; 1m-6m-7m; 1m-6m-8m; 1m-6m-9m; 1m-6m-10m; 1m-6m-11m; 1m-6m-12m; 1m-6m-13m; 1m-6m-14m; 1m-6m-15m; 1m-7m-8m; 1m-7m-9m; 1m-7m-10m; 1m-7m-11m; 1m-7m-12m; 1m-7m-13m; 1m-7m-14m; 1m-7m-15m; 1m-8m-9m; 1m-8m-10m; 1m-8m-11m; 1m-8m-12m; 1m-8m-13m; 1m-8m-14m; 1m-8m-15m; 1m-9m-10m; 1m-9m-11m; 1m-9m-12m; 1m-9m-13m; 1m-9m-14m; 1m-9m-15m; 1m-10m-11m; 1m-10m-12m; 1m-10m-13m; 1m-10m-14m; 1m-10m-15m; 1m-11m-12m; 1m-11m-13m; 1m-11m-14m; 1m-11m-15m; 1m-12m-13m; 1m-12m-14m; 1m-12m-15m; 1m-13m-14m; 1m-13m-15m; 1m-14m-15m; 2m-3m-4m; 2m-3m-5m; 2m-3m-6m; 2m-3m-7m; 2m-3m-8m; 2m-3m-9m; 2m-3m-10m; 2m-3m-11m; 2m-3m-12m; 2m-3m-13m; 2m-3m-14m; 2m-3m-15m; 2m-4m-5m; 2m-4m-6m; 2m-4m-7m; 2m-4m-8m; 2m-4m-9m; 2m-4m-10m; 2m-4m-11m; 2m-4m-12m; 2m-4m-13m; 2m-4m-14m; 2m-4m-15m; 2m-5m-6m; 2m-5m-7m; 2m-5m-8m; 2m-5m-9m; 2m-5m-10m; 2m-5m-11m; 2m-5m-12m; 2m-5m-13m; 2m-5m-14m; 2m-5m-15m; 2m-6m-7m; 2m-6m-8m; 2m-6m-9m; 2m-6m-10m; 2m-6m-11m; 2m-6m-12m; 2m-6m-13m; 2m-6m-14m; 2m-6m-15m; 2m-7m-8m; 2m-7m-9m; 2m-7m-10m; 2m-7m-11m; 2m-7m-12m; 2m-7m-13m; 2m-7m-14m; 2m-7m-15m; 2m-8m-9m; 2m-8m-10m; 2m-8m-11m; 2m-8m-12m; 2m-8m-13m; 2m-8m-14m; 2m-8m-15m; 2m-9m-10m; 2m-9m-11m; 2m-9m-12m; 2m-9m-13m; 2m-9m-14m; 2m-9m-15m; 2m-10m-11m; 2m-10m-12m; 2m-10m-13m; 2m-10m-14m; 2m-10m-15m; 2m-11m-12m; 2m-11m-13m; 2m-11m-14m; 2m-11m-15m; 2m-12m-13m; 2m-12m-14m; 2m-12m-15m; 2m-13m-14m; 2m-13m-15m; 2m-14m-15m; 3m-4m-5m; 3m-4m-6m; 3m-4m-7m; 3m-4m-8m; 3m-4m-9m; 3m-4m-10m; 3m-4m-11m; 3m-4m-12m; 3m-4m-13m; 3m-4m-14m; 3m-4m-15m; 3m-5m-6m; 3m-5m-7m; 3m-5m-8m; 3m-5m-9m; 3m-5m-10m; 3m-5m-11m; 3m-5m-12m; 3m-5m-13m; 3m-5m-14m; 3m-5m-15m; 3m-6m-7m; 3m-6m-8m; 3m-6m-9m; 3m-6m-10m; 3m-6m-11m; 3m-6m-12m; 3m-6m-13m; 3m-6m-14m; 3m-6m-15m; 3m-7m-8m; 3m-7m-9m; 3m-7m-10m; 3m-7m-11m; 3m-7m-12m; 3m-7m-13m; 3m-7m-14m; 3m-7m-15m; 3m-8m-9m; 3m-8m-10m; 3m-8m-11m; 3m-8m-12m; 3m-8m-13m; 3m-8m-14m; 3m-8m-15m; 3m-9m-10m; 3m-9m-11m; 3m-9m-12m; 3m-9m-13m; 3m-9m-14m; 3m-9m-15m; 3m-10m-11m; 3m-10m-12m; 3m-10m-13m; 3m-10m-14m; 3m-10m-15m; 3m-11m-12m; 3m-11m-13m; 3m-11m-14m; 3m-11m-15m; 3m-12m-13m; 3m-12m-14m; 3m-12m-15m; 3m-13m-14m; 3m-13m-15m; 3m-14m-15m; 4m-5m-6m; 4m-5m-7m; 4m-5m-8m; 4m-5m-9m; 4m-5m-10m; 4m-5m-11m; 4m-5m-12m; 4m-5m-13m; 4m-5m-14m; 4m-5m-15m; 4m-6m-7m; 4m-6m-8m; 4m-6m-9m; 4m-6m-10m; 4m-6m-11m; 4m-6m-12m; 4m-6m-13m; 4m-6m-14m; 4m-6m-15m; 4m-7m-8m; 4m-7m-9m; 4m-7m-10m; 4m-7m-11m; 4m-7m-12m; 4m-7m-13m; 4m-7m-14m; 4m-7m-15m; 4m-8m-9m; 4m-8m-10m; 4m-8m-11m; 4m-8m-12m; 4m-8m-13m; 4m-8m-14m; 4m-8m-15m; 4m-9m-10m; 4m-9m-11m; 4m-9m-12m; 4m-9m-13m; 4m-9m-14m; 4m-9m-15m; 4m-10m-11m; 4m-10m-12m; 4m-10m-13m; 4m-10m-14m; 4m-10m-15m; 4m-11m-12m; 4m-11m-13m; 4m-11m-14m; 4m-11m-15m; 4m-12m-13m; 4m-12m-14m; 4m-12m-15m; 4m-13m-14m; 4m-13m-15m; 4m-14m-15m; 5m-6m-7m; 5m-6m-8m; 5m-6m-9m; 5m-6m-10m; 5m-6m-11m; 5m-6m-12m; 5m-6m-13m; 5m-6m-14m; 5m-6m-15m; 5m-7m-8m; 5m-7m-9m; 5m-7m-10m; 5m-7m-11m; 5m-7m-12m; 5m-7m-13m; 5m-7m-14m; 5m-7m-15m; 5m-8m-9m; 5m-8m-10m; 5m-8m-11m; 5m-8m-12m; 5m-8m-13m; 5m-8m-14m; 5m-8m-15m; 5m-9m-10m; 5m-9m-11m; 5m-9m-12m; 5m-9m-13m; 5m-9m-14m; 5m-9m-15m; 5m-10m-11m; 5m-10m-12m; 5m-10m-13m; 5m-10m-14m; 5m-10m-15m; 5m-11m-12m; 5m-11m-13m; 5m-11m-14m; 5m-11m-15m; 5m-12m-13m; 5m-12m-14m; 5m-12m-15m; 5m-13m-14m; 5m-13m-15m; 5m-14m-15m; 6m-7m-8m; 6m-7m-9m; 6m-7m-10m; 6m-7m-11m; 6m-7m-12m; 6m-7m-13m; 6m-7m-14m; 6m-7m-15m; 6m-8m-9m; 6m-8m-10m; 6m-8m-11m; 6m-8m-12m; 6m-8m-13m; 6m-8m-14m; 6m-8m-15m; 6m-9m-10m; 6m-9m-11m; 6m-9m-12m; 6m-9m-13m; 6m-9m-14m; 6m-9m-15m; 6m-10m-11m; 6m-10m-12m; 6m-10m-13m; 6m-10m-14m; 6m-10m-15m; 6m-11m-12m; 6m-11m-13m; 6m-11m-14m; 6m-11m-15m; 6m-12m-13m; 6m-12m-14m; 6m-12m-15m; 6m-13m-14m; 6m-13m-15m; 6m-14m-15m; 7m-8m-9m; 7m-8m-10m; 7m-8m-11m; 7m-8m-12m; 7m-8m-13m; 7m-8m-14m; 7m-8m-15m; 7m-9m-10m; 7m-9m-11m; 7m-9m-12m; 7m-9m-13m; 7m-9m-14m; 7m-9m-15m; 7m-10m-11m; 7m-10m-12m; 7m-10m-13m; 7m-10m-14m; 7m-10m-15m; 7m-11m-12m; 7m-11m-13m; 7m-11m-14m; 7m-11m-15m; 7m-12m-13m; 7m-12m-14m; 7m-12m-15m; 7m-13m-14m; 7m-13m-15m; 7m-14m-15m; 8m-9m-10m; 8m-9m-11m; 8m-9m-12m; 8m-9m-13m; 8m-9m-14m; 8m-9m-15m; 8m-10m-11m; 8m-10m-12m; 8m-10m-13m; 8m-10m-14m; 8m-10m-15m; 8m-11m-12m; 8m-11m-13m; 8m-11m-14m; 8m-11m-15m; 8m-12m-13m; 8m-12m-14m; 8m-12m-15m; 8m-13m-14m; 8m-13m-15m; 8m-14m-15m; 9m-10m-11m; 9m-10m-12m; 9m-10m-13m; 9m-10m-14m; 9m-10m-15m; 9m-11m-12m; 9m-11m-13m; 9m-11m-14m; 9m-11m-15m; 9m-12m-13m; 9m-12m-14m; 9m-12m-15m; 9m-13m-14m; 9m-13m-15m; 9m-14m-15m; 10m-11m-12m; 10m-11m-13m; 10m-11m-14m; 10m-11m-15m; 10m-12m-13m; 10m-12m-14m; 10m-12m-15m; 10m-13m-14m; 10m-13m-15m; 10m-14m-15m; 11m-12m-13m; 11m-12m-14m; 11m-12m-15m; 11m-13m-14m; 11m-13m-15m; 11m-14m-15m; 12m-13m-14m; 12m-13m-15m; 12m-14m-15m; and 13m-14m-15m.

The siRNA two-way and three-way combinations are useful, for example, to treat HBV and/or HDV infection in humans, and to ameliorate at least one symptom associated with the HBV infection and/or HDV infection.

In certain embodiments, the siRNA is administered via nucleic acid lipid particle.

In certain embodiments, with respect to methods that include the use of a cocktail of siRNAs encapsulated within lipid particles, the different siRNA molecules are co-encapsulated in the same lipid particle.

In certain embodiments, the with respect to methods that include the use of a cocktail of siRNAs encapsulated within lipid particles, each type of siRNA species present in the cocktail is encapsulated in its own particle.

In certain embodiments, the with respect to methods that include the use of a cocktail of siRNAs encapsulated within lipid particles, some siRNA species are coencapsulated in the same particle while other siRNA species are encapsulated in different particles.

### Formulation and Administration of Two or More Agents

It will be understood that the agents can be formulated together in a single preparation or that they can be formulated separately and, thus, administered separately, either simultaneously or sequentially. In one embodiment, when the agents are administered sequentially (e.g. at different times), the agents may be administered so that their biological effects overlap (i.e. each agent is producing a biological effect at a single given time).

The agents can be formulated for and administered using any acceptable route of administration depending on the agent selected. For example, suitable routes include, but are not limited to, oral, sublingual, buccal, topical, transdermal, parenteral, subcutaneous, intraperitoneal, intrapulmonary, and intranasal, and, if desired for local treatment, intralesional administration. In one embodiment, the small molecule agents identified herein can be administered orally. In another embodiment, the oligomeric nucleotides can be administered by injection (e.g., into a blood vessel, such as a vein), or subcutaneously. In some embodiments, a subject in need thereof is administered one or more agent orally (e.g., in pill form), and also one or more oligomeric nucleotides by injection or subcutaneously.

Typically, the oligomeric nucleotides targeted to the Hepatitis B genome are administered intravenously, for example in a lipid nanoparticle formulation, however, the present invention is not limited to intravenous formulations comprising the oligomeric nucleotides or to treatment methods wherein an oligomeric nucleotide is administered intravenously.

The agents can be individually formulated by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may typically range anywhere from about 3 to about 8. The agents ordinarily will be stored as a solid composition, although lyophilized formulations or aqueous solutions are acceptable.

Compositions comprising the agents can be formulated, dosed, and administered in a fashion consistent with good medical practice. Factors for consideration in this context include the particular disorder being treated, the particular human being treated, the clinical condition of the individual patient, the cause of the disorder, the site of administration, the method of administration, the scheduling of administration, and other factors known to medical practitioners.

The agents may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug or aid in the manufacturing of the pharmaceutical product (i.e., medicament).

The agents are typically dosed at least at a level to reach the desired biological effect. Thus, an effective dosing regimen will dose at least a minimum amount that reaches the desired biological effect, or biologically effective dose, however, the dose should not be so high as to outweigh the benefit of the biological effect with unacceptable side effects. Therefore, an effective dosing regimen will dose no more than the maximum tolerated dose (“MTD”). The maximum tolerated dose is defined as the highest dose that produces an acceptable incidence of dose-limiting toxicities (“DLT”). Doses that cause an unacceptable rate of DLT are considered non-tolerated. Typically, the MTD for a particular schedule is established in phase 1 clinical trials. These are usually conducted in patients by starting at a safe starting dose of 1/10 the severe toxic dose (“STD10”) in rodents (on a mg/m² basis) and accruing patients in cohorts of three, escalating the dose according to a modified Fibonacci sequence in which ever higher escalation steps have ever decreasing relative increments (e.g., dose increases of 100%, 65%, 50%, 40%, and 30% to 35% thereafter). The dose escalation is continued in cohorts of three patients until a non-tolerated dose is reached. The next lower dose level that produces an acceptable rate of DLT is considered to be the MTD.

The amount of the agents administered will depend upon the particular agent used, the strain of HBV being treated, the age, weight, and condition of the patient, and the judgment of the clinician, but will generally be between about 0.2 to 2.0 grams per day.

### Kits

One embodiment provides a kit. The kit may comprise a container comprising the combination. Suitable containers include, for example, bottles, vials, syringes, blister pack, etc. The container may be formed from a variety of materials such as glass or plastic. The container may hold the combination which is effective for treating the condition and may have a sterile access port (for example, the container may be an intravenous solution bag or a vial having a stopper pierceable by a hypodermic injection needle).

The kit may further comprise a label or package-insert on or associated with the container. The term “package-insert” is used to refer to instructions customarily included in commercial packages of therapeutic agents that contain information about the indications, usage, dosage, administration, contraindications and/or warnings concerning the use of such therapeutic agents. In one embodiment, the label or package inserts indicates that the therapeutic agents can be used to treat a viral infection, such as Hepatitis B.

In certain embodiments, the kits are suitable for the delivery of solid oral forms of the therapeutic agents, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to another embodiment, a kit may comprise (a) a first container with one agent contained therein; and (b) a second container with a second agent contained therein. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the therapeutic agents. For example, the kit may further comprise directions for the simultaneous, sequential or separate administration of the therapeutic agents to a patient in need thereof.

In certain other embodiments, the kit may comprise a container for containing separate compositions such as a divided bottle or a divided foil packet, however, the separate compositions may also be contained within a single, undivided container. In certain embodiments, the kit comprises directions for the administration of the separate therapeutic agents. The kit form is particularly advantageous when the separate therapeutic agents are preferably administered in different dosage forms (e.g., oral and parenteral), are administered at different dosage intervals, or when titration of the individual therapeutic agents of the combination is desired by the prescribing physician.

The ability of a combination of therapeutic agents to treat Hepatitis B may be determined using pharmacological models which are well known to the art.

The invention will now be illustrated by the following non-limiting Examples.

### EXAMPLES

The following compounds are referenced in the Examples. Compounds 3-4 can be prepared using known procedures. International Patent Applications Publication Numbers WO2014/106019 and WO2013/006394 also describe synthetic methods that can be used to prepare Compounds 3-4.

| Compound | |
|---|---|
| Number | |
| or Name | Structure |
| 3 | 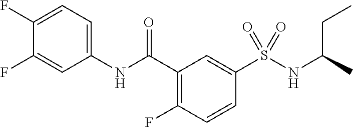 [SMILES: CC[C@@H](C)NS(=O)(=O)c1ccc(F)c(C(=O)Nc2ccc(F)c(F)c2)c1] |
| 4 | 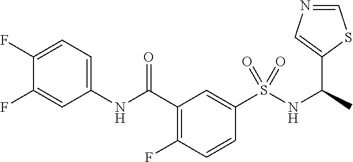 [SMILES: C[C@@H](NS(=O)(=O)c1ccc(F)c(C(=O)Nc2ccc(F)c(F)c2)c1)c1cncs1] |
| Entecavir | 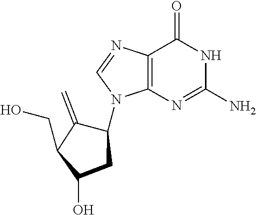 [SMILES: C=C1[C@H](CO)[C@H](O)C[C@@H]1n1cnc2c(=O)[nH]c(N)nc21] |
| Lamivudine | 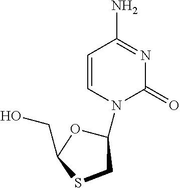 |

### Example 1

A mouse model of hepatitis B virus (HBV) was used to assess the anti-HBV effects of an immune stimulant and HBV-targeting siRNAs, both as independent treatments and in combination with each other.

The following lipid nanoparticle (LNP) formulation was used to deliver the HBV siRNAs. The values shown in the table are mole percentages. The abbreviation DSPC means distearoylphosphatidylcholine.

| PEG(2000)-C-DMA | Cationic lipid | Cholesterol | DSPC |
|---|---|---|---|
| 1.1 | 55.0 | 33.0 | 11.0 |

The cationic lipid had the following structure (13):

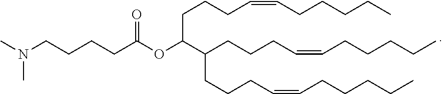 [SMILES: CCCCC/C=C\CCCC(CCC/C=C\CCCCC)C(CCC/C=C\CCCCC)OC(=O)CCCCN(C)C]

A mixture of three siRNAs targeting the HBV genome were used. The sequences of the three siRNAs are shown below.

| Sense Sequence (5′-3′) | Antisense Sequence (5′-3′) |
|---|---|
| CCGUguGCACUuCGCuuCAUU | UGAAGCGAAGUgCACACgGUU |
| CuggCUCAGUUUACuAgUGUU | CACUAgUAAACUgAgCCAGUU |
| GCCgAuCCAUACugCGgAAUU | UUCCGCAgUAUGgAUCGgCUU |

lower case = 2′-O-methyl modification
Underline = unlocked nucleobase analogue (UNA) moiety

On Day −27, 10 micrograms of the plasmid pAAV/HBV1.2 (obtained from Dr. Pei-Jer Chen, originally described in Huang, L R et al., Proceedings of the National Academy of Sciences, 2006, 103(47): 17862-17867)) was administered to C3H/HeN mice via hydrodynamic injection (HDI; rapid 1.3 mL injection into the tail vein). This plasmid carries a 1.2-fold overlength copy of a HBV genome and expresses HBV surface antigen (HBsAg) amongst other HBV products. Serum HBsAg expression in mice was monitored using an enzyme immunoassay. Animals were sorted (randomized) into groups based on serum HBsAg levels such that a) all animals were confirmed to express HBsAg and b) HBsAg group means were similar to each other prior to initiation of treatments.

Animals were treated with immune stimulant as follows: On Day 0, 20 micrograms of high molecular weight polyinosinic:polycytidylic acid (poly(I:C)) was administered via HDI. Animals were treated with lipid nanoparticle (LNP)-encapsulated HBV-targeting siRNAs as follows: On each of Days 0, 7 & 14, an amount of test article equivalent to 1 mg/kg siRNA was administered intravenously. A negative control group was included as the HBsAg expression level is not completely stable in this mouse model of HBV; the absolute concentration of serum HBsAg generally declines over time in individual animals. To demonstrate treatment-specific effects, the treated groups were compared against negative control animals.

The effect of the treatments was determined by collecting a small amount of blood on Days 0 (pre-treatment), 3, 7, 14 & 21 and analyzing it for serum HBsAg content. Samples were diluted as appropriate to generate values within the assay range of quantitation where possible. Individual values falling below the lower limit of quantitation (LLOQ) were set as one-half the LLOQ. Table 1 shows the treatment group mean (n=4 or 5; ±standard error of the mean) serum HBsAg concentration expressed as a percentage of the individual animal pre-treatment baseline value at Day 0.

The data demonstrate the degree of HBsAg reduction in response to the combination of HBV siRNA and poly(I:C), as well as the duration of the reductive effect. The combination of the two treatments resulted in greater effect than either treatment alone.

TABLE 1
Single and Combination Treatment Effect of Three
HBV siRNAs and Immune Stimulant Poly(I:C) on
Serum HBsAg in a Mouse Model of HBV Infection

| | Day 0 | Day 3 | Day 7 | Day 14 | Day 21 |
|---|---|---|---|---|---|
| Negative | 100 ± 0 | 82 ± 4 | 65 ± 9 | 50 ± 10 | 36 ± 11 |
| Control | | | | | |
| HBV | 100 ± 0 | 0.2 ± 0.1 | 4.1 ± 1.3 | 1.6 ± 0.6 | 1.7 ± 0.6 |
| siRNA | | | | | |
| HBV | 100 ± 0 | 0.5 ± 0.2 | 0.4 ± 0.2 | 0.3 ± 0.2 | 0.4 ± 0.2 |
| siRNA ± | | | | | |
| Poly(I:C) | | | | | |
| Poly(I:C) | 100 ± 0 | 6.1 ± 1.1 | 3.5 ± 1.1 | 3.9 ± 1.4 | 4.7 ± 2.3 |

### Example 2

A mouse model of hepatitis B virus (HBV) was used to assess the anti-HBV effects of a small molecule inhibitor of HBV encapsidation (Compound 3) and HBV-targeting siRNAs, both as independent treatments and in combination with each other.

The following lipid nanoparticle (LNP) formulation was used to deliver the HBV siRNAs. The values shown in the table are mole percentages. The abbreviation DSPC means distearoylphosphatidylcholine.

| PEG(2000)-C-DMA | Cationic lipid | Cholesterol | DSPC |
|---|---|---|---|
| 1.6 | 54.6 | 32.8 | 10.9 |

The cationic lipid had the following structure (7):

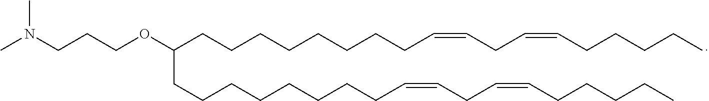 [SMILES: CCCCC/C=C\C/C=C\CCCCCCCCC(CCCCCCCC/C=C\C/C=C\CCCCC)OCCCN(C)C]

A mixture of three siRNAs targeting the HBV genome were used. The sequences of the three siRNAs are shown below.

| Sense Sequence (5′-3′) | Antisense Sequence (5′-3′) |
|---|---|
| CCGUguGCACUuCGCuuCAUU | UGAAGCGAAGUgCACACgGUU |
| CuggCUCAGUUUACuAgUGUU | CACUAgUAAACUgAgCCAGUU |
| GCCgAuCCAUACugCGgAAUU | UUCCGCAgUAUGgAUCGgCUU |

lower case = 2′-O-methyl modification
Underline = unlocked nucleobase analogue (UNA) moiety

On Day −7, 10 micrograms of the plasmid pHBV1.3 (as per Guidotti, L., et al., Journal of Virology, 1995, 69(10): 6158-6169) was administered to NOD.CB17-Prkdc^{scid}/J mice via hydrodynamic injection (HDI; rapid 1.6 mL injection into the tail vein). This plasmid carries a 1.3-fold overlength copy of a HBV genome which, when expressed, generates hepatitis B viral particles including HBV DNA amongst other HBV products. As a readout of the anti-HBV effect of various treatments, serum HBV DNA concentration in mice was measured from total extracted DNA using a quantitative PCR assay (primer/probe sequences from Tanaka, Y., et al., Journal of Medical Virology, 2004, 72: 223-229).

Animals were treated with Compound 3 as follows: Starting on Day 0, a 50 mg/kg or 100 mg/kg dosage of Compound 3 was administered orally to animals on a twice-daily frequency for a total of fourteen doses between Days 0 and 7. Compound 3 was dissolved in a co-solvent formulation for administration. Negative control animals were administered either the co-solvent formulation alone, or saline. Animals were treated with lipid nanoparticle (LNP)-encapsulated HBV-targeting siRNAs as follows: On Day 0, an amount of test article equivalent to 0.1 mg/kg siRNA was administered intravenously. The HBV expression level is not completely stable in this mouse model of HBV; to demonstrate treatment-specific effects, here the treated groups are compared against negative control animals.

The effect of these treatments was determined by collecting blood on Days 0 (pre-treatment), 4 & 7 and analyzing it for serum HBV DNA content. Table 2 shows the treatment group mean (n=7 or 8; ±standard error of the mean) serum HBV DNA concentration expressed as a percentage of the individual animal pre-treatment baseline value at Day 0.

The data demonstrate the degree of serum HBV DNA reduction in response to the combination of Compound 3 and HBV siRNA, as well as the duration of the reductive effect. The combination of the two treatments resulted in greater effect than either treatment alone.

TABLE 2
Single and Combination Treatment Effect of
Compound 3 and Three HBV siRNAs on Serum
HBV DNA in a Mouse Model of HBV Infection

| Treatment 1 | Treatment 2 | | | |
|---|---|---|---|---|
| (Oral) | (IV) | Day 0 | Day 4 | Day 7 |
| Saline | (none) | 100 ± 0 | 69 ± 16 | 70 ± 14 |
| Vehicle | (none) | 100 ± 0 | 56 ± 15 | 47 ± 9 |
| formulation | | | | |
| Compound 3, | (none) | 100 ± 0 | 13 ± 4 | 33 ± 9 |
| 50 mg/kg | | | | |
| Compound 3, | (none) | 100 ± 0 | 8.6 ± 1.5 | 12 ± 5 |
| 100 mg/kg | | | | |
| (none) | HBV siRNA, | 100 ± 0 | 9.4 ± 5.3 | 5.6 ± 1.2 |
| | 0.1 mg/kg | | | |
| Compound 3, | HBV siRNA, | 100 ± 0 | 1.9 ± 0.5 | 1.9 ± 0.4 |
| 50 mg/kg | 0.1 mg/kg | | | |
| Compound 3, | HBV siRNA, | 100 ± 0 | 0.77 ± 0.15 | 0.88 ± 0.28 |
| 100 mg/kg | 0.1 mg/kg | | | |

### Example 3

A mouse model of hepatitis B virus (HBV) was used to assess the anti-HBV effects of a small molecule inhibitor of HBV encapsidation (Compound 3), both as an independent treatment and in combination with the approved compound entecavir (ETV).

On Day −7, 10 micrograms of the plasmid pHBV1.3 (as per Guidotti, L., et al., Journal of Virology, 1995, 69(10): 6158-6169) was administered to NOD.CB17-Prkdc^{scid}/J mice via hydrodynamic injection (HDI; rapid 1.6 mL injection into the tail vein). This plasmid carries a 1.3-fold overlength copy of a HBV genome which, when expressed, generates hepatitis B viral particles including HBV DNA amongst other HBV products. As a readout of the anti-HBV effect of various treatments, serum HBV DNA concentration in mice was measured from total extracted DNA using a quantitative PCR assay (primer/probe sequences from Tanaka, Y., et al., Journal of Medical Virology, 2004, 72: 223-229).

Animals were treated with Compound 3 as follows: Starting on Day 0, a 100 mg/kg dosage of Compound 3 was administered orally to animals on a twice-daily frequency for a total of fourteen doses between Days 0 and 7. Compound 3 was dissolved in a co-solvent formulation for administration. Negative control animals were administered either the co-solvent formulation alone, or saline. Animals were treated with ETV as follows: Starting on Day 0, either a 100 ng/kg or a 300 ng/kg dosage of ETV was administered orally to animals on a once-daily frequency for a total of seven doses between Days 0 and 6. ETV was dissolved in DMSO to 2 mg/mL and then diluted in saline for administration. The HBV expression level is not completely stable in this mouse model of HBV; to demonstrate treatment-specific effects, here the treated groups are compared against negative control animals.

The effect of these treatments was determined by collecting blood on Days 0 (pre-treatment), 4 & 7 and analyzing it for serum HBV DNA content. Samples with Ct values below the lower limit of quantitation (LLOQ) were set to one-half LLOQ for calculation of group means. Table 3 shows the treatment group mean (n=5-8; ±standard error of the mean) serum HBV DNA concentration expressed as a percentage of the individual animal pre-treatment baseline value at Day 0.

The data demonstrate the degree of serum HBV DNA reduction in response to the combination of Compound 3 and ETV, as well as the duration of the reductive effect. The combination of the two treatments resulted in greater effect than either treatment alone.

TABLE 3
Single and Combination Treatment Effect of Compound 3 and
ETV on Serum HBV DNA in a Mouse Model of HBV Infection

| Treatment 1 | Treatment 2 | Day 0 | Day 4 | Day 7 |
|---|---|---|---|---|
| Saline | (none) | 100 ± 0 | 67 ± 18 | 22 ± 8 |
| Vehicle | (none) | 100 ± 0 | 41 ± 7 | 14 ± 3 |
| formulation | | | | |
| Compound 3, | (none) | 100 ± 0 | 9.3 ± 2.5 | 1.2 ± 0.3 |
| 100 mg/kg | | | | |
| (none) | ETV, 100 ng/kg | 100 ± 0 | 21 ± 5 | 3.5 ± 0.7 |
| (none) | ETV, 300 ng/kg | 100 ± 0 | 1.6 ± 0.3 | 0.88 ± 0.31 |
| Compound 3, | ETV, 100 ng/kg | 100 ± 0 | 1.4 ± 0.4 | 0.48 ± 0.18 |
| 100 mg/kg | | | | |
| Compound 3, | ETV, 300 ng/kg | 100 ± 0 | 0.70 ± 0.16 | 0.32 ± 0.07 |
| 100 mg/kg | | | | |

### Examples 4-6

### In Vitro Combination Study Goal:

To determine whether two drug combinations of a small molecule inhibitor of HBV encapsidation (Compound 3), Entecavir (ETV), a reverse transcriptase inhibitorinhibitor of HBV polymerase and SIRNA-NP, an siRNA intended to facilitate potent knockdown of all viral mRNA transcripts and viral antigens, is additive, synergistic or antagonistic in vitro using an HBV cell culture model system.

### Composition of SIRNA-NP:

SIRNA-NP is a lipid nanoparticle formulation of a mixture of three siRNAs targeting the HBV genome. The following lipid nanoparticle (LNP) formulation was used to deliver the HBV siRNAs in the experiments reported herein. The values shown in the table are mole percentages. The abbreviation DSPC means distearoylphosphatidylcholine.

| PEG(20000)-C-DMA | Cationic lipid | Cholesterol | DSPC |
|---|---|---|---|
| 1.6 | 54.6 | 32.8 | 10.9 |

The cationic lipid had the following structure (7):

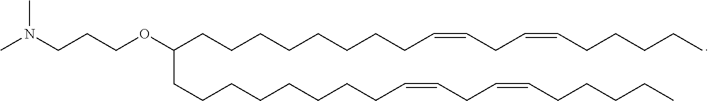 [SMILES: CCCCC/C=C\C/C=C\CCCCCCCCC(CCCCCCCC/C=C\C/C=C\CCCCC)OCCCN(C)C]

The sequences of the three siRNAs are shown below.

| Sense Sequence (5′-3′) | Antisense Sequence (5′-3′) |
|---|---|
| rCrCmGrUmGmUrGrCrArCr | rUrGrArAmGrCmGrArArGmUmGrCr |
| UmUrCmGrCmUmUrCrArUrU | AmCrAmCmGrGrUrU |
| rCmUmGmGrCmUrCrArGmUr | rCrArCrUrAmGmUrArArAmCrUmGr |
| UmUrAmCmUrAmGmUmGrUrU | AmGrCmCrArGrUrU |
| rAmCrCmUrCmUrGmCrCmUr | rGrArGrArUrGmArUmUrArGrGmCr |
| AmArUmCrArUrCrUrCrUrU | AmGrAmGrGrUrUrU |

rN = RNA of base N
mN = 2′O-methyl modification of base N

### In Vitro Combination Experimental Protocol:

In vitro combination studies were conducted using the method of Prichard and Shipman (Prichard M N, and Shipman C Jr., Antiviral Research, 1990, 14 (4-5), 181-205; and Prichard M N, et. al., MacSynergy II). The AML12-HBV10 cell line was developed as described in Campagna et al. (Campagna et. al., J. Virology, 2013, 87(12), 6931-6942). It is a mouse hepatocyte cell line stably transfected with the HBV genome, and which can express HBV pregenomic RNA and support HBV rcDNA (relaxed circular DNA) synthesis in a tetracycline-regulated manner. AML12-HBV10 cells were plated in 96 well tissue-culture treated microtiter plates in DMEM/F12 medium supplemented with 10% fetal bovine serum +1% penicillin-streptomycin without tetracycline and incubated in a humidified incubator at 37° C. and 5% CO₂ overnight. Next day, the cells were switched to fresh medium and treated with inhibitor A and inhibitor B, at concentration range in the vicinity of their respective EC₅₀ values, and incubated for a duration of 48 hrs in a humidified incubator at 37° C. and 5% CO₂. The inhibitors were either diluted in 100% DMSO (ETV and Compound 3) or growth medium (SIRNA-NP) and the final DMSO concentration in the assay was ≤0.5%. The two inhibitors were tested both singly as well as in combinations in a checkerboard fashion such that each concentration of inhibitor A was combined with each concentration of inhibitor B to determine their combination effects on inhibition of rcDNA production. Following a 48 hour-incubation, the level of rcDNA present in the inhibitor-treated wells was measured using a bDNA assay (Affymetrix) with HBV specific custom probe set and manufacturer's instructions. The RLU data generated from each well was calculated as % inhibition of the untreated control wells and analyzed using the MacSynergy II program to determine whether the combinations were synergistic, additive or antagonistic using the interpretive guidelines established by Prichard and Shipman as follows: synergy volumes <25 μM²% (log volume <2) at 95% CI=probably insignificant; 25-50 μM²% (log volume >2 and <5)=minor but significant 50-100 μM²% (log volume >5 and <9)=moderate, may be important in vivo; Over 100 μM²% (log volume >9)=strong synergy, probably important in vivo; volumes approaching 1000 μM²% (log volume >90)=unusually high, check data. Concurrently, the effect of inhibitor combinations on cell viability was assessed using replicate plates that were used to determine the ATP content as a measure of cell viability using the cell-titer glo reagent (Promega) as per manufacturer's instructions.

### Example 4: In Vitro Combination of Compound 3 and Entecavir

Compound 3 (concentration range of 2.5 μM to 0.01 μM in a 2-fold dilution series and 9 point titration) was tested in combination with Entecavir (concentration range of 0.075 μM to 0.001 μM in a 3-fold dilution series and 5 point titration). The average % inhibition in rcDNA and standard deviations of 4 replicates observed either with compound 3 or Entecavir treatments alone or in combination is shown in Table 1. The EC₅₀ values of compound 3 and Entecavir are shown in Table 4. When the observed values of two inhibitor combination were compared to what is expected from additive interaction (Table 1) for the above concentration range, the combinations were found to be additive (Table 4) as per MacSynergy II analysis and using the interpretive criteria described above by Prichard and Shipman (1992).

### Example 5: In Vitro Combination of Compound 3 and SIRNA-NP

Compound 3 (concentration range of 2.5 μM to 0.01 μM in a 2-fold dilution series and 9 point titration) was tested in combination with SIRNA-NP (concentration range of 0.5 μg/mL to 0.006 μg/mL in a 3-fold dilution series and 5 point titration). The average % inhibition in rcDNA and standard deviations of 4 replicates observed either with Compound 3 or SIRNA-NP treatments alone or in combination is shown in Table 2. The EC₅₀ values of Compound 3 and SIRNA-NP are shown in Table 4. When the observed values of two inhibitor combination were compared to what is expected from additive interaction (Table 2) for the above concentration range, the combinations were found to be additive (Table 4) as per MacSynergy II analysis and using the interpretive criteria described above by Prichard and Shipman (1992).

### Example 6: In Vitro Combination of Entecavir and SIRNA-NP

Entecavir (concentration range of 0.075 μM to 0.001 μM in a 3-fold dilution series and 5 point titration) was tested in combination with SIRNA-NP (concentration range of 0.5 μg/mL to 0.002 μg/mL in a 2-fold dilution series and 9 point titration). The average % inhibition in rcDNA and standard deviations of 4 replicates observed either with Entecavir or SIRNA-NP treatments alone or in combination is shown in Table 3. The EC₅₀ values of Entecavir and SIRNA-NP are shown in Table 4. When the observed values of two inhibitor combination were compared to what is expected from additive interaction (Table 3) for the above concentration range, the combinations were found to be additive (Table 4) as per MacSynergy II analysis and using the interpretive criteria described above by Prichard and Shipman (1992).

TABLE 1
In vitro Combination of Entecavir (ETV) and Compound 3

| [DRUG] | | | | | | | | | | | AVERAGE % | |
|---|---|---|---|---|---|---|---|---|---|---|---|---|
| ETV | | | | | | | | | | | INHIBITION | Compound 3 |
| (μM) | 0 | 0.010 | 0.020 | 0.039 | 0.078 | 0.156 | 0.313 | 0.625 | 1.250 | 2.500 | of rcDNA | (μM) |
| 0.075 | 74.32 | 68.07 | 68.39 | 75.14 | 76.89 | 87.27 | 88.19 | 91.48 | 92.88 | 92.19 | | |
| 0.025 | 63.25 | 64.7 | 58.68 | 63.39 | 64.91 | 75.73 | 86.18 | 89.9 | 91.41 | 93.94 | | |
| 0.008 | 48.01 | 49.89 | 54.26 | 52.73 | 62.62 | 74.73 | 82.42 | 85.21 | 89.65 | 90.77 | | |
| 0.003 | 18.71 | 11.06 | 25.23 | 22.45 | 46.04 | 57.94 | 77.01 | 85.49 | 86.6 | 90.86 | | — |
| 0.001 | 21.63 | −4.69 | −0.73 | 9.56 | 30.07 | 52.94 | 74.38 | 83.54 | 89.68 | 91.05 | | |
| 0 | 0 | −3.19 | 0.62 | 7.38 | −1.81 | 35.53 | 70.96 | 80.76 | 86.73 | 90.47 | | |
| [DRUG] | | | | | | | | | | | STANDARD | |
| ETV | | | | | | | | | | | DEVIATION | Compound 3 |
| (μM) | 0 | 0.010 | 0.020 | 0.039 | 0.078 | 0.156 | 0.313 | 0.625 | 1.25 | 2.5 | (%) | (μM) |
| 0.075 | 8.58 | 8.77 | 16.02 | 8.3 | 7.66 | 7.17 | 4.93 | 3.16 | 1.57 | 3.14 | | |
| 0.025 | 13.67 | 10.43 | 13.89 | 13.82 | 12.17 | 7.61 | 3.09 | 2.63 | 1.7 | 0.94 | | |
| 0.008 | 18.71 | 22.38 | 19.17 | 15.26 | 9.56 | 8.73 | 4.65 | 1.94 | 3.91 | 0.91 | | |
| 0.003 | 35.13 | 24.05 | 20.09 | 22.35 | 16.24 | 10.98 | 7.82 | 4.84 | 3.77 | 3.64 | | — |
| 0.001 | 26.12 | 20.67 | 22.56 | 24.1 | 15.68 | 8.42 | 2.57 | 4.25 | 1.74 | 2.61 | | |
| 0 | 0 | 42.74 | 22.32 | 20.39 | 23.53 | 22.08 | 7.85 | 2.94 | 1.46 | 2.2 | | |

| [DRUG] | | | | | | | | | | | | |
|---|---|---|---|---|---|---|---|---|---|---|---|---|
| ETV | | | | | | | | | | | ADDITIVE | Compound 3 |
| (μM) | 0 | 0.010 | 0.020 | 0.039 | 0.078 | 0.156 | 0.3125 | 0.625 | 1.25 | 2.5 | INHIBITION | (μM) |
| 0.075 | 74.32 | 73.5 | 74.48 | 76.22 | 73.86 | 83.44 | 92.54 | 95.06 | 96.59 | 97.55 | | |
| 0.025 | 63.25 | 62.08 | 63.48 | 65.96 | 62.58 | 76.31 | 89.33 | 92.93 | 95.12 | 96.5 | | |
| 0.008 | 48.01 | 46.35 | 48.33 | 51.85 | 47.07 | 66.48 | 84.9 | 90 | 93.1 | 95.05 | | |
| 0.003 | 18.71 | 16.12 | 19.21 | 24.71 | 17.24 | 47.59 | 76.39 | 84.36 | 89.21 | 92.25 | | — |
| 0.001 | 21.63 | 19.13 | 22.12 | 27.41 | 20.21 | 49.47 | 77.24 | 84.92 | 89.6 | 92.53 | | |
| 0 | 0 | −3.19 | 0.62 | 7.38 | −1.81 | 35.53 | 70.96 | 80.76 | 86.73 | 90.47 | | |

| [DRUG] | | | | | | | | | | | SYNERGY | |
|---|---|---|---|---|---|---|---|---|---|---|---|---|
| ETV | | | | | | | | | | | PLOT (99.9%) | |
| (μM) | 0 | 0.010 | 0.020 | 0.039 | 0.078 | 0.156 | 0.313 | 0.625 | 1.25 | 2.5 | Bonferroni Adj. | 96% |
| 0.075 | 0 | 0 | 0 | 0 | 0 | 0 | 0 | 0 | 0 | 0 | SYNERGY | 0 |
| 0.025 | 0 | 0 | 0 | 0 | 0 | 0 | 0 | 0 | 0 | 0 | log volume | 0 |
| 0.008 | 0 | 0 | 0 | 0 | 0 | 0 | 0 | 0 | 0 | −1.28519 | | |
| 0.003 | 0 | 0 | 0 | 0 | 0 | 0 | 0 | 0 | 0 | 0 | ANTAGONISM | −1.29 |
| 0.001 | 0 | 0 | 0 | 0 | 0 | 0 | 0 | 0 | 0 | 0 | log volume | −0.19 |
| 0 | 0 | 0 | 0 | 0 | 0 | 0 | 0 | 0 | 0 | 0 | | |

TABLE 2
In vitro Combination of Compound 3 and SIRNA-NP

| [DRUG] | | | | | | | | | | | AVERAGE % | |
|---|---|---|---|---|---|---|---|---|---|---|---|---|
| SIRNA-NP | | | | | | | | | | | INHIBITION | Compound 3 |
| μg/mL | 0 | 0.010 | 0.020 | 0.039 | 0.078 | 0.156 | 0.313 | 0.625 | 1.250 | 2.5 | of rcDNA | μM |
| 0.5 | 96.25 | 95.01 | 95.45 | 96.35 | 95.83 | 96.38 | 96.15 | 97.02 | 96.88 | 96.9 | | |
| 0.167 | 92.38 | 90.74 | 91.26 | 92.35 | 90.9 | 94.41 | 95.28 | 95.7 | 96.58 | 96.63 | | |
| 0.056 | 68.59 | 66.89 | 75.99 | 72.21 | 81.66 | 83.57 | 90.29 | 92.61 | 94.84 | 95.99 | | |
| 0.019 | 29.18 | 30.74 | 29.09 | 33.68 | 43.8 | 68.05 | 83.12 | 87.88 | 93.48 | 94.35 | | — |
| 0.006 | 14.92 | 0.31 | −4.48 | 6.12 | 19.44 | 49.81 | 78.77 | 85.37 | 90.66 | 92.09 | | |
| 0 | 0 | −1.98 | −20.54 | −16.95 | 20.07 | 37.11 | 59.39 | 79.86 | 88.12 | 89.67 | | |

| [DRUG] | | | | | | | | | | | STANDARD | |
|---|---|---|---|---|---|---|---|---|---|---|---|---|
| SIRNA-NP | | | | | | | | | | | DEVIATION | Compound 3 |
| μg/mL | 0 | 0.010 | 0.020 | 0.039 | 0.078 | 0.156 | 0.313 | 0.625 | 1.25 | 2.5 | (%) | μM |
| 0.5 | 1.42 | 1.64 | 1.15 | 0.66 | 0.89 | 1.23 | 1.26 | 1.22 | 1.07 | 0.87 | | |
| 0.167 | 3.23 | 3.02 | 1.2 | 3.25 | 1.88 | 1.47 | 1.05 | 0.87 | 0.9 | 1.16 | | |
| 0.056 | 9.74 | 8.53 | 3.59 | 6.15 | 5.55 | 3.84 | 2.37 | 2.44 | 1.82 | 1.48 | | |
| 0.019 | 31.44 | 16.24 | 17.69 | 9.21 | 14.48 | 11.22 | 6.35 | 5.11 | 1.1 | 1.48 | | — |
| 0.006 | 25.79 | 18.47 | 16.92 | 29.8 | 15.19 | 13.5 | 4.32 | 0.73 | 3.01 | 3.58 | | |
| 0 | 0 | 16.14 | 29.67 | 32.34 | 27.28 | 28.62 | 12.94 | 5.47 | 5.83 | 2.5 | | |

| [DRUG] | | | | | | | | | | | | |
|---|---|---|---|---|---|---|---|---|---|---|---|---|
| SIRNA-NP | | | | | | | | | | | ADDITIVE | Compound 3 |
| μg/mL | 0 | 0.010 | 0.020 | 0.039 | 0.078 | 0.156 | 0.313 | 0.625 | 1.25 | 2.5 | INHIBITION | μM |
| 0.5 | 96.25 | 96.18 | 95.48 | 95.61 | 97 | 97.64 | 98.48 | 99.24 | 99.55 | 99.61 | | |
| 0.167 | 92.38 | 92.23 | 90.81 | 91.09 | 93.91 | 95.21 | 96.91 | 98.47 | 99.09 | 99.21 | | |
| 0.056 | 68.59 | 67.97 | 62.14 | 63.27 | 74.89 | 80.25 | 87.24 | 93.67 | 96.27 | 96.76 | | |
| 0.019 | 29.18 | 27.78 | 14.63 | 17.18 | 43.39 | 55.46 | 71.24 | 85.74 | 91.59 | 92.68 | | — |
| 0.006 | 14.92 | 13.24 | −2.56 | 0.5 | 32 | 46.49 | 65.45 | 82.86 | 89.89 | 91.21 | | |
| 0 | 0 | −1.98 | −20.54 | −16.95 | 20.07 | 37.11 | 59.39 | 79.86 | 88.12 | 89.67 | | |

| [DRUG] | | | | | | | | | | | SYNERGY | |
|---|---|---|---|---|---|---|---|---|---|---|---|---|
| SIRNA-NP | | | | | | | | | | | PLOT (99.9%) | |
| μg/mL | 0 | 0.010 | 0.020 | 0.039 | 0.078 | 0.156 | 0.313 | 0.625 | 1.25 | 2.5 | Bonferroni Adj. | 96% |
| 0.500 | 0 | 0 | 0 | 0 | 0 | 0 | 0 | 0 | 0 | 0 | SYNERGY | 2.14 |
| 0.167 | 0 | 0 | 0 | 0 | 0 | 0 | 0 | 0 | 0 | 0 | log volume | 0.31 |
| 0.056 | 0 | 0 | 2.03531 | 0 | 0 | 0 | 0 | 0 | 0 | 0 | | |
| 0.019 | 0 | 0 | 0 | 0 | 0 | 0 | 0 | 0 | 0 | 0 | ANTAGONISM | 0 |
| 0.006 | 0 | 0 | 0 | 0 | 0 | 0 | 0 | 0.10757 | 0 | 0 | log volume | 0 |
| 0 | 0 | 0 | 0 | 0 | 0 | 0 | 0 | 0 | 0 | 0 | | |

TABLE 3
In vitro Combination of Entecavir and SIRNA-NP

| | | | | | | | | | | | AVERAGE % | |
|---|---|---|---|---|---|---|---|---|---|---|---|---|
| [DRUG] | | | | | | | | | | | INHIBITION | |
| ETV | | | | | | | | | | | of rcDNA | |
| μM | 0 | 0.002 | 0.004 | 0.008 | 0.016 | 0.032 | 0.063 | 0.125 | 0.250 | 0.500 | SIRNA-NP | μg/mL |
| 0.075 | 74.9 | 77.52 | 75.42 | 79.02 | 85.16 | 86.59 | 92.73 | 95.09 | 96.6 | 96.66 | | |
| 0.025 | 64.1 | 64.59 | 65.95 | 68.92 | 75.31 | 80.87 | 90.12 | 93.84 | 95.54 | 96.72 | | |
| 0.008 | 37.88 | 42.67 | 48.08 | 54.27 | 70.87 | 75.26 | 85.26 | 92.63 | 95.6 | 96.12 | | |
| 0.003 | 37.81 | 25.05 | 31.15 | 33.55 | 48.32 | 68.45 | 81.86 | 91 | 94.63 | 96.08 | | — |
| 0.001 | 9.06 | 11.49 | 1.57 | 22.41 | 33.41 | 61.88 | 77.03 | 90.37 | 93.93 | 95.14 | | |
| 0 | 0 | −8.95 | −7.86 | 20.89 | 32.43 | 46.05 | 72.94 | 87.4 | 93.31 | 95.02 | | |
| | | | | | | | | | | | STANDARD | |
| [DRUG] | | | | | | | | | | | DEVIATION | |
| ETV | | | | | | | | | | | (%) | |
| μM | 0 | 0.002 | 0.004 | 0.008 | 0.016 | 0.032 | 0.063 | 0.125 | 0.25 | 0.5 | SIRNA-NP | μg/mL |
| 0.075 | 5.4 | 2.5 | 2.4 | 3.43 | 3.56 | 4.59 | 1.42 | 0.92 | 1.29 | 1.35 | | |
| 0.025 | 8.24 | 8.69 | 2.67 | 5.28 | 1.81 | 3.19 | 0.79 | 1.39 | 1.72 | 1.28 | | |
| 0.008 | 5.43 | 9.21 | 4.64 | 3.19 | 7.48 | 2.52 | 0.29 | 2.33 | 0.59 | 0.95 | | |
| 0.003 | 8.11 | 11.06 | 14.06 | 2.97 | 7.32 | 2.97 | 1.89 | 1.3 | 0.73 | 0.7 | | — |
| 0.001 | 9.35 | 11.3 | 8.13 | 9.32 | 7.82 | 3.96 | 3.32 | 1.43 | 0.81 | 1.16 | | |
| 0 | 0 | 17.52 | 8.77 | 13.87 | 26.87 | 5.59 | 5.05 | 1.56 | 1.06 | 1.33 | | |
| [DRUG] | | | | | | | | | | | ADDITIVE | |
| ETV | | | | | | | | | | | INHIBITION | |
| μM | 0 | 0.002 | 0.004 | 0.008 | 0.016 | 0.032 | 0.063 | 0.125 | 0.25 | 0.5 | SIRNA-NP | μg/mL |
| 0.075 | 74.9 | 72.65 | 72.93 | 80.14 | 83.04 | 86.46 | 93.21 | 96.84 | 98.32 | 98.75 | | |
| 0.025 | 64.1 | 60.89 | 61.28 | 71.6 | 75.74 | 80.63 | 90.29 | 95.48 | 97.6 | 98.21 | | |
| 0.008 | 37.88 | 32.32 | 33 | 50.86 | 58.03 | 66.49 | 83.19 | 92.17 | 95.84 | 96.91 | | |
| 0.003 | 37.81 | 32.24 | 32.92 | 50.8 | 57.98 | 66.45 | 83.17 | 92.16 | 95.84 | 96.9 | | — |
| 0.001 | 9.06 | 0.92 | 1.91 | 28.06 | 38.55 | 50.94 | 75.39 | 88.54 | 93.92 | 95.47 | | |
| 0 | 0 | −8.95 | −7.86 | 20.89 | 32.43 | 46.05 | 72.94 | 87.4 | 93.31 | 95.02 | | |

| [DRUG] | | | | | | | | | | | SYNERGY | |
|---|---|---|---|---|---|---|---|---|---|---|---|---|
| ETV | | | | | | | | | | | PLOT (99.9%) | |
| μM | 0 | 0.002 | 0.004 | 0.008 | 0.016 | 0.032 | 0.063 | 0.125 | 0.25 | 0.5 | Bonferroni Adj. | 96% |
| 0.075 | 0 | 0 | 0 | 0 | 0 | 0 | 0 | 0 | 0 | 0 | SYNERGY | 1.59 |
| 0.025 | 0 | 0 | 0 | 0 | 0 | 0 | 0 | 0 | 0 | 0 | log volume | 0.23 |
| 0.008 | 0 | 0 | 0 | 0 | 0 | 0.47668 | 1.11561 | 0 | 0 | 0 | | |
| 0.003 | 0 | 0 | 0 | −7.47573 | 0 | 0 | 0 | 0 | 0 | 0 | ANTAGONISM | −7.48 |
| 0.001 | 0 | 0 | 0 | 0 | 0 | 0 | 0 | 0 | 0 | 0 | log volume | −1.07 |
| 0 | 0 | 0 | 0 | 0 | 0 | 0 | 0 | 0 | 0 | 0 | | |

TABLE 4
Summary of results of in vitro combination studies in AML12-HBV10
cell culture system with rcDNA quantitation using bDNA assay:

| | | | Inhibitor B | Synergy | Synergy | Antagonism | Antagonism | |
|---|---|---|---|---|---|---|---|---|
| | | Inhibitor A | EC₅₀ (μM | Volume | Log | Volume | Log | |
| Inhibitor A | Inhibitor B | EC₅₀ (μM) | or μg/mL) | (μM² %)* | Volume | (μM² %)* | Volume | Conclusion |
| Compound 3 | Entecavir | 0.231 | 0.012 | 0 | 0 | −1.29 | −0.19 | Additive |
| | (ETV) | | | | | | | |
| Compound 3 | SIRNA-NP** | 0.250 | 0.032 | 2.14 | 0.31 | 0 | 0 | Additive |
| Entecavir | SIRNA-NP** | 0.012 | 0.031 | 1.59 | 0.23 | −7.48 | −1.07 | Additive |
| (ETV) | | | | | | | | |

*at 99.9% confidence interval
**μg/mL

### Examples 7-9

### In Vitro Combination Study Goal:

To determine the effects of combination treatment with two-compound combinations on the process of HBV DNA replication, cccDNA formation, and cccDNA expression and stability. Compounds 3 and 4, two small molecule inhibitors of HBV encapsidation; entecavir (ETV) and lamivudine (3TC), two FDA-approved reverse transcriptase inhibitorinhibitors of HBV polymerase; and SIRNA-NP, a lipid nanoparticle (LNP)-formulated siRNA inhibitor of viral mRNA and viral antigen expression were investigated. The studies were aimed at determining whether the combinations are additive, synergistic or antagonistic in vitro using an HBV cell culture model system.

LNP formulation:

SIRNA-NP is a lipid nanoparticle formulation of a mixture of three siRNAs targeting the HBV genome. The following lipid nanoparticle (LNP) formulation was used to deliver the HBV siRNAs in the experiments reported herein. The values shown in the table are mole percentages. The abbreviation DSPC means distearoylphosphatidylcholine.

| PEG(2000)-C-DMA | Cationic lipid | Cholesterol | DSPC |
|---|---|---|---|
| 1.6 | 54.6 | 32.8 | 10.9 |

The cationic lipid had the following structure (7):

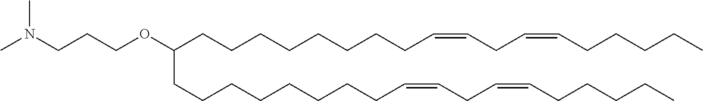 [SMILES: CCCCC/C=C\C/C=C\CCCCCCCCC(CCCCCCCC/C=C\C/C=C\CCCCC)OCCCN(C)C]

siRNA

The sequences of the three siRNAs are shown below.

| Sense Sequence (5′-3′) | Antisense Sequence (5′-3′) |
|---|---|
| rCrCmGrUmGmUrGrCrArCr | rUrGrArAmGrCmGrArArGmUmGr |
| UmUrCmGrCmUmUrCrArUrU | CrAmCrAmCmGrGrUrU |
| rCmUmGmGrCmUrCrArGmUr | rCrArCrUrAmGmUrArArAmCrUm |
| UmUrAmCmUrAmGmUmGrUrU | GrAmGrCmCrArGrUrU |
| rAmCrCmUrCmUrGmCrCmUr | rGrArGrArUrGmArUmUrArGrGm |
| AmArUmCrArUrCrUrCrUrU | CrAmGrAmGrGrUrUrU |

rN = RNA of base N
mN = 2′O-methyl modification of base N

### In Vitro Combination Experimental Protocol:

In vitro combination studies were conducted using a modification of the assay system described in Cai et al (Antimicrobial Agents Chemotherapy, 2012. Vol 56(8):4277-88). A previously developed HepDE19 cell culture system (Guo et al. J. Virology (2007) 81(22): 12472-12484) supports HBV DNA replication and cccDNA formation in a tetracycline (Tet)-regulated manner, and produces a detectable reporter molecule which is dependent upon the production and maintenance of cccDNA.

In the HepDE19 cell culture system, the reporters are the precore RNA and its cognate protein product, the secreted HBV “e antigen” (HBeAg). In HepDE19 cells, precore RNA and HBeAg are only produced from the cccDNA circular template, because the ORF of HBeAg and its 5′ RNA leader are separated between the opposite ends of the integrated viral genome, and only become contiguous with the formation of cccDNA. Although an assay based on the HepDE19 cell culture system is effective for determining activity, the results of high throughput screening may be complicated because the HBeAg ELISA cross reacts with a viral HBeAg homologue, which is the core antigen (HBcAg) expressed largely in a cccDNA-independent fashion in HepDE19 cells. To overcome this complication, an alternative cell culture system (designated herein as DESHAe82 cell culture system and described in PCT/EP/2015/06838) has been developed which includes an in-frame HA epitope tag in the N-terminal coding sequence of HBeAg in the transgene of DESHAe82 cells, without disrupting any cis-element critical for HBV replication, cccDNA transcription, and HBeAg secretion.

A chemiluminescence ELISA assay (CLIA) for the detection of HA-tagged HBeAg with HA antibody serving as capture antibody and HBeAg serving as detection antibody has been developed, eliminating the contaminating signal from HBcAg. The DESHAe82 cell line coupled with HA-HBeAg CLIA assay exhibits high levels of cccDNA synthesis and HA-HBeAg production and secretion, and high specific readout signals with low noise. In addition, a protocol for quantitative reverse transcription and polymerase chain reaction (qRT-PCR) that is specific for detection of precore RNA in either DE19 or DESHAe82 cells was developed and is also used for the detection of the cccDNA-dependent mRNA (precore RNA) that is translated to produce HBeAg or HA-HBeAg.

To test the compound combinations, DESHAe82 or DE19 cells (as indicated in examples) were plated in 96 well tissue-culture treated microtiter plates in DMEM/F12 medium supplemented with 10% fetal bovine serum+1% penicillin-streptomycin with Tet, and incubated in a humidified incubator at 37° C. and 5% CO₂ overnight. Next day, the cells were switched to fresh medium without Tet and treated with inhibitor A and inhibitor B, at concentration range in the vicinity of their respective EC₅₀ values, and incubated for a duration of 48h in a humidified incubator at 37° C. and 5% CO₂. The inhibitors were either diluted in 100% DMSO (ETV, 3TC, Compound 3 and Compound 4) or growth medium (SIRNA-NP) and the final DMSO concentration in the assay was 0.5%. The two inhibitors were tested both singly as well as in combinations in a checkerboard fashion such that each test concentration of inhibitor A was combined with each test concentration of inhibitor B to determine their combination effects on inhibition of cccDNA formation and expression. Untreated negative control samples (0.5% DMSO or media only) were included on each plate in multiple wells. Following a 9 day-incubation, media was removed and cells were subjected to RNA extraction to measure the cccDNA-dependent precore mRNA level. Total cellular RNAs were extracted using a 96-well format total RNA isolation kit (MACHEREY-NAGEL, Cat. 740466.4) by following the instruction of manufacturer (vacuum manifold processing, two more extra washes of Buffer RA4). RNA samples were eluted in RNAase-free water. Quantitative real-time RT-PCR was performed with a Roche LightCycler480 and RNA Master Hydrolysis probe (Catalog number 04991885001, Roche) using primers and conditions for specific detection of cccDNA-dependent precore RNA. GAPDH mRNA levels were also detected by standard methods and used to normalize the precore RNA levels. Inhibition of precore RNA levels, and therefore cccDNA expression, was calculated as % inhibition of the untreated control wells and analyzed using the Prichard-Shipman combination model using the MacSynergyII program (Prichard M N, Shipman C Jr. Antiviral Research, 1990. Vol 14(4-5):181-205; Prichard M N, Aseltine K R, and Shipman, C. MacSynergy II. University of Michigan 1992) to determine whether the combinations were synergistic, additive or antagonistic using the interpretive guidelines established by Prichard and Shipman as follows: synergy volumes <25 μM²% (log volume <2) at 95% CI=probably insignificant; 25-50 (log volume >2 and <5)=minor but significant 50-100 (log volume >5 and <9)=moderate, may be important in vivo; Over 100 (log volume >9)=strong synergy, probably important in vivo; volumes approaching 1000 (log volume >90)=unusually high, check data.

Concurrently, the effect of inhibitor combinations on cell viability and proliferation was assessed in two ways: 1) Direct microscopic observation of test wells, and 2) using replicate plates seeded at 10-20% cell density that, after 4 days, were assayed for intracellular ATP content using the Cell-Titer Glo reagent (Promega) as per manufacturer's instructions. Cell viability and density was calculated as a percentage of the untreated negative control wells.

### Example 7: In vitro combination of Compound 3 and entecavir

Compound 3 (concentration range of 10 μM to 0.0316 μM in a half-log dilution series and 6 point titration) was tested in combination with entecavir (concentration range of 0.010 μM to 0.00003 μM in a half-log, 3.16-fold) dilution series and 6 point titration. The antiviral activity of this combination is shown in Table 7a; synergy and antagonism volumes are shown in Table 7b. The combination results from 2 replicates of measurements of synergy and antagonism volumes according to Prichard and Shipman, and interpretation, are shown in Table 9d. In this assay system, this combination results in synergistic inhibition of precore RNA expression. No significant inhibition of cell viability or proliferation was observed by microscopy.

TABLE 7a
Antiviral Activity of Compound 3 and Entecavir Combination: Average percent
inhibition versus negative control (n = 2 samples per data point)

| | | | | | | | | |
|---|---|---|---|---|---|---|---|---|
| ETV, μM | 0.01 | 86.940 | 97.010 | 97.490 | 95.900 | 97.120 | 98.240 | 99.220 |
| | 0.0032 | 81.510 | 61.730 | 69.510 | 62.570 | 98.550 | 97.820 | 97.690 |
| | 0.001 | 73.320 | 77.600 | 86.990 | 66.700 | 94.490 | 89.590 | 91.710 |
| | 0.0003 | 69.090 | 78.290 | 58.730 | 55.160 | 92.360 | 91.290 | 93.110 |
| | 0.0001 | −8.990 | 39.460 | 55.700 | 44.430 | 45.680 | 73.420 | 91.580 |
| | 3E−05 | −133.220 | −313.960 | 20.870 | 49.930 | 8.740 | 68.590 | 72.590 |
| | 0 | 0.000 | −26.280 | −86.920 | 36.240 | 67.120 | 90.600 | 84.340 |
| | | 0 | 0.032 | 0.100 | 0.317 | 1.001 | 3.165 | 10 |

| | |
|---|---|
| Compounds | Compound 3, μM |

TABLE 7b
MacSynergy Volume Calculations Compound 3 and Entecavir Combination: “Greater
than additive” inhibition level at 99.99% confidence level

| | | | | | | | | |
|---|---|---|---|---|---|---|---|---|
| ETV, μM | 0.01 | 0 | 3.75864 | 13.8041 | 1.86048 | 0 | 0 | 0.74344 |
| | 0.0032 | 0 | 0 | 0 | 0 | 0.87826 | 0 | 0 |
| | 0.001 | 0 | 9.05212 | 27.6452 | 0 | 0 | 0 | 0 |
| | 0.0003 | 0 | 0.40426 | 6.01171 | 0 | 0 | 0 | 0 |
| | 0.0001 | 0 | 75.9052 | 125.983 | 0 | 0 | 0 | 0 |
| | 3E−05 | 0 | 0 | 322.705 | 90.4025 | 0 | 0 | 0 |
| | 0 | 0 | 0 | 0 | 0 | 0 | 0 | 0 |
| | | 0 | 0.032 | 0.100 | 0.317 | 1.001 | 3.165 | 10 |

| | |
|---|---|
| Compounds | Compound 3, μM |

### Example 8: In Vitro Combination of Compound 4 and Entecavir

Compound 4 (concentration range of 10 μM to 0.0316 μM in a half-log dilution series and 6 point titration) was tested in combination with entecavir (concentration range of 0.010 μM to 0.00003 μM in a half-log, 3.16-fold dilution series and 6 point titration). The antiviral activity of this combination is shown in Table 8a; synergy and antagonism volumes are shown in Table 8b. Combination results from 2 replicates of measurements of synergy and antagonism volumes according to Prichard and Shipman and interpretation, are shown in Table 9d. In this assay system, this combination results in synergistic inhibition of precore RNA expression. No significant inhibition of cell viability or proliferation was observed by microscopy.

TABLE 8a
Antiviral Activity Compound 4 and Entecavir Combination: Average percent
inhibition versus negative control (n = 2 samples per data point)

| | | | | | | | | |
|---|---|---|---|---|---|---|---|---|
| ETV, μM | 0.01 | 96 | 92.03 | 89.04 | 98.02 | 97.16 | 97.18 | 96.46 |
| | 0.0032 | 95.31 | 93.96 | 93.11 | 89.34 | 91.81 | 97.7 | 97.74 |
| | 0.001 | 80.83 | 94.74 | 94.25 | 95.49 | 98.64 | 98.14 | 98.7 |
| | 0.0003 | 39.01 | 95.61 | 92.25 | 97.73 | 97.85 | 97.68 | 95.26 |
| | 0.0001 | 64.23 | 78.08 | 98.62 | 96.63 | 89.34 | 98.87 | 95.3 |
| | 3E−05 | −32.56 | −53.69 | 58.53 | 97.04 | 97.7 | 96.9 | 95.1 |
| | 0 | 0 | −49.48 | 66.78 | 94.67 | 93.92 | 97.88 | 97.53 |
| | | 0 | 0.032 | 0.100 | 0.317 | 1.001 | 3.165 | 10 |

| | |
|---|---|
| Compounds | Compound 4, μM |

TABLE 8b
MacSynergy Volume Calculations Compound 4 and Entecavir Combination: “Greater
than additive” inhibition level at 99.99% confidence interval

| | | | | | | | | |
|---|---|---|---|---|---|---|---|---|
| ETV, μM | 0.01 | 0 | −1.99 | −9.63 | −1.77 | −2.6 | −2.74 | −3.44 |
| | 0.0032 | 0 | 0.97 | −5.33 | −10.41 | −7.9 | −2.2 | −2.14 |
| | 0.001 | 0 | 23.4 | 0.62 | −3.49 | −0.19 | −1.45 | −0.83 |
| | 0.0003 | 0 | 86.78 | 12.51 | 0.98 | 1.56 | −1.03 | −3.23 |
| | 0.0001 | 0 | 31.55 | 10.5 | −1.46 | −8.49 | −0.37 | −3.82 |
| | 3E−05 | 0 | 44.46 | 2.57 | 4.11 | 5.76 | −0.29 | −1.63 |
| | 0 | 0 | 0 | 0 | 0 | 0 | 0 | 0 |
| | | 0 | 0.032 | 0.100 | 0.317 | 1.001 | 3.165 | 10 |

| | |
|---|---|
| Compounds | Compound 4, μM |

### Example 9: In Vitro Combination of Compound 3 and SIRNA-NP

Compound 3 (concentration range of 10 μM to 0.0316 μM in a half-log dilution series and 6 point titration) was tested in combination with SIRNA-NP (concentration range of 0.10 μM to 0.000 μg/ml in a half-log, 3.16-fold) dilution series and 6 point titration. The antiviral activity of this combination is shown in Table 9a; synergy and antagonism volumes are shown in Table 9b. The combination results from 4 replicates of measurements of synergy and antagonism volumes according to Prichard and Shipman, and interpretation, are shown in Table 9d. In this assay system, this combination results in synergistic inhibition of precore RNA expression. No significant inhibition of cell viability or proliferation was observed by microscopy or Cell-Titer Glo assay (Table 9c).

TABLE 9a
Antiviral Activity of Compound 3 and SIRNA-NP Combination: Average percent
inhibition versus negative control (n = 4 samples per data point)

| | | | | | | | | |
|---|---|---|---|---|---|---|---|---|
| Compound 3, | 10.000 | 76.180 | 76.580 | 93.330 | 97.170 | 94.670 | 97.120 | 98.640 |
| μM | 3.165 | 73.120 | 93.950 | 95.500 | 97.730 | 98.120 | 99.160 | 98.620 |
| | 1.001 | 88.510 | 95.740 | 97.340 | 97.880 | 98.620 | 99.410 | 98.150 |
| | 0.317 | 77.070 | 96.440 | 93.720 | 98.340 | 98.390 | 99.260 | 97.820 |
| | 0.100 | 71.330 | 87.960 | 91.490 | 87.110 | 97.700 | 97.790 | 95.920 |
| | 0.032 | 35.570 | −56.280 | 64.870 | 86.080 | 90.920 | 86.330 | 89.560 |
| | 0 | 0.000 | 3.930 | −46.460 | 35.730 | 87.370 | 72.720 | 99.230 |
| | | 0 | 0.0003 | 0.001 | 0.003 | 0.010 | 0.032 | 0.100 |

| | |
|---|---|
| Compounds | SIRNA-NP (μg/mL) |

TABLE 9b
MacSynergy Volume Calculations Compound 3 and SIRNA-NP Combination: “Greater
than additive” inhibition level at 99.99% confidence level

| | | | | | | | | |
|---|---|---|---|---|---|---|---|---|
| Compound 3, | 10.000 | 0.000 | 0.000 | 13.805 | 4.977 | 0.000 | 0.000 | −0.061 |
| μM | 3.165 | 0.000 | 2.558 | 28.321 | 9.580 | 0.000 | 4.779 | 0.000 |
| | 1.001 | 0.000 | 1.416 | 10.254 | 1.969 | 0.000 | 0.697 | 0.000 |
| | 0.317 | 0.000 | 11.954 | 12.984 | 9.921 | 0.000 | 3.677 | 0.000 |
| | 0.100 | 0.000 | 0.000 | 1.985 | 0.000 | 0.000 | 3.438 | 0.000 |
| | 0.032 | 0.000 | 0.000 | 0.000 | 0.000 | 0.000 | 0.000 | 0.000 |
| | 0 | 0.000 | 0.000 | 0.000 | 0.000 | 0.000 | 0.000 | 0.000 |
| | | 0 | 0.0003 | 0.001 | 0.003 | 0.010 | 0.032 | 0.100 |

| | |
|---|---|
| Compounds | SIRNA-NP (μg/mL) |

TABLE 9c
Cytotoxicity of Compound 3 and SIRNA-NP Combination:
Average percent of cell viability vs control

| | | | | | | | |
|---|---|---|---|---|---|---|---|
| Compound 3, | 10.000 | 110.5 | 112.6 | 120.6 | 124.0 | 115.0 | 89.1 |
| μM | 3.165 | 105.9 | 116.1 | 119.5 | 120.6 | 117.3 | 95.1 |
| | 1.001 | 109.0 | 118.6 | 115.9 | 114.9 | 116.3 | 91.5 |
| | 0.317 | 110.0 | 111.8 | 119.7 | 117.2 | 109.7 | 90.3 |
| | 0.100 | 99.3 | 107.2 | 115.1 | 119.5 | 119.9 | 93.5 |
| | 0.032 | 99.3 | 107.7 | 122.6 | 127.1 | 123.0 | 85.9 |
| Compounds | | 0.0003 | 0.001 | 0.003 | 0.010 | 0.032 | 0.100 |

TABLE 9d
Summary of results of in vitro combination studies in DESHAe82 cell culture
system with cccDNA-derived precore RNA quantitation by qRT-PCR

| Inhibitor A | | Synergy | Synergy | | Antagonism | |
|---|---|---|---|---|---|---|
| (Compound | | Volume | Log | Antagonism | Log | |
| Number) | Inhibitor B | (μM² %) | Volume | (μM² %) | Volume | Interpretation |
| 3 | Entecavir | 679.15 | 169.58 | 0 | 0 | Synergy |
| | (ETV) | | | | | |
| 4 | Entecavir | 225.77 | 56.44 | −76.43 | −19.11 | Synergy |
| | (ETV) | | | | | |
| 3 | SIRNA-NP | 122.31 | 30.54 | −0.06 | −0.01 | Synergy |

### Example 10

The object of this example was to compare the anti-HBV activity of various combination treatments including Compound 3, a small molecule inhibitor of HBV encapsidation and SIRNA-NP, a lipid nanoparticle formulation encapsulating HBV-targeting siRNAs, as well as established HBV standard of care treatments: Entecavir (ETV), a nucleos(t)ide analogue inhibiting HBV DNA polymerase activity (de Man R A et al., Hepatology, 34(3), 578-82 (2001)) and pegylated interferon alpha-2a (pegINF α-2a), which limits viral dissemination via a type 1 interferon receptor activation (Marcellin et al., N Engl J Med., 51(12), 1206-17 (2004)). Potency of these combinations was compared to monotherapy treatments with Compound 3, SIRNA-NP and ETV alone, as well as to a negative control treatment condition with Vehicle for Compound 3.

This work was conducted in a well-established humanized liver chimeric mouse model of chronic hepatitis B virus (HBV) infection (Tsuge et al., Hepatology, 42(5), 1046-54 (2005)). A persistent level of HBV infection was established in the animals prior to the treatment phase which started at Day 0. Test articles dosages were as follows: Compound 3, oral 100 mg/kg twice daily; SIRNA-NP, intravenous 3 mg/kg every 2 weeks; ETV, oral 1.2 μg/kg daily; pegIFN α-2a, subcutaneous 30 μg/kg twice a week.

The anti-HBV effects were assessed based on serum HBsAg levels using the GS HBsAg EIA 3.0 enzyme linked immunosorbent assay kit from Bio-Rad Laboratories as per manufacturer instructions; and serum HBV DNA levels measured from total extracted DNA using a quantitative PCR assay (primer/probe sequences from Tanaka et al., Journal of Medical Virology, 72, 223-229 (2004)).

Dual and triple combination treatments resulted in more anti-viral activity as exemplified by stronger reductions in serum HBV DNA levels relative to the monotherapy treatments investigated. Particularly, at Day 28, serum HBV DNA levels were reduced over 2.5 log 10 upon treatment with a combination of Compound 3 and SIRNA-LNP or Compound 3 and pegIFN α-2a, and 2 log 10 upon treatment with a combination of Compound 3 and ETV, as compared to the 1.0 to 1.5 log 10 reductions observed with monotherapy treatments of ETV or Compound 3 or SIRNA-LNP. Triple combination treatment with Compound 3 and SIRNA-NP and ETV or Compound 3 and SIRNA-NP and pegINF α-2a demonstrated slightly improved effect on HBV DNA levels relative to the dual combination treatments out to Day 28. The ability of SIRNA-NP to inhibit hepatitis B protein (antigen) production, as exemplified by serum HBsAg levels, was maintained (when co-administered in combination with the other antiviral treatments).

TABLE 10a
Effect of Combinatorial and Monotherapy Treatments on Serum HBV DNA Levels

| | | Serum HBV DNA | | | | |
|---|---|---|---|---|---|---|
| Group | | (Copies/mL ± SEM) | | | | |
| No. | Treatment | Day 0 | Day 7 | Day 14 | Day 21 | Day 28 |
| 1 | Vehicle Control | 1.50E+08 ± | 1.65E+08 ± | 1.45E+08 ± | 2.13E+08 ± | 2.13E+08 ± |
| | for Compound 3 | 1.82E+07 | 2.78E+07 | 1.50E+07 | 3.01E+07 | 2.63E+07 |
| 2 | Compound 3 | 1.70E+08 ± | 1.33E+07 ± | 1.28E+07 ± | 1.02E+07 ± | 1.17E+07 ± |
| | | 2.16E+07 | 1.85E+06 | 1.78E+06 | 4.24E+06 | 5.20E+06 |
| 3 | SIRNA-NP | 1.88E+08 ± | 5.18E+06 ± | 6.40E+06 ± | 2.24E+06 ± | 6.86E+06 ± |
| | | 4.52E+07 | 1.50E+06 | 9.67E+05 | 5.51E+05 | 2.26E+06 |
| 4 | Compound 3 + | 1.56E+08 ± | 8.64E+06 ± | 2.02E+06 ± | 4.36E+05 ± | 3.64E+05 ± |
| | SIRNA-NP | 2.25E+07 | 2.48E+06 | 5.08E+05 | 1.18E+05 | 1.00E+05 |
| 5 | Compound 3 + | 1.66E+08 ± | 6.82E+06 ± | 1.57E+06 ± | 3.70E+05 ± | 1.68E+05 ± |
| | SIRNA-NP + | 1.33E+07 | 1.64E+06 | 2.19E+05 | 8.96E+04 | 4.00E+04 |
| | ETV | | | | | |
| 6 | Compound 3 + | 2.42E+08 ± | 7.75E+06 ± | 1.79E+06 ± | 5.48E+05 ± | 2.90E+05 ± |
| | SIRNA-NP + | 5.70E+07 | 2.03E+06 | 4.53E+05 | 1.12E+05 | 2.52E+04 |
| | pegIFN α-2a | | | | | |
| 7 | Compound 3 + | 1.96E+08 ± | 1.70E+07 ± | 5.22E+06 ± | 2.34E+06 ± | 1.80E+06 ± |
| | ETV | 2.46E+07 | 4.13E+06 | 1.06E+06 | 4.06E+05 | 3.67E+05 |
| 8 | Compound 3 + | 1.67E+08 ± | 8.50E+06 ± | 1.39E+06 ± | 4.98E+05 ± | 3.01E+05 ± |
| | pegIFN α-2a | 2.54E+07 | 1.64E+06 | 3.71E+05 | 1.25E+05 | 8.11E+04 |
| 9 | ETV | 1.48E+08 ± | 2.35E+07 ± | 1.38E+07 ± | 1.35E+07 ± | 9.33E+06 ± |
| | | 1.18E+07 | 2.47E+06 | 1.65E+06 | 6.45E+05 | 3.20E+05 |

TABLE 10b
Effect of Combinatorial and Monotherapy
Treatments on Serum HBsAg Levels

| | |
|---|---|
| | Serum HBsAg |
| Group | (IU/mL ± SEM) |

| No. | Treatment | Day 0 | Day 21 |
|---|---|---|---|
| 1 | Vehicle Control | 2761 ± 388 | 4065 ± 338 |
| | for Compound 3 | | |
| 2 | Compound 3 | 2965 ± 616 | 4158 ± 355 |
| 3 | SIRNA-NP | 3352 ± 812 | 44 ± 11 |
| 4 | Compound 3 + | 3436 ± 498 | 58 ± 8 |
| | SIRNA-NP | | |
| 5 | Compound 3 + | 2795 ± 309 | 96 ± 24 |
| | SIRNA-NP + | | |
| | ETV | | |
| 6 | Compound 3 + | 3965 ± 734 | 37 ± 4 |
| | SIRNA-NP + | | |
| | pegIFN α-2a | | |
| 7 | Compound 3 + | 3965 ± 779 | 5822 ± 1490 |
| | ETV | | |
| 8 | Compound 3 + | 3154 ± 521 | 3621 ± 683 |
| | pegIFN α-2a | | |
| 9 | ETV | 2649 ± 282 | 2975 ± 629 |

### Example 11

### In Vitro Combination Study Goal:

To determine whether two drug combinations of a small molecule inhibitor of HBV encapsidation (Compound 3) and tenofovir (TDF), a nucleoside analog inhibitor of HBV polymerase is additive, synergistic or antagonistic in vitro using an HBV cell culture model system.

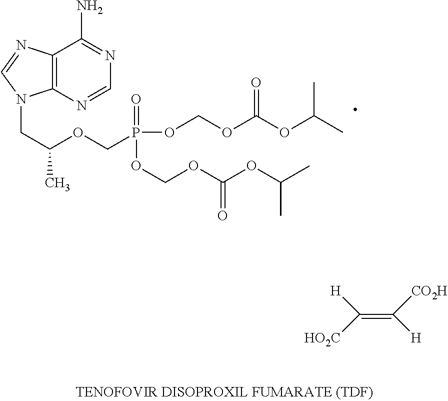 [SMILES: CC(C)OC(=O)OCOP(=O)(CO[C@H](C)Cn1cnc2c(N)ncnc21)OCOC(=O)OC(C)C ; O=C(O)/C=C/C(=O)O]

### In Vitro Combination Experimental Protocol:

In vitro combination studies were conducted using the method of Prichard and Shipman (Prichard M N, and Shipman C Jr., Antiviral Research, 1990, 14 (4-5), 181-205; and Prichard M N, et. al., MacSynergy II). HepDE19 cell culture system is a HepG2 (human hepatocarcinoma) derived cell line that supports HBV DNA replication and cccDNA formation in a tetracycline (Tet)-regulated manner and produces HBV rcDNA and a detectable reporter molecule dependent on the production and maintenance of cccDNA (Guo et al 2007. J. Virol 81:12472-12484). HepDE19 (50,000 cells/well) were plated in 96 well collagen-coated tissue-culture treated microtiter plates in DMEM/F12 medium supplemented with 10% fetal bovine serum, 1% penicillin-streptomycin and 1 μg/ml tetracycline and incubated in a humidified incubator at 37° C. and 5% CO₂ overnight. Next day, the cells were switched to fresh medium without tetracycline and incubated for 4 hrs at 37° C. and 5% CO₂. The cells were then switched to fresh medium without tetracycline and treated with inhibitor A and inhibitor B, at concentration range in the vicinity of their respective EC₅₀ values, and incubated for a duration of 7 days in a humidified incubator at 37° C. and 5% CO₂. The inhibitors tenofovir (TDF) and Compound 3 were diluted in 100% DMSO and the final DMSO concentration in the assay was ≤0.5%. The two inhibitors were tested both singly as well as in combinations in a checkerboard fashion such that each concentration of inhibitor A was combined with each concentration of inhibitor B to determine their combination effects on inhibition of rcDNA production. Following a 7 day-incubation of cells with compound combinations, the level of rcDNA present in the inhibitor-treated wells was measured using a Quantigene 2.0 bDNA assay kit (Affymetrix, Santa Clara, Calif.) with HBV specific custom probe set and manufacturers instructions. The plates were read using a Victor luminescence plate reader (PerkinElmer Model 1420 Multilabel counter) and the relative luminescence units (RLU) data generated from each well was calculated as % inhibition of the untreated control wells and analyzed using the MacSynergy II program to determine whether the combinations were synergistic, additive or antagonistic using the interpretive guidelines established by Prichard and Shipman as follows: synergy volumes <25 μMN (log volume <2) at 95% CI=probably insignificant; 25-50 μM²% (log volume >2 and <5)=minor but significant 50-100 μM²% (log volume >5 and <9)=moderate, may be important in vivo; Over 100 μM²% (log volume >9)=strong synergy, probably important in vivo; volumes approaching 1000 μM²% (log volume >90)=unusually high, check data. The RLU data from the single compound treated cells were analyzed using XL-Fit module in Microsoft Excel to determine EC₅₀ values using a 4-parameter curve fitting algorithm. Concurrently, the effect of compounds on cell viability was assessed using replicate plates, plated at a density of 5,000 cells/well and incubated for 4 days, to determine the ATP content as a measure of cell viability using the cell-titer glo reagent (CTG; Promega Corporation, Madison, Wis.) as per manufacturer's instructions.

### In Vitro Combination of Compound 3 and Tenofovir (TDF):

Compound 3 (concentration range of 3 μM to 0.037 μM in a 3-fold dilution series and 5 point titration) was tested in combination with tenofovir (concentration range of 1 μM to 0.004 μM in a 2-fold dilution series and 9 point titration). The average % inhibition in rcDNA and standard deviations of 4 replicates observed either with compound 3 or TDF treatments alone or in combination is shown in Table 11a. The EC₅₀ values of compound 3 and TDF determined in this experiment are shown in Table 11b. When the observed values of two inhibitor combination were compared to what is expected from additive interaction (Table 11b) for the above concentration range based on the individual contributions of each compound, the combinations were found to be additive (Table 11a and b) as per MacSynergy II analysis and using the interpretive criteria of Prichard and Shipman (1992) as described above.

TABLE 11a
Antiviral Activity of Compound 3 and TDF Combination in HepDE19 cell culture model with rcDNA quantitation
using bDNA assay: Average percent inhibition versus negative control (n = 4 samples per data point)

| [DRUG] | | | | | | | | | | | AVERAGE % | |
|---|---|---|---|---|---|---|---|---|---|---|---|---|
| COMPOUND 3 | | | | | | | | | | | INHIBITION | TDF |
| MM | 0 | 0.004 | 0.008 | 0.016 | 0.031 | 0.063 | 0.125 | 0.250 | 0.500 | 1.000 | OF RCDNA | MM |
| 3 | 92.69 | 93.87 | 96.01 | 94.57 | 94.17 | 94.9 | 91.84 | 94.52 | 97.28 | 97.37 | | |
| 1 | 83.1 | 87.98 | 90.45 | 91.88 | 89.45 | 89.19 | 94.59 | 98.01 | 95.27 | 97.85 | | |
| 0.333 | 34.59 | 47.53 | 50.34 | 45.48 | 64.69 | 70.4 | 83.95 | 92.17 | 94.85 | 96.43 | | |
| 0.111 | −50.41 | −47.53 | −31.05 | −44.75 | 13.61 | 50.62 | 62.26 | 82.59 | 92.55 | 97.17 | | |
| 0.037 | −63.72 | −41.93 | −56.49 | −41.81 | −0.16 | 29.03 | 56.86 | 82.15 | 90.11 | 95.65 | | |
| 0 | 0 | −47.04 | −39.77 | −25.59 | 36.74 | 37.05 | 65.03 | 84.2 | 91.21 | 95.51 | | |

| [DRUG] | | | | | | | | | | | STANDARD | |
|---|---|---|---|---|---|---|---|---|---|---|---|---|
| COMPOUND 3 | | | | | | | | | | | DEVIATION | TDF |
| MM | 0 | 0.004 | 0.008 | 0.016 | 0.031 | 0.063 | 0.125 | 0.250 | 0.500 | 1.000 | (%) | MM |
| 3 | 4.43 | 3.98 | 1.83 | 2.37 | 3.8 | 1.33 | 5.51 | 4.26 | 1.13 | 1.29 | | |
| 1 | 8.73 | 5.43 | 2.73 | 1.92 | 4.32 | 5.01 | 2.65 | 0.84 | 4.58 | 1.21 | | |
| 0.333 | 40.25 | 28.76 | 24.89 | 31.4 | 20.3 | 18.56 | 11.45 | 4.78 | 1.74 | 3.48 | | |
| 0.111 | 96.02 | 90.94 | 47.03 | 93.37 | 79.11 | 18.14 | 25.2 | 8.38 | 5.39 | 1.34 | | |
| 0.037 | 93 | 74.31 | 74.12 | 109.98 | 55.89 | 47.04 | 33.37 | 11.7 | 8.7 | 2.09 | | |
| 0 | 0 | 100.83 | 88.61 | 115.48 | 19.81 | 57.3 | 23.34 | 11.86 | 7 | 3.21 | | |

| [DRUG] | | | | | | | | | | | | |
|---|---|---|---|---|---|---|---|---|---|---|---|---|
| COMPOUND 3 | | | | | | | | | | | ADDITIVE | TDF |
| MM | 0 | 0.004 | 0.008 | 0.016 | 0.031 | 0.063 | 0.125 | 0.250 | 0.500 | 1.000 | INHIBITION | MM |
| 3 | 92.69 | 89.25 | 89.78 | 90.82 | 95.38 | 95.4 | 97.44 | 98.85 | 99.36 | 99.67 | | |
| 1 | 83.1 | 75.15 | 76.38 | 78.78 | 89.31 | 89.36 | 94.09 | 97.33 | 98.51 | 99.24 | | |
| 0.333 | 34.59 | 3.82 | 8.58 | 17.85 | 58.62 | 58.82 | 77.13 | 89.67 | 94.25 | 97.06 | | |
| 0.111 | −50.41 | −121.16 | −110.23 | −88.9 | 4.85 | 5.32 | 47.4 | 76.24 | 86.78 | 93.25 | | |
| 0.037 | −63.72 | −140.73 | −128.83 | −105.62 | −3.57 | −3.06 | 42.75 | 74.13 | 85.61 | 92.65 | | |
| 0 | 0 | −47.04 | −39.77 | −25.59 | 36.74 | 37.05 | 65.03 | 84.2 | 91.21 | 95.51 | | |

| [DRUG] | | | | | | | | | | | SYNERGY | TDF |
|---|---|---|---|---|---|---|---|---|---|---|---|---|
| COMPOUND 3 | | | | | | | | | | | PLOT (99.9%) | MM |
| MM | 0 | 0.004 | 0.008 | 0.016 | 0.031 | 0.063 | 0.125 | 0.250 | 0.500 | 1.000 | BONFERRONI ADJ. | 96% |
| 3 | 0 | 0 | 0.20747 | 0 | 0 | 0 | 0 | 0 | 0 | 0 | SYNERGY | 12.07 |
| 1 | 0 | 0 | 5.08557 | 6.78128 | 0 | 0 | 0 | 0 | 0 | 0 | LOG VOLUME | 1.73 |
| 0.333 | 0 | 0 | 0 | 0 | 0 | 0 | 0 | 0 | 0 | 0 | | |
| 0.111 | 0 | 0 | 0 | 0 | 0 | 0 | 0 | 0 | 0 | 0 | ANTAGONISM | 0 |
| 0.037 | 0 | 0 | 0 | 0 | 0 | 0 | 0 | 0 | 0 | 0 | LOG VOLUME | 0 |
| 0 | 0 | 0 | 0 | 0 | 0 | 0 | 0 | 0 | 0 | 0 | | |

TABLE 11b
Summary of results of in vitro combination studies in HepDE19 cell culture system with rcDNA quantitation using bDNA assay:

| | | | | SYNERGY | SYNERGY | ANTAGONISM | ANTAGONISM | |
|---|---|---|---|---|---|---|---|---|
| | | INHIBITOR A | INHIBITOR B | VOLUME | LOG | VOLUME | LOG | |
| INHIB A | INHIB B | EC₅₀ (MM) | EC₅₀ (MM) | (μM² %)* | VOLUME | (μM² %)* | VOLUME | CONCLUSION |
| CMPD 3 | TDF | 0.454 | 0.088 | 12.07 | 1.73 | 0 | 0 | ADDITIVE |

*AT 99.9% CONFIDENCE INTERVAL

### Example 12

### In Vitro Combination Study Goal:

To determine whether two compounds in a combination treatment would result in a synergistic, antagonistic, or additive effect in a hepatitis B virus (HBV) transfected cell culture. The compound, Compound 5, is a small molecule inhibitor of hepatitis B surface antigen (HBsAg) secretion and SIRNA-NP is a lipid nanoparticle (LNP) encapsulated RNAi inhibitor, which targets viral mRNA and viral antigen expression. An HBV cell culture system was used to determine the effect of combination treatment in this in vitro study.

### Small Molecule Chemical Structure:

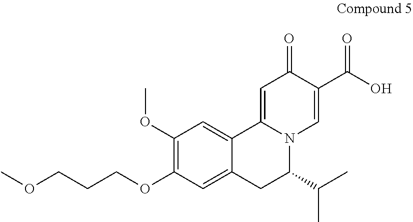 [SMILES: COCCCOc1cc2c(cc1OC)-c1cc(=O)c(C(=O)O)cn1[C@H](C(C)C)C2]

### Lnp Formulation:

SIRNA-NP is a lipid nanoparticle formulation of a mixture of three siRNAs targeting the HBV genome. The following lipid nanoparticle (LNP) product was used to deliver the HBV siRNAs in the experiments reported herein. The values shown in the table are mole percentages. Distearoylphosphatidylcholine is abbreviated as DSPC.

| PEG(2000)-C-DMA | Cationic lipid | Cholesterol | DSPC |
|---|---|---|---|
| 1.6 | 54.6 | 32.8 | 10.9 |

The cationic lipid had the following structure:

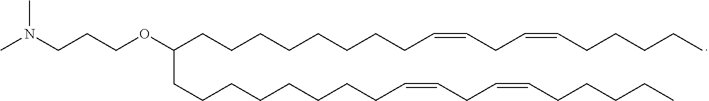 [SMILES: CCCCC/C=C\C/C=C\CCCCCCCCC(CCCCCCCC/C=C\C/C=C\CCCCC)OCCCN(C)C]

siRNA
The sequences of the three siRNAs are shown below.

| Sense Sequence (5′-3′) | Antisense Sequence (5′-3′) |
|---|---|
| rCrCmGrUmGmUrGrCrArCrUm | rUrGrArAmGrCmGrArArGmUm |
| UrCmGrCmUmUrCrArUrU | GrCrAmCrAmCmGrGrUrU |
| rCmUmGmGrCmUrCrArGmUrUm | rCrArCrUrAmGmUrArArAmCr |
| UrAmCmUrAmGmUmGrUrU | UmGrAmGrCmCrArGrUrU |
| rAmCrCmUrCmUrGmCrCmUr | rGrArGrArUrGmArUmUrArGr |
| AmArUmCrArUrCrUrCrUrU | GmCrAmGrAmGrGrUrUrU |

rN = RNA of base N
mN = 2′O-methyl modification of base N

### In Vitro Combination Experimental Protocol:

In vitro combination studies were conducted using the method of Prichard and Shipman (Prichard M N, and Shipman C Jr., Antiviral Research, 1990, 14 (4-5), 181-205; and Prichard M N, et. al., MacSynergy II). The HepG2.2.15 cell culture system is a cell line derived from human hepatoblastoma HepG2 cells that have been stably transfected with the adw2-subtype HBV genome as previously explained in Sells et al. (Proc. Natl. Acad. Sci. U. S. A, 1987. Vol 84:1005-1009). HepG2.2.15 cells secrete Dane-like viral particles, produce HBV DNA, and also produce the viral proteins, hepatitis B e antigen (HBeAg) and hepatitis B surface antigen (HBsAg).

To test the compound combinations, HepG2.2.15 (30,000 cells/well) were plated in 96 well tissue-culture treated microtiter plates in RPMI+L-Glutamine medium supplemented with 1% penicillin-streptomycin, 20 μg/mL geneticin (G418), 10% fetal bovine serum, and incubated in a humidified incubator at 37° C. and 5% CO₂ overnight. The next day, the cells were replenished with fresh medium followed by the addition of Compound 5, dissolved in 100% DMSO, at a concentration range of 0.1 μM to 0.000015 μM. SIRNA-NP was dissolved in 100% RPMI medium and added to cells at a concentration range of 2.5 nM to 0.025 nM. The microtiter cell plates were incubated for a duration of 6 days in a humidified incubator at 37° C. and 5% CO₂. The serial dilutions spanned concentration ranges respective to the EC₅₀ value of each compound, with the final DMSO concentration of the assay being 0.5%. In addition to combination testing of the compounds in a checkerboard fashion, both Compound 5 and SIRNA-NP were also tested alone.

Untreated positive control samples (0.5% DMSO in media) were included on each plate in multiple wells. Following a 6 day-incubation, media was removed from treated cells for use in an HBsAg chemiluminescence immunoassay (CLIA) (Autobio Diagnostics, Cat No. CL0310-2). An HBsAg standard curve was generated to verify that the levels of HBsAg quantification were within the detection limits of the assay. The remaining inhibitor-treated cells were assessed for cytotoxicity by determination of the intracellular adenosine triphosphate (ATP) using a Cell-Titer Glo reagent (Promega) as per manufacturers instructions and by microscopic analysis of the cells throughout the duration of inhibitor treatment. Cell viability was calculated as a percentage of the untreated positive control wells.

The plates were read using an EnVision multimode plate reader (PerkinElmer Model 2104). The relative luminescence units (RLU) data generated from each well was used to calculate HBsAg levels as percent inhibition of the untreated positive control wells and analyzed using the Prichard-Shipman combination model using the MacSynergyll program (Prichard M N, Shipman C Jr. Antiviral Research, 1990. Vol 14(4-5):181-205; Prichard M N, Aseltine K R, and Shipman, C. MacSynergy II. University of Michigan 1992) to determine whether the combinations were synergistic, additive or antagonistic using the interpretive guidelines established by Prichard and Shipman as follows: synergy volumes <25 μM²% (log volume <2) at 95% CI=probably insignificant; 25-50 (log volume >2 and <5)=minor but significant 50-100 (log volume >5 and <9)=moderate, may be important in vivo; Over 100 (log volume >9)=strong synergy, probably important in vivo; volumes approaching 1000 (log volume >90)=unusually high, check data. The RLU data from the single compound treated cells were analyzed using XL-Fit module in Microsoft Excel to determine EC₅₀ values using a 4-parameter curve fitting algorithm.

Compound 5 (concentration range of 0.1 μM to 0.000015 μM in a half-log, 3.16-fold dilution series and 8-point titration) was tested in combination with SIRNA-NP (concentration range of 2.5 nM to 0.025 nM in a half-log, 3.16-fold dilution series and 6-point titration). The combination results were completed in triplicate with each assay consisting of 4 technical repeats. The measurements of synergy and antagonism volumes according to Prichard and Shipman, and interpretation, are shown in Table 12e. The antiviral activity of this combination is shown in Table 12a1, 12a2, and 12a3; synergy and antagonism volumes are shown in Table 12b1, 12b2, and 12b3. The additive inhibition activity of this combination is shown in Table 12d1, 12d2, and 12d3. In this assay system, the combination results in additive inhibition of HBsAg secretion. No significant inhibition of cell viability or proliferation was observed by microscopy or Cell-Titer Glo assay (Table 12c1, 12c2, and 12c3).

### Trial 1

TABLE 12a1
Antiviral Activity of Compound 5 and SIRNA-NP Combination: Average percent
inhibition versus negative control (n = 4 samples per data point)

| | | | | | | | | | | | |
|---|---|---|---|---|---|---|---|---|---|---|---|
| SIRNA-NP, | 0.0025 | 86.52 | 85.69 | 87.32 | 88.31 | 89.63 | 90.42 | 90.86 | 89.67 | 91.42 | 86.52 |
| μM | 0.00079 | 77.54 | 77.93 | 78.77 | 80.65 | 85.38 | 87.61 | 88.97 | 89.29 | 90.33 | 77.54 |
| Avg % | 0.00025 | 58.33 | 51.65 | 58.01 | 66.99 | 71.54 | 78.68 | 82.99 | 85.31 | 85.23 | 58.33 |
| Inhibition | 7.9E−05 | 32.28 | 31.08 | 41.8 | 56.24 | 67.66 | 74.98 | 81.22 | 85.88 | 85 | 32.28 |
| | 2.5E−05 | 23.11 | 23.81 | 29.3 | 46.54 | 60.92 | 70.18 | 78.45 | 80.94 | 82.53 | 23.11 |
| | 0 | 10.26 | 15.09 | 25.37 | 37.06 | 55.53 | 66.43 | 75.94 | 80.86 | 79.69 | 10.26 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12b1
MacSynergy Volume Calculations of Compound 5 and SIRNA-NP Combination:
99.99% confidence interval (Bonferroni Adj. 96%)

| | | | | | | | | | | | |
|---|---|---|---|---|---|---|---|---|---|---|---|
| SIRNA-NP, | 0.0025 | 0 | 0 | 0 | 0 | 0 | 0 | −0.47 | −0.92 | 0 | 0 |
| μM | 0.00079 | 0 | 0 | 0 | 0 | 0 | 0 | −1.51 | 0 | 0 | 0.79 |
| SYNERGY 0 | 0.00025 | 0 | 0 | 0 | 0 | 0 | 0 | 0 | 0 | 0 | 0 |
| Log volume 0 | 7.9E−05 | 0 | 0 | 0 | 0 | 0 | 0 | 0 | 0 | 0 | 0 |
| Antagonism −3.69 | 2.5E−05 | 0 | 0 | 0 | 0 | 0 | 0 | 0 | 0 | 0 | 0 |
| Log volume −0.92 | 0 | 0 | 0 | 0 | 0 | 0 | 0 | 0 | 0 | 0 | 0 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12c1
Cytotoxicity of Compound 5 and SIRNA-NP Combination: Average percent of cell viability vs control

| | | | | | | | | | | | |
|---|---|---|---|---|---|---|---|---|---|---|---|
| SIRNA-NP, | 0.0025 | 103 | 97 | 102 | 102 | 100 | 101 | 105 | 109 | 107 | 123 |
| μM | 0.00079 | 103 | 92 | 99 | 96 | 105 | 106 | 101 | 109 | 101 | 98 |
| Avg % Cell | 0.00025 | 101 | 47 | 122 | 107 | 59 | 109 | 100 | 115 | 104 | 104 |
| Viability | 7.9E−05 | 104 | 128 | 120 | 109 | 152 | 107 | 109 | 106 | 95 | 101 |
| | 2.5E−05 | 95 | 100 | 111 | 107 | 95 | 96 | 100 | 102 | 98 | 115 |
| | 0 | 100 | 113 | 109 | 99 | 100 | 92 | 111 | 112 | 110 | 136 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12d1
Antiviral Activity of Compound 5 and SIRNA-NP Combination: Additive percent
inhibition versus negative control (n = 4 samples per data point)

| | | | | | | | | | | | |
|---|---|---|---|---|---|---|---|---|---|---|---|
| SIRNA-NP, | 0.0025 | 83.86 | 85.52 | 86.3 | 87.95 | 89.84 | 92.82 | 94.58 | 96.12 | 96.91 | 96.72 |
| μM | 0.00079 | 73.95 | 76.62 | 77.88 | 80.56 | 83.6 | 88.42 | 91.26 | 93.73 | 95.01 | 94.71 |
| Additive % | 0.00025 | 49.38 | 54.57 | 57.02 | 62.22 | 68.14 | 77.49 | 83.01 | 87.82 | 90.31 | 89.72 |
| Inhibition | 7.9E−05 | 23.95 | 31.75 | 35.43 | 43.24 | 52.13 | 66.18 | 74.47 | 81.7 | 85.44 | 84.55 |
| | 2.5E−05 | 12.12 | 21.14 | 25.38 | 34.42 | 44.69 | 60.92 | 70.5 | 78.86 | 83.18 | 82.15 |
| | 0 | 0 | 10.26 | 15.09 | 25.37 | 37.06 | 55.53 | 66.43 | 75.94 | 80.86 | 79.69 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

### Trial 2

TABLE 12a2
Antiviral Activity of Compound 5 and SIRNA-NP Combination: Average percent
inhibition versus negative control (n = 4 samples per data point)

| | | | | | | | | | | | |
|---|---|---|---|---|---|---|---|---|---|---|---|
| SIRNA- | 0.0025 | 77.7 | 81.95 | 80.51 | 81.58 | 84.83 | 83.97 | 84.26 | 87.08 | 86.03 | 84.01 |
| NP, μM | 0.00079 | 69.06 | 70.21 | 58.33 | 75.38 | 79.52 | 83.66 | 85.31 | 87.4 | 86.12 | 86.83 |
| Avg % | 0.00025 | 43.84 | 47.41 | 58.38 | 58.03 | 67.92 | 76.4 | 79.69 | 82.57 | 84.39 | 86.46 |
| Inhibition | 7.9E−05 | 25.14 | 44.78 | 40.61 | 46.87 | 58.4 | 70.57 | 73.31 | 84.9 | 88.29 | 87.05 |
| | 2.5E−05 | 14.38 | 27.11 | 38.49 | 45.73 | 55.88 | 65.5 | 77.37 | 78.71 | 83.62 | 86.14 |
| | 0 | 0 | 6.2 | 22.15 | 31.5 | 43.61 | 50.19 | 69.21 | 79.59 | 83.32 | 82.36 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12b2
MacSynergy Volume Calculations of Compound 5 and SIRNA-NP Combination:
99.9% confidence interval (Bonferroni Adj. 96%)

| | | | | | | | | | | | |
|---|---|---|---|---|---|---|---|---|---|---|---|
| SIRNA-NP, | 0.0025 | 0 | 0 | 0 | 0 | 0 | 0 | 0 | 0 | 0 | 0 |
| μM | 0.00079 | 0 | 0 | 0 | 0 | 0 | 0 | 0 | 0 | −3.6 | 0 |
| SYNERGY 0 | 0.00025 | 0 | 0 | 0 | 0 | 0 | 0 | 0 | 0 | 0 | 0 |
| Log volume 0 | 7.9E−05 | 0 | 0 | 0 | 0 | 0 | 0 | 0 | 0 | 0 | 0 |
| Antagonism −3.62 | 2.5E−05 | 0 | 0 | 0 | 0 | 0 | 0 | 0 | 0 | 0 | 0 |
| Log volume −0.9 | 0 | 0 | 0 | 0 | 0 | 0 | 0 | 0 | 0 | 0 | 0 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12c2
Cytotoxicity of Compound 5 and SIRNA-NP Combination: Average percent of cell viability vs control

| | | | | | | | | | | | |
|---|---|---|---|---|---|---|---|---|---|---|---|
| SIRNA- | 0.0025 | 88 | 90 | 74 | 76 | 82 | 77 | 74 | 75 | 90 | 108 |
| NP, μM | 0.00079 | 77 | 72 | 67 | 68 | 65 | 67 | 68 | 65 | 71 | 110 |
| Avg % Cell | 0.00025 | 79 | 75 | 66 | 73 | 71 | 67 | 64 | 63 | 74 | 114 |
| Viability | 7.9E−05 | 88 | 75 | 73 | 76 | 55 | 68 | 68 | 64 | 79 | 116 |
| | 2.5E−05 | 90 | 84 | 68 | 74 | 69 | 71 | 66 | 66 | 80 | 110 |
| | 0 | 100 | 94 | 92 | 113 | 90 | 98 | 109 | 108 | 112 | 133 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12d2
Antiviral Activity of Compound 5 and SIRNA-NP Combination: Additive percent
inhibition versus negative control (n = 4 samples per data point)

| | | | | | | | | | | | |
|---|---|---|---|---|---|---|---|---|---|---|---|
| SIRNA- | 0.0025 | 77.7 | 79.08 | 82.64 | 84.72 | 87.43 | 88.89 | 93.13 | 95.45 | 96.28 | 96.07 |
| NP, μM | 0.00079 | 69.06 | 70.98 | 75.91 | 78.81 | 82.55 | 84.59 | 90.47 | 93.69 | 94.84 | 94.54 |
| Additive % | 0.00025 | 43.84 | 47.32 | 56.28 | 61.53 | 68.33 | 72.03 | 82.71 | 88.54 | 90.63 | 90.09 |
| Inhibition | 7.9E−05 | 9.82 | 15.41 | 29.79 | 38.23 | 49.15 | 55.08 | 72.23 | 81.59 | 84.96 | 84.09 |
| | 2.5E−05 | 23.02 | 27.79 | 40.07 | 47.27 | 56.59 | 61.66 | 76.3 | 84.29 | 87.16 | 86.42 |
| | 0 | 0 | 6.2 | 22.15 | 31.5 | 43.61 | 50.19 | 69.21 | 79.59 | 83.32 | 82.36 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

### Trial 3

TABLE 12a3
Antiviral Activity of Compound 5 and SIRNA-NP Combination: Average percent
inhibition versus negative control (n = 4 samples per data point)

| | | | | | | | | | | | |
|---|---|---|---|---|---|---|---|---|---|---|---|
| SIRNA- | 0.0025 | 89.74 | 92.07 | 93.25 | 94.5 | 95.52 | 96.92 | 98.19 | 98.87 | 99 | 98.59 |
| NP, μM | 0.00079 | 76.48 | 81.81 | 84.52 | 87.38 | 89.73 | 92.94 | 95.86 | 97.42 | 97.71 | 96.77 |
| Avg % | 0.00025 | 52.46 | 63.24 | 68.71 | 74.5 | 79.24 | 85.73 | 91.63 | 94.78 | 95.37 | 93.47 |
| Inhibition | 7.9E−05 | 33.52 | 48.6 | 56.24 | 64.34 | 70.97 | 80.05 | 88.29 | 92.69 | 93.52 | 90.87 |
| | 2.5E−05 | 19.26 | 37.57 | 46.86 | 56.69 | 64.75 | 75.77 | 85.78 | 91.13 | 92.14 | 88.91 |
| | 0 | 0 | 22.68 | 34.18 | 46.36 | 56.34 | 69.99 | 82.39 | 89.01 | 90.26 | 86.26 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12b3
MacSynergy Volume Calculations of Compound 5 and SIRNA-NP Combination:
99.99% confidence interval (Bonferroni Adj. 96%)

| | | | | | | | | | | | |
|---|---|---|---|---|---|---|---|---|---|---|---|
| SIRNA-NP, | 0.0025 | 0 | 0 | 0 | 0 | 0 | 0 | 0 | 0 | 0 | 0 |
| μM | 0.00079 | 0 | 0 | 0 | 0 | 0 | 0 | 0 | 0 | 0 | 0 |
| SYNERGY 0 | 0.00025 | 0 | 0 | 0 | 0 | 0 | 0 | 0 | 0 | 0 | 0 |
| Log volume 0 | 7.9E−05 | 0 | 0 | 0 | 0 | 0 | 0 | 0 | 0 | 0 | 0 |
| Antagonism 0 | 2.5E−05 | 0 | 0 | 0 | 0 | 0 | 0 | 0 | 0 | 0 | 0 |
| Log volume 0 | 0 | 0 | 0 | 0 | 0 | 0 | 0 | 0 | 0 | 0 | 0 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12c3
Cytotoxicity of Compound 5 and SIRNA-NP Combination: Average percent of cell viability vs control

| | | | | | | | | | | | |
|---|---|---|---|---|---|---|---|---|---|---|---|
| SIRNA- | 0.0025 | 97 | 116 | 112 | 124 | 112 | 126 | 124 | 122 | 122 | 95 |
| NP, μM | 0.00079 | 103 | 115 | 112 | 123 | 109 | 118 | 125 | 127 | 126 | 124 |
| Avg % Cell | 0.00025 | 115 | 135 | 129 | 140 | 119 | 135 | 129 | 148 | 136 | 122 |
| Viability | 7.9E−05 | 113 | 129 | 131 | 133 | 130 | 139 | 131 | 138 | 146 | 130 |
| | 2.5E−05 | 113 | 153 | 140 | 140 | 131 | 134 | 137 | 147 | 143 | 124 |
| | 0 | 100 | 131 | 127 | 140 | 131 | 128 | 131 | 141 | 127 | 99 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12d3
Antiviral Activity of Compound 5 and SIRNA-NP Combination: Additive percent
inhibition versus negative control (n = 4 samples per data point)

| | | | | | | | | | | | |
|---|---|---|---|---|---|---|---|---|---|---|---|
| SIRNA- | 0.0025 | 89.74 | 92.07 | 93.25 | 94.5 | 95.52 | 96.92 | 98.19 | 98.87 | 99 | 98.59 |
| NP, μM | 0.00079 | 76.48 | 81.81 | 84.52 | 87.38 | 89.73 | 92.94 | 95.86 | 97.42 | 97.71 | 96.77 |
| Additive % | 0.00025 | 52.46 | 63.24 | 68.71 | 74.5 | 79.24 | 85.73 | 91.63 | 94.78 | 95.37 | 93.47 |
| Inhibition | 7.9E−05 | 33.52 | 48.6 | 56.24 | 64.34 | 70.97 | 80.05 | 88.29 | 92.69 | 93.52 | 90.87 |
| | 2.5E−05 | 19.26 | 37.57 | 46.86 | 56.69 | 64.75 | 75.77 | 85.78 | 91.13 | 92.14 | 88.91 |
| | 0 | 0 | 22.68 | 34.18 | 46.36 | 56.34 | 69.99 | 82.39 | 89.01 | 90.26 | 86.26 |
| | | 0 | 1.00E−06 | 3.16E−06 | 1.0E−05 | 3.17E−05 | 0.0001 | 0.000316 | 0.001 | 0.00316 | 0.1 |

| | |
|---|---|
| Compound | Compound 5, μM |

TABLE 12e
Summary of results of in vitro combination studies in HepG2.2.15
cell culture system with HBsAg quantitation by CLIA

| | Compound 5 | SIRNA-NP | Synergy | Synergy | | | |
|---|---|---|---|---|---|---|---|
| | EC₅₀ | EC₅₀ | Volume | Log | Antagonism | Antagonism | |
| Trial 1 | (μM) | (nM) | (μM² %) | Volume | (μM² %) | Log Volume | Interpretation |
| 1 | 0.002 | 0.00026 | 0 | 0 | −3.69 | −0.92 | Additive |
| 2 | 0.005 | 0.00035 | 0 | 0 | −3.62 | −0.9 | Additive |
| 3 | 0.002 | 0.00020 | 0 | 0 | 0 | 0 | Additive |

*at 99.9% confidence interval

### Example 13

In vitro Combination Study Goal

A goal of this study was to determine whether two drug combinations of tenofovir (in the form of the prodrug tenofovir disoproxil fumarate, or TDF, a nucleotide analog inhibitor of HBV polymerase), or entecavir (in the form of entecavir hydrate, or ETV, a nucleoside analog inhibitor of HBV polymerase), and SIRNA-NP, an siRNA intended to facilitate potent knockdown of all viral mRNA transcripts and viral antigens, is additive, synergistic or antagonistic in vitro using an HBV cell culture model system.

### Chemical Structures of Tenofovir and Entecavir:

### Composition of SIRNA-NP:

SIRNA-NP is a lipid nanoparticle formulation of a mixture of three siRNAs targeting the HBV genome. The following lipid nanoparticle (LNP) formulation was used to deliver the HBV siRNAs. The values shown in the table are mole percentages. The abbreviation DSPC means distearoylphosphatidylcholine, and the PEG was PEG 2000.

| PEG(2000)-C-DMA | Cationic lipid | Cholesterol | DSPC |
|---|---|---|---|
| 1.6 | 54.6 | 32.8 | 10.9 |

The cationic lipid had the following structure:

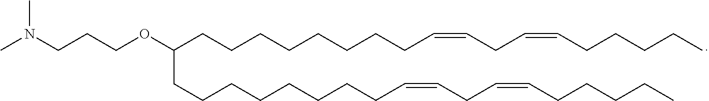 [SMILES: CCCCC/C=C\C/C=C\CCCCCCCCC(CCCCCCCC/C=C\C/C=C\CCCCC)OCCCN(C)C]

The sequences of the three siRNAs are shown below.

| Sense Sequence (5′-3′) | Antisense Sequence (5′-3′) |
|---|---|
| rCrCmGrUmGmUrGrCrArCr | rUrGrArAmGrCmGrArArGmUmGr |
| UmUrCmGrCmUmUrCrArUrU | CrAmCrAmCmGrGrUrU |
| rCmUmGmGrCmUrCrArGmUr | rCrArCrUrAmGmUrArArAmCrUm |
| UmUrAmCmUrAmGmUmGrUrU | GrAmGrCmCrArGrUrU |
| rAmCrCmUrCmUrGmCrCmUr | rGrArGrArUrGmArUmUrArGrGm |
| AmArUmCrArUrCrUrCrUrU | CrAmGrAmGrGrUrUrU |

rN = RNA of base N
mN = 2′O-methyl modification of base N

### In Vitro Combination Experimental Protocol:

In vitro combination studies were conducted using the method of Prichard and Shipman (Prichard M N, Shipman C, Jr., Antiviral Res, 14, 181-205 (1990)). The HepDE19 cell line was developed as described in Guo et al. (Guo et al., J Virol, 81, 12472-12484 (2007)). It is a human hepatoma cell line stably transfected with the HBV genome, and which can express HBV pregenomic RNA and support HBV rcDNA (relaxed circular DNA) synthesis in a tetracycline-regulated manner. HepDE19 cells were plated in 96 well tissue-culture treated microtiter plates in DMEM/F12 medium supplemented with 10% fetal bovine serum+1% penicillin-streptomycin without tetracycline and incubated in a humidified incubator at 37° C. and 5% CO₂ overnight. The next day, the cells were switched to fresh medium and treated with inhibitor A and inhibitor B, at concentration range in the vicinity of their respective EC₅₀ values, and incubated for a duration of 7 days in a humidified incubator at 37° C. and 5% CO₂. The inhibitors were either diluted in 100% DMSO (ETV and TDF) or growth medium (SIRNA-NP) and the final DMSO concentration in the assay was ≤0.5%. The two inhibitors were tested both singly as well as in combinations in a checkerboard fashion such that each concentration of inhibitor A was combined with each concentration of inhibitor B to determine their combination effects on inhibition of rcDNA production. Following a 48 hour-incubation, the level of rcDNA present in the inhibitor-treated wells was measured using a bDNA assay (Affymetrix) with HBV specific custom probe set and manufacturers instructions. The RLU data generated from each well was calculated as % inhibition of the untreated control wells and analyzed using the MacSynergy II program to determine whether the combinations were synergistic, additive or antagonistic using the interpretive guidelines established by Prichard and Shipman as follows: synergy volumes <25 μM²% (log volume <2) at 95% CI=probably insignificant; 25-50 μM²% (log volume >2 and <5)=minor but significant 50-100 μM²% (log volume >5 and <9)=moderate, may be important in vivo; Over 100 μM²% (log volume >9)=strong synergy, probably important in vivo; volumes approaching 1000 μM²% (log volume >90)=unusually high, check data. Concurrently, the effect of inhibitor combinations on cell viability was assessed using replicate plates that were used to determine the ATP content as a measure of cell viability using the Cell-TiterGlo reagent (Promega) as per manufacturer's instructions.

### Results and Conclusion:

### In Vitro Combination of TDF and SIRNA-NP:

TDF (concentration range of 1.0 μM to 0.004 μM in a 2-fold dilution series and 10 point titration) was tested in combination with SIRNA-NP (concentration range of 25 ng/mL to 0.309 ng/mL in a 3-fold dilution series and 5 point titration). The average % inhibition in rcDNA and standard deviations of 4 replicates observed either with TDF or SIRNA-NP treatments alone or in combination is shown in Table 13a. The EC₅₀ values of TDF and SIRNA-NP are shown in Table 13c. When the observed values of two inhibitor combination were compared to what is expected from additive interaction (Table 13a) for the above concentration range, the combinations were found to be additive (Table 13c) as per MacSynergy II analysis and using the interpretive criteria described above by Prichard and Shipman (Prichard M N. 1992. MacSynergy II, University of Michigan).

### In Vitro Combination of Entecavir and SIRNA-NP:

Entecavir (concentration range of 4.0 nM to 0.004 μM in a 2-fold dilution series and 10 point titration) was tested in combination with SIRNA-NP (concentration range of 25 ng/mL to 0.309m/mL in a 3-fold dilution series and 5 point titration). The average % inhibition in rcDNA and standard deviations of 4 replicates observed either with ETV or SIRNA-NP treatments alone or in combination is shown in Table 13b. The EC₅₀ values of ETV and SIRNA-NP are shown in Table 13c. When the two inhibitors were combined in the above concentration range, the concentration combinations were found to be additive as per MacSynergy II analysis and using the interpretive criteria described above by Prichard and Shipman (1992).

TABLE 13a
In vitro Combination of Tenofovir Dipovoxil Fumarate and SIRNA-NP

| [DRUG] | | | | | | | | | | | |
|---|---|---|---|---|---|---|---|---|---|---|---|
| SIRNA-NP | | | | | | | | | | | AVERAGE % INHIBITION |
| ng/mL | 0 | 0.004 | 0.008 | 0.016 | 0.031 | 0.063 | 0.125 | 0.250 | 0.500 | 1.000 | TDF (μM) |
| 25 | 93.58 | 90.91 | 94.48 | 93.31 | 93.59 | 95.77 | 92.36 | 95.99 | 94.11 | 94.97 | |
| 8.333 | 88.63 | 88.81 | 88.05 | 92.79 | 92.07 | 92.97 | 95.69 | 95.77 | 94.62 | 97.07 | |
| 2.778 | 80.6 | 72.21 | 73.62 | 74.95 | 84.77 | 86.4 | 91.21 | 93.53 | 95.62 | 97.56 | |
| 0.926 | 44.48 | 36.07 | 41.83 | 44.94 | 60.31 | 76.81 | 82.62 | 91.36 | 95 | 97.09 | |
| 0.309 | 26.53 | 13.83 | 9.48 | 26.5 | 32.64 | 53.59 | 73.58 | 82.75 | 90.84 | 96.66 | |
| 0 | 0 | −5.27 | 0.67 | 2.82 | 6.57 | 41.67 | 66.08 | 81.55 | 90.85 | 94.55 | |

| [DRUG] | | | | | | | | | | | |
|---|---|---|---|---|---|---|---|---|---|---|---|
| SIRNA-NP | | | | | | | | | | | STANDARD DEVIATION (%) |
| ng/mL | 0 | 0.003906 | 0.00781 | 0.01563 | 0.03125 | 0.0625 | 0.125 | 0.25 | 0.5 | 1 | TDF (μM) |
| 25 | 8.23 | 5.78 | 2.1 | 5.36 | 4.44 | 2.77 | 5.57 | 2.31 | 3.88 | 1.7 | |
| 8.333 | 5.38 | 4.15 | 6.01 | 5.32 | 3.97 | 1.82 | 2.89 | 3.61 | 3.13 | 2.02 | |
| 2.778 | 13.12 | 12 | 6.21 | 14.12 | 12.42 | 5.29 | 3.1 | 1.92 | 1.12 | 1.34 | |
| 0.926 | 16.91 | 9.73 | 29.33 | 16.45 | 21.14 | 5.83 | 6.39 | 2.16 | 2.29 | 1.2 | |
| 0.309 | 12.04 | 43.02 | 33.58 | 19.89 | 52.19 | 25.65 | 17.47 | 6.61 | 5.58 | 1.79 | |
| 0 | 0 | 23.5 | 44.96 | 26.95 | 54.17 | 21.72 | 15.9 | 12.86 | 1.64 | 0.99 | |

| [DRUG] | | | | | | | | | | | |
|---|---|---|---|---|---|---|---|---|---|---|---|
| SIRNA-NP | | | | | | | | | | | ADDITIVE INHIBITION |
| ng/mL | 0 | 0.003906 | 0.00781 | 0.01563 | 0.03125 | 0.0625 | 0.125 | 0.25 | 0.5 | 1 | TDF (μM) |
| 25 | 93.58 | 93.24 | 93.62 | 93.76 | 94 | 96.26 | 97.82 | 98.82 | 99.41 | 99.65 | |
| 8.333 | 88.63 | 88.03 | 88.71 | 88.95 | 89.38 | 93.37 | 96.14 | 97.9 | 98.96 | 99.38 | |
| 2.778 | 80.6 | 79.58 | 80.73 | 81.15 | 81.87 | 88.68 | 93.42 | 96.42 | 98.22 | 98.94 | |
| 0.926 | 44.48 | 41.55 | 44.85 | 46.05 | 48.13 | 67.62 | 81.17 | 89.76 | 94.92 | 96.97 | |
| 0.309 | 26.53 | 22.66 | 27.02 | 28.6 | 31.36 | 57.14 | 75.08 | 86.44 | 93.28 | 96 | |
| 0 | 0 | −5.27 | 0.67 | 2.82 | 6.57 | 41.67 | 66.08 | 81.55 | 90.85 | 94.55 | |

| [DRUG] | | | | | | | | | | | | |
|---|---|---|---|---|---|---|---|---|---|---|---|---|
| SIRNA-NP | | | | | | | | | | | SYNERGY PLOT (99.9%) | |
| ng/mL | 0 | 0.00 | 0.01 | 0.02 | 0.03 | 0.06 | 0.13 | 0.25 | 0.5 | 1 | Bonferroni Adj. | 96% |
| 25.0 | 0 | 0 | 0 | 0 | 0 | 0 | 0 | 0 | 0 | 0 | SYNERGY | 0 |
| 8.333 | 0 | 0 | 0 | 0 | 0 | 0 | 0 | 0 | 0 | 0 | log volume | 0 |
| 2.778 | 0 | 0 | 0 | 0 | 0 | 0 | 0 | 0 | 0 | 0 | | |
| 0.926 | 0 | 0 | 0 | 0 | 0 | 0 | 0 | 0 | 0 | 0 | ANTAGONISM | 0 |
| 0.309 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 13b
In vitro Combination of Entecavir and SIRNA-NP

| [DRUG] | | | | | | | | | | | |
|---|---|---|---|---|---|---|---|---|---|---|---|
| SIRNA-NP | | | | | | | | | | | AVERAGE % INHIBITION |
| ng/mL | 0 | 0.016 | 0.031 | 0.063 | 0.125 | 0.250 | 0.500 | 1.000 | 2.000 | 4.000 | ETV (nM) |
| 25 | 94.31 | 92.38 | 93.86 | 94.7 | 93.85 | 95.21 | 92.88 | 95.49 | 94.28 | 95.63 | |
| 8.333 | 90.41 | 91.44 | 91.71 | 89.9 | 90.91 | 92.34 | 94.03 | 94.33 | 95.37 | 95.21 | |
| 2.778 | 76.74 | 74.61 | 62.81 | 75.26 | 79.3 | 82.04 | 86.38 | 90.76 | 92.08 | 91.18 | |
| 0.926 | 46.84 | 39.16 | 41.37 | 58.29 | 48.88 | 49.94 | 62.39 | 72.82 | 82.81 | 86.42 | |
| 0.309 | 28.68 | 27.78 | 12.18 | 5.93 | 8.2 | 19.18 | 27.55 | 56.01 | 73.11 | 79.23 | |
| 0 | 0 | −43.72 | −50.07 | −30.26 | −23.47 | −21.25 | 10.24 | 41.71 | 58.35 | 69.99 | |

| [DRUG] | | | | | | | | | | | |
|---|---|---|---|---|---|---|---|---|---|---|---|
| SIRNA-NP | | | | | | | | | | | STANDARD DEVIATION (%) |
| ng/mL | 0 | 0.015625 | 0.03125 | 0.0625 | 0.125 | 0.25 | 0.5 | 1 | 2 | 4 | ETV (nM) |
| 25 | 1.67 | 6.18 | 2.63 | 3.7 | 2.4 | 3.83 | 4.89 | 3.65 | 3.15 | 1.22 | |
| 8.333 | 2.86 | 5.03 | 5.65 | 8.63 | 2.5 | 1.68 | 2.18 | 3.07 | 1.44 | 2.47 | |
| 2.778 | 12.28 | 9.55 | 32.22 | 11.86 | 9.31 | 4.95 | 4.87 | 2.25 | 2.64 | 6.79 | |
| 0.926 | 17.81 | 22.17 | 35.16 | 8.83 | 25.87 | 17.46 | 17.5 | 7.78 | 4.36 | 5.46 | |
| 0.309 | 12.26 | 11.58 | 26.87 | 28.27 | 30.31 | 21.45 | 38.93 | 12.99 | 12.27 | 6.89 | |
| 0 | 0 | 47.73 | 38.3 | 41.27 | 25.17 | 56.81 | 65.13 | 33.53 | 17.75 | 11.48 | |

| [DRUG] | | | | | | | | | | | |
|---|---|---|---|---|---|---|---|---|---|---|---|
| SIRNA-NP | | | | | | | | | | | ADDITIVE INHIBITION |
| ng/mL | 0 | 0.015625 | 0.03125 | 0.0625 | 0.125 | 0.25 | 0.5 | 1 | 2 | 4 | ETV (nM) |
| 25 | 94.31 | 91.82 | 91.46 | 92.59 | 92.97 | 93.1 | 93.73 | 96.68 | 97.63 | 98.29 | |
| 8.333 | 90.41 | 86.22 | 85.61 | 87.51 | 88.16 | 88.37 | 89.43 | 94.41 | 96.01 | 97.12 | |
| 2.778 | 76.74 | 66.57 | 65.09 | 69.7 | 71.28 | 71.8 | 74.36 | 86.44 | 90.31 | 93.02 | |
| 0.926 | 46.84 | 23.6 | 20.22 | 30.75 | 34.36 | 35.54 | 41.4 | 69.01 | 77.86 | 84.05 | |
| 0.309 | 28.68 | −2.5 | −7.03 | 7.1 | 11.94 | 13.52 | 21.38 | 58.43 | 70.3 | 78.6 | |
| 0 | 0 | −43.72 | −50.07 | −30.26 | −23.47 | −21.25 | 10.24 | 41.71 | 58.35 | 69.99 | |

| [DRUG] | | | | | | | | | | | | |
|---|---|---|---|---|---|---|---|---|---|---|---|---|
| SIRNA-NP | | | | | | | | | | | SYNERGY PLOT (99.9%) | |
| ng/mL | 0 | 0.02 | 0.03 | 0.06 | 0.13 | 0.25 | 0.50 | 1 | 2 | 4 | Bonferroni Adj. | 96% |
| 25.0 | 0 | 0 | 0 | 0 | 0 | 0 | 0 | 0 | 0 | 0 | SYNERGY | 0 |
| 8.333 | 0 | 0 | 0 | 0 | 0 | 0 | 0 | 0 | 0 | 0 | log volume | 0 |
| 2.778 | 0 | 0 | 0 | 0 | 0 | 0 | 0 | 0 | 0 | 0 | | |
| 0.926 | 0 | 0 | 0 | 0 | 0 | 0 | 0 | 0 | 0 | 0 | ANTAGONISM | 0 |
| 0.309 | 0 | 0 | 0 | 0 | 0 | 0 | 0 | 0 | 0 | 0 | log volume | 0 |
| 0 | 0 | 0 | 0 | 0 | 0 | 0 | 0 | 0 | 0 | 0 | | |

TABLE 13c
Summary of results of in vitro combination studies in AML12-HBV10
cell culture system with rcDNA quantitation using bDNA assay:

| | | Inhibitor A | | Synergy | Synergy | Antagonism | | |
|---|---|---|---|---|---|---|---|---|
| | | EC₅₀ | Inhibitor B | Volume | Log | Volume | Antagonism | |
| Inhibitor A | Inhibitor B | (ng/mL) | EC₅₀ | (μM² %)* | Volume | (μM² %)* | Log Volume | Conclusion |
| SIRNA-NP | Tenofovir | 0.947 | 0.089 | 0 | 0 | 0 | 0 | Additive |
| | (TDF, μM) | | | | | | | |
| SIRNA-NP | Entecavir | 0.906 | 1.780 | 5.37 | 0.77 | 0 | 0 | Additive |
| | (ETV, nM) | | | | | | | |

*at 99.9% confidence interval

### Example 14

The following compound is referenced in the Examples. Compound 20 can be prepared using known procedures. For example, Compound 20 can be prepared as described in International Patent Application Publication Number WO2015113990.

| Compound Number | |
|---|---|
| or Name | Structure |
| 20 | 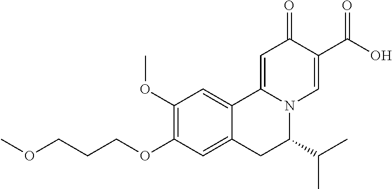 [SMILES: COCCCOc1cc2c(cc1OC)-c1cc(=O)c(C(=O)O)cn1[C@H](C(C)C)C2] |

A mouse model of hepatitis B virus (HBV) was used to assess the anti-HBV effects of a small molecule inhibitor of sAg production and HBV-targeting siRNAs (SIRNA-NP), both as independent treatments and in combination with each other.

The following lipid nanoparticle (LNP) formulation was used to deliver the HBV siRNAs. The values shown in the table are mole percentages. The abbreviation DSPC means distearoylphosphatidylcholine.

| PEG(2000)-C-DMA | Cationic lipid | Cholesterol | DSPC |
|---|---|---|---|
| 1.6 | 54.6 | 32.8 | 9 |

The cationic lipid had the following structure:

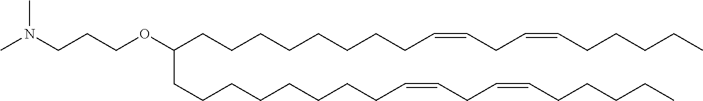 [SMILES: CCCCC/C=C\C/C=C\CCCCCCCCC(CCCCCCCC/C=C\C/C=C\CCCCC)OCCCN(C)C]

1E11 viral genomes of AAV1.2 (described in Huang, L R et al. Gastroenterology, 2012, 142(7):1447-50) was administered to C57/B16 mice via tail vein injection. This viral vector contains a 1.2-fold overlength copy of the HBV genome and expresses HBV surface antigen (HBsAg) amongst other HBV products. Serum HBsAg expression in mice was monitored using an enzyme immunoassay. Animals were sorted (randomized) into groups based on serum HBsAg levels such that a) all animals were confirmed to express HBsAg and b) HBsAg group means were similar to each other prior to initiation of treatments.

Animals were treated with Compound 20 as follows: Starting on Day 0, a 3.0 mg/kg dosage of Compound 20 was administered orally to animals on a twice-daily frequency for a total of 56 doses between Days 0 and 28. Compound 20 was dissolved in a co-solvent formation for administration. Negative control animals were administered either the co-solvent formulation alone, or were not treated with any test article. Animals were treated with lipid nanoparticle (LNP)-encapsulated HBV-targeting siRNAs as follows: On Day 0, an amount of test article equivalent to 0.3 mg/kg siRNA was administered intravenously. The HBsAg expression levels for each treatment were compared against the Day 0 (pre-dose) values for that group.

The effect of these treatments was determined by collecting blood on Days 0 (pre-treatment), 7, 14, and 28 and analyzing it for serum HBsAg content. Table 14 shows the treatment group mean (n=5 (n=4 for siHBV and vehicle combination treatment); ±standard error of the mean) serum HBsAg concentration expressed as a percentage of the individual animal pre-treatment baseline value at Day 0.

The data demonstrate the degree of serum HBsAg reduction in response to the combination of Compound 20 and HBV siRNA, both alone and in combination. At every time point tested, the combination of Compound 20 and HBV siRNA treatments yielded reduction of serum HBsAg that was as good or better than any of the individual monotherapy treatments.

TABLE 14
Single and Combination Treatment Effect of Compound 20 and Three
HBV siRNAs on Serum HBV sAg in a Mouse Model of HBV Infection

| Treatment 1 (Oral) | Treatment 2 (IV) | D 0 | D 7 | D 14 | D 21 | D 28 |
|---|---|---|---|---|---|---|
| None | None | 100 ± 0 | 80 ± 12 | 100 ± 18 | 72 ± 15 | 72 ± 16 |
| Vehicle | None | 100 ± 0 | 37 ± 8 | 62 ± 9 | 112 ± 66 | 127 ± 67 |
| Compound 20 | None | 100 ± 0 | 7 ± 1 | 8 ± 2 | 7 ± 2 | 8 ± 2 |
| Vehicle | HBV siRNA | 100 ± 0 | 1 ± 0 | 3 ± 2 | 19 ± 9 | 45 ± 25 |
| Compound 20 | HBV siRNA | 100 ± 0 | 1 ± 0 | 2 ± 1 | 5 ± 2 | 8 ± 2 |

### Examples 15-24

### Materials and Methods for Studies in Primary Human Hepatocytes

### Animals

FRG mice were purchased from Yecuris (Tualatin, Oreg., USA). Detailed information of the mice is shown in the table below. The study was approved by the WuXi IACUC (Institutional Animal Care and Use Committee, IACUC protocol R20160314-Mouse). Mice are allowed to acclimate to the new environment for 7 days. The mice were monitored for general health and any signs of physiological and behavioral anomaly daily.

FRG mouse technical data

| Cage | Mouse | Donor | Date of | Date of | Albumin (μg/ml) | | BW (g) - |
|---|---|---|---|---|---|---|---|
| ID | ID | ID | birth | transplant | preshipment | Gender | preshipment |
| 1 | 37094 | HHM30017 | Dec. 15, 2015 | Jan. 28, 2016 | 4887 | male | 24.9 |
| 2 | 37211 | HHM27018 | Jan. 3, 2016 | Feb. 24, 2016 | 4284 | male | 23.5 |
| 3 | 37258 | HHM27018 | Jan. 6, 2016 | Feb. 24, 2016 | 4282 | male | 29.4 |
| 4 | 37611 | HHM30017 | Feb. 22, 2016 | Apr. 6, 2016 | 6627 | male | 25.3 |
| 5 | 37955 | HHM27018 | Mar. 31, 2016 | May 19, 2016 | 5990 | male | 25.5 |
| 6 | 37900 | HHM27018 | Mar. 23, 2016 | May 4, 2016 | 4802 | male | 27.1 |
| 7 | 37976 | HHM27018 | Mar. 20, 2016 | May 4, 2016 | 4520 | male | 24.7 |

### Test Articles

Compounds 3, 22, 23, 24 and 25 were provided by Arbutus Biopharma. Peg-interferon alfa-2a (Roche, 180 μg/0.5 ml) was provided by WuXi. TAF, Entecavir, Tenofovir, Lamivudine and TDF were provided in DMSO solution by WuXi. Information on the compounds is shown in the table below.

Information of the test articles

| Compound name | Vial ID | M. Wt. | Size | | Vendor |
|---|---|---|---|---|---|
| 25 | 031NH | 401.19 | 3.1 | mg | Arbutus |
| 3 | 031NR | 386.4 | 3.8 | mg | Arbutus |
| 22 | 031NP | 398.4 | 2.9 | mg | Arbutus |
| 23 | 031NT | 379.3 | 2.9 | mg | Arbutus |
| 24 | 031NV | 396.8 | 2.6 | mg | Arbutus |
| | | Catalog | | | |
| Compound name | Vendor | No. | Stock Conc. | | |
| Peginterferon | Roche | | 180 μg/0.5 ml | | Provide by |
| alfa-2a | | | (5040000 | | WuXi |
| | | | IU/ml) | | |
| TAF | SelleckChem | S7856 | 10 | mM | Provide by |
| | | | | | WuXi |
| TDF | Shanghai | | 20 | mM | Provide by |
| | Sphchem Co., | | | | WuXi |
| | Ltd. | | | | |

### Viruses

D type HBV was concentrated from HepG2.2.15 culture supernatants. The information of the viruses is shown in the table below.

Information of the HBV

| | | HBV titer | | |
|---|---|---|---|---|
| | | in serum | | |
| Virus ID | Lot# | (GE*/ml) | Genotype | Source |
| HBV_2.2.15 | HBV20160407 | 2.00E+09 | D | HepG2.2.15 |
| | | GE/ml | (Genebank | supernatants |
| | | | ID: U95551) | |
| HBV_2.2.15 | B161011 | 1.9E+09 | D | HepG2.2.15 |
| | | GE/ml | (Genebank | supernatants |
| | | | ID: U95551) | |
| HBV_2.2.15 | B161129 | 1.5E+09 | D | HepG2.2.15 |
| | | GE/ml | (Genebank | supernatants |
| | | | ID: U95551) | |

*GE, HBV genome equivalent.

### Reagents

The major reagents used in the study were QIAamp 96 DNA Blood Kit (QIAGEN #51161), FastStart Universal Probe Master (Roche #04914058001), Cell Counting Kit-8 (CCK-8) (Biolite #35004), HBeAg ELISA kit (Antu #CL 0312) and HBsAg ELISA kit (Antu #CL 0310).

### Instruments

The major instruments used in the study were BioTek Synergy 2, SpectraMax (Molecular Devices), 7900HT Fast Real-Time PCR System (ABI) and Quantistudio 6 Real-Time PCR System (ABI).

### Harvest of Primary Human Hepatocytes (PHH)

The mouse liver perfusion was applied to isolate PHHs. The isolated hepatocytes were further purified by Percoll. The cells were resuspended with culture media and seeded into the 96-well plates (6×10⁴ cell/well) or 48-well plates (1.2×10⁵ cell/well). The PHHs were infected with a D type HBV one day post seeding (day 1).

### Culture and Treatment of PHHs.

On day 2, the test compounds were diluted and added into the cell culture plates. The culture media containing the compounds were refreshed every other day. The cell culture supernatants were collected on day 8 for the HBV DNA and antigen determinations.

### Determination of EC₅₀ Values.

The compounds were tested at 7 concentrations, 3-fold dilution, in triplicate.

### Double Combination Study.

Two compounds were tested at 5×5 matrix, in triplicate plates.

### Assay for Cytotoxicity by Cell Counting Kit-8 at Day 8

The culture media was removed from the cell culture plate, and then CCK8 (Biolite #35004) working solution was added to the cells. The plate was incybated at 37° C., and the absorbance was measured at 450 nm wavelength and reference absorbance was measured at 650 nm wavelength by SpectraMax.

Quantification of HBV DNA in the Culture Supernatants by qPCR

DNA in the culture supernatants harvested on days 8 were isolated with QIAamp 96 DNA Blood Kit (Qiagen-51161). For each sample, 100 μl of the culture supernatants was used to extract DNA. The DNA was eluted with 100 μl, 150 μl or 180 μl of AE. HBV DNA in the culture supernatants was quantified by qPCR. The combination effect was analyzed by the MacSynergy software. The primers are described below.

### Primer Information

Primer R
GACAAACGGGCAACATACCTT
Primer F
GTGTCTGCGGCGTTTTATCA
Probe
5′FAM CCTCTKCATCCTGCTGCTATGCCTCATC 3′TAMRA

### Measurement of HBsAg and HBeAg in the Culture Supernatants by ELISA

HBsAg/HBeAg in the culture supernatants harvested on days 8 were measured by the HBsAg/HBeAg ELISA kit (Autobio) according to the manual. The samples were diluted with PBS to get the signal in the range of the standard curve. The inhibition rates were calculated with the formula below. The combination effect was analyzed by the MacSynergy software.

% Inh. HBsAg=[1-HBsAg quantity of sample/HBV quantity of DMSO control]×100.

% Inh. HBeAg=[1-HBeAg quantity of sample/HBV quantity of DMSO control]×100.

### SIRNA-NP

SIRNA-NP is a lipid nanoparticle formulation of a mixture of three siRNAs targeting the HBV genome. The following lipid nanoparticle (LNP) formulation was used to deliver the HBV siRNAs. The values shown in the table are mole percentages. The abbreviation DSPC means distearoylphosphatidylcholine.

| PEG-C-DMA | Cationic lipid | Cholesterol | DSPC |
|---|---|---|---|
| 1.6 | 54.6 | 32.8 | 10.9 |

The cationic lipid had the following structure:

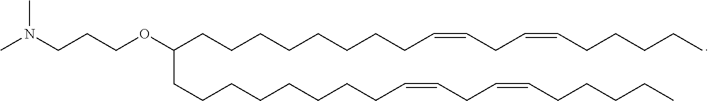 [SMILES: CCCCC/C=C\C/C=C\CCCCCCCCC(CCCCCCCC/C=C\C/C=C\CCCCC)OCCCN(C)C]

The sequences of the three siRNAs are shown below.

| Sense Sequence (5′-3′) | Antisense Sequence (5′-3′) |
|---|---|
| rCrCmGrUmGmUrGrCrArCr | rUrGrArAmGrCmGrArArGmUmGr |
| UmUrCmGrCmUmUrCrArUrU | CrAmCrAmCmGrGrUrU |
| rCmUmGmGrCmUrCrArGmUr | rCrArCrUrAmGmUrArArAmCrUm |
| UmUrAmCmUrAmGmUmGrUrU | GrAmGrCmCrArGrUrU |
| rAmCrCmUrCmUrGmCrCmUr | rGrArGrArUrGmArUmUrArGrGm |
| AmArUmCrArUrCrUrCrUrU | CrAmGrAmGrGrUrUrU |

rN = RNA of base N
mN = 2′O-methyl modification of base N

### Composition of Pegylated Interferon Alpha 2a (IFNα2a):

This agent was purchased from a commercial source:

| Sample ID | Vendor | Size | Lot No. | Stock Conc. |
|---|---|---|---|---|
| Peginterferon | Roche | 180 ug/0.5 ml | B1370 | 5040000 IU/mL |
| alfa-2a | | | | |

The following compounds were also used.

| Compound Name or ID number | Structure |
|---|---|
| 3 | 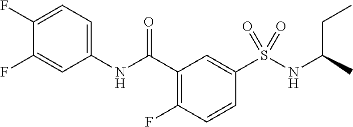 [SMILES: CC[C@@H](C)NS(=O)(=O)c1ccc(F)c(C(=O)Nc2ccc(F)c(F)c2)c1] |
| 22 | 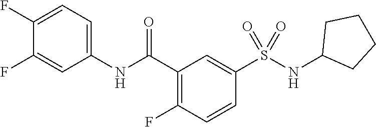 [SMILES: O=C(Nc1ccc(F)c(F)c1)c1cc(S(=O)(=O)NC2CCCC2)ccc1F] |
| 23 | 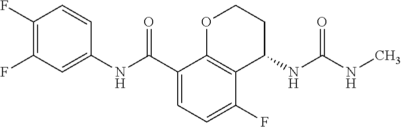 [SMILES: CNC(=O)N[C@H]1CCOc2c(C(=O)Nc3ccc(F)c(F)c3)ccc(F)c21] |
| 24 | 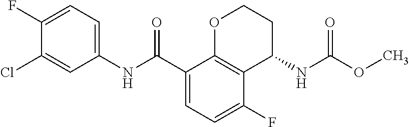 [SMILES: COC(=O)N[C@H]1CCOc2c(C(=O)Nc3ccc(F)c(Cl)c3)ccc(F)c21] |
| 25 | 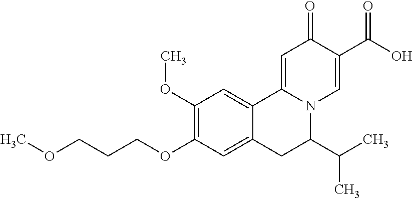 [SMILES: COCCCOc1cc2c(cc1OC)-c1cc(=O)c(C(=O)O)cn1C(C(C)C)C2] |
| Tenofovir Disoproxil Fumarate (TDF) | 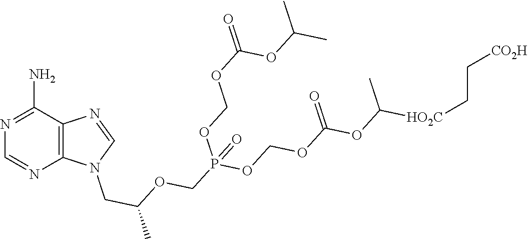 |
| Tenofovir Alafenamide (TAF) | 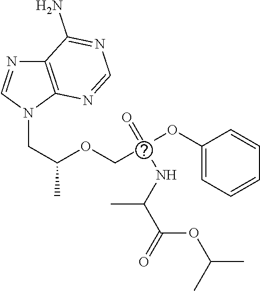 |
| GLS4 (HAP) | 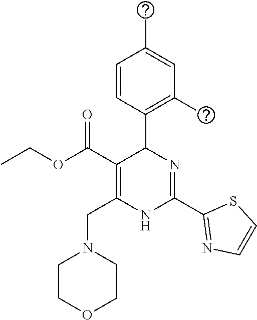 [SMILES: CCOC(=O)C1=C(CN2CCOCC2)NC(c2nccs2)=NC1c1ccc(C)cc1C] |

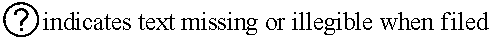

### Example 15

### In Vitro Combination of Compound 24 and TDF

### Study Goal

To determine whether a two-drug combination of compound 24 (a small molecule inhibitor of HBV encapsidation belonging to the amino chroman chemical class), and tenofovir (in the form of the prodrug tenofovir disoproxil fumarate, or TDF, a nucleotide analog inhibitor of HBV polymerase), is additive, synergistic or antagonistic in vitro using HBV-infected human primary hepatocytes in a cell culture model system.

### Results and Conclusion

TDF (concentration range of 10.0 nM to 0.12 nM in a 3-fold dilution series and 5 point titration) was tested in combination with 24 (concentration range of 1000 nM to 12.36 nM in a 3-fold dilution series and 5 point titration). The average % inhibition in HBV DNA, HBsAg, and HBeAg and standard deviations of 3 replicates observed either with 24 or TDF treatments alone or in combination are shown in Tables 15a, 15b and 15c as indicated below. The EC₅₀ values of TDF and 24 were determined in an earlier experiment and are shown in Table 15d; some variance was observed from different lots of PHH cells.

When the observed values of a two-inhibitor combination were compared to what is expected from additive interaction for the above concentration range, the combinations were found to be synergistic or additive, with no antagonism (Table 15d) as per MacSynergy II analysis and using the interpretive criteria described above by Prichard and Shipman (1992). No significant inhibition of cell viability or proliferation was observed by microscopy or CCK8 assay.

TABLE 15a
Effect on HBV DNA in In Vitro Combination of Compound 24 and TDF

| [DRUG] | | | | | | | AVERAGE % INHIBITION |
|---|---|---|---|---|---|---|---|
| TDF (nM) | 0.00 | 12.35 | 37.04 | 111.11 | 333.33 | 1000.00 | COMPOUND 24 (nM) |
| 10.00 | −15.4 | 16.23 | 29.76 | 33.41 | 71.99 | 82.62 | |
| 3.33 | 3.15 | 24.17 | 30.1 | 39.55 | 71.87 | 87.48 | |
| 1.11 | 2.49 | 24.8 | 24.74 | 33.45 | 73.25 | 86.58 | |
| 0.37 | −6.24 | 17.61 | 35.35 | 37.05 | 68.52 | 87.29 | |
| 0.12 | −1.16 | 25.72 | 25.72 | 33.54 | 75.06 | 86.83 | |
| 0.00 | 0 | −37.82 | −31.86 | −4.54 | 59.29 | 81.07 | |

| [DRUG] | | | | | | | STANDARD DEVIATION (%) |
|---|---|---|---|---|---|---|---|
| TDF (nM) | 0.00 | 12.35 | 37.04 | 111.11 | 333.33 | 1000.00 | COMPOUND 24 (nM) |
| 10.00 | 20.95 | 6.25 | 5.54 | 19.51 | 4.45 | 1.41 | |
| 3.33 | 2.99 | 2.31 | 4.58 | 5.73 | 2.67 | 0.32 | |
| 1.11 | 16.52 | 2.03 | 9.07 | 11.11 | 3.67 | 1.02 | |
| 0.37 | 19.46 | 1.06 | 13.17 | 2.12 | 3.62 | 0.7 | |
| 0.12 | 21.38 | 7.78 | 1.65 | 6.37 | 1.38 | 1.46 | |
| 0.00 | 15.8 | 17.16 | 15.42 | 5.85 | 4.36 | 3.8 | |

| [DRUG] | | | | | | | ADDITIVE INHIBITION |
|---|---|---|---|---|---|---|---|
| TDF (nM) | 0.00 | 12.35 | 37.04 | 111.11 | 333.33 | 1000.00 | Compound 24 (nM) |
| 10.00 | −15.4 | −59.04 | −52.17 | −20.64 | 53.02 | 78.15 | |
| 3.33 | 3.15 | −33.48 | −27.71 | −1.25 | 60.57 | 81.67 | |
| 1.11 | 2.49 | −34.39 | −28.58 | −1.94 | 60.3 | 81.54 | |
| 0.37 | −6.24 | −46.42 | −40.09 | −11.06 | 56.75 | 79.89 | |
| 0.12 | −1.16 | −39.42 | −33.39 | −5.75 | 58.82 | 80.85 | |
| 0.00 | 0 | −37.82 | −31.86 | −4.54 | 59.29 | 81.07 | |

| [DRUG] | | | | | | | SYNERGY PLOT (99.9%) | |
|---|---|---|---|---|---|---|---|---|
| TDF (nM) | 0 | 12.346 | 37.037 | 111.11 | 333.33 | 1000 | Bonferroni Adj. | 98% |
| 10.00 | 0 | 54.7013 | 63.6979 | 0 | 4.32505 | 0 | SYNERGY | 586.54 |
| 3.33 | 0 | 50.0478 | 42.7372 | 21.9426 | 2.51303 | 4.75688 | log volume | 133.52 |
| 1.11 | 0 | 52.5093 | 23.4706 | 0 | 0.87203 | 1.68318 | | |
| 0.37 | 0 | 60.5415 | 32.0975 | 41.1331 | 0 | 5.0963 | ANTAGONISM | 0 |
| 0.12 | 0 | 39.536 | 53.6799 | 18.3263 | 11.6984 | 1.17514 | log volume | 0 |
| 0.00 | 0 | 0 | 0 | 0 | 0 | 0 | | |

TABLE 15b
Effect on HBsAg in In Vitro Combination of Compound 24 and TDF

| [DRUG] | | | | | | | AVERAGE % INHIBITION |
|---|---|---|---|---|---|---|---|
| TDF (nM) | 0.00 | 12.35 | 37.04 | 111.11 | 333.33 | 1000.00 | COMPOUND 24 |
| 10.00 | −6.65 | −16.37 | −4.65 | −5.16 | −9.32 | 22.45 | |
| 3.33 | −1.28 | 15.87 | 17.06 | 10.42 | 10.42 | 34.58 | |
| 1.11 | −3.54 | 11.17 | 11.06 | 14.54 | 15.54 | 33.45 | |
| 0.37 | −3.32 | 1.98 | 7.61 | 5.28 | 8.04 | 35.4 | |
| 0.12 | −3.31 | 8.08 | −0.03 | −1.09 | −3.03 | 29.86 | |
| 0.00 | 0 | −17.92 | −21.67 | −27.16 | −25.87 | 12.01 | |

| [DRUG] | | | | | | | STANDARD DEVIATION (%) |
|---|---|---|---|---|---|---|---|
| TDF (nM) | 0.00 | 12.35 | 37.04 | 111.11 | 333.33 | 1000.00 | COMPOUND 24 |
| 10.00 | 8.24 | 26.3 | 12.4 | 23.19 | 9.78 | 5.74 | |
| 3.33 | 8.1 | 15.58 | 17.2 | 4.68 | 3.18 | 4.67 | |
| 1.11 | 15.01 | 5.34 | 11.05 | 6.21 | 3.77 | 6.92 | |
| 0.37 | 11.33 | 16.35 | 16.52 | 12.36 | 17.13 | 6.72 | |
| 0.12 | 14.19 | 5.14 | 4.68 | 11.32 | 17.36 | 5.67 | |
| 0.00 | 11.69 | 23.43 | 8.53 | 22.25 | 22.43 | 14.73 | |

| [DRUG] | | | | | | | ADDITIVE INHIBITION |
|---|---|---|---|---|---|---|---|
| TDF (nM) | 0.00 | 12.35 | 37.04 | 111.11 | 333.33 | 1000.00 | COMPOUND 24 |
| 10.00 | −6.65 | −25.76 | −29.76 | −35.62 | −34.24 | 6.16 | |
| 3.33 | −1.28 | −19.43 | −23.23 | −28.79 | −27.48 | 10.88 | |
| 1.11 | −3.54 | −22.09 | −25.98 | −31.66 | −30.33 | 8.9 | |
| 0.37 | −3.32 | −21.83 | −25.71 | −31.38 | −30.05 | 9.09 | |
| 0.12 | −3.31 | −21.82 | −25.7 | −31.37 | −30.04 | 9.1 | |
| 0.00 | 0 | −17.92 | −21.67 | −27.16 | −25.87 | 12.01 | |

| | |
|---|---|
| [DRUG] | SYNERGY PLOT (99.9%) |

| TDF (nM) | 0 | 12.346 | 37.037 | 111.11 | 333.33 | 1000 | Bonferroni Adj. | 98% |
|---|---|---|---|---|---|---|---|---|
| 10.00 | 0 | 0 | 0 | 0 | 0 | 0 | SYNERGY | 166.48 |
| 3.33 | 0 | 0 | 0 | 23.8081 | 27.4346 | 8.33103 | log volume | 37.9 |
| 1.11 | 0 | 15.6861 | 0.67445 | 25.7629 | 33.4629 | 1.77628 | | |
| 0.37 | 0 | 0 | 0 | 0 | 0 | 4.19448 | ANTAGONISM | 0 |
| 0.12 | 0 | 12.9843 | 10.2681 | 0 | 0 | 2.10003 | log volume | 0 |
| 0.00 | 0 | 0 | 0 | 0 | 0 | 0 | | |

TABLE 15c
Effect on HBeAg in In Vitro Combination of Compound 24 and TDF

| [DRUG] | | | | | | | AVERAGE % INHIBITION |
|---|---|---|---|---|---|---|---|
| TDF (nM) | 0.00 | 12.35 | 37.04 | 111.11 | 333.33 | 1000.00 | COMPOUND 24 (nM) |
| 10.00 | 6.07 | −1.44 | 0.13 | 0.82 | −2.83 | 22.98 | |
| 3.33 | 11.61 | 19.99 | 11.19 | 11.4 | 7.62 | 30.62 | |
| 1.11 | 12.04 | 7.84 | 11.45 | 7.21 | 14.03 | 29.45 | |
| 0.37 | 6.23 | 1.33 | 7.42 | 10.84 | 16.24 | 27.43 | |
| 0.12 | 7.02 | 7.72 | 1.74 | 10.06 | 7.19 | 22.39 | |
| 0.00 | 0 | −2.71 | −12.8 | −12.74 | −14.74 | 13.04 | |

| [DRUG] | | | | | | | STANDARD DEVIATION (%) |
|---|---|---|---|---|---|---|---|
| TDF (nM) | 0.00 | 12.35 | 37.04 | 111.11 | 333.33 | 1000.00 | COMPOUND 24 (nM) |
| 10.00 | 9.14 | 8.12 | 21.17 | 12.08 | 22.45 | 15.84 | |
| 3.33 | 23.08 | 21.21 | 19.61 | 18.06 | 17.88 | 17.08 | |
| 1.11 | 15.35 | 5.04 | 7.68 | 10.99 | 16.1 | 14.46 | |
| 0.37 | 14.75 | 11.05 | 13.95 | 12.33 | 18.21 | 15.12 | |
| 0.12 | 19.96 | 7.42 | 4.94 | 17.01 | 19.91 | 14.21 | |
| 0.00 | 19.34 | 6.37 | 3.32 | 18.69 | 25.64 | 18.52 | |

| [DRUG] | | | | | | | ADDITIVE INHIBITION |
|---|---|---|---|---|---|---|---|
| TDF (nM) | 0.00 | 12.35 | 37.04 | 111.11 | 333.33 | 1000.00 | COMPOUND 24 (nM) |
| 10.00 | 6.07 | 3.52 | −5.95 | −5.9 | −7.78 | 18.32 | |
| 3.33 | 11.61 | 9.21 | 0.3 | 0.35 | −1.42 | 23.14 | |
| 1.11 | 12.04 | 9.66 | 0.78 | 0.83 | −0.93 | 23.51 | |
| 0.37 | 6.23 | 3.69 | −5.77 | −5.72 | −7.59 | 18.46 | |
| 0.12 | 7.02 | 4.5 | −4.88 | −4.83 | −6.69 | 19.14 | |
| 0.00 | 0 | −2.71 | −12.8 | −12.74 | −14.74 | 13.04 | |

| [DRUG] | | | | | | | SYNERGY PLOT (99.9%) | |
|---|---|---|---|---|---|---|---|---|
| TDF (nM) | 0 | 12.346 | 37.037 | 111.11 | 333.33 | 1000 | Bonferroni Adj. | 98% |
| 10.00 | 0 | 0 | 0 | 0 | 0 | 0 | SYNERGY | 0 |
| 3.33 | 0 | 0 | 0 | 0 | 0 | 0 | log volume | 0 |
| 1.11 | 0 | 0 | 0 | 0 | 0 | 0 | | |
| 0.37 | 0 | 0 | 0 | 0 | 0 | 0 | ANTAGONISM | 0 |
| 0.12 | 0 | 0 | 0 | 0 | 0 | 0 | log volume | 0 |
| 0.00 | 0 | 0 | 0 | 0 | 0 | 0 | | |

TABLE 15d
Summary of results of in vitro combination studies of Compound 24 and TDF in PHH cell culture system:

| | | | Inhibitor | Inhibitor | Synergy | Synergy | Antagonism | | |
|---|---|---|---|---|---|---|---|---|---|
| HBV Assay | Inhibitor | Inhibitor | A EC₅₀ | B EC₅₀ | Volume | Log | Volume | Antagonism | |
| Endpoint | A | B | (nM)# | (nM)# | (μM² %)* | Volume | (μM² %)* | Log Volume | Conclusion |
| HBV DNA | TDF | 24 | 5.16 | 181.6 | 586.54 | 133.52 | 0 | 0 | Synergy |
| HBsAg | TDF | 24 | >100 | ~1104 | 166.48 | 37.9 | 0 | 0 | Synergy |
| HBeAg | TDF | 24 | >100 | 1087 | 0 | 0 | 0 | 0 | Additive |

*at 99.9% confidence interval
#determined in an earlier separate experiment

### Example 16

### In Vitro Combination of Compound 23 and TDF

### Study Goal

To determine whether a two-drug combination of compound 23 (a small molecule inhibitor of HBV encapsidation belonging to the amino chroman chemical class), and tenofovir (in the form of the prodrug tenofovir disoproxil fumarate, or TDF, a nucleotide analog inhibitor of HBV polymerase), is additive, synergistic or antagonistic in vitro using HBV-infected human primary hepatocytes in a cell culture model system

### Results and Conclusion

TDF (concentration range of 10.0 nM to 0.12 nM in a 3-fold dilution series and 5 point titration) was tested in combination with compound 23 (concentration range of 2000 nM to 24.69 nM in a 3-fold dilution series and 5 point titration). The average % inhibition in HBV DNA, HBsAg and HBeAg and standard deviations of 3 replicates observed either with compound 23 or TDF treatments alone or in combination are shown in Tables 16a, 16b and 16c as indicated below. The EC₅₀ values of TDF and compound 23 were determined in an earlier experiment and are shown in Table 16d; some variance was observed from different lots of PHH cells.

When the observed values of a two-inhibitor combination were compared to what is expected from additive interaction for the above concentration range, the combinations were found to be synergistic or additive, with no antagonism (Table 16d) as per MacSynergy II analysis and using the interpretive criteria described above by Prichard and Shipman (1992). No significant inhibition of cell viability or proliferation was observed by microscopy or CCK8 assay.

TABLE 16a
Effect on HBV DNA in In Vitro Combination of Compound 23 and TDF

| [DRUG] | | | | | | | AVERAGE % INHIBITION |
|---|---|---|---|---|---|---|---|
| TDF (nM) | 0.00 | 24.69 | 74.07 | 222.22 | 666.67 | 2000.00 | COMPOUND 23 (nM) |
| 10.00 | 22.03 | 15.63 | 21.53 | 50.31 | 68.1 | 83.45 | |
| 3.33 | 24.89 | 11.9 | 25.62 | 42.03 | 71.16 | 83.1 | |
| 1.11 | 24.36 | 27.41 | 25.29 | 49.82 | 71.34 | 85.27 | |
| 0.37 | −4.64 | 15.87 | 10.9 | 25.68 | 66.73 | 79.5 | |
| 0.12 | −19.34 | 14.57 | 16.57 | 17.02 | 55.55 | 74.8 | |
| 0.00 | 0 | −15.33 | −36.58 | 0.78 | 30.45 | 69.97 | |

| [DRUG] | | | | | | | STANDARD DEVIATION (%) |
|---|---|---|---|---|---|---|---|
| TDF (nM) | 0.00 | 24.69 | 74.07 | 222.22 | 666.67 | 2000.00 | COMPOUND 23 (nM) |
| 10.00 | 19.88 | 12.93 | 22.36 | 13.69 | 10.17 | 4.55 | |
| 3.33 | 25.42 | 13.82 | 12.76 | 20.92 | 9.9 | 3.38 | |
| 1.11 | 26.88 | 5.72 | 8.4 | 18.61 | 10.85 | 1 | |
| 0.37 | 22.45 | 24.04 | 16.71 | 27.51 | 6.72 | 2.89 | |
| 0.12 | 30.56 | 14.7 | 14.28 | 32.63 | 13.67 | 7.16 | |
| 0.00 | 28.21 | 25.59 | 43.45 | 19.95 | 15.55 | 7.23 | |

| [DRUG] | | | | | | | ADDITIVE INHIBITION |
|---|---|---|---|---|---|---|---|
| TDF (nM) | 0.00 | 24.69 | 74.07 | 222.22 | 666.67 | 2000.00 | COMPOUND 23 (nM) |
| 10.00 | 22.03 | 10.08 | −6.49 | 22.64 | 45.77 | 76.59 | |
| 3.33 | 24.89 | 13.38 | −2.59 | 25.48 | 47.76 | 77.44 | |
| 1.11 | 24.36 | 12.76 | −3.31 | 24.95 | 47.39 | 77.29 | |
| 0.37 | −4.64 | −20.68 | −42.92 | −3.82 | 27.22 | 68.58 | |
| 0.12 | −19.34 | −37.63 | −62.99 | −18.41 | 17 | 64.16 | |
| 0.00 | 0 | −15.33 | −36.58 | 0.78 | 30.45 | 69.97 | |

| [DRUG] | | | | | | | SYNERGY PLOT (99.9%) | |
|---|---|---|---|---|---|---|---|---|
| TDF (nM) | 0 | 24.691 | 74.074 | 222.22 | 666.67 | 2000 | Bonferroni Adj. | 98% |
| 10.00 | 0 | 0 | 0 | 0 | 0 | 0 | SYNERGY | 60.83 |
| 3.33 | 0 | 0 | 0 | 0 | 0 | 0 | log volume | 13.85 |
| 1.11 | 0 | 0 | 0.9556 | 0 | 0 | 4.689 | | |
| 0.37 | 0 | 0 | 0 | 0 | 17.3945 | 1.40901 | ANTAGONISM | 0 |
| 0.12 | 0 | 3.8223 | 32.5645 | 0 | 0 | 0 | log volume | 0 |
| 0.00 | 0 | 0 | 0 | 0 | 0 | 0 | | |

TABLE 16b
Effect on HBsAg in In Vitro Combination of Compound 23 and TDF

| [DRUG] | | | | | | | AVERAGE % INHIBITION |
|---|---|---|---|---|---|---|---|
| TDF (nM) | 0.00 | 24.69 | 74.07 | 222.22 | 666.67 | 2000.00 | COMPOUND 23 (nM) |
| 10.00 | −5.9 | −11.76 | −17.98 | −10.56 | −12.07 | −5.13 | |
| 3.33 | −0.9 | −8.32 | 2.74 | 5.75 | 3.08 | 8.14 | |
| 1.11 | 0.79 | 6.63 | 9.86 | 9.96 | 9.87 | 13.55 | |
| 0.37 | −0.3 | 10.94 | 6.53 | 9.48 | 6.86 | 12.89 | |
| 0.12 | 3.39 | 8.13 | 3.59 | 3.99 | 1.92 | 13.78 | |
| 0.00 | 0 | −13.89 | −10.9 | −11.64 | −4.45 | 0.48 | |

| [DRUG] | | | | | | | STANDARD DEVIATION (%) |
|---|---|---|---|---|---|---|---|
| TDF (nM) | 0.00 | 24.69 | 74.07 | 222.22 | 666.67 | 2000.00 | COMPOUND 23 (nM) |
| 10.00 | 11.44 | 13.32 | 9.26 | 9.99 | 12.92 | 6.2 | |
| 3.33 | 16.11 | 12.81 | 5.08 | 1.71 | 3.38 | 5.79 | |
| 1.11 | 19.99 | 5.11 | 10.31 | 10.32 | 3.11 | 4.85 | |
| 0.37 | 21.73 | 2.38 | 8.21 | 5.77 | 9.18 | 7.38 | |
| 0.12 | 9.05 | 3.32 | 4.82 | 11.75 | 7.08 | 9.54 | |
| 0.00 | 14.56 | 6.27 | 5.47 | 14.27 | 11.74 | 9.35 | |

| [DRUG] | | | | | | | ADDITIVE INHIBITION |
|---|---|---|---|---|---|---|---|
| TDF (nM) | 0.00 | 24.69 | 74.07 | 222.22 | 666.67 | 2000.00 | COMPOUND 23 (nM) |
| 10.00 | −5.9 | −20.61 | −17.44 | −18.23 | −10.61 | −5.39 | |
| 3.33 | −0.9 | −14.92 | −11.9 | −12.64 | −5.39 | −0.42 | |
| 1.11 | 0.79 | −12.99 | −10.02 | −10.76 | −3.62 | 1.27 | |
| 0.37 | −0.3 | −14.23 | −11.23 | −11.97 | −4.76 | 0.18 | |
| 0.12 | 3.39 | −10.03 | −7.14 | −7.86 | −0.91 | 3.85 | |
| 0.00 | 0 | −13.89 | −10.9 | −11.64 | −4.45 | 0.48 | |

| | |
|---|---|
| [DRUG] | SYNERGY PLOT (99.9%) |

| TDF (nM) | 0 | 24.691 | 74.074 | 222.22 | 666.67 | 2000 | Bonferroni Adj. | 98% |
|---|---|---|---|---|---|---|---|---|
| 10.00 | 0 | 0 | 0 | 0 | 0 | 0 | SYNERGY | 45.85 |
| 3.33 | 0 | 0 | 0 | 12.7624 | 0 | 0 | log volume | 10.44 |
| 1.11 | 0 | 2.80299 | 0 | 0 | 3.25499 | 0 | | |
| 0.37 | 0 | 17.3374 | 0 | 2.46093 | 0 | 0 | ANTAGONISM | 0 |
| 0.12 | 0 | 7.23388 | 0 | 0 | 0 | 0 | log volume | 0 |
| 0.00 | 0 | 0 | 0 | 0 | 0 | 0 | | |

TABLE 16c
Effect on HBeAg in In Vitro Combination of Compound 23 and TDF

| [DRUG] | | | | | | | AVERAGE % INHIBITION |
|---|---|---|---|---|---|---|---|
| TDF (nM) | 0.00 | 24.69 | 74.07 | 222.22 | 666.67 | 2000.00 | COMPOUND 23 (nM) |
| 10.00 | 0.72 | −10.51 | −5.28 | −10.54 | −11.8 | −5.49 | |
| 3.33 | 9.05 | −5.09 | 3.12 | 2.98 | 4.06 | 4.28 | |
| 1.11 | 12.78 | 7.59 | 9.52 | 0.77 | 7 | 7.24 | |
| 0.37 | 6.74 | 4.37 | 2.31 | 6.35 | 6.4 | 10.51 | |
| 0.12 | 4.4 | 8.09 | 5.22 | −0.68 | 6.5 | 7.62 | |
| 0.00 | 0 | −8.82 | −6.36 | −12.71 | −7.94 | −1.09 | |

| [DRUG] | | | | | | | STANDARD DEVIATION (%) |
|---|---|---|---|---|---|---|---|
| TDF (nM) | 0.00 | 24.69 | 74.07 | 222.22 | 666.67 | 2000.00 | COMPOUND 23 (nM) |
| 10.00 | 10.78 | 17.81 | 6.61 | 8.4 | 11.1 | 15.98 | |
| 3.33 | 9.99 | 19.89 | 13.21 | 11.51 | 7.78 | 17.13 | |
| 1.11 | 8.33 | 4.36 | 8.23 | 7.06 | 4.64 | 6.33 | |
| 0.37 | 12.35 | 9.41 | 8.19 | 15.07 | 13.35 | 17.74 | |
| 0.12 | 5.94 | 2.55 | 2.72 | 11.85 | 7.25 | 9.82 | |
| 0.00 | 16.27 | 1.94 | 6.49 | 8.83 | 9.47 | 7.31 | |

| [DRUG] | | | | | | | ADDITIVE INHIBITION |
|---|---|---|---|---|---|---|---|
| TDF (nM) | 0.00 | 24.69 | 74.07 | 222.22 | 666.67 | 2000.00 | COMPOUND 23 (nM) |
| 10.00 | 0.72 | −8.04 | −5.59 | −11.9 | −7.16 | −0.36 | |
| 3.33 | 9.05 | 1.03 | 3.27 | −2.51 | 1.83 | 8.06 | |
| 1.11 | 12.78 | 5.09 | 7.23 | 1.69 | 5.85 | 11.83 | |
| 0.37 | 6.74 | −1.49 | 0.81 | −5.11 | −0.66 | 5.72 | |
| 0.12 | 4.4 | −4.03 | −1.68 | −7.75 | −3.19 | 3.36 | |
| 0.00 | 0 | −8.82 | −6.36 | −12.71 | −7.94 | −1.09 | |

| [DRUG] | | | | | | | SYNERGY PLOT (99.9%) | |
|---|---|---|---|---|---|---|---|---|
| TDF (nM) | 0 | 24.691 | 74.074 | 222.22 | 666.67 | 2000 | Bonferroni Adj. | 98% |
| 10.00 | 0 | 0 | 0 | 0 | 0 | 0 | SYNERGY | 3.73 |
| 3.33 | 0 | 0 | 0 | 0 | 0 | 0 | log volume | 0.85 |
| 1.11 | 0 | 0 | 0 | 0 | 0 | 0 | | |
| 0.37 | 0 | 0 | 0 | 0 | 0 | 0 | ANTAGONISM | 0 |
| 0.12 | 0 | 3.72795 | 0 | 0 | 0 | 0 | log volume | 0 |
| 0.00 | 0 | 0 | 0 | 0 | 0 | 0 | | |

TABLE 16d
Summary of results of in vitro combination studies of compound 23 and TDF in PHH cell culture system:

| | | | Inhibitor | Inhibitor | Synergy | Synergy | Antagonism | | |
|---|---|---|---|---|---|---|---|---|---|
| HBV Assay | Inhibitor | Inhibitor | A EC₅₀ | B EC₅₀ | Volume | Log | Volume | Antagonism | |
| Endpoint | A | B | (nM)# | (nM)# | (μM² %)* | Volume | (μM² %)* | Log Volume | Conclusion |
| HBV | TDF | COMPOUND | 5.62 | 229.6 | 60.83 | 13.85 | 0 | 0 | Synergy |
| DNA | | 23 | | | | | | | |
| HBsAg | TDF | COMPOUND | >100 | 4.36 | 45.85 | 10.44 | 0 | 0 | Synergy |
| | | 23 | | | | | | | |
| HBeAg | TDF | COMPOUND | >100 | 4.53 | 3.73 | 0.85 | 0 | 0 | Additive |
| | | 23 | | | | | | | |

*at 99.9% confidence interval
#determined in an earlier separate experiment

### Example 17

### In Vitro Combination of Compound 23 and TAF

### In Vitro Combination Study Goal

To determine whether a two-drug combination of compound 23 (a small molecule inhibitor of HBV encapsidation belonging to the amino chroman chemical class), and tenofovir (in the form of the prodrug tenofovir alafenamide, or TAF, a nucleotide analog inhibitor of HBV polymerase), is additive, synergistic or antagonistic in vitro using HBV-infected human primary hepatocytes in a cell culture model system

### Results and Conclusion

TAF (concentration range of 10.0 nM to 0.12 nM in a 3-fold dilution series and 5 point titration) was tested in combination with compound 23 (concentration range of 2000 nM to 24.69 nM in a 3-fold dilution series and 5 point titration). The average % inhibition in HBV DNA and HBsAg and standard deviations of 3 replicates observed either with compound 23 or TAF treatments alone or in combination are shown in Tables 17a and 17b as indicated below. The EC₅₀ values of TAF and compound 23 were determined in an earlier experiment and are shown in Table 17c; some variance was observed from different lots of PHH cells.

When the observed values of a two-inhibitor combination were compared to what is expected from additive interaction for the above concentration range, the combinations were found to be additive, with no antagonism (Table 17c) as per MacSynergy II analysis and using the interpretive criteria described above by Prichard and Shipman (1992). No significant inhibition of cell viability or proliferation was observed by microscopy or CCK8 assay.

TABLE 17a
Effect on HBV DNA in In Vitro Combination of Compound 23 and TAF

| [DRUG] | | | | | | | AVERAGE % INHIBITION |
|---|---|---|---|---|---|---|---|
| TAF (nM) | 0 | 24.691 | 74.074 | 222.22 | 666.67 | 2000 | COMPOUND 23 (nM) |
| 0.00 | | | | | | | |
| 10.00 | 43.33 | 52.66 | 53.67 | 61.85 | 59.03 | 65.33 | |
| 3.33 | 42.6 | 41.59 | 42.58 | 42.01 | 55.87 | 53.47 | |
| 1.11 | 2.73 | 26 | 24.84 | 30.46 | 45.15 | 52.57 | |
| 0.37 | 11.59 | 10.66 | 15.11 | 15.55 | 38.82 | 64.27 | |
| 0.12 | 6.36 | 12.62 | −10.64 | 15.2 | 36.81 | 52.56 | |
| 0.00 | 0 | −4.57 | −2.49 | 11.13 | 30.46 | 58.13 | |
| [DRUG] | | | | | | | STANDARD DEVIATION (%) |
| TAF (nM) | 0 | 24.691 | 74.074 | 222.22 | 666.67 | 2000 | COMPOUND 23 (nM) |
| 10.00 | 19.23 | 6.09 | 16.14 | 6.57 | 11.43 | 9.3 | |
| 3.33 | 5.15 | 11.48 | 8.01 | 13.55 | 8.93 | 4.21 | |
| 1.11 | 16.85 | 19.39 | 8.78 | 4.56 | 12.22 | 8.28 | |
| 0.37 | 14.07 | 2.95 | 9.65 | 20.83 | 4.73 | 0.79 | |
| 0.12 | 4.65 | 9.48 | 19.93 | 6.28 | 0.72 | 12.12 | |
| 0.00 | 0.02 | 8.18 | 25.79 | 14.9 | 9.52 | 3.29 | |

| [DRUG] | | | | | | | ADDITIVE INHIBITION |
|---|---|---|---|---|---|---|---|
| TAF (nM) | 0 | 24.691 | 74.074 | 222.22 | 666.67 | 2000 | COMPOUND 23 (nM) |
| 10.00 | 43.33 | 40.74 | 41.92 | 49.64 | 60.59 | 76.27 | |
| 3.33 | 42.6 | 39.98 | 41.17 | 48.99 | 60.08 | 75.97 | |
| 1.11 | 2.73 | −1.72 | 0.31 | 13.56 | 32.36 | 59.27 | |
| 0.37 | 11.59 | 7.55 | 9.39 | 21.43 | 38.52 | 62.98 | |
| 0.12 | 6.36 | 2.08 | 4.03 | 16.78 | 34.88 | 60.79 | |
| 0.00 | 0 | −4.57 | −2.49 | 11.13 | 30.46 | 58.13 | |

| [DRUG] | | | | | | | SYNERGY PLOT (99.9%) | |
|---|---|---|---|---|---|---|---|---|
| TAF (nM) | 0 | 24.691 | 74.074 | 222.22 | 666.67 | 2000 | Bonferroni Adj. | 98% |
| 10.00 | 0 | 0 | 0 | 0 | 0 | 0 | SYNERGY | 1.89 |
| 3.33 | 0 | 0 | 0 | 0 | 0 | −8.6449 | log volume | 0.43 |
| 1.11 | 0 | 0 | 0 | 1.89304 | 0 | 0 | | |
| 0.37 | 0 | 0 | 0 | 0 | 0 | 0 | ANTAGONISM | −8.64 |
| 0.12 | 0 | 0 | 0 | 0 | 0 | 0 | log volume | −1.97 |
| 0.00 | 0 | 0 | 0 | 0 | 0 | 0 | | |

TABLE 17b
Effect on HBsAg in In Vitro Combination of Compound 23 and TAF

| [DRUG] | | | | | | | AVERAGE % INHIBITION |
|---|---|---|---|---|---|---|---|
| TAF (nM) | 0 | 24.691 | 74.074 | 222.22 | 666.67 | 2000 | COMPOUND 23 (nM) |
| 0.00 | | | | | | | |
| 10.00 | 6.38 | 6.2 | 3.4 | −8.69 | 5.26 | 23.51 | |
| 3.33 | 16.12 | 9.58 | 9.56 | 0.37 | 11.57 | 28.58 | |
| 1.11 | 29.66 | 18.05 | 20.47 | 9.86 | 18.31 | 33.24 | |
| 0.37 | 7.04 | 11.13 | 3.66 | −2.3 | −2.92 | 22.22 | |
| 0.12 | 22.99 | 21.29 | 19.81 | 16.79 | 8.85 | 28.41 | |
| 0.00 | 0 | 0.77 | −5.27 | 4.83 | 1.95 | 22.77 | |
| [DRUG] | | | | | | | STANDARD DEVIATION (%) |
| TAF (nM) | 0 | 24.691 | 74.074 | 222.22 | 666.67 | 2000 | COMPOUND 23 (nM) |
| 10.00 | 3.03 | 12.27 | 11.65 | 6.93 | 7.08 | 10.25 | |
| 3.33 | 3.66 | 2.28 | 2.6 | 17.49 | 9.97 | 8.2 | |
| 1.11 | 9.01 | 17.67 | 8.37 | 8.64 | 8.88 | 5.26 | |
| 0.37 | 9.67 | 9.77 | 10.47 | 17.15 | 5.76 | 1.93 | |
| 0.12 | 3.68 | 9.92 | 15.76 | 11.59 | 9.9 | 13.42 | |
| 0.00 | 1.83 | 21.63 | 8.58 | 26.08 | 6.99 | 7.12 | |

| [DRUG] | | | | | | | ADDITIVE INHIBITION |
|---|---|---|---|---|---|---|---|
| TAF (nM) | 0 | 24.691 | 74.074 | 222.22 | 666.67 | 2000 | COMPOUND 23 (nM) |
| 10.00 | 6.38 | 7.1 | 1.45 | 10.9 | 8.21 | 27.7 | |
| 3.33 | 16.12 | 16.77 | 11.7 | 20.17 | 17.76 | 35.22 | |
| 1.11 | 29.66 | 30.2 | 25.95 | 33.06 | 31.03 | 45.68 | |
| 0.37 | 7.04 | 7.76 | 2.14 | 11.53 | 8.85 | 28.21 | |
| 0.12 | 22.99 | 23.58 | 18.93 | 26.71 | 24.49 | 40.53 | |
| 0.00 | 0 | 0.77 | −5.27 | 4.83 | 1.95 | 22.77 | |

| [DRUG] | | | | | | | SYNERGY PLOT (99.9%) | |
|---|---|---|---|---|---|---|---|---|
| TAF (nM) | 0 | 24.691 | 74.074 | 222.22 | 666.67 | 2000 | Bonferroni Adj. | 98% |
| 10.00 | 0 | 0 | 0 | 0 | 0 | 0 | SYNERGY | 0 |
| 3.33 | 0 | 0 | 0 | 0 | 0 | 0 | log volume | 0 |
| 1.11 | 0 | 0 | 0 | 0 | 0 | 0 | | |
| 0.37 | 0 | 0 | 0 | 0 | 0 | 0 | ANTAGONISM | 0 |
| 0.12 | 0 | 0 | 0 | 0 | 0 | 0 | log volume | 0 |
| 0.00 | 0 | 0 | 0 | 0 | 0 | 0 | | |

TABLE 17c
Summary of results of in vitro combination studies of Compound 23 and TAF in PHH cell culture system:

| | | | Inhibitor | Inhibitor | Synergy | Synergy | Antagonism | | |
|---|---|---|---|---|---|---|---|---|---|
| HBV Assay | Inhibitor | Inhibitor | A EC₅₀ | B EC₅₀ | Volume | Log | Volume | Antagonism | |
| Endpoint | A | B | (nM)# | (nM)# | (μM² %)* | Volume | (μM² %)* | Log Volume | Conclusion |
| HBV | TAF | COMPOUND | 0.405 | 229.6 | 1.89 | 0.43 | −8.64 | −1.97 | Additive |
| DNA | | 23 | | | | | | | |
| HBsAg | TAF | COMPOUND | >100 | 4.36 | 0 | 0 | 0 | 0 | Addtiive |
| | | 23 | | | | | | | |

*at 99.9% confidence interval
#determined in an earlier separate experiment

### Example 18

### In Vitro Combination of IFNα2a and Compound 25

### Study Goal

To determine whether a two-drug combination of compound 25 (a small molecule inhibitor of HBV DNA, HBsAg and HBeAg, belonging to the dihydroquinolizinone chemical class), and pegylated interferon alpha 2a (IFNα2a, an antiviral cytokine that activates innate immunity pathways in hepatocytes), is additive, synergistic or antagonistic in vitro using HBV-infected human primary hepatocytes in a cell culture model system.

### Results and Conclusion

IFNα2a (concentration range of 10.0 IU/mL to 0.123 IU/mL in a 3-fold dilution series and 5 point titration) was tested in combination with compound 25 (concentration range of 10.0 nM to 0.12 nM in a 3-fold dilution series and 5 point titration). The average % inhibition in HBV DNA, HBsAg and HBeAg, and standard deviations of 3 replicates observed either with IFNa2a or compound 25 treatments alone or in combination are shown in Table 18a, 18b, and 18c as indicated below. The EC₅₀ values of IFNα2a and compound 25 were determined in an earlier experiment and are shown in Table 18d; some variance was observed from different lots of PHH cells.

When the observed values of a two-inhibitor combination were compared to what is expected from additive interaction for the above concentration range, the combinations were found to be synergistic, with no antagonism (Table 18d) as per MacSynergy II analysis and using the interpretive criteria described above by Prichard and Shipman (1992). No significant inhibition of cell viability or proliferation was observed by microscopy or CCK8 assay.

TABLE 18a
Effect on HBV DNA in In Vitro Combination of IFNα2a and Compound 25

| [DRUG] | | | | | | | |
|---|---|---|---|---|---|---|---|
| IFNα2a | | | | | | | AVERAGE % INHIBITION |
| IU/mL | 0.00 | 0.12 | 0.37 | 1.11 | 3.33 | 10.00 | COMPOUND 25 (μM) |
| 10.00 | 58.66 | 72.01 | 77.55 | 74.4 | 74.57 | 75.72 | |
| 3.33 | 46.92 | 70.84 | 75.67 | 71.52 | 79.37 | 81.47 | |
| 1.11 | 32.66 | 60.24 | 64.08 | 65.29 | 76.55 | 76.76 | |
| 0.37 | 22.81 | 48.83 | 55.68 | 55.85 | 71.09 | 75.44 | |
| 0.12 | −19.84 | 40.19 | 39.08 | 36.54 | 65.34 | 64.9 | |
| 0.00 | 0 | −14.4 | −9.87 | 4.3 | 32.64 | 53.78 | |

| [DRUG] | | | | | | | |
|---|---|---|---|---|---|---|---|
| IFNα2a | | | | | | | STANDARD DEVIATION (%) |
| IU/mL | 0.00 | 0.12 | 0.37 | 1.11 | 3.33 | 10.00 | COMPOUND 25 (μM) |
| 10.00 | 8.37 | 0.96 | 1.44 | 5.16 | 6.13 | 9.02 | |
| 3.33 | 6.8 | 2.16 | 3.21 | 1.91 | 3.01 | 4.5 | |
| 1.11 | 7.03 | 10.58 | 6.34 | 2.57 | 2.47 | 2.79 | |
| 0.37 | 6.72 | 4.66 | 7.04 | 12.83 | 7.17 | 1.6 | |
| 0.12 | 15.09 | 10.34 | 11.46 | 15.82 | 5.84 | 3.79 | |
| 0.00 | 26.83 | 27.99 | 12.43 | 13.96 | 21.25 | 5.81 | |

| [DRUG] | | | | | | | |
|---|---|---|---|---|---|---|---|
| IFNα2a | | | | | | | ADDITIVE INHIBITION |
| IU/mL | 0.00 | 0.12 | 0.37 | 1.11 | 3.33 | 10.00 | COMPOUND 25 (μM) |
| 10.00 | 58.66 | 52.71 | 54.58 | 60.44 | 72.15 | 80.89 | |
| 3.33 | 46.92 | 39.28 | 41.68 | 49.2 | 64.25 | 75.47 | |
| 1.11 | 32.66 | 22.96 | 26.01 | 35.56 | 54.64 | 68.88 | |
| 0.37 | 22.81 | 11.69 | 15.19 | 26.13 | 48 | 64.32 | |
| 0.12 | −19.84 | −37.1 | −31.67 | −14.69 | 19.28 | 44.61 | |
| 0.00 | 0 | −14.4 | −9.87 | 4.3 | 32.64 | 53.78 | |

| [DRUG] | | | | | | | | |
|---|---|---|---|---|---|---|---|---|
| IFNα2a | | | | | | | SYNERGY PLOT (99.9%) | |
| IU/mL | 0 | 0.1235 | 0.3704 | 1.1111 | 3.3333 | 10 | Bonferroni Adj. | 98% |
| 10.00 | 0 | 16.1406 | 18.231 | 0 | 0 | 0 | SYNERGY | 314.15 |
| 3.33 | 0 | 24.4514 | 23.4259 | 16.0342 | 5.21409 | 0 | log volume | 71.51 |
| 1.11 | 0 | 2.46122 | 17.2051 | 21.2721 | 13.7812 | 0 | | |
| 0.37 | 0 | 21.8039 | 17.3214 | 0 | 0 | 5.8544 | ANTAGONISM | 0 |
| 0.12 | 0 | 43.2611 | 33.0351 | 0 | 26.8406 | 7.81711 | log volume | 0 |
| 0.00 | 0 | 0 | 0 | 0 | 0 | 0 | | |

TABLE 18b
Effect on HBsAg in In Vitro Combination of IFNα2a and Compound 25

| [DRUG] | | | | | | | |
|---|---|---|---|---|---|---|---|
| IFNα2a | | | | | | | AVERAGE % INHIBITION |
| IU/mL | 0.00 | 0.12 | 0.37 | 1.11 | 3.33 | 10.00 | COMPOUND 25 (μM) |
| 10.00 | 22.77 | 27.23 | 22.41 | 30.25 | 37.23 | 63.56 | |
| 3.33 | 18.32 | 28.86 | 27.09 | 35.53 | 43.71 | 66.88 | |
| 1.11 | 10.57 | 27 | 31.57 | 31.22 | 38.7 | 66.37 | |
| 0.37 | 2.74 | 18.78 | 15.98 | 25.14 | 34.24 | 60.44 | |
| 0.12 | −4.08 | 11.87 | 10.92 | 14.5 | 34.52 | 56.65 | |
| 0.00 | 0 | −5.64 | −7.52 | −7.33 | 8.81 | 42.49 | |

| [DRUG] | | | | | | | |
|---|---|---|---|---|---|---|---|
| IFNα2a | | | | | | | STANDARD DEVIATION (%) |
| IU/mL | 0.00 | 0.12 | 0.37 | 1.11 | 3.33 | 10.00 | COMPOUND 25 (μM) |
| 10.00 | 8.68 | 6.97 | 2.29 | 4.73 | 7.98 | 4.01 | |
| 3.33 | 9.52 | 6.19 | 6.14 | 6.04 | 6.94 | 4.47 | |
| 1.11 | 2.72 | 4.07 | 4.71 | 1.23 | 4.72 | 0.28 | |
| 0.37 | 8.08 | 2.56 | 1.27 | 2.26 | 2.05 | 4.7 | |
| 0.12 | 6.17 | 2.65 | 2.53 | 0.54 | 1.95 | 2.99 | |
| 0.00 | 7 | 8.29 | 12.25 | 8.62 | 8.49 | 4.98 | |

| [DRUG] | | | | | | | |
|---|---|---|---|---|---|---|---|
| IFNα2a | | | | | | | ADDITIVE INHIBITION |
| IU/mL | 0.00 | 0.12 | 0.37 | 1.11 | 3.33 | 10.00 | COMPOUND 25 (μM) |
| 10.00 | 22.77 | 18.41 | 16.96 | 17.11 | 29.57 | 55.59 | |
| 3.33 | 18.32 | 13.71 | 12.18 | 12.33 | 25.52 | 53.03 | |
| 1.11 | 10.57 | 5.53 | 3.84 | 4.01 | 18.45 | 48.57 | |
| 0.37 | 2.74 | −2.75 | −4.57 | −4.39 | 11.31 | 44.07 | |
| 0.12 | −4.08 | −9.95 | −11.91 | −11.71 | 5.09 | 40.14 | |
| 0.00 | 0 | −5.64 | −7.52 | −7.33 | 8.81 | 42.49 | |

| [DRUG] | | | | | | | | |
|---|---|---|---|---|---|---|---|---|
| IFNα2a | | | | | | | SYNERGY PLOT (99.9%) | |
| IU/mL | 0 | 0.1235 | 0.3704 | 1.1111 | 3.3333 | 10 | Bonferroni Adj. | 98% |
| 10.00 | 0 | 0 | 0 | 0 | 0 | 0 | SYNERGY | 218.76 |
| 3.33 | 0 | 0 | 0 | 3.32236 | 0 | 0 | log volume | 49.8 |
| 1.11 | 0 | 8.07563 | 12.2294 | 23.1621 | 4.71648 | 16.8785 | | |
| 0.37 | 0 | 13.105 | 16.3704 | 22.0923 | 16.1835 | 0.9023 | ANTAGONISM | 0 |
| 0.12 | 0 | 13.0989 | 14.5038 | 24.4329 | 23.0126 | 6.66991 | log volume | 0 |
| 0.00 | 0 | 0 | 0 | 0 | 0 | 0 | | |

TABLE 18c
Effect on HBeAg in In Vitro Combination of IFNα2a and Compound 25

| | | | | | | | AVERAGE % INHIBITION |
|---|---|---|---|---|---|---|---|
| [DRUG] | 0.00 | 0.12 | 0.37 | 1.11 | 3.33 | 10.00 | COMPOUND 25 (μM) |
| 10.00 | 17.32 | 33.64 | 22.73 | 25.58 | 32.72 | 51.97 | |
| 3.33 | 6.47 | 24.71 | 20.71 | 19.06 | 27.19 | 49 | |
| 1.11 | −1.13 | 21.52 | 18.25 | 15.99 | 19.2 | 47.55 | |
| 0.37 | −12.17 | 10.2 | 8.56 | 12.48 | 14.45 | 40.46 | |
| 0.12 | −21.05 | 1.9 | 3.84 | 0.95 | 15.57 | 36.99 | |
| 0.00 | 0 | −11.25 | −13.81 | −16.8 | −7.31 | 22.04 | |

| [DRUG] | | | | | | | |
|---|---|---|---|---|---|---|---|
| IFNα2a | | | | | | | STANDARD DEVIATION (%) |
| IU/mL | 0.00 | 0.12 | 0.37 | 1.11 | 3.33 | 10.00 | COMPOUND 25 (μM) |
| 10.00 | 11.74 | 4.4 | 2.35 | 5.73 | 3.68 | 4.09 | |
| 3.33 | 19.07 | 8.01 | 1.6 | 5.75 | 14.7 | 8.73 | |
| 1.11 | 17.14 | 4.93 | 2.29 | 7.45 | 9.68 | 6.75 | |
| 0.37 | 26.1 | 2.4 | 6.51 | 8.28 | 5.47 | 9 | |
| 0.12 | 25.35 | 7.46 | 12.09 | 14.46 | 9.05 | 10.23 | |
| 0.00 | 19.06 | 11.33 | 16.27 | 24 | 19.27 | 14.29 | |

| [DRUG] | | | | | | | |
|---|---|---|---|---|---|---|---|
| IFNα2a | | | | | | | ADDITIVE INHIBITION |
| IU/mL | 0.00 | 0.12 | 0.37 | 1.11 | 3.33 | 10.00 | COMPOUND 25 (μM) |
| 10.00 | 17.32 | 8.02 | 5.9 | 3.43 | 11.28 | 35.54 | |
| 3.33 | 6.47 | −4.05 | −6.45 | −9.24 | −0.37 | 27.08 | |
| 1.11 | −1.13 | −12.51 | −15.1 | −18.12 | −8.52 | 21.16 | |
| 0.37 | −12.17 | −24.79 | −27.66 | −31.01 | −20.37 | 12.55 | |
| 0.12 | −21.05 | −34.67 | −37.77 | −41.39 | −29.9 | 5.63 | |
| 0.00 | 0 | −11.25 | −13.81 | −16.8 | −7.31 | 22.04 | |

| [DRUG] | | | | | | | | |
|---|---|---|---|---|---|---|---|---|
| IFNα2a | | | | | | | SYNERGY PLOT (99.9%) | |
| IU/mL | 0 | 0.1235 | 0.3704 | 1.1111 | 3.3333 | 10 | Bonferroni Adi. | 98% |
| 10.00 | 0 | 11.1396 | 9.09615 | 3.29257 | 9.32912 | 2.96981 | SYNERGY | 231.36 |
| 3.33 | 0 | 2.39909 | 21.8944 | 9.37675 | 0 | 0 | log volume | 52.67 |
| 1.11 | 0 | 17.8054 | 25.8136 | 9.59205 | 0 | 4.17575 | | |
| 0.37 | 0 | 27.0916 | 14.7956 | 16.2405 | 16.8182 | 0 | ANTAGONISM | 0 |
| 0.12 | 0 | 12.0191 | 1.82181 | 0 | 15.6865 | 0 | log volume | 0 |
| 0.00 | 0 | 0 | 0 | 0 | 0 | 0 | | |

TABLE 18d
Summary of results of in vitro combination studies of IFNα2a and Compound 25in PHH cell culture system:

| | | | Inhibitor | Inhibitor | Synergy | Synergy | Antagonism | | |
|---|---|---|---|---|---|---|---|---|---|
| HBV Assay | Inhibitor | Inhibitor | A EC₅₀ | B EC₅₀ | Volume | Log | Volume | Antagonism | |
| Endpoint | A | B | (IU/mL)# | (nM)# | (μM² %)* | Volume | (μM² %)* | Log Volume | Conclusion |
| HBV | IFNα2a | COMPOUND | 2.154 | 0.654 | 314.15 | 71.51 | 0 | 0 | Synergy |
| DNA | | 25 | | | | | | | |
| HBsAg | IFNα2a | COMPOUND | 13.8 | 4.503 | 218.76 | 49.8 | 0 | 0 | Synergy |
| | | 25 | | | | | | | |
| HBeAg | IFNα2a | COMPOUND | 10.24 | 5.75 | 231.36 | 52.67 | 0 | 0 | Synergy |
| | | 25 | | | | | | | |

*at 99.9% confidence interval
#determined in an earlier separate experiment

### Example 19

### In Vitro Combination of Compound 25 and Compound 3

### Study Goal

To determine whether a two-drug combination of compound 3 (a small molecule inhibitor of HBV encapsidation belonging to the sulfamoyl benzamide chemical class), and compound 25 (a small molecule inhibitor of HBV DNA, HBsAg and HBeAg, belonging to the dihydroquinolizinone chemical class), is additive, synergistic or antagonistic in vitro using HBV-infected human primary hepatocytes in a cell culture model system.

### Results and Conclusion

Compound 25 (concentration range of 10.0 nM to 0.12 nM in a 3-fold dilution series and 5 point titration) was tested in combination with compound 3 (concentration range of 5000 nM to 61.73 nM in a 3-fold dilution series and 5 point titration). The average % inhibition in HBV DNA, HBsAg and HBeAg, and standard deviations of 3 replicates observed either with compound 25 or compound 3 treatments alone or in combination are shown in Tables 19a, 19b, and 19c as indicated below. The EC₅₀ values of compound 25 and compound 3 were determined in an earlier experiment and are shown in Table 19d; some variance was observed from different lots of PHH cells.

When the observed values of a two-inhibitor combination were compared to what is expected from additive interaction for the above concentration range, the combinations were found to be synergistic, with no antagonism (Table 19d) as per MacSynergy II analysis and using the interpretive criteria described above by Prichard and Shipman (1992). No significant inhibition of cell viability or proliferation was observed by microscopy or CCK8 assay in the analyzed samples.

TABLE 19a
Effect on HBV DNA in In Vitro Combination of Compound 25 and Compound 3

| [DRUG] | | | | | | | |
|---|---|---|---|---|---|---|---|
| COMPOUND 25 | | | | | | | AVERAGE % INHIBITION |
| nM | 0.00 | 61.73 | 185.19 | 555.56 | 1666.67 | 5000.00 | COMPOUND 3 (nM) |
| 10.00 | 28 | 53.83 | 59.64 | 61.48 | 75.31 | 83.32 | |
| 3.33 | 15.24 | 52.77 | 48.78 | 55.49 | 77.84 | 86.19 | |
| 1.11 | 5.69 | 32.55 | 40.25 | 48.87 | 68.73 | 85.52 | |
| 0.37 | −51.8 | 21.47 | 28.54 | 33.37 | 64.04 | 83.77 | |
| 0.12 | −20.98 | 17.18 | 18.75 | 27.78 | 58.12 | 84.2 | |
| 0.00 | 0 | −28.13 | −25.93 | −3.32 | 25.94 | 74.78 | |

| [DRUG] | | | | | | | |
|---|---|---|---|---|---|---|---|
| COMPOUND 25 | | | | | | | STANDARD DEVIATION (%) |
| nM | 0.00 | 61.73 | 185.19 | 555.56 | 1666.67 | 5000.00 | COMPOUND 3 (nM) |
| 10.00 | 13.66 | 6.65 | 4.22 | 12.05 | 4.52 | 3.89 | |
| 3.33 | 15.64 | 6.92 | 3.55 | 5.98 | 2.73 | 2.62 | |
| 1.11 | 2.13 | 7.36 | 12.67 | 3.75 | 8.6 | 1.07 | |
| 0.37 | 7.11 | 11.02 | 11.57 | 16.68 | 4.9 | 3.83 | |
| 0.12 | 6.37 | 6.92 | 8.03 | 5.44 | 6.89 | 1.72 | |
| 0.00 | 37.96 | 6.82 | 12.75 | 12.54 | 6.98 | 2.18 | |

| [DRUG] | | | | | | | |
|---|---|---|---|---|---|---|---|
| COMPOUND 25 | | | | | | | ADDITIVE INHIBITION |
| nM | 0.00 | 61.73 | 185.19 | 555.56 | 1666.67 | 5000.00 | COMPOUND 3 (nM) |
| 10.00 | 28 | 7.75 | 9.33 | 25.61 | 46.68 | 81.84 | |
| 3.33 | 15.24 | −8.6 | −6.74 | 12.43 | 37.23 | 78.62 | |
| 1.11 | 5.69 | −20.84 | −18.76 | 2.56 | 30.15 | 76.22 | |
| 0.37 | −51.8 | −94.5 | −91.16 | −56.84 | −12.42 | 61.72 | |
| 0.12 | −20.98 | −55.01 | −52.35 | −25 | 10.4 | 69.49 | |
| 0.00 | 0 | −28.13 | −25.93 | −3.32 | 25.94 | 74.78 | |

| [DRUG] | | | | | | | | |
|---|---|---|---|---|---|---|---|---|
| COMPOUND 25 | | | | | | | SYNERGY PLOT (99.9%) | |
| nM | 0 | 61.728 | 185.19 | 555.56 | 1666.7 | 5000 | Bonferroni Adj. | 98% |
| 10.00 | 0 | 24.1949 | 36.422 | 0 | 13.7547 | 0 | SYNERGY | 737.8 |
| 3.33 | 0 | 38.5963 | 43.837 | 23.3798 | 31.6256 | 0 | log volume | 167.96 |
| 1.11 | 0 | 29.1682 | 17.313 | 33.9688 | 10.2774 | 5.77863 | | |
| 0.37 | 0 | 79.7032 | 81.6231 | 35.3161 | 60.3341 | 9.44547 | ANTAGONISM | 0 |
| 0.12 | 0 | 49.4163 | 44.6733 | 34.877 | 25.045 | 9.04948 | log volume | 0 |
| 0.00 | 0 | 0 | 0 | 0 | 0 | 0 | | |

TABLE 19b
Effect on HBsAg in In Vitro Combination of Compound 25 and Compound 3

| [DRUG] | | | | | | | |
|---|---|---|---|---|---|---|---|
| COMPOUND 25 | | | | | | | AVERAGE % INHIBITION |
| nM | 0.00 | 61.73 | 185.19 | 555.56 | 1666.67 | 5000.00 | COMPOUND 3 (nM) |
| 10.00 | 32.99 | 40.09 | 41.48 | 45.13 | 52.34 | 64.84 | |
| 3.33 | 13.12 | 26.32 | 28.85 | 30.97 | 34.56 | 54.59 | |
| 1.11 | −3.18 | 21.32 | 20.73 | 21.99 | 32.47 | 56.21 | |
| 0.37 | −5.09 | 13.81 | 9.92 | 8.14 | 27.4 | 51.59 | |
| 0.12 | 3.68 | 7.53 | 8.59 | 12.88 | 22.46 | 48.46 | |
| 0.00 | 0 | −20.02 | −17.32 | −13.99 | 1.44 | 28.25 | |

| [DRUG] | | | | | | | |
|---|---|---|---|---|---|---|---|
| COMPOUND 25 | | | | | | | STANDARD DEVIATION (%) |
| nM | 0.00 | 61.73 | 185.19 | 555.56 | 1666.67 | 5000.00 | COMPOUND 3 (nM) |
| 10.00 | 11.76 | 5.21 | 4.72 | 1.8 | 3.51 | 4.34 | |
| 3.33 | 6.7 | 5 | 8.24 | 5.49 | 2.08 | 2.72 | |
| 1.11 | 2.66 | 0.74 | 5.4 | 3.5 | 4.64 | 4.3 | |
| 0.37 | 3.17 | 7.51 | 16.06 | 12.02 | 5.09 | 4.62 | |
| 0.12 | 2.76 | 6.34 | 8.52 | 9.71 | 4.5 | 5.28 | |
| 0.00 | 26.63 | 3.49 | 15.37 | 12.95 | 14.94 | 14.17 | |

| [DRUG] | | | | | | | |
|---|---|---|---|---|---|---|---|
| COMPOUND 25 | | | | | | | ADDITIVE INHIBITION |
| nM | 0.00 | 61.73 | 185.19 | 555.56 | 1666.67 | 5000.00 | COMPOUND 3 (nM) |
| 10.00 | 32.99 | 19.57 | 21.38 | 23.62 | 33.95 | 51.92 | |
| 3.33 | 13.12 | −4.27 | −1.93 | 0.97 | 14.37 | 37.66 | |
| 1.11 | −3.18 | −23.84 | −21.05 | −17.61 | −1.69 | 25.97 | |
| 0.37 | −5.09 | −26.13 | −23.29 | −19.79 | −3.58 | 24.6 | |
| 0.12 | 3.68 | −15.6 | −13 | −9.8 | 5.07 | 30.89 | |
| 0.00 | 0 | −20.02 | −17.32 | −13.99 | 1.44 | 28.25 | |

| [DRUG] | | | | | | | | |
|---|---|---|---|---|---|---|---|---|
| COMPOUND 25 | | | | | | | SYNERGY PLOT (99.9%) | |
| nM | 0 | 61.728 | 185.19 | 555.56 | 1666.7 | 5000 | Bonferroni Adj. | 98% |
| 10.00 | 0 | 3.37389 | 4.56648 | 15.5862 | 6.83859 | 0 | SYNERGY | 257.49 |
| 3.33 | 0 | 14.135 | 3.66216 | 11.9324 | 13.3447 | 7.97848 | log volume | 58.62 |
| 1.11 | 0 | 42.7247 | 24.0086 | 28.0815 | 18.8898 | 16.0887 | | |
| 0.37 | 0 | 15.2246 | 0 | 0 | 14.2288 | 11.7856 | ANTAGONISM | 0 |
| 0.12 | 0 | 2.26506 | 0 | 0 | 2.5805 | 0.19352 | log volume | 0 |
| 0.00 | 0 | 0 | 0 | 0 | 0 | 0 | | |

TABLE 19c
Effect on HBeAg in In Vitro Combination of Compound 25 and Compound 3

| [DRUG] | | | | | | | |
|---|---|---|---|---|---|---|---|
| COMPOUND 25 | | | | | | | AVERAGE % INHIBITION |
| nM | 0.00 | 61.73 | 185.19 | 555.56 | 1666.67 | 5000.00 | COMPOUND 3 (nM) |
| 10.00 | 33.17 | 29.55 | 32.13 | 35.12 | 45.53 | 56.72 | |
| 3.33 | 8.74 | 17.06 | 14.55 | 17.58 | 28.19 | 41.81 | |
| 1.11 | 4.51 | 14.84 | 10.85 | 17.54 | 27.32 | 48.49 | |
| 0.37 | −0.51 | 7.18 | 2.63 | 7.03 | 20.64 | 40.75 | |
| 0.12 | 5.33 | 4.76 | −1.23 | 8.26 | 17.34 | 42.34 | |
| 0.00 | 0 | −11.35 | −16 | −5.39 | 2.34 | 27.3 | |

| [DRUG] | | | | | | | |
|---|---|---|---|---|---|---|---|
| COMPOUND 25 | | | | | | | STANDARD DEVIATION (%) |
| nM | 0.00 | 61.73 | 185.19 | 555.56 | 1666.67 | 5000.00 | COMPOUND 3 (nM) |
| 10.00 | 19.04 | 7.64 | 7.38 | 3.04 | 5.15 | 8.44 | |
| 3.33 | 17.53 | 4.42 | 4.02 | 3.71 | 1.17 | 3.68 | |
| 1.11 | 11.69 | 1.61 | 6.69 | 4.6 | 2.82 | 2.79 | |
| 0.37 | 17.52 | 6.16 | 9.21 | 11.25 | 2.17 | 4.33 | |
| 0.12 | 17.42 | 6.48 | 8.81 | 8.1 | 1.87 | 4.94 | |
| 0.00 | 27.36 | 3.5 | 5.88 | 8.38 | 7.46 | 13.79 | |

| [DRUG] | | | | | | | |
|---|---|---|---|---|---|---|---|
| COMPOUND 25 | | | | | | | ADDITIVE INHIBITION |
| nM | 0.00 | 61.73 | 185.19 | 555.56 | 1666.67 | 5000.00 | COMPOUND 3 (nM) |
| 10.00 | 33.17 | 25.58 | 22.48 | 29.57 | 34.73 | 51.41 | |
| 3.33 | 8.74 | −1.62 | −5.86 | 3.82 | 10.88 | 33.65 | |
| 1.11 | 4.51 | −6.33 | −10.77 | −0.64 | 6.74 | 30.58 | |
| 0.37 | −0.51 | −11.92 | −16.59 | −5.93 | 1.84 | 26.93 | |
| 0.12 | 5.33 | −5.42 | −9.82 | 0.23 | 7.55 | 31.17 | |
| 0.00 | 0 | −11.35 | −16 | −5.39 | 2.34 | 27.3 | |

| [DRUG] | | | | | | | | |
|---|---|---|---|---|---|---|---|---|
| COMPOUND 25 | | | | | | | SYNERGY PLOT (99.9%) | |
| nM | 0 | 61.728 | 185.19 | 555.56 | 1666.7 | 5000 | Bonferroni Adj. | 98% |
| 10.00 | 0 | 0 | 0 | 0 | 0 | 0 | SYNERGY | 80.56 |
| 3.33 | 0 | 4.13378 | 7.18018 | 1.55039 | 13.4595 | 0 | log volume | 18.34 |
| 1.11 | 0 | 15.8715 | 0 | 3.0414 | 11.2994 | 8.72811 | | |
| 0.37 | 0 | 0 | 0 | 0 | 11.6585 | 0 | ANTAGONISM | 0 |
| 0.12 | 0 | 0 | 0 | 0 | 3.63583 | 0 | log volume | 0 |
| 0.00 | 0 | 0 | 0 | 0 | 0 | 0 | | |

TABLE 19d
Summary of results of in vitro combination studies of Compound 25 and Compound 3 in PHH cell culture system:

| HBV | | | Inhibitor | Inhibitor | Synergy | Synergy | Antagonism | Antagonism | |
|---|---|---|---|---|---|---|---|---|---|
| Assay | Inhibitor | Inhibitor | A EC₅₀ | B EC₅₀ | Volume | Log | Volume | Log | |
| Endpoint | A | B | (nM)# | (nM)# | (μM² %)* | Volume | (μM² %)* | Volume | Conclusion |
| HBV DNA | COMPOUND 25 | COMPOUND 3 | 0.654 | 876.5 | 737.8 | 167.96 | 0 | 0 | Synergy |
| HBsAg | COMPOUND 25 | COMPOUND 3 | 4.503 | 7793 | 257.49 | 58.62 | 0 | 0 | Synergy |
| HBeAg | COMPOUND 25 | COMPOUND 3 | 5.75 | 8850 | 80.56 | 18.34 | 0 | 0 | Synergy |

*at 99.9% confidence interval
#determined in an earlier separate experiment

### Example 20

### In Vitro Combination of Compound 3 and TAF

### Study Goal

To determine whether a two-drug combination of compound 3 (a small molecule inhibitor of HBV encapsidation belonging to the sulfamoyl benzamide chemical class), and tenofovir (in the form of the prodrug tenofovir alafenamide, or TAF, a nucleotide analog inhibitor of HBV polymerase), is additive, synergistic or antagonistic in vitro using HBV-infected human primary hepatocytes in a cell culture model system.

### Results and Conclusion

TAF (concentration range of 10.0 nM to 0.12 nM in a 3-fold dilution series and 5 point titration) was tested in combination with compound 3 (concentration range of 5560 nM to 68.64 nM in a 3-fold dilution series and 5 point titration). The average % inhibition in HBV DNA, HBsAg and HBeAg, and standard deviations of 3 replicates observed either with TAF or compound 3 treatments alone or in combination are shown in Tables 20a, 20b, and 20c as indicated below. The EC₅₀ values of TAF and compound 3 were determined in an earlier experiment and are shown in Table 20d; some variance was observed from different lots of PHH cells.

When the observed values of a two-inhibitor combination were compared to what is expected from additive interaction for the above concentration range, the combinations were found to be additive or synergistic, with no antagonism (Table 20d) as per MacSynergy II analysis and using the interpretive criteria described above by Prichard and Shipman (1992). No significant inhibition of cell viability or proliferation was observed by microscopy or CCK8 assay in the analyzed samples.

TABLE 20a
Effect on HBV DNA in In Vitro Combination of TAF and Compound 3

| [DRUG] | | | | | | | AVERAGE % INHIBITION |
|---|---|---|---|---|---|---|---|
| TAF (nM) | 0.00 | 68.64 | 205.93 | 617.78 | 1853.33 | 5560.00 | COMPOUND 3 (nM) |
| 3.70 | 78.31 | 76.66 | 75.83 | 84 | 83 | 87.22 | |
| 1.23 | 63.39 | 66.71 | 65.36 | 76.33 | 81.98 | 88.21 | |
| 0.41 | 28.78 | 50.25 | 43.6 | 56.51 | 77.12 | 86.4 | |
| 0.14 | 3.84 | 22.99 | 19.73 | 44.15 | 74.08 | 86.53 | |
| 0.05 | −8.77 | 15.84 | 18.49 | 40.03 | 71.93 | 83.56 | |
| 0.00 | 0 | −2.79 | −5.02 | 34.78 | 66.43 | 85.32 | |
| [DRUG] | | | | | | | STANDARD DEVIATION (%) |
| TAF (nM) | 0.00 | 68.64 | 205.93 | 617.78 | 1853.33 | 5560.00 | COMPOUND 3 (nM) |
| 3.70 | 4.13 | 5.74 | 5.65 | 2.86 | 6.34 | 3.87 | |
| 1.23 | 6.26 | 4 | 1.75 | 3.36 | 2.15 | 1.61 | |
| 0.41 | 15.82 | 4.83 | 4.35 | 8.44 | 2.31 | 0.77 | |
| 0.14 | 5.2 | 10.08 | 12.17 | 5.9 | 2.81 | 1.93 | |
| 0.05 | 11.46 | 2.67 | 13.74 | 8.32 | 4 | 6.05 | |
| 0.00 | 19.74 | 24.58 | 16.02 | 21.37 | 3.11 | 3.19 | |

| [DRUG] | | | | | | | ADDITIVE INHIBITION |
|---|---|---|---|---|---|---|---|
| TAF (nM) | 0.00 | 68.64 | 205.93 | 617.78 | 1853.33 | 5560.00 | COMPOUND 3 (nM) |
| 3.70 | 78.31 | 77.7 | 77.22 | 85.85 | 92.72 | 96.82 | |
| 1.23 | 63.39 | 62.37 | 61.55 | 76.12 | 87.71 | 94.63 | |
| 0.41 | 28.78 | 26.79 | 25.2 | 53.55 | 76.09 | 89.54 | |
| 0.14 | 3.84 | 1.16 | −0.99 | 37.28 | 67.72 | 85.88 | |
| 0.05 | −8.77 | −11.8 | −14.23 | 29.06 | 63.49 | 84.03 | |
| 0.00 | 0 | −2.79 | −5.02 | 34.78 | 66.43 | 85.32 | |

| | |
|---|---|
| [DRUG] | SYNERGY PLOT (99.9%) |

| TAF (nM) | 0 | 68.642 | 205.93 | 617.78 | 1853.3 | 5560 | Bonferroni Adj. | 98% |
|---|---|---|---|---|---|---|---|---|
| 3.70 | 0 | 0 | 0 | 0 | 0 | 0 | SYNERGY | 30.5 |
| 1.23 | 0 | 0 | 0 | 0 | 0 | −1.1215 | log volume | 6.94 |
| 0.41 | 0 | 7.56447 | 4.08415 | 0 | 0 | −0.6059 | | |
| 0.14 | 0 | 0 | 0 | 0 | 0 | 0 | ANTAGONISM | −1.73 |
| 0.05 | 0 | 18.853 | 0 | 0 | 0 | 0 | log volume | −0.39 |
| 0.00 | 0 | 0 | 0 | 0 | 0 | 0 | | |

TABLE 20b
Effect on HBsAg in In Vitro Combination of TAF and Compound 3

| [DRUG] | | | | | | | AVERAGE % INHIBITION |
|---|---|---|---|---|---|---|---|
| TAF (nM) | 0.00 | 68.64 | 205.93 | 617.78 | 1853.33 | 5560.00 | COMPOUND 3 (nM) |
| 3.70 | −6.72 | 6.49 | 7.67 | 0.89 | 29.25 | 52.65 | |
| 1.23 | 10.97 | 13.51 | 15.13 | 15.13 | 27.31 | 58.97 | |
| 0.41 | 11.29 | 12.8 | 10.81 | 11.93 | 27.47 | 49.79 | |
| 0.14 | 12.83 | 3.2 | 5.03 | 3.13 | 16.78 | 48.23 | |
| 0.05 | −7.35 | −0.27 | 0.03 | 7.65 | 24.53 | 50.59 | |
| 0.00 | 0 | −16.35 | −21.58 | −5.12 | 14.6 | 43.83 | |

| [DRUG] | | | | | | | STANDARD DEVIATION (%) |
|---|---|---|---|---|---|---|---|
| TAF (nM) | 0.00 | 68.64 | 205.93 | 617.78 | 1853.33 | 5560.00 | COMPOUND 3 (nM) |
| 3.70 | 3.91 | 5.1 | 5.03 | 8.91 | 7.06 | 8.33 | |
| 1.23 | 3.52 | 5.17 | 5.31 | 13 | 7.04 | 5.03 | |
| 0.41 | 8.18 | 13.14 | 3.12 | 11.46 | 12.56 | 2.98 | |
| 0.14 | 10.96 | 14.74 | 11.52 | 2.55 | 6.84 | 7.2 | |
| 0.05 | 11.13 | 9.98 | 4.72 | 15.21 | 8.94 | 3.8 | |
| 0.00 | 22.17 | 16.06 | 23.58 | 14.67 | 9.83 | 6.94 | |

| [DRUG] | | | | | | | ADDITIVE INHIBITION |
|---|---|---|---|---|---|---|---|
| TAF (nM) | 0.00 | 68.64 | 205.93 | 617.78 | 1853.33 | 5560.00 | COMPOUND 3 (nM) |
| 3.70 | −6.72 | −24.17 | −29.75 | −12.18 | 8.86 | 40.06 | |
| 1.23 | 10.97 | −3.59 | −8.24 | 6.41 | 23.97 | 49.99 | |
| 0.41 | 11.29 | −3.21 | −7.85 | 6.75 | 24.24 | 50.17 | |
| 0.14 | 12.83 | −1.42 | −5.98 | 8.37 | 25.56 | 51.04 | |
| 0.05 | −7.35 | −24.9 | −30.52 | −12.85 | 8.32 | 39.7 | |
| 0.00 | 0 | −16.35 | −21.58 | −5.12 | 14.6 | 43.83 | |

| [DRUG] | | | | | | | SYNERGY PLOT (99.9%) | |
|---|---|---|---|---|---|---|---|---|
| TAF (nM) | 0.00 | 68.64 | 205.93 | 617.78 | 1853.33 | 5560.00 | Bonferroni Adj. | 98% |
| 3.70 | 0 | 13.8759 | 20.8663 | 0 | 0 | 0 | SYNERGY | 64.13 |
| 1.23 | 0 | 0.08553 | 5.89479 | 0 | 0 | 0 | log volume | 14.6 |
| 0.41 | 0 | 0 | 8.39208 | 0 | 0 | 0 | | |
| 0.14 | 0 | 0 | 0 | 0 | 0 | 0 | ANTAGONISM | 0 |
| 0.05 | 0 | 0 | 15.0165 | 0 | 0 | 0 | log volume | 0 |
| 0.00 | 0 | 0 | 0 | 0 | 0 | 0 | | |

TABLE 20c
Effect on HBeAg in In Vitro Combination of TAF and Compound 3

| [DRUG] | | | | | | | AVERAGE % INHIBITION |
|---|---|---|---|---|---|---|---|
| TAF (nM) | 0.00 | 68.59 | 205.76 | 617.28 | 1851.85 | 5555.56 | COMPOUND 3 (nM) |
| 3.70 | 11.87 | 6.27 | 25.76 | 19.94 | 27.49 | 61.6 | |
| 1.23 | 9.91 | 11.39 | 10.58 | 18.23 | 26.38 | 55.2 | |
| 0.41 | 1.76 | 1.32 | −4.69 | 15.28 | 22.07 | 48.25 | |
| 0.14 | −2.78 | −3.24 | 1.07 | 13.95 | 18.72 | 46.8 | |
| 0.05 | 1.17 | 3.04 | 0.21 | 10.48 | 17.05 | 49.18 | |
| 0.00 | 0 | −5.05 | −6.33 | 2.77 | 29.66 | 40.38 | |
| [DRUG] | | | | | | | STANDARD DEVIATION (%) |
| TAF (nM) | 0.00 | 68.59 | 205.76 | 617.28 | 1851.85 | 5555.56 | COMPOUND 3 (nM) |
| 3.70 | 8.54 | 19.25 | 17.35 | 14.39 | 11.4 | 3.56 | |
| 1.23 | 13.91 | 1.05 | 5.26 | 6.23 | 11.06 | 5.69 | |
| 0.41 | 18.44 | 7.35 | 8.98 | 4.02 | 7.19 | 2.75 | |
| 0.14 | 11.41 | 19.4 | 4.08 | 12.99 | 5.4 | 4.89 | |
| 0.05 | 16.36 | 9.09 | 6.96 | 4.15 | 9.2 | 7.01 | |
| 0.00 | 28.29 | 2.3 | 6.31 | 5.64 | 11.69 | 6.37 | |

| [DRUG] | | | | | | | ADDITIVE INHIBITION |
|---|---|---|---|---|---|---|---|
| TAF (nM) | 0.00 | 68.59 | 205.76 | 617.28 | 1851.85 | 5555.56 | COMPOUND 3 (nM) |
| 3.70 | 11.87 | 7.42 | 6.29 | 14.31 | 38.01 | 47.46 | |
| 1.23 | 9.91 | 5.36 | 4.21 | 12.41 | 36.63 | 46.29 | |
| 0.41 | 1.76 | −3.2 | −4.46 | 4.48 | 30.9 | 41.43 | |
| 0.14 | −2.78 | −7.97 | −9.29 | 0.07 | 27.7 | 38.72 | |
| 0.05 | 1.17 | −3.82 | −5.09 | 3.91 | 30.48 | 41.08 | |
| 0.00 | 0 | −5.05 | −6.33 | 2.77 | 29.66 | 40.38 | |

| | |
|---|---|
| [DRUG] | SYNERGY PLOT (99.9%) |

| TAF (nM) | 0.00 | 68.59 | 205.76 | 617.28 | 1851.85 | 5555.56 | Bonferroni Adj. | 98% |
|---|---|---|---|---|---|---|---|---|
| 3.70 | 0 | 0 | 0 | 0 | 0 | 2.42404 | SYNERGY | 5 |
| 1.23 | 0 | 2.57445 | 0 | 0 | 0 | 0 | log volume | 1.14 |
| 0.41 | 0 | 0 | 0 | 0 | 0 | 0 | | |
| 0.14 | 0 | 0 | 0 | 0 | 0 | 0 | ANTAGONISM | 0 |
| 0.05 | 0 | 0 | 0 | 0 | 0 | 0 | log volume | 0 |
| 0.00 | 0 | 0 | 0 | 0 | 0 | 0 | | |

TABLE 20d
Summary of results of in vitro combination studies of TAF and Compound 3 in PHH cell culture system:

| HBV | | | Inhibitor | Inhibitor | Synergy | Synergy | Antagonism | Antagonism | |
|---|---|---|---|---|---|---|---|---|---|
| Assay | Inhibitor | Inhibitor | A EC₅₀ | B EC₅₀ | Volume | Log | Volume | Log | |
| Endpoint | A | B | (nM)# | (nM)# | (μM² %)* | Volume | (μM² %)* | Volume | Conclusion |
| HBV DNA | TAF | COMPOUND 3 | 0.405 | 876.5 | 30.5 | 6.94 | −1.73 | −0.39 | Synergy |
| HBsAg | TAF | COMPOUND 3 | >100 | 7793 | 64.13 | 14.6 | 0 | 0 | Synergy |
| HBeAg | TAF | COMPOUND 3 | >100 | 8850 | 5.0 | 1.14 | 0 | 0 | Additive |

*at 99.9% confidence interval
#determined in an earlier separate experiment

### Example 21

### In Vitro Combination of IFNα2a and Compound 22

### Study Goal

To determine whether a two-drug combination of compound 22 (a small molecule inhibitor of HBV encapsidation belonging to the sulfamoyl benzamide chemical class), and pegylated interferon alpha 2a (IFNα2a, an antiviral cytokine that activates innate immunity pathways in hepatocytes), is additive, synergistic or antagonistic in vitro using HBV-infected human primary hepatocytes in a cell culture model system.

### Results and Conclusion

IFNα2a (concentration range of 10.0 IU/mL to 0.123 IU/mL in a 3-fold dilution series and 5 point titration) was tested in combination with compound 22 (concentration range of 5000 nM to 61.721 nM in a 3-fold dilution series and 5 point titration). The average % inhibition in HBV DNA, HBsAg and HBeAg, and standard deviations of 3 replicates observed either with IFNa2a or compound 22 treatments alone or in combination are shown in Tables 21a, 21b, and 21c as indicated below. The EC₅₀ values of IFNα2a and compound 22 were determined in an earlier experiment and are shown in Table 21d; some variance was observed from different lots of PHH cells.

When the observed values of a two-inhibitor combination were compared to what is expected from additive interaction for the above concentration range, the combinations were found to be additive to synergistic, with no antagonism (Table 21d) as per MacSynergy II analysis and using the interpretive criteria described above by Prichard and Shipman (1992). No significant inhibition of cell viability or proliferation was observed by microscopy or CCK8 assay in the analyzed samples.

TABLE 21a
Effect on HBV DNA in In Vitro Combination of IFNα2a and Compound 22

| [DRUG] | | | | | | | |
|---|---|---|---|---|---|---|---|
| IFNα2a | | | | | | | AVERAGE % INHIBITION |
| IU/mL | 0.00 | 61.73 | 185.19 | 555.56 | 1666.67 | 5000.00 | COMPOUND 22 (μM) |
| 10.00 | 59 | 70.58 | 67.36 | 66.34 | 75.72 | 83.3 | |
| 3.33 | 52.39 | 69.79 | 71.36 | 68.3 | 72.01 | 84.76 | |
| 1.11 | 28.08 | 59.77 | 59.61 | 55.17 | 63.22 | 80.59 | |
| 0.37 | 6.59 | 44.09 | 42.48 | 42.82 | 61.33 | 75.33 | |
| 0.12 | −18.56 | 29.97 | 23.99 | 27.7 | 45.63 | 78.65 | |
| 0.00 | 0 | −9.02 | −33.53 | −13.72 | 22.31 | 69.19 | |

| [DRUG] | | | | | | | |
|---|---|---|---|---|---|---|---|
| IFNα2a | | | | | | | STANDARD DEVIATION (%) |
| IU/mL | 0.00 | 61.73 | 185.19 | 555.56 | 1666.67 | 5000.00 | COMPOUND 22 (μM) |
| 10.00 | 7.24 | 3.95 | 0.56 | 10.17 | 3.06 | 4.6 | |
| 3.33 | 11.43 | 3.1 | 4.52 | 7.4 | 11.11 | 4.42 | |
| 1.11 | 16.44 | 2.71 | 2.78 | 22.26 | 6.66 | 1.34 | |
| 0.37 | 33.49 | 11.81 | 2.73 | 7.7 | 14.25 | 1.86 | |
| 0.12 | 23.97 | 16.1 | 11.97 | 10.1 | 9.2 | 2.49 | |
| 0.00 | 35.38 | 12.95 | 29.16 | 24.96 | 22.77 | 2.75 | |

| [DRUG] | | | | | | | |
|---|---|---|---|---|---|---|---|
| IFNα2a | | | | | | | ADDITIVE INHIBITION |
| IU/mL | 0.00 | 61.73 | 185.19 | 555.56 | 1666.67 | 5000.00 | COMPOUND 22 (μM) |
| 10.00 | 59 | 55.3 | 45.25 | 53.37 | 68.15 | 87.37 | |
| 3.33 | 52.39 | 48.1 | 36.43 | 45.86 | 63.01 | 85.33 | |
| 1.11 | 28.08 | 21.59 | 3.97 | 18.21 | 44.13 | 77.84 | |
| 0.37 | 6.59 | −1.84 | −24.73 | −6.23 | 27.43 | 71.22 | |
| 0.12 | −18.56 | −29.25 | −58.31 | −34.83 | 7.89 | 63.47 | |
| 0.00 | 0 | −9.02 | −33.53 | −13.72 | 22.31 | 69.19 | |

| | |
|---|---|
| [DRUG] | |
| IFNα2a | SYNERGY PLOT (99.9%) |

| IU/mL | 0 | 61.728 | 185.19 | 555.56 | 1666.7 | 5000 | Bonferroni Adj. | 98% |
|---|---|---|---|---|---|---|---|---|
| 10.00 | 0 | 2.28055 | 20.267 | 0 | 0 | 0 | SYNERGY | 311.72 |
| 3.33 | 0 | 11.4879 | 20.0547 | 0 | 0 | 0 | log volume | 70.96 |
| 1.11 | 0 | 29.2614 | 46.491 | 0 | 0 | 0 | | |
| 0.37 | 0 | 7.06329 | 58.2256 | 23.7093 | 0 | 0 | ANTAGONISM | 0 |
| 0.12 | 0 | 6.2349 | 42.9067 | 29.2909 | 7.4628 | 6.98541 | log volume | 0 |
| 0.00 | 0 | 0 | 0 | 0 | 0 | 0 | | |

TABLE 21b
Effect on HBsAg in In Vitro Combination of IFNα2a and Compound 22

| [DRUG] | | | | | | | |
|---|---|---|---|---|---|---|---|
| IFNα2a | | | | | | | AVERAGE % INHIBITION |
| IU/mL | 0.00 | 61.73 | 185.19 | 555.56 | 1666.67 | 5000.00 | COMPOUND 22 (μM) |
| 10.00 | 20.1 | 23.86 | 18.44 | 23.51 | 32.47 | 46.3 | |
| 3.33 | 10.2 | 21.09 | 18.86 | 23.8 | 32.72 | 40.92 | |
| 1.11 | 8.8 | 17.52 | 19.02 | 18.44 | 29.23 | 41.77 | |
| 0.37 | 4.6 | 10.38 | 12.89 | 12.73 | 19.64 | 32.99 | |
| 0.12 | −1.67 | 10.33 | 10.48 | 16.18 | 20.01 | 33.22 | |
| 0.00 | 0 | −13.83 | −10.58 | −5.08 | 10.34 | 23.09 | |

| [DRUG] | | | | | | | |
|---|---|---|---|---|---|---|---|
| IFNα2a | | | | | | | STANDARD DEVIATION (%) |
| IU/mL | 0.00 | 61.73 | 185.19 | 555.56 | 1666.67 | 5000.00 | COMPOUND 22 (μM) |
| 10.00 | 13.1 | 8.15 | 6.53 | 2.24 | 6.55 | 3.24 | |
| 3.33 | 11.12 | 8.23 | 8.23 | 3.93 | 10.55 | 10.22 | |
| 1.11 | 14.56 | 12.01 | 8.75 | 8.2 | 12.13 | 11.6 | |
| 0.37 | 9.75 | 7.48 | 17.42 | 8.47 | 12.45 | 15.01 | |
| 0.12 | 20.23 | 10.68 | 5.97 | 9.82 | 12.81 | 14.29 | |
| 0.00 | 18.63 | 16.23 | 12.6 | 17.72 | 16.11 | 15.81 | |

| [DRUG] | | | | | | | |
|---|---|---|---|---|---|---|---|
| IFNα2a | | | | | | | ADDITIVE INHIBITION |
| IU/mL | 0.00 | 61.73 | 185.19 | 555.56 | 1666.67 | 5000.00 | COMPOUND 22 (μM) |
| 10.00 | 20.1 | 9.05 | 11.65 | 16.04 | 28.36 | 38.55 | |
| 3.33 | 10.2 | −2.22 | 0.7 | 5.64 | 19.49 | 30.93 | |
| 1.11 | 8.8 | −3.81 | −0.85 | 4.17 | 18.23 | 29.86 | |
| 0.37 | 4.6 | −8.59 | −5.49 | −0.25 | 14.46 | 26.63 | |
| 0.12 | −1.67 | −15.73 | −12.43 | −6.83 | 8.84 | 21.81 | |
| 0.00 | 0 | −13.83 | −10.58 | −5.08 | 10.34 | 23.09 | |

| [DRUG] | | | | | | | | |
|---|---|---|---|---|---|---|---|---|
| IFNα2a | | | | | | | SYNERGY PLOT (99.9%) | |
| IU/mL | 0 | 61.728 | 185.19 | 555.56 | 1666.7 | 5000 | Bonferroni Adj. | 98% |
| 10.00 | 0 | 0 | 0 | 0.09816 | 0 | 0 | SYNERGY | 8.59 |
| 3.33 | 0 | 0 | 0 | 5.22637 | 0 | 0 | log volume | 1.96 |
| 1.11 | 0 | 0 | 0 | 0 | 0 | 0 | | |
| 0.37 | 0 | 0 | 0 | 0 | 0 | 0 | ANTAGONISM | 0 |
| 0.12 | 0 | 0 | 3.26273 | 0 | 0 | 0 | log volume | 0 |
| 0.00 | 0 | 0 | 0 | 0 | 0 | 0 | | |

TABLE 21c
Effect on HBeAg in In Vitro Combination of IFNα2a and Compound 22

| [DRUG] | | | | | | | |
|---|---|---|---|---|---|---|---|
| IFNα2a | | | | | | | AVERAGE % INHIBITION |
| IU/mL | 0.00 | 61.73 | 185.19 | 555.56 | 1666.67 | 5000.00 | COMPOUND 22 (μM) |
| 10.00 | 30.13 | 29.1 | 26.43 | 26.51 | 34.37 | 48.55 | |
| 3.33 | 17.16 | 17.85 | 18 | 19.33 | 29.38 | 39.7 | |
| 1.11 | 15.27 | 14.17 | 15.76 | 10.98 | 26.67 | 41.95 | |
| 0.37 | 1.78 | 2.04 | 2.11 | −0.99 | 10.34 | 27.11 | |
| 0.12 | 7.42 | 11.7 | 10.2 | 8.06 | 14.8 | 34.39 | |
| 0.00 | 0 | −7.2 | −9.57 | −8.17 | 5.92 | 20.93 | |
| [DRUG] | | | | | | | |
| IFNα2a | | | | | | | STANDARD DEVIATION (%) |
| IU/mL | 0.00 | 61.73 | 185.19 | 555.56 | 1666.67 | 5000.00 | COMPOUND 22 (μM) |
| 10.00 | 0.33 | 8.25 | 5.02 | 1.12 | 4.12 | 3.3 | |
| 3.33 | 5.51 | 6.25 | 6.16 | 6.03 | 3.41 | 4.79 | |
| 1.11 | 2.91 | 12.64 | 3.52 | 11.08 | 6.97 | 8.93 | |
| 0.37 | 3.5 | 12.74 | 8.62 | 13.47 | 7.91 | 4.93 | |
| 0.12 | 6.9 | 9.72 | 7.43 | 4.72 | 11.46 | 7.25 | |
| 0.00 | 7.86 | 5.83 | 6.88 | 13.23 | 8.51 | 9.89 | |

| [DRUG] | | | | | | | |
|---|---|---|---|---|---|---|---|
| IFNα2a | | | | | | | ADDITIVE INHIBITION |
| IU/mL | 0.00 | 61.73 | 185.19 | 555.56 | 1666.67 | 5000.00 | COMPOUND 22 (μM) |
| 10.00 | 30.13 | 25.1 | 23.44 | 24.42 | 34.27 | 44.75 | |
| 3.33 | 17.16 | 11.2 | 9.23 | 10.39 | 22.06 | 34.5 | |
| 1.11 | 15.27 | 9.17 | 7.16 | 8.35 | 20.29 | 33 | |
| 0.37 | 1.78 | −5.29 | −7.62 | −6.24 | 7.59 | 22.34 | |
| 0.12 | 7.42 | 0.75 | −1.44 | −0.14 | 12.9 | 26.8 | |
| 0.00 | 0 | −7.2 | −9.57 | −8.17 | 5.92 | 20.93 | |

| [DRUG] | | | | | | | | |
|---|---|---|---|---|---|---|---|---|
| IFNα2a | | | | | | | SYNERGY PLOT (99.9%) | |
| IU/mL | 0 | 61.728 | 185.19 | 555.56 | 1666.7 | 5000 | Bonferroni Adj. | 98% |
| 10.00 | 0 | 0 | 0 | 0 | 0 | 0 | SYNERGY | 0 |
| 3.33 | 0 | 0 | 0 | 0 | 0 | 0 | log volume | 0 |
| 1.11 | 0 | 0 | 0 | 0 | 0 | 0 | | |
| 0.37 | 0 | 0 | 0 | 0 | 0 | 0 | ANTAGONISM | 0 |
| 0.12 | 0 | 0 | 0 | 0 | 0 | 0 | log volume | 0 |
| 0.00 | 0 | 0 | 0 | 0 | 0 | 0 | | |

TABLE 21d
Summary of results of in vitro combination studies of IFNα2a and Compound 22 in PHH cell culture system:

| HBV | | | Inhibitor | Inhibitor | Synergy | Synergy | Antagonism | Antagonism | |
|---|---|---|---|---|---|---|---|---|---|
| Assay | Inhibitor | Inhibitor | A EC₅₀ | B EC₅₀ | Volume | Log | Volume | Log | |
| Endpoint | A | B | (IU/mL)# | (nM)# | (μM² %)* | Volume | (μM² %)* | Volume | Conclusion |
| HBV DNA | IFNα2a | COMPOUND 22 | 2.154 | 1020 | 311.72 | 70.96 | 0 | 0 | Synergy |
| HBsAg | IFNα2a | COMPOUND 22 | 13.8 | 12,800 | 8.59 | 1.96 | 0 | 0 | Additive |
| HBeAg | IFNα2a | COMPOUND 22 | 10.24 | 10,740 | 0 | 0 | 0 | 0 | Additive |

*at 99.9% confidence interval
#determined in an earlier separate experiment

### Example 22

### In Vitro Combination of Compound 22 and TAF

### Study Goal

To determine whether a two-drug combination of compound 22 (a small molecule inhibitor of HBV encapsidation belonging to the sulfamoyl benzamide chemical class), and tenofovir (in the form of the prodrug tenofovir alafenamide, or TAF, a nucleotide analog inhibitor of HBV polymerase), is additive, synergistic or antagonistic in vitro using HBV-infected human primary hepatocytes in a cell culture model system.

### Results and Conclusion

TAF (concentration range of 10.0 nM to 0.12 nM in a 3-fold dilution series and 5 point titration) was tested in combination with compound 22 (concentration range of 5000 nM to 61.721 nM in a 3-fold dilution series and 5 point titration). The average % inhibition in HBV DNA, HBsAg and HBeAg, and standard deviations of 3 replicates observed either with compound 22 or TAF treatments alone or in combination are shown in Tables 22a, 22b, and 22c as indicated below. The EC₅₀ values of TAF and compound 22 were determined in an earlier experiment and are shown in Table 22d; some variance was observed from different lots of PHH cells.

When the observed values of a two-inhibitor combination were compared to what is expected from additive interaction for the above concentration range, the combinations were found to be additive, with no antagonism (Table 22d) as per MacSynergy II analysis and using the interpretive criteria described above by Prichard and Shipman (1992). No significant inhibition of cell viability or proliferation was observed by microscopy or CCK8 assay in the analyzed samples.

TABLE 22a
Effect on HBV DNA in In Vitro Combination of Compound 22 and TAF

| [DRUG] | | | | | | | AVERAGE % INHIBITION |
|---|---|---|---|---|---|---|---|
| TAF (nM) | 0 | 61.728 | 185.19 | 555.56 | 1666.7 | 5000 | COMPOUND 22 (nM) |
| 10.00 | 50.21 | 60.62 | 59.41 | 66.66 | 65.77 | 71.26 | |
| 3.33 | 40.16 | 51.09 | 48.53 | 60.14 | 55.25 | 70.85 | |
| 1.11 | 4.95 | 25.5 | 30.09 | 25.21 | 42.82 | 62.1 | |
| 0.37 | −1.92 | 5.92 | 11.85 | 14.68 | 29.37 | 54.24 | |
| 0.12 | −2.6 | −5.22 | 5.12 | 11.67 | 36.5 | 52.5 | |
| 0.00 | 0 | 2.38 | −3.33 | 8.01 | 27.98 | 54.66 | |
| [DRUG] | | | | | | | STANDARD DEVIATION (%) |
| TAF (nM) | 0 | 61.728 | 185.19 | 555.56 | 1666.7 | 5000 | COMPOUND 22 (nM) |
| 10.00 | 1.32 | 8.36 | 3.9 | 10.1 | 3.39 | 11.57 | |
| 3.33 | 12.34 | 15.26 | 5.42 | 4.38 | 13.68 | 7.66 | |
| 1.11 | 25.38 | 8.61 | 20.31 | 18.26 | 6.64 | 11.33 | |
| 0.37 | 8.11 | 10.64 | 16.41 | 12.37 | 11.31 | 8.93 | |
| 0.12 | 3.28 | 6.41 | 13.44 | 11.64 | 0.94 | 10.76 | |
| 0.00 | 0.19 | 7.49 | 13.42 | 18.44 | 0.83 | 17.12 | |

| [DRUG] | | | | | | | ADDITIVE INHIBITION |
|---|---|---|---|---|---|---|---|
| TAF (nM) | 0 | 61.728 | 185.19 | 555.56 | 1666.7 | 5000 | COMPOUND 22 (nM) |
| 10.00 | 50.21 | 51.4 | 48.55 | 54.2 | 64.14 | 77.43 | |
| 3.33 | 40.16 | 41.58 | 38.17 | 44.95 | 56.9 | 72.87 | |
| 1.11 | 4.95 | 7.21 | 1.78 | 12.56 | 31.54 | 56.9 | |
| 0.37 | −1.92 | 0.51 | −5.31 | 6.24 | 26.6 | 53.79 | |
| 0.12 | −2.6 | −0.16 | −6.02 | 5.62 | 26.11 | 53.48 | |
| 0.00 | 0 | 2.38 | −3.33 | 8.01 | 27.98 | 54.66 | |

| [DRUG] | | | | | | | SYNERGY PLOT (99.9%) | |
|---|---|---|---|---|---|---|---|---|
| TAF (nM) | 0 | 61.728 | 185.19 | 555.56 | 1666.7 | 5000 | Bonferroni Adj. | 98% |
| 10.00 | 0 | 0 | 0 | 0 | 0 | 0 | SYNERGY | 8.07 |
| 3.33 | 0 | 0 | 0 | 0.77542 | 0 | 0 | log volume | 1.84 |
| 1.11 | 0 | 0 | 0 | 0 | 0 | 0 | | |
| 0.37 | 0 | 0 | 0 | 0 | 0 | 0 | ANTAGONISM | 0 |
| 0.12 | 0 | 0 | 0 | 0 | 7.29646 | 0 | log volume | 0 |
| 0.00 | 0 | 0 | 0 | 0 | 0 | 0 | | |

TABLE 22b
Effect on HBsAg in In Vitro Combination of Compound 22 and TAF

| [DRUG] | | | | | | | AVERAGE % INHIBITION |
|---|---|---|---|---|---|---|---|
| TAF (nM) | 0 | 61.728 | 185.19 | 555.56 | 1666.7 | 5000 | COMPOUND 22 (nM) |
| 10 | 7.97 | 3.97 | 21.33 | 7.89 | 24.84 | 38.54 | |
| 3.3333333 | 9.06 | −6.48 | 16.7 | 16.53 | 24.27 | 44.07 | |
| 1.1111111 | 20.81 | 13.85 | 21.8 | 20.98 | 27.18 | 46.11 | |
| 0.3703704 | 10.78 | −3.62 | 10.04 | 10.32 | 23.21 | 45.05 | |
| 0.1234568 | 29.82 | 19.99 | 14.56 | 21.8 | 21.67 | 48.57 | |
| 0 | 0 | −0.32 | 2.37 | −2.17 | 17.68 | 20.73 | |
| [DRUG] | | | | | | | STANDARD DEVIATION (%) |
| TAF (nM) | 0 | 61.728 | 185.19 | 555.56 | 1666.7 | 5000 | COMPOUND 22 (nM) |
| 10 | 5.77 | 2.84 | 10.6 | 6.45 | 2.33 | 6.64 | |
| 3.3333333 | 13.78 | 10.12 | 9.21 | 7.53 | 7.28 | 4.26 | |
| 1.1111111 | 5.53 | 6.36 | 15.1 | 9.66 | 4.2 | 2.72 | |
| 0.3703704 | 4.42 | 15.44 | 4.26 | 7.98 | 7.62 | 2.68 | |
| 0.1234568 | 3.67 | 4.25 | 4.49 | 3.91 | 8.82 | 1.51 | |
| 0 | 0.59 | 19.01 | 7.88 | 14.89 | 15.32 | 16.75 | |

| [DRUG] | | | | | | | ADDITIVE INHIBITION |
|---|---|---|---|---|---|---|---|
| TAF (nM) | 0 | 61.728 | 185.19 | 555.56 | 1666.7 | 5000 | COMPOUND 22 (nM) |
| 10 | 7.97 | 7.68 | 10.15 | 5.97 | 24.24 | 27.05 | |
| 3.3333333 | 9.06 | 8.77 | 11.22 | 7.09 | 25.14 | 27.91 | |
| 1.1111111 | 20.81 | 20.56 | 22.69 | 19.09 | 34.81 | 37.23 | |
| 0.3703704 | 10.78 | 10.49 | 12.89 | 8.84 | 26.55 | 29.28 | |
| 0.1234568 | 29.82 | 29.6 | 31.48 | 28.3 | 42.23 | 44.37 | |
| 0 | 0 | −0.32 | 2.37 | −2.17 | 17.68 | 20.73 | |

| [DRUG] | | | | | | | SYNERGY PLOT (99.9%) | |
|---|---|---|---|---|---|---|---|---|
| TAF (nM) | 0 | 61.728 | 185.19 | 555.56 | 1666.7 | 5000 | Bonferroni Adj. | 98% |
| 0 | | | | | | | | |
| 10 | 0 | 0 | 0 | 0 | 0 | 0 | SYNERGY | 9.09 |
| 3.3333333 | 0 | 0 | 0 | 0 | 0 | 2.14034 | log volume | 2.07 |
| 1.1111111 | 0 | 0 | 0 | 0 | 0 | 0 | | |
| 0.3703704 | 0 | 0 | 0 | 0 | 0 | 6.95012 | ANTAGONISM | −2.14 |
| 0.1234568 | 0 | 0 | 2.1434 | 0 | 0 | 0 | log volume | −0.49 |
| 0 | 0 | 0 | 0 | 0 | 0 | 0 | | |

TABLE 22c
Effect on HBeAg in In Vitro Combination of Compound 22 and TAF

| [DRUG] | | | | | | | AVERAGE % INHIBITION |
|---|---|---|---|---|---|---|---|
| TAF (nM) | 0 | 61.728 | 185.19 | 555.56 | 1666.7 | 5000 | COMPOUND 22 (nM) |
| 10.00 | 22.85 | −0.79 | 17.72 | 8.41 | 23.95 | 42.03 | |
| 3.33 | 19.69 | −14.8 | 8.11 | 3.2 | 24.12 | 36.2 | |
| 1.11 | 22.56 | 1.31 | 15.81 | 20.43 | 22.71 | 49.56 | |
| 0.37 | 9.9 | −14.54 | −2.63 | 10.7 | 21.6 | 42.03 | |
| 0.12 | 26.61 | 17.84 | 15.03 | 21.04 | 26.27 | 50.3 | |
| 0.00 | 0 | −6.71 | −12.41 | −5.06 | 10.1 | 29.74 | |
| [DRUG] | | | | | | | STANDARD DEVIATION (%) |
| TAF (nM) | 0 | 61.728 | 185.19 | 555.56 | 1666.7 | 5000 | COMPOUND 22 (nM) |
| 10.00 | 19.83 | 13.7 | 2.25 | 17.67 | 11.95 | 8.64 | |
| 3.33 | 9.59 | 13.32 | 15.74 | 3.59 | 14.71 | 9.54 | |
| 1.11 | 8.99 | 14.21 | 16.19 | 10.78 | 1.53 | 2.78 | |
| 0.37 | 5.26 | 34.36 | 16.86 | 12.05 | 12.45 | 7.4 | |
| 0.12 | 4.71 | 14.39 | 8.61 | 5.08 | 4.18 | 4.19 | |
| 0.00 | 0.63 | 20.55 | 10.69 | 17.17 | 20.78 | 11.65 | |

| [DRUG] | | | | | | | ADDITIVE INHIBITION |
|---|---|---|---|---|---|---|---|
| TAF (nM) | 0 | 61.728 | 185.19 | 555.56 | 1666.7 | 5000 | COMPOUND 22 (nM) |
| 10.00 | 22.85 | 17.67 | 13.28 | 18.95 | 30.64 | 45.79 | |
| 3.33 | 19.69 | 14.3 | 9.72 | 15.63 | 27.8 | 43.57 | |
| 1.11 | 22.56 | 17.36 | 12.95 | 18.64 | 30.38 | 45.59 | |
| 0.37 | 9.9 | 3.85 | −1.28 | 5.34 | 19 | 36.7 | |
| 0.12 | 26.61 | 21.69 | 17.5 | 22.9 | 34.02 | 48.44 | |
| 0.00 | 0 | −6.71 | −12.41 | −5.06 | 10.1 | 29.74 | |

| [DRUG] | | | | | | | SYNERGY PLOT (99.9%) | |
|---|---|---|---|---|---|---|---|---|
| TAF (nM) | 0 | 61.728 | 185.19 | 555.56 | 1666.7 | 5000 | Bonferroni Adi. | 98% |
| 10.00 | 0 | 0 | 0 | 0 | 0 | 0 | SYNERGY | 0 |
| 3.33 | 0 | 0 | 0 | 0.6153 | 0 | 0 | log volume | 0 |
| 1.11 | 0 | 0 | 0 | 0 | 2.6348 | 0 | | |
| 0.37 | 0 | 0 | 0 | 0 | 0 | 0 | ANTAGONISM | −3.25 |
| 0.12 | 0 | 0 | 0 | 0 | 0 | 0 | log volume | −0.74 |
| 0.00 | 0 | 0 | 0 | 0 | 0 | 0 | | |

TABLE 22d
Summary of results of in vitro combination studies of Compound 22 and TAF in PHH cell culture system:

| HBV | | | Inhibitor | Inhibitor | Synergy | Synergy | Antagonism | Antagonism | |
|---|---|---|---|---|---|---|---|---|---|
| Assay | Inhibitor | Inhibitor | A EC₅₀ | B EC₅₀ | Volume | Log | Volume | Log | |
| Endpoint | A | B | (nM)# | (nM)# | (μM² %)* | Volume | (μM² %)* | Volume | Conclusion |
| HBV DNA | TAF | COMPOUND 22 | 0.405 | 1020 | 8.07 | 1.84 | 0 | 0 | Additive |
| HBsAg | TAF | COMPOUND 22 | >100 | 12,800 | 9.09 | 2.07 | −2.14 | −0.49 | Additive |
| HBeAg | TAF | COMPOUND 22 | >100 | 10,740 | 0 | 0 | −3.25 | −0.74 | Additive |

*at 99.9% confidence interval
#determined in an earlier separate experiment

### Example 23

### In Vitro Combination of Compound 22 and Compound 25

### Study Goal

To determine whether a two-drug combination of compound 22 (a small molecule inhibitor of HBV encapsidation belonging to the sulfamoyl benzamide chemical class), and compound 25 (a small molecule inhibitor of HBV DNA, HBsAg and HBeAg, belonging to the dihydroquinolizinone chemical class), is additive, synergistic or antagonistic in vitro using HBV-infected human primary hepatocytes in a cell culture model system.

### Results and Conclusion

Compound 25 (concentration range of 10.0 nM to 0.12 nM in a 3-fold dilution series and 5 point titration) was tested in combination with compound 22 (concentration range of 5000 nM to 61.73 nM in a 3-fold dilution series and 5 point titration). The average % inhibition in HBV DNA, HBsAg and HBeAg, and standard deviations of 3 replicates observed either with compound 25 or compound 22 treatments alone or in combination are shown in Tables 23a, 23b, and 23c as indicated below. The EC₅₀ values of compound 25 and compound 22 were determined in an earlier experiment and are shown in Table 23d; some variance was observed from different lots of PHH cells.

When the observed values of a two-inhibitor combination were compared to what is expected from additive interaction for the above concentration range, the combinations were found to be synergistic or additive, with no antagonism (Table 23d) as per MacSynergy II analysis and using the interpretive criteria described above by Prichard and Shipman (1992). No significant inhibition of cell viability or proliferation was observed by microscopy or CCK8 assay in the analyzed samples.

TABLE 23a
Effect on HBV DNA in In Vitro Combination of Compound 22 and Compound 25

| [DRUG] | | | | | | | |
|---|---|---|---|---|---|---|---|
| COMPOUND 25 | | | | | | | AVERAGE % INHIBITION |
| (nM) | 0.00 | 61.73 | 185.19 | 555.56 | 1666.67 | 5000.00 | COMPOUND 22 (nM) |
| 10.00 | 37.39 | 54.71 | 52.49 | 63.54 | 67.1 | 85.44 | |
| 3.33 | 16.41 | 50.43 | 52.25 | 53.21 | 62.83 | 82.08 | |
| 1.11 | −19.21 | 32.08 | 42.5 | 41.58 | 57.56 | 80.93 | |
| 0.37 | −46.48 | 30.71 | 23.72 | 21.3 | 52.22 | 73.23 | |
| 0.12 | −42.82 | 26.46 | 16.46 | 27.69 | 42.07 | 74.04 | |
| 0.00 | 0 | −11.75 | −9.12 | −12.7 | 17.94 | 63.06 | |

| [DRUG] | | | | | | | |
|---|---|---|---|---|---|---|---|
| COMPOUND 25 | | | | | | | STANDARD DEVIATION (%) |
| (nM) | 0.00 | 61.73 | 185.19 | 555.56 | 1666.67 | 5000.00 | COMPOUND 22 (nM) |
| 10.00 | 7.33 | 4.11 | 2.57 | 4.94 | 5.09 | 1.95 | |
| 3.33 | 6.98 | 4.36 | 7.16 | 4.68 | 3.23 | 3.21 | |
| 1.11 | 35.51 | 7.87 | 0.68 | 13.48 | 7.26 | 1.34 | |
| 0.37 | 51.6 | 6.46 | 0.9 | 21 | 7.5 | 1.71 | |
| 0.12 | 21.05 | 7.83 | 6 | 5.3 | 0.16 | 2.16 | |
| 0.00 | 40.03 | 5.71 | 4.36 | 11.48 | 8.67 | 2.92 | |

| [DRUG] | | | | | | | |
|---|---|---|---|---|---|---|---|
| COMPOUND 25 | | | | | | | ADDITIVE INHIBITION |
| (nM) | 0.00 | 61.73 | 185.19 | 555.56 | 1666.67 | 5000.00 | COMPOUND 22 (nM) |
| 10.00 | 37.39 | 30.03 | 31.68 | 29.44 | 48.62 | 76.87 | |
| 3.33 | 16.41 | 6.59 | 8.79 | 5.79 | 31.41 | 69.12 | |
| 1.11 | −19.21 | −33.22 | −30.08 | −34.35 | 2.18 | 55.96 | |
| 0.37 | −46.48 | −63.69 | −59.84 | −65.08 | −20.2 | 45.89 | |
| 0.12 | −42.82 | −59.6 | −55.85 | −60.96 | −17.2 | 47.24 | |
| 0.00 | 0 | −11.75 | −9.12 | −12.7 | 17.94 | 63.06 | |

| | |
|---|---|
| [DRUG] | |
| COMPOUND 25 | SYNERGY PLOT (99.9%) |

| (nM) | 0 | 61.728 | 185.19 | 555.56 | 1666.7 | 5000 | Bonferroni Adj. | 98% |
|---|---|---|---|---|---|---|---|---|
| 10.00 | 0 | 11.154 | 12.3521 | 17.8425 | 1.72881 | 2.15255 | SYNERGY | 846.13 |
| 3.33 | 0 | 29.4912 | 19.8964 | 32.0181 | 20.7901 | 2.39589 | log volume | 192.62 |
| 1.11 | 0 | 39.3998 | 70.3421 | 31.5673 | 31.4873 | 20.5601 | | |
| 0.37 | 0 | 73.1401 | 80.5981 | 17.269 | 47.7375 | 21.7124 | ANTAGONISM | 0 |
| 0.12 | 0 | 60.2915 | 52.564 | 71.2077 | 58.7434 | 19.6914 | log volume | 0 |
| 0.00 | 0 | 0 | 0 | 0 | 0 | 0 | | |

TABLE 23b
Effect on HBsAg in In Vitro Combination of Compound 22 and Compound 25

| | | | | | | | AVERAGE % INHIBITION |
|---|---|---|---|---|---|---|---|
| [DRUG] | 0.00 | 61.73 | 185.19 | 555.56 | 1666.67 | 5000.00 | COMPOUND 22 (nM) |
| 10.00 | 42.95 | 47.96 | 44.95 | 47.05 | 56.23 | 64.25 | |
| 3.33 | 20.81 | 33.92 | 29.53 | 31.58 | 44.61 | 49.94 | |
| 1.11 | 26.4 | 29.53 | 17.24 | 26.62 | 40.43 | 49.49 | |
| 0.37 | 12.93 | 20.99 | 10.45 | 16.99 | 34.42 | 42.55 | |
| 0.12 | 9.32 | 13.24 | 11.87 | 15.52 | 33.87 | 42.69 | |
| 0.00 | 0 | −9.16 | −10.21 | −3.82 | 20.61 | 30.96 | |

| [DRUG] | | | | | | | |
|---|---|---|---|---|---|---|---|
| COMPOUND 25 | | | | | | | STANDARD DEVIATION (%) |
| (nM) | 0.00 | 61.73 | 185.19 | 555.56 | 1666.67 | 5000.00 | COMPOUND 22 (nM) |
| 10.00 | 6.31 | 7.49 | 10.92 | 7.96 | 3.9 | 3.54 | |
| 3.33 | 6.77 | 3.56 | 12.39 | 9.02 | 3.89 | 7.17 | |
| 1.11 | 6.88 | 5.71 | 15.84 | 10.95 | 8.57 | 9.32 | |
| 0.37 | 1.49 | 4.56 | 17.71 | 9.5 | 7.06 | 8.21 | |
| 0.12 | 7.25 | 4.15 | 9.26 | 8.38 | 9.2 | 6.29 | |
| 0.00 | 14.86 | 17.38 | 15.2 | 14.87 | 11.14 | 12.14 | |

| [DRUG] | | | | | | | |
|---|---|---|---|---|---|---|---|
| COMPOUND 25 | | | | | | | ADDITIVE INHIBITION |
| (nM) | 0.00 | 61.73 | 185.19 | 555.56 | 1666.67 | 5000.00 | COMPOUND 22 (nM) |
| 10.00 | 42.95 | 37.72 | 37.13 | 40.77 | 54.71 | 60.61 | |
| 3.33 | 20.81 | 13.56 | 12.72 | 17.78 | 37.13 | 45.33 | |
| 1.11 | 26.4 | 19.66 | 18.89 | 23.59 | 41.57 | 49.19 | |
| 0.37 | 12.93 | 4.95 | 4.04 | 9.6 | 30.88 | 39.89 | |
| 0.12 | 9.32 | 1.01 | 0.06 | 5.86 | 28.01 | 37.39 | |
| 0.00 | 0 | −9.16 | −10.21 | −3.82 | 20.61 | 30.96 | |

| [DRUG] | | | | | | | | |
|---|---|---|---|---|---|---|---|---|
| COMPOUND 25 | | | | | | | SYNERGY PLOT (99.9%) | |
| (nM) | 0 | 61.728 | 185.19 | 555.56 | 1666.7 | 5000 | Bonferroni Adj. | 98% |
| 10.00 | 0 | 0 | 0 | 0 | 0 | 0 | SYNERGY | 9.68 |
| 3.33 | 0 | 8.64404 | 0 | 0 | 0 | 0 | log volume | 2.2 |
| 1.11 | 0 | 0 | 0 | 0 | 0 | 0 | | |
| 0.37 | 0 | 1.03304 | 0 | 0 | 0 | 0 | ANTAGONISM | 0 |
| 0.12 | 0 | 0 | 0 | 0 | 0 | 0 | log volume | 0 |
| 0.00 | 0 | 0 | 0 | 0 | 0 | 0 | | |

TABLE 23c
Effect on HBeAg in In Vitro Combination of Compound 22 and Compound 25

| | | | | | | | AVERAGE % INHIBITION |
|---|---|---|---|---|---|---|---|
| [DRUG] | 0.00 | 61.73 | 185.19 | 555.56 | 1666.67 | 5000.00 | COMPOUND 22 (nM) |
| 10.00 | 28.42 | 45.7 | 39.35 | 42.74 | 42.65 | 52.75 | |
| 3.33 | 13.94 | 29.09 | 24.19 | 23.42 | 24.67 | 39.67 | |
| 1.11 | 14.98 | 23.14 | 18.39 | 20.55 | 25.39 | 36.15 | |
| 0.37 | 2.9 | 7.24 | 7.64 | 4.51 | 17.83 | 27.05 | |
| 0.12 | 4.8 | 7.81 | 10.06 | 9.31 | 20.68 | 33.46 | |
| 0.00 | 0 | −16.81 | −14.59 | −7.23 | 8.5 | 21.68 | |

| [DRUG] | | | | | | | |
|---|---|---|---|---|---|---|---|
| COMPOUND 25 | | | | | | | STANDARD DEVIATION (%) |
| (nM) | 0.00 | 61.73 | 185.19 | 555.56 | 1666.67 | 5000.00 | COMPOUND 22 (nM) |
| 10.00 | 3.97 | 4.42 | 6.62 | 8.31 | 4.59 | 2.23 | |
| 3.33 | 9.3 | 1.4 | 6.29 | 15.17 | 11.71 | 2.03 | |
| 1.11 | 6.16 | 7.56 | 9.8 | 11.54 | 8.18 | 9.71 | |
| 0.37 | 10.44 | 7.8 | 10.09 | 14.23 | 7.82 | 10.34 | |
| 0.12 | 14.29 | 8.35 | 1.66 | 17.07 | 9.08 | 4.79 | |
| 0.00 | 10.71 | 11.88 | 5.84 | 11.39 | 4.94 | 6.86 | |

| [DRUG] | | | | | | | |
|---|---|---|---|---|---|---|---|
| COMPOUND 25 | | | | | | | ADDITIVE INHIBITION |
| (nM) | 0.00 | 61.73 | 185.19 | 555.56 | 1666.67 | 5000.00 | COMPOUND 22 (nM) |
| 10.00 | 28.42 | 16.39 | 17.98 | 23.24 | 34.5 | 43.94 | |
| 3.33 | 13.94 | −0.53 | 1.38 | 7.72 | 21.26 | 32.6 | |
| 1.11 | 14.98 | 0.69 | 2.58 | 8.83 | 22.21 | 33.41 | |
| 0.37 | 2.9 | −13.42 | −11.27 | −4.12 | 11.15 | 23.95 | |
| 0.12 | 4.8 | −11.2 | −9.09 | −2.08 | 12.89 | 25.44 | |
| 0.00 | 0 | −16.81 | −14.59 | −7.23 | 8.5 | 21.68 | |

| | |
|---|---|
| [DRUG] | |
| COMPOUND 25 | SYNERGY PLOT (99.9%) |

| (nM) | 0 | 61.728 | 185.19 | 555.56 | 1666.7 | 5000 | Bonferroni Adj. | 98% |
|---|---|---|---|---|---|---|---|---|
| 10.00 | 0 | 14.7638 | 0 | 0 | 0 | 1.47107 | SYNERGY | 57.43 |
| 3.33 | 0 | 25.0126 | 2.10961 | 0 | 0 | 0.38927 | log volume | 13.07 |
| 1.11 | 0 | 0 | 0 | 0 | 0 | 0 | | |
| 0.37 | 0 | 0 | 0 | 0 | 0 | 0 | ANTAGONISM | 0 |
| 0.12 | 0 | 0 | 13.6869 | 0 | 0 | 0 | log volume | 0 |
| 0.00 | 0 | 0 | 0 | 0 | 0 | 0 | | |

TABLE 23d
Summary of results of in vitro combination studies of Compound 22 and Compound 25 in PHH cell culture system:

| HBV | | | Inhibitor | Inhibitor | Synergy | Synergy | Antagonism | Antagonism | |
|---|---|---|---|---|---|---|---|---|---|
| Assay | Inhibitor | Inhibitor | A EC₅₀ | B EC₅₀ | Volume | Log | Volume | Log | |
| Endpoint | A | B | (nM)# | (nM)# | (μM² %)* | Volume | (μM² %)* | Volume | Conclusion |
| HBV DNA | COMPOUND 25 | COMPOUND 22 | 0.6535 | 1020 | 846.13 | 19.62 | 0 | 0 | Synergy |
| HBsAg | COMPOUND 25 | COMPOUND 22 | 4.503 | 12,800 | 9.68 | 2.2 | 0 | 0 | Additive |
| HBeAg | COMPOUND 25 | COMPOUND 22 | 5.75 | 10,740 | 57.43 | 13.07 | 0 | 0 | Synergy |

*at 99.9% confidence interval
#determined in an earlier separate experiment

### Example 24

### In Vitro Combination of IFNα2a and Compound 3

### Study Goal

To determine whether a two-drug combination of compound 3, and pegylated interferon alpha 2a (IFNα2a, an antiviral cytokine that activates innate immunity pathways in hepatocytes), is additive, synergistic or antagonistic in vitro using HBV-infected human primary hepatocytes in a cell culture model system.

### Results and Conclusion

FNα2a (concentration range of 10.0 IU/mL to 0.123 IU/mL in a 3-fold dilution series and 5 point titration) was tested in combination with compound 3 (concentration range of 5000 nM to 61.73 nM in a 3-fold dilution series and 5 point titration). The average % inhibition in HBV DNA, HBsAg and HBeAg, and standard deviations of 3 replicates observed either with IFNa2a or compound 3 treatments alone or in combination are shown in Tables 24a, 24b, and 24c as indicated below. The EC₅₀ values of IFNα2a and compound 3 were determined in an earlier experiment and are shown in Table 24d; some variance was observed from different lots of PHH cells.

When the observed values of a two-inhibitor combination were compared to what is expected from additive interaction for the above concentration range, the combinations were found to be synergistic, with no antagonism (Table 24d) as per MacSynergy II analysis and using the interpretive criteria described above by Prichard and Shipman (1992). No significant inhibition of cell viability or proliferation was observed by microscopy or CCK8 assay in the analyzed samples.

TABLE 24a
Effect on HBV DNA in In Vitro Combination of IFNα2a and Compound 3

| [DRUG] | | | | | | | |
|---|---|---|---|---|---|---|---|
| IFNα2a | | | | | | | AVERAGE % INHIBITION |
| IU/mL | 0.00 | 61.73 | 185.19 | 555.56 | 1666.67 | 5000.00 | COMPOUND 3 (μM) |
| 10.00 | 61.87 | 71.15 | 80.53 | 79.65 | 80.46 | 83.13 | |
| 3.33 | 59.88 | 65.87 | 74.49 | 76.8 | 84.91 | 87.1 | |
| 1.11 | 43.03 | 53.87 | 58.69 | 73.59 | 83.9 | 86.29 | |
| 0.37 | 38.46 | 40.68 | 50.62 | 61.26 | 79.98 | 87.96 | |
| 0.12 | 8.4 | 28.63 | 36.65 | 50.15 | 78.43 | 86.51 | |
| 0.00 | 0 | −11.71 | 4.14 | 26.47 | 69.39 | 84.26 | |

| [DRUG] | | | | | | | |
|---|---|---|---|---|---|---|---|
| IFNα2a | | | | | | | STANDARD DEVIATION (%) |
| IU/mL | 0.00 | 61.73 | 185.19 | 555.56 | 1666.67 | 5000.00 | COMPOUND 3 (μM) |
| 10.00 | 5.9 | 5.47 | 5.52 | 2.67 | 4.37 | 2.84 | |
| 3.33 | 5.53 | 2.04 | 2.64 | 4.62 | 3.33 | 1.29 | |
| 1.11 | 6.9 | 8.86 | 6.4 | 0.85 | 1.88 | 1.74 | |
| 0.37 | 4.9 | 5.86 | 4.86 | 5.2 | 2.07 | 0.96 | |
| 0.12 | 10.36 | 7.77 | 6.24 | 3.95 | 6.78 | 1.78 | |
| 0.00 | 15.33 | 3.13 | 10.75 | 14.76 | 3.99 | 2.26 | |

| [DRUG] | | | | | | | |
|---|---|---|---|---|---|---|---|
| IFNα2a | | | | | | | ADDITIVE INHIBITION |
| IU/mL | 0.00 | 61.73 | 185.19 | 555.56 | 1666.67 | 5000.00 | COMPOUND 3 (μM) |
| 10.00 | 61.87 | 57.4 | 63.45 | 71.96 | 88.33 | 94 | |
| 3.33 | 59.88 | 55.18 | 61.54 | 70.5 | 87.72 | 93.69 | |
| 1.11 | 43.03 | 36.36 | 45.39 | 58.11 | 82.56 | 91.03 | |
| 0.37 | 38.46 | 31.25 | 41.01 | 54.75 | 81.16 | 90.31 | |
| 0.12 | 8.4 | −2.33 | 12.19 | 32.65 | 71.96 | 85.58 | |
| 0.00 | 0 | −11.71 | 4.14 | 26.47 | 69.39 | 84.26 | |

| | |
|---|---|
| [DRUG] | |
| IFNα2a | SYNERGY PLOT (99.9%) |

| IU/mL | 0 | 61.728 | 185.19 | 555.56 | 1666.7 | 5000 | Bonferroni Adj. | 98% |
|---|---|---|---|---|---|---|---|---|
| 10.00 | 0 | 0 | 0 | 0 | 0 | −1.5236 | SYNERGY | 34.73 |
| 3.33 | 0 | 3.97636 | 4.26176 | 0 | 0 | −2.3446 | log volume | 7.91 |
| 1.11 | 0 | 0 | 0 | 12.6827 | 0 | 0 | | |
| 0.37 | 0 | 0 | 0 | 0 | 0 | 0 | ANTAGONISM | −3.87 |
| 0.12 | 0 | 5.38893 | 3.92416 | 4.50055 | 0 | 0 | log volume | −0.88 |
| 0.00 | 0 | 0 | 0 | 0 | 0 | 0 | | |

TABLE 24b
Effect on HBsAg in In Vitro Combination of IFNα2a and Compound 3

| [DRUG] | | | | | | | |
|---|---|---|---|---|---|---|---|
| IFNα2a | | | | | | | AVERAGE % INHIBITION |
| IU/mL | 0.00 | 61.73 | 185.19 | 555.56 | 1666.67 | 5000.00 | COMPOUND 3 (μM) |
| 10.00 | 24.94 | 33.38 | 33.93 | 39.7 | 49.84 | 67.18 | |
| 3.33 | 12.8 | 24.89 | 29.71 | 34.46 | 45.53 | 64.96 | |
| 1.11 | 14.91 | 22.82 | 26.09 | 36.42 | 44.97 | 67.18 | |
| 0.37 | 6.9 | 9.75 | 17.87 | 27.62 | 42.09 | 61.42 | |
| 0.12 | 1.56 | 10.13 | 19.07 | 22.18 | 42.08 | 62.05 | |
| 0.00 | 0 | −5.49 | −1.46 | 4.63 | 22.4 | 51.86 | |
| [DRUG] | | | | | | | |
| IFNα2a | | | | | | | STANDARD DEVIATION (%) |
| IU/mL | 0.00 | 61.73 | 185.19 | 555.56 | 1666.67 | 5000.00 | COMPOUND 3 (μM) |
| 10.00 | 24.86 | 7.76 | 9.93 | 15.02 | 12.81 | 9.59 | |
| 3.33 | 18.96 | 8.14 | 7.22 | 2.01 | 3.5 | 5.21 | |
| 1.11 | 20.01 | 4.74 | 6.41 | 3.05 | 5.38 | 4.26 | |
| 0.37 | 15.28 | 4.3 | 7.35 | 8.74 | 6.16 | 2.9 | |
| 0.12 | 16.47 | 3.75 | 5.07 | 7.78 | 7.65 | 6.59 | |
| 0.00 | 20.27 | 8.81 | 11.41 | 18.39 | 12.21 | 10.78 | |

| [DRUG] | | | | | | | |
|---|---|---|---|---|---|---|---|
| IFNα2a | | | | | | | ADDITIVE INHIBITION |
| IU/mL | 0.00 | 61.73 | 185.19 | 555.56 | 1666.67 | 5000.00 | COMPOUND 3 (μM) |
| 10.00 | 24.94 | 20.82 | 23.84 | 28.42 | 41.75 | 63.87 | |
| 3.33 | 12.8 | 8.01 | 11.53 | 16.84 | 32.33 | 58.02 | |
| 1.11 | 14.91 | 10.24 | 13.67 | 18.85 | 33.97 | 59.04 | |
| 0.37 | 6.9 | 1.79 | 5.54 | 11.21 | 27.75 | 55.18 | |
| 0.12 | 1.56 | −3.84 | 0.12 | 6.12 | 23.61 | 52.61 | |
| 0.00 | 0 | −5.49 | −1.46 | 4.63 | 22.4 | 51.86 | |

| [DRUG] | | | | | | | | |
|---|---|---|---|---|---|---|---|---|
| IFNα2a | | | | | | | SYNERGY PLOT (99.9%) | |
| IU/mL | 0 | 61.728 | 185.19 | 555.56 | 1666.7 | 5000 | Bonferroni Adj. | 98% |
| 10.00 | 0 | 0 | 0 | 0 | 0 | 0 | SYNERGY | 24.11 |
| 3.33 | 0 | 0 | 0 | 11.0051 | 1.6815 | 0 | log volume | 5.49 |
| 1.11 | 0 | 0 | 0 | 7.53245 | 0 | 0 | | |
| 0.37 | 0 | 0 | 0 | 0 | 0 | 0 | ANTAGONISM | 0 |
| 0.12 | 0 | 1.62875 | 2.26463 | 0 | 0 | 0 | log volume | 0 |
| 0.00 | 0 | 0 | 0 | 0 | 0 | 0 | | |

TABLE 24c
Effect on HBeAg in In Vitro Combination of IFNα2a and Compound 3

| | | | | | | | AVERAGE % INHIBITION |
|---|---|---|---|---|---|---|---|
| [DRUG] | 0.00 | 61.73 | 185.19 | 555.56 | 1666.67 | 5000.00 | COMPOUND 3 (μM) |
| 10.00 | 32.8 | 31.53 | 34.56 | 37.16 | 47.83 | 64.68 | |
| 3.33 | 14.38 | 25.43 | 28.01 | 30.3 | 39.42 | 61.88 | |
| 1.11 | 19.32 | 21.29 | 25.66 | 31.93 | 40.01 | 62.09 | |
| 0.37 | −2.24 | 6.43 | 9.53 | 18.94 | 28.32 | 53.12 | |
| 0.12 | −9.5 | 6.23 | 12.46 | 18.03 | 30.27 | 54.05 | |
| 0.00 | 0 | −11.14 | −4.9 | −1.02 | 12.42 | 42.06 | |

| [DRUG] | | | | | | | |
|---|---|---|---|---|---|---|---|
| IFNα2a | | | | | | | STANDARD DEVIATION (%) |
| IU/mL | 0.00 | 61.73 | 185.19 | 555.56 | 1666.67 | 5000.00 | COMPOUND 3 (μM) |
| 10.00 | 6.87 | 6.73 | 5.55 | 4.84 | 7.1 | 3.14 | |
| 3.33 | 4.07 | 5.37 | 7.42 | 9.41 | 7.15 | 1.79 | |
| 1.11 | 7.88 | 5.45 | 5.22 | 7.63 | 7.94 | 3.23 | |
| 0.37 | 1.9 | 2.87 | 4.47 | 11.64 | 7.71 | 1.12 | |
| 0.12 | 15.48 | 5.14 | 3.22 | 4.52 | 1.47 | 2.18 | |
| 0.00 | 8.69 | 17.68 | 3.21 | 3.3 | 4.7 | 7 | |

| [DRUG] | | | | | | | |
|---|---|---|---|---|---|---|---|
| IFNα2a | | | | | | | ADDITIVE INHIBITION |
| IU/mL | 0.00 | 61.73 | 185.19 | 555.56 | 1666.67 | 5000.00 | COMPOUND 3 (μM) |
| 10.00 | 32.8 | 25.31 | 29.51 | 32.11 | 41.15 | 61.06 | |
| 3.33 | 14.38 | 4.84 | 10.18 | 13.51 | 25.01 | 50.39 | |
| 1.11 | 19.32 | 10.33 | 15.37 | 18.5 | 29.34 | 53.25 | |
| 0.37 | −2.24 | −13.63 | −7.25 | −3.28 | 10.46 | 40.76 | |
| 0.12 | −9.5 | −21.7 | −14.87 | −10.62 | 4.1 | 36.56 | |
| 0.00 | 0 | −11.14 | −4.9 | −1.02 | 12.42 | 42.06 | |

| [DRUG] | | | | | | | | |
|---|---|---|---|---|---|---|---|---|
| IFNα2a | | | | | | | SYNERGY PLOT (99.9%) | |
| IU/mL | 0 | 61.728 | 185.19 | 555.56 | 1666.7 | 5000 | Bonferroni Adj. | 98% |
| 10.00 | 0 | 0 | 0 | 0 | 0 | 0 | SYNERGY | 103.04 |
| 3.33 | 0 | 2.91733 | 0 | 0 | 0 | 5.59911 | log volume | 23.46 |
| 1.11 | 0 | 0 | 0 | 0 | 0 | 0 | | |
| 0.37 | 0 | 10.6148 | 2.06923 | 0 | 0 | 8.67408 | ANTAGONISM | 0 |
| 0.12 | 0 | 11.0143 | 16.733 | 13.7747 | 21.3322 | 10.3156 | log volume | 0 |
| 0.00 | 0 | 0 | 0 | 0 | 0 | 0 | | |

TABLE 24d
Summary of results of in vitro combination studies of IFNα2a and Compound 3 in PHH cell culture system:

| HBV | | | Inhibitor | Inhibitor | Synergy | Synergy | Antagonism | Antagonism | |
|---|---|---|---|---|---|---|---|---|---|
| Assay | Inhibitor | Inhibitor | A EC₅₀ | B EC₅₀ | Volume | Log | Volume | Log | |
| Endpoint | A | B | (IU/mL)# | (nM)# | (μM² %)* | Volume | (μM² %)* | Volume | Conclusion |
| HBV DNA | IFNα2a | COMPOUND 3 | 2.154 | 876.5 | 34.73 | 7.91 | −3.87 | −0.88 | Synergy |
| HBsAg | IFNα2a | COMPOUND 3 | 13.8 | 7793 | 24.11 | 5.49 | 0 | 0 | Synergy |
| HBeAg | IFNα2a | COMPOUND 3 | 10.24 | 8580 | 103.04 | 23.46 | 0 | 0 | Synergy |

*at 99.9% confidence interval
#determined in an earlier separate experiment

### Example 25

### In Vitro Combination of TAF and SIRNA-NP

### Study Goal

To determine whether two drug combinations of tenofovir (in the form of the prodrug tenofovir alafenamide, or TAF, a nucleotide analog inhibitor of HBV polymerase), and SIRNA-NP, an siRNA intended to facilitate potent knockdown of all viral mRNA transcripts and viral antigens, is additive, synergistic or antagonistic in vitro using an HBV cell culture model system.

### In Vitro Combination in HepDE19 Experimental Protocol

In vitro combination studies were conducted using the method of Prichard and Shipman (1990) (Prichard M N, Shipman C, Jr. 1990. A three-dimensional model to analyze drug-drug interactions. Antiviral Res 14:181-205 AND Prichard M N. 1992. MacSynergy II, University of Michigan). The HepDE19 cell line was developed as described in Guo et al. (2007)(Guo H, Jiang D, Zhou T, Cuconati A, Block T M, Guo J T. 2007. Characterization of the intracellular deproteinized relaxed circular DNA of hepatitis B virus: an intermediate of covalently closed circular DNA formation. J Virol 81:12472-12484). It is a human hepatoma cell line stably transfected with the HBV genome, and which can express HBV pregenomic RNA and support HBV rcDNA (relaxed circular DNA) synthesis in a tetracycline-regulated manner. HepDE19 cells were plated in 96 well tissue-culture treated microtiter plates in DMEM/F12 medium supplemented with 10% fetal bovine serum+1% penicillin-streptomycin without tetracycline and incubated in a humidified incubator at 37° C. and 5% CO₂ overnight. Next day, the cells were switched to fresh medium and treated with inhibitor A and inhibitor B, at concentration range in the vicinity of their respective EC₅₀ values, and incubated for a duration of 7 days in a humidified incubator at 37° C. and 5% CO₂. The inhibitors were either diluted in 100% DMSO (TAF) or growth medium (SIRNA-NP) and the final DMSO concentration in the assay was ≤0.5%. The two inhibitors were tested both singly as well as in combinations in a checkerboard fashion such that each concentration of inhibitor A was combined with each concentration of inhibitor B to determine their combination effects on inhibition of rcDNA production. Following a 48 hour-incubation, the level of rcDNA present in the inhibitor-treated wells was measured using a bDNA assay (Affymetrix) with HBV specific custom probe set and manufacturer's instructions. The RLU data generated from each well was calculated as % inhibition of the untreated control wells and analyzed using the MacSynergy II program to determine whether the combinations were synergistic, additive or antagonistic using the interpretive guidelines established by Prichard and Shipman as follows: Synergy volumes <25 μM²% (log volume <2) at 95% CI=probably insignificant; 25-50 μM²% (log volume >2 and <5)=minor but significant 50-100 μM²% (log volume >5 and <9)=moderate, may be important in vivo; Over 100 μM²% (log volume >9)=strong synergy, probably important in vivo; volumes approaching 1000 μM²% (log volume >90)=unusually high, check data. Concurrently, the effect of inhibitor combinations on cell viability was assessed using replicate plates that were used to determine the ATP content as a measure of cell viability using the Cell-TiterGlo reagent (Promega) as per manufacturer's instructions.

### Results and Conclusion

TAF (concentration range of 200.0 nM to 0.781 nM in a 2-fold dilution series and 9 point titration) was tested in combination with SIRNA-NP (concentration range of 60 ng/mL to 0.741 ng/mL in a 3-fold dilution series and 5 point titration). The average % inhibition in rcDNA and standard deviations of 4 replicates observed either with TAF or SIRNA-NP treatments alone or in combination is shown in Table 25A. The EC₅₀ values of TAF and SIRNA-NP are shown in Table 25B. When the observed values of two inhibitor combination were compared to what is expected from additive interaction (Table 25A) for the above concentration range, the combinations were found to be additive, with no antagonism (Table 25B) as per MacSynergy II analysis and using the interpretive criteria described above by Prichard and Shipman (1992). No significant inhibition of cell viability or proliferation was observed by microscopy or Cell-TiterGlo assay in the analyzed samples.

TABLE 25A
In vitro Combination of Tenofovir Alafenamide and SIRNA-NP

| [DRUG] | | | | | | | | | | | |
|---|---|---|---|---|---|---|---|---|---|---|---|
| SIRNA-NP | | | | | | | | | | | AVERAGE % INHIBITION |
| (ng/mL) | 0 | 0.781 | 1.563 | 3.125 | 6.250 | 12.500 | 25.000 | 50.000 | 100.000 | 200.000 | TAF (nM) |
| 60 | 98.19 | 98.66 | 98.73 | 98.87 | 99.33 | 99.41 | 99.4 | 99.5 | 99.58 | 99.59 | |
| 20.000 | 96.42 | 95.42 | 96.67 | 97.25 | 98.09 | 98.71 | 98.41 | 98.86 | 99.28 | 99.49 | |
| 6.667 | 88.02 | 88.65 | 91.24 | 91.67 | 94.4 | 95.11 | 95.04 | 95.97 | 98.26 | 98.98 | |
| 2.222 | 80.18 | 72.86 | 78.16 | 81.28 | 82.7 | 87.98 | 87.09 | 91.03 | 95.81 | 98.08 | |
| 0.741 | 53.05 | 55.46 | 55.43 | 62.01 | 63.65 | 78.75 | 72.62 | 82.47 | 90.47 | 96.24 | |
| 0 | 0 | −4.76 | 3.49 | 0.6 | 10.59 | 28.61 | 20.04 | 53.2 | 77.59 | 89.93 | |

| [DRUG] | | | | | | | | | | | |
|---|---|---|---|---|---|---|---|---|---|---|---|
| SIRNA-NP | | | | | | | | | | | STANDARD DEVIATION (%) |
| (ng/mL) | 0 | 0.7813 | 1.5625 | 3.125 | 6.25 | 12.5 | 25 | 50 | 100 | 200 | TAF (nM) |
| 60 | 0.64 | 0.46 | 0.63 | 0.55 | 0.17 | 0.23 | 0.1 | 0.06 | 0.04 | 0.1 | |
| 20.000 | 1.07 | 2.02 | 1.82 | 1.42 | 0.82 | 0.32 | 0.56 | 0.14 | 0.1 | 0.06 | |
| 6.667 | 2.35 | 3.56 | 4.19 | 5.97 | 1.68 | 0.94 | 1.45 | 0.87 | 0.51 | 0.12 | |
| 2.222 | 3.54 | 7.95 | 10.29 | 9.62 | 3.94 | 3.27 | 3.67 | 1.49 | 0.57 | 0.48 | |
| 0.741 | 12.82 | 16.97 | 11.3 | 11.62 | 9.42 | 10.02 | 1.77 | 3.4 | 0.5 | 0.83 | |
| 0 | 0 | 15.54 | 15.63 | 12.12 | 19.07 | 9.89 | 8.58 | 4.92 | 2.79 | 2.12 | |

| [DRUG] | | | | | | | | | | | |
|---|---|---|---|---|---|---|---|---|---|---|---|
| SIRNA-NP | | | | | | | | | | | ADDITIVE INHIBITION |
| (ng/mL) | 0 | 0.7813 | 1.5625 | 3.125 | 6.25 | 12.5 | 25 | 50 | 100 | 200 | TAF (nM) |
| 60 | 98.19 | 98.1 | 98.25 | 98.2 | 98.38 | 98.71 | 98.55 | 99.15 | 99.59 | 99.82 | |
| 20.000 | 96.42 | 96.25 | 96.54 | 96.44 | 96.8 | 97.44 | 97.14 | 98.32 | 99.2 | 99.64 | |
| 6.667 | 88.02 | 87.45 | 88.44 | 88.09 | 89.29 | 91.45 | 90.42 | 94.39 | 97.32 | 98.79 | |
| 2.222 | 80.18 | 79.24 | 80.87 | 80.3 | 82.28 | 85.85 | 84.15 | 90.72 | 95.56 | 98 | |
| 0.741 | 53.05 | 50.82 | 54.69 | 53.33 | 58.02 | 66.48 | 62.46 | 78.03 | 89.48 | 95.27 | |
| 0 | 0 | −4.76 | 3.49 | 0.6 | 10.59 | 28.61 | 20.04 | 53.2 | 77.59 | 89.93 | |

| [DRUG] | | | | | | | | | | | | |
|---|---|---|---|---|---|---|---|---|---|---|---|---|
| SIRNA-NP | | | | | | | | | | | SYNERGY PLOT (99.9%) | |
| (ng/mL) | 0 | 0.78 | 1.56 | 3.13 | 6.25 | 12.50 | 25.00 | 50 | 100 | 200 | Bonferroni Adj. | 96% |
| 60.0 | 0 | 0 | 0 | 0 | 0.390 | 0 | 0.520 | 0.152 | 0 | 0 | SYNERGY | 6.26 |
| 20.000 | 0 | 0 | 0 | 0 | 0 | 0.216 | 0 | 0.079 | 0 | 0 | log volume | 0.9 |
| 6.667 | 0 | 0 | 0 | 0 | 0 | 0.566 | 0 | 0 | 0 | 0 | | |
| 2.222 | 0 | 0 | 0 | 0 | 0 | 0 | 0 | 0 | 0 | 0 | ANTAGONISM | 0 |
| 0.741 | 0 | 0 | 0 | 0 | 0 | 0 | 4.334 | 0 | 0 | 0 | log volume | 0 |
| 0 | 0 | 0 | 0 | 0 | 0 | 0 | 0 | 0 | 0 | 0 | | |

TABLE 25B
Summary of results of in vitro combination studies in DE19 cell
culture system with rcDNA Quantitation using bDNA assay:

| | | Inhibitor | Inhibitor | Synergy | Synergy | Antagonism | Antagonism | |
|---|---|---|---|---|---|---|---|---|
| Inhibitor | Inhibitor | A EC₅₀ | B EC₅₀ | Volume | Log | Volume | Log | |
| A | B | (ng/mL) | (nM) | (μM² %)* | Volume | (μM² %)* | Volume | Conclusion |
| SIRNA-NP | TAF | 0.624 | 44.52 | 6.26 | 0.9 | 0 | 0 | Additive |

*at 99.9% confidence interval

### Example 26

### In Vitro Combination of Compound 3 and GLS4

### Study Goal

To determine whether a two-drug combination of compound 3 (a small molecule inhibitor of HBV encapsidation belonging to the sulfamoyl benzamide chemical class), and GLS4 (a small molecule inhibitor of HBV encapsidation belonging to the heteroaryldihydropyrimidine, or HAP, chemical class) is additive, synergistic or antagonistic in vitro using an HBV cell culture model system.

### In Vitro Combination in HepDE19 Experimental Protocol

In vitro combination studies were conducted using the method of Prichard and Shipman (1990). The HepDE19 cell line was developed as described in Guo et al. (2007). It is a human hepatoma cell line stably transfected with the HBV genome, and which can express HBV pregenomic RNA and support HBV rcDNA (relaxed circular DNA) synthesis in a tetracycline-regulated manner. HepDE19 cells were plated in 96 well tissue-culture treated microtiter plates in DMEM/F12 medium supplemented with 10% fetal bovine serum+1% penicillin-streptomycin without tetracycline and incubated in a humidified incubator at 37° C. and 5% CO₂ overnight. Next day, the cells were switched to fresh medium and treated with inhibitor A and inhibitor B, at a concentration range in the vicinity of their respective EC₅₀ values, and incubated for a duration of 7 days in a humidified incubator at 37° C. and 5% CO₂. Both inhibitors were diluted in 100% DMSO and the final DMSO concentration in the assay was ≤0.5%. The two inhibitors were tested both singly as well as in combinations in a checkerboard fashion such that each concentration of inhibitor A was combined with each concentration of inhibitor B to determine their combination effects on inhibition of rcDNA production. Following a 48 hour-incubation, the level of rcDNA present in the inhibitor-treated wells was measured using a bDNA assay (Affymetrix) with HBV specific custom probe set and manufacturer instructions. The RLU data generated from each well was calculated as % inhibition of the untreated control wells and analyzed using the MacSynergy II program to determine whether the combinations were synergistic, additive or antagonistic using the interpretive guidelines established by Prichard and Shipman as follows: synergy volumes <25 μM²% (log volume <2) at 95% CI=probably insignificant; 25-50 μM²% (log volume >2 and <5)=minor but significant 50-100 μM²% (log volume >5 and <9)=moderate, may be important in vivo; Over 100 μM²% (log volume >9)=strong synergy, probably important in vivo; volumes approaching 1000 μM²% (log volume >90)=unusually high, check data. Concurrently, the effect of inhibitor combinations on cell viability was assessed using replicate plates that were used to determine the ATP content as a measure of cell viability using the Cell-TiterGlo reagent (Promega) as per manufacturer's instructions.

### Results and Conclusion

Compound 3 (concentration range of 3.0 μM to 0.04 μM in a 3-fold dilution series and 5 point titration) was tested in combination with GLS4 (concentration range of 2.0 μM to 0.008 μM in a 2-fold dilution series and 9 point titration). The average % inhibition in rcDNA and standard deviations of 4 replicates observed either with compound 3 or GLS4 treatments alone or in combination is shown in Table 26a. The EC₅₀ values of compound 3 and GLS4 are shown in Table 26b. When the observed values of two inhibitor combination were compared to what is expected from additive interaction (Table 26a) for the above concentration range, the combination was found to be largely additive, and very slightly antagonistic (Table 26b); as per MacSynergy II analysis and using the interpretive criteria described above by Prichard and Shipman (1992), the degree of antagonism is minor but significant. No significant inhibition of cell viability or proliferation was observed by microscopy or Cell-TiterGlo assay in the analyzed samples.

TABLE 26a
In vitro Combination of Compound 3 and GLS4

| [DRUG] | | | | | | | | | | | |
|---|---|---|---|---|---|---|---|---|---|---|---|
| COMPOUND 3 | | | | | | | | | | | AVERAGE % INHIBITION |
| μM | 0 | 0.008 | 0.016 | 0.031 | 0.063 | 0.125 | 0.250 | 0.500 | 1.000 | 2.000 | GLS-4 (μM) |
| 3.000 | 94.49 | 94.6 | 93.75 | 93.69 | 93.74 | 93.46 | 91.72 | 96.86 | 97 | 98.08 | |
| 1.000 | 86.87 | 85.25 | 87.63 | 86.08 | 84.96 | 87.22 | 92.9 | 96.99 | 97.48 | 97.01 | |
| 0.330 | 56.68 | 56.86 | 55.08 | 58.52 | 73.47 | 81.88 | 93.03 | 97.63 | 97.52 | 95.96 | |
| 0.110 | 19.99 | 13.03 | 18.43 | 23.81 | 53.91 | 74.43 | 95.32 | 97.65 | 97.52 | 98.12 | |
| 0.040 | 11.14 | −4.48 | −1.03 | 14.94 | 28.97 | 73.14 | 93.01 | 97.99 | 97.76 | 97.47 | |
| 0.000 | 0 | −1.17 | −5.82 | 7.03 | 38.95 | 77.82 | 94.65 | 97.48 | 98.51 | 98.22 | |

| [DRUG] | | | | | | | | | | | |
|---|---|---|---|---|---|---|---|---|---|---|---|
| COMPOUND 3 | | | | | | | | | | | STANDARD DEVIATION (%) |
| μM | 0 | 0.007813 | 0.01563 | 0.03125 | 0.0625 | 0.125 | 0.25 | 0.5 | 1 | 2 | GLS-4 (μM) |
| 3 | 1.29 | 1.34 | 1.38 | 0.33 | 0.71 | 0.37 | 1.37 | 0.57 | 0.95 | 1.25 | |
| 1.000 | 3.95 | 5.47 | 1.98 | 1.54 | 3.07 | 2.38 | 0.89 | 0.56 | 0.8 | 1.43 | |
| 0.330 | 6.93 | 11.7 | 7.92 | 5.09 | 4.36 | 5.69 | 1.6 | 0.73 | 1.02 | 2.33 | |
| 0.110 | 15.95 | 12.76 | 10.23 | 4.24 | 14.05 | 5.6 | 1.61 | 0.83 | 0.72 | 0.31 | |
| 0.040 | 22.92 | 26.91 | 6.36 | 31.59 | 16.09 | 5.54 | 1.82 | 0.68 | 0.72 | 0.31 | |
| 0 | 0 | 17.17 | 15.42 | 15.34 | 10.95 | 6.65 | 1.39 | 1.47 | 0.59 | 0.35 | |

| [DRUG] | | | | | | | | | | | |
|---|---|---|---|---|---|---|---|---|---|---|---|
| COMPOUND 3 | | | | | | | | | | | ADDITIVE INHIBITION |
| μM | 0 | 0.007813 | 0.01563 | 0.03125 | 0.0625 | 0.125 | 0.25 | 0.5 | 1 | 2 | GLS-4 (μM) |
| 3 | 94.49 | 94.43 | 94.17 | 94.88 | 96.64 | 98.78 | 99.71 | 99.86 | 99.92 | 99.9 | |
| 1.000 | 86.87 | 86.72 | 86.11 | 87.79 | 91.98 | 97.09 | 99.3 | 99.67 | 99.8 | 99.77 | |
| 0.330 | 56.68 | 56.17 | 54.16 | 59.73 | 73.55 | 90.39 | 97.68 | 98.91 | 99.35 | 99.23 | |
| 0.110 | 19.99 | 19.05 | 15.33 | 25.61 | 51.15 | 82.25 | 95.72 | 97.98 | 98.81 | 98.58 | |
| 0.040 | 11.14 | 10.1 | 5.97 | 17.39 | 45.75 | 80.29 | 95.25 | 97.76 | 98.68 | 98.42 | |
| 0 | 0 | −1.17 | −5.82 | 7.03 | 38.95 | 77.82 | 94.65 | 97.48 | 98.51 | 98.22 | |

| [DRUG] | | | | | | | | | | | | |
|---|---|---|---|---|---|---|---|---|---|---|---|---|
| COMPOUND 3 | | | | | | | | | | | SYNERGY PLOT (95%) | |
| μM | 0 | 0.007813 | 0.01563 | 0.03125 | 0.0625 | 0.125 | 0.25 | 0.5 | 1 | 2 | Bonferroni Adj. | — |
| 3 | 0 | 0 | 0 | −0.543 | −1.508 | −4.594 | −5.304 | −1.882 | −1.058 | 0 | SYNERGY | 0 |
| 1.000 | 0 | 0 | 0 | 0 | −1.002 | −5.205 | −4.655 | −1.582 | −0.752 | 0 | log volume | 0 |
| 0.330 | 0 | 0 | 0 | 0 | 0 | 0 | −1.514 | 0 | 0 | 0 | | |
| 0.110 | 0 | 0 | 0 | 0 | 0 | 0 | 0 | 0 | 0 | 0 | ANTAGONISM | −29.95 |
| 0.040 | 0 | 0 | 0 | 0 | 0 | 0 | 0 | 0 | 0 | −0.342 | log volume | −4.13 |
| 0 | 0 | 0 | 0 | 0 | 0 | 0 | 0 | 0 | 0 | 0 | | |

| [DRUG] | | | | | | | | | | | | |
|---|---|---|---|---|---|---|---|---|---|---|---|---|
| COMPOUND 3 | | | | | | | | | | | SYNERGY PLOT (99.9%) | |
| μM | 0 | 0.01 | 0.02 | 0.03 | 0.06 | 0.13 | 0.25 | 0.5 | 1 | 2 | Bonferroni Adj. | 96% |
| 3.0 | 0 | 0 | 0 | −0.103 | −0.563 | −4.102 | −3.481 | −1.124 | 0 | 0 | SYNERGY | 0 |
| 1.000 | 0 | 0 | 0 | 0 | 0 | −2.037 | −3.471 | −0.837 | 0 | 0 | log volume | 0 |
| 0.330 | 0 | 0 | 0 | 0 | 0 | 0 | 0 | 0 | 0 | 0 | | |
| 0.110 | 0 | 0 | 0 | 0 | 0 | 0 | 0 | 0 | 0 | 0 | ANTAGONISM | −15.72 |
| 0.040 | 0 | 0 | 0 | 0 | 0 | 0 | 0 | 0 | 0 | 0 | log volume | −2.17 |
| 0 | 0 | 0 | 0 | 0 | 0 | 0 | 0 | 0 | 0 | 0 | | |

TABLE 26b
Summary of results of in vitro combination studies in DE19 cell
culture system with rcDNA quantitation using bDNA assay:

| | | Inhibitor | Inhibitor | Synergy | Synergy | Antagonism | Antagonism | |
|---|---|---|---|---|---|---|---|---|
| Inhibitor | Inhibitor | A EC₅₀ | B EC₅₀ | Volume | Log | Volume | Log | |
| A | B | (μM) | (μM) | (μM² %)* | Volume | (μM² %)* | Volume | Conclusion |
| Compound 3 | GLS4 | 0.272 | 0.077 | 0 | 0 | −15.72 | −2.17 | Additive* |
| Compound 3 | GLS4 | 0.272 | 0.077 | 0 | 0 | −29.95 | −4.13 | Minor |
| | | | | | | | | Antagonism^{#} |

*at 99.9% confidence interval
^{#}at 95% confidence interval

### Example 27

Developing a cure for chronic HBV is challenged by the ability of the virus to suppress the host immune response, and the presence of a cccDNA reservoir. A cure for chronic HBV should address multiple factors involved in viral persistence and may require drug combinations with different mechanisms of action. One such combination strategy is examined in this Example.

A mouse model of hepatitis B virus (HBV) was used to assess the anti-HBV effects of a combination treatment with immune enhancers, immune stimulants and HBV-targeting siRNAs primarily as antigen reducers.

A mixture of three siRNAs targeting the HBV genome were used. The sequences of the three siRNAs are shown below.

| Sense Sequence (5′-3′) | Antisense Sequence (5′-3′) |
|---|---|
| CCgUguGCACUuCgCuuCAUU | UGAAgCgAAGugCAcAcgGUU |
| CuggCuCAGuUuAcuAgugUU | CACUAguAAAcUgAgCcAGUU |
| AcCuCuGcCuAaUcAUCUCUU | GAGAUGaUuAGGcAgAgGUUU |

lower case = 2′-O-methyl modification

This mixture of three HBV-targeting siRNAs was administered as a lipid nanoparticle (LNP) formulation.
The following lipid nanoparticle (LNP) formulation was used to deliver the HBV siRNAs in the experiment reported herein. The values shown in the table are mole percentages. The abbreviation DSPC means distearoylphosphatidylcholine.

| PEG2000-C-DMA | Cationic lipid | Cholesterol | DSPC |
|---|---|---|---|
| 1.6 | 54.6 | 32.8 | 10.9 |

The cationic lipid had the following structure:

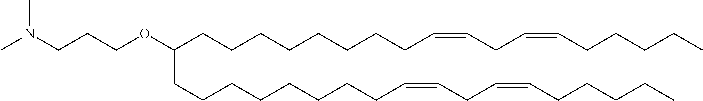 [SMILES: CCCCC/C=C\C/C=C\CCCCCCCCC(CCCCCCCC/C=C\C/C=C\CCCCC)OCCCN(C)C]

Prior to treatment start, 1×10¹¹ viral genomes of an adeno-associated virus (AAV) vector carrying a 1.2-fold overlength copy of a HBV genome (originally described in Dion, S et al., Journal of Virology, 2013, 87(10): 5554-5563) was administered to C57BL/6 mice via intravenous injection. Introduction of this viral vector results in the expression of HBV surface antigen (HBsAg) amongst other HBV products and generates a state of immune tolerance to HBV. A subset of animals was not administered this HBV-carrying AAV vector and were used as a negative control to demonstrate baseline HBV-specific immune response when no HBV exposure or anti-HBV treatment had occurred. Serum HBsAg expression and anti-HBsAg antibody levels in mice were monitored using enzyme immunoassays. Serum HBV DNA was monitored using a quantitative polymerase chain reaction (QPCR) assay. Animals were sorted (randomized) into groups based on a negative antibody response and serum HBsAg levels such that a) all animals were confirmed to express HBsAg and b) HBsAg group means were similar to each other prior to initiation of treatments.

Animals were treated with lipid nanoparticle (LNP)-encapsulated HBV-targeting siRNAs as follows: On each of Days 0, 7, 14, 21, 28 and 35 an amount of test article equivalent to 1 mg/kg siRNA was administered intravenously. Concurrently, animals were treated with immune enhancer as follows: On Day 0 and every three or four days until Day 41, 200 micrograms of an antibody against murine Programmed death-ligand 1 (PD-L1, clone 10F.9G2, rat anti-mouse PD-L1, obtained from BioXCell, catalog no. BP0101) was administered via intraperitoneal injection. Following combination treatment with siRNA and anti-PD-L1, an immune stimulant was administered which consisted of two micrograms of recombinant HBsAg vaccine (Engerix-B, consisting of yeast recombinant HBsAg adsorbed onto aluminum hydroxide, obtained from GlaxoSmithKline, National Drug Code no. 58160-821-11) administered concurrently with 50 micrograms of adjuvant consisting of cytidine-guanosine (CpG) dinucleotides (mouse Class B TLR9 ligand, sequence 5′-TCCATGACGTTCCTGACGTT-3′ of phosphorothioate bases, obtained from Invivogen, catalog no. tlrl-1826).

The effect of treatments on HBV immune responses was determined by sacrificing animals and isolating liver lymphocytes to identify T cell responses to HBV by the production of cytokines IFN-gamma and IL-2 in an enzyme-linked immunospot (Elispot) assay. Table 27A shows the Day 42 treatment responses (pool of n=4; ±standard deviation of technical replicates). To demonstrate treatment-specific effects, the treated groups were compared against negative control animals. Treatment with anti-PD-L1 or HBV siRNA alone induced some HBV immune response; the greatest effect was observed following combination treatment with these two agents.

The effect on serum HBsAg and serum HBV DNA during and after treatment cessation was determined by collecting a small amount of blood on Days 0 (pre-treatment), 14, 21, 28, 42, 56, 70, 91, 112, and 140. Table 27B shows the treatment group pooled results (pool of n=8) serum HBsAg concentration expressed as a percentage of the group pooled pre-treatment baseline value at Day 0. The data demonstrate that HBsAg reduction was a result of HBV siRNA treatment but not a result of anti-PD-L1 treatment by itself. Combination treatment of HBV siRNA and anti-PD-L1 resulted in HBsAg reduction while on-treatment but did not result in lasting control of HBsAg after treatment cessation. In contrast, combination treatment with HBV siRNA and anti-PD-L1 which was followed by the addition of an immune stimulant vaccine did result in control of HBsAg for a considerable length of time after treatment was stopped. This off-treatment viral control coincided with the elevated production of serum anti-HBsAg antibodies. Table 27C shows the treatment group mean (n=8; ±standard error of the mean) serum anti-HBsAg antibody levels expressed in International Units per millilitre. Analysis of serum HBV DNA resulted in substantively similar trends as describe for serum HBsAg with regards to responses to the various combination treatments. Reduction of serum HBV DNA of approximately at least one and a half log 10, to below the assay limit of detection, was measured at Day 42 in any treatment group that included HBV siRNA treatment but not when anti-PD-L1 was administered in the absence of HBV siRNA. Partial post-treatment-cessation control of HBV DNA was achieved only in the case of immune stimulant vaccine combination with HBV siRNA and anti-PD-L1.

The data demonstrate that HBsAg and HBV DNA reduction was caused by HBV siRNA treatment, that HBV immune responses were greater when an agent used to control HBV antigenemia (HBV siRNA) and an immune enhancer agent (anti-PD-L1) were combined, and the reductive effect on HBsAg and HBV DNA remained durable even after treatment cessation (sustained through to the end of the post-treatment observation period) when a third agent, an immune stimulant (vaccine) was added subsequent to treatment with the other two agents. The combination of the three treatments resulted in greater and more sustained anti-HBV effect than either treatment alone or as a combination of two agents (antigen reducer plus immune enhancer).

TABLE 27A
Single and Combination Treatment Effect of Antigen
Reducing HBV-targeting siRNAs and Immune Enhancer
anti-PD-L1 Antibody on HBV T Cell Cytokine Responses
at Day 42 in a Mouse Model of HBV Infection

| | IFN-gamma | IL-2 |
|---|---|---|
| | (No. Spots/ | (No. Spots/ |
| Treatments | 10⁶ cells) | 10⁶ cells) |
| Untreated and AAV-Naïve | 44 ± 16 | 41 ± 1 |
| (not exposed to HBV) | | |
| Antibody Isotype Negative | 12 ± 12 | 48 ± 10 |
| Control | | |
| HBV siRNA-LNP + | 33 ± 20 | 72 ± 0 |
| Antibody Isotype Negative | | |
| Control | | |
| Anti-PD-L1 Antibody | 19 ± 4 | 91 ± 6 |
| HBV siRNA-LNP + | 265 ± 30 | 175 ± 36 |
| Anti-PD-L1 Antibody | | |

TABLE 27B
Single and Combination Treatment Effect of Antigen Reducing HBV-targeting siRNAs, Immune Enhancer
anti-PD-L1 Antibody and Immune Stimulant Vaccine on Serum HBsAg in a Mouse Model of HBV Infection

| | Day | Day | Day | Day | Day | Day | Day | Day | Day | Day |
|---|---|---|---|---|---|---|---|---|---|---|
| Treatments | 0 | 14 | 21 | 28 | 42 | 56 | 70 | 91 | 112 | 140 |
| HBV siRNA-LNP + | 100 | 0.023 | 0.015 | 0.013 | 0.011 | 0.11 | 10 | 91 | 101 | 98 |
| Antibody Isotype | | | | | | | | | | |
| Negative Control | | | | | | | | | | |
| Anti-PD-L1 | 100 | 94 | 105 | 122 | 128 | 121 | 122 | 125 | 128 | 114 |
| Antibody + | | | | | | | | | | |
| Luciferase siRNA- | | | | | | | | | | |
| LNP Negative | | | | | | | | | | |
| Control | | | | | | | | | | |
| HBV siRNA-LNP + | 100 | 0.065 | 0.013 | 0.013 | 0.015 | 0.23 | 14 | 133 | 114 | 114 |
| Anti-PD-L1 | | | | | | | | | | |
| Antibody | | | | | | | | | | |
| HBV siRNA-LNP + | 100 | 0.021 | 0.0065 | 0.0077 | 0.0099 | 0.0055 | 0.18 | 0.50 | 1.2 | 1.3 |
| Anti-PD-L1 | | | | | | | | | | |
| Antibody + | | | | | | | | | | |
| Vaccine | | | | | | | | | | |

TABLE 27C
Single and Combination Treatment Effect of Antigen Reducing HBV-targeting siRNAs, Immune Enhancer anti-PD-L1 Antibody
and Immune Stimulant Vaccine on Production of Serum anti-HBsAg Antibodies in a Mouse Model of HBV Infection

| | Day | Day | Day | Day | Day | Day | Day | Day | Day | Day |
|---|---|---|---|---|---|---|---|---|---|---|
| Treatments | 0 | 14 | 21 | 28 | 42 | 56 | 70 | 91 | 112 | 140 |
| Untreated | N/A | N/A | 14 | 20 | 19 | 20 | 25 | 17 | 20 | 17 |
| and AAV- | | | | | | | | | | |
| Naïve¹ (not | | | | | | | | | | |
| exposed | | | | | | | | | | |
| to HBV) | | | | | | | | | | |
| HBV | 30 ± 1 | 22 ± 1 | 57 ± 33 | 79 ± 35 | 359 ± 106 | 293 ± 105 | 309 ± 129 | 605 ± 349 | 610 ± 397 | 479 ± 342 |
| siRNA-LNP + | | | | | | | | | | |
| Antibody | | | | | | | | | | |
| Isotype | | | | | | | | | | |
| Negative | | | | | | | | | | |
| Control | | | | | | | | | | |
| Anti-PD-L1 | 30 ± 1 | 22 ± 1 | 17 ± 2.5 | 31 ± 9.4 | 95 ± 30 | 67 ± 22 | 42 ± 8.4 | 41 ± 10 | 70 ± 37 | 69 ± 25 |
| Antibody + | | | | | | | | | | |
| Luciferase | | | | | | | | | | |
| siRNA-LNP | | | | | | | | | | |
| Negative | | | | | | | | | | |
| Control | | | | | | | | | | |
| HBV | 30 ± 1 | 22 ± 1 | 26 ± 11 | 35 ± 9 | 81 ± 18 | 66 ± 18 | 42 ± 9 | 44 ± 11 | 64 ± 23 | 67 ± 18 |
| siRNA-LNP + | | | | | | | | | | |
| Anti-PD-L1 | | | | | | | | | | |
| Antibody | | | | | | | | | | |
| HBV | 30 ± 1 | 22 ± 1 | 63 ± 43 | 55 ± 29 | 142 ± 79 | 323 ± 123 | 2715 ± 293 | 14202 ± 2604 | 27917 ± 12969 | 17907 ± 8245 |
| siRNA-LNP + | | | | | | | | | | |
| Anti-PD-L1 | | | | | | | | | | |
| Antibody + | | | | | | | | | | |
| Vaccine | | | | | | | | | | |

¹Group pooled serum was assessed, thus no error calculation available
N/A = Not available

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treating hepatitis B in a human, comprising administering to the human:
an siRNA that targets a portion of the HBV genome;
an inhibitor of PD-L1; and
an anti-HBV vaccine.

2-15. (canceled)

16. A method for treating hepatitis B in a human, comprising administering to the human at least one agent from at least three of the following categories of agents:
(A) an agent that controls viral replication;
(B) an agent that reduces viral Ags;
(C) an immune enhancer; and
(D) an immune stimulant.

17-29. (canceled)

30. The method of claim 16, wherein the agent that reduces viral Ags and the immune enhancer are administered, concurrently.

31. The method of claim 30, wherein the immune stimulant is administered, subsequent to the administration of the agent that reduces viral Ags and the immune enhancer.

32. The method of claim 16, wherein the agent that controls viral replication is administered, concurrently with or prior to the administration of the agent that reduces viral Ags and the immune enhancer.

33. The method of claim 32, wherein the agent that controls viral replication is administered concurrently with the administration of the agent that reduces viral Ags and the immune enhancer.

34. The method of claim 32, wherein the agent that controls viral replication is administered prior to the administration of the agent that reduces viral Ags and the immune enhancer.

35. The method of claim 16, wherein the agent that reduces viral Ags, the immune enhancer and the immune stimulant are administered, concurrently.

36. The method of claim 35, wherein an agent that controls viral replication is administered concurrently with or prior to the administration of the agent that reduces viral Ags, the immune enhancer, and the immune stimulant.

37. The method of claim 35, wherein an agent that controls viral replication is administered concurrently with the administration of the agent that reduces viral Ags, the immune enhancer, and the immune stimulant.

38. The method of claim 35, wherein an agent that controls viral replication is administered prior to the administration of the agent that reduces viral Ags, the immune enhancer, and the immune stimulant.

39. The method of claim 16, wherein the agent that reduces viral Ags is administered, administration of the immune enhancer is started subsequent to the start of agent that reduces viral Ags administration, and administration of the immune stimulant is started subsequent to the start of to the immune enhancer.

40. (canceled)

41. A method for treating hepatitis B in a human, comprising first administering to the human an agent that reduces viral Ags and then administering an agent that improves the immune response to the hepatitis B virus.

42-43. (canceled)

44. The method of claim 41, further comprising administering an agent that controls viral replication.

45-46. (canceled)
